(19) United States
(12) Patent Application Publication
(10) Pub. No.: US 2019/0224193 A1
(43) Pub. Date: Jul. 25, 2019

(54) Compositions of Natural Extracts and Use Thereof in Methods for Preventing or Treating Diseases

(71) Applicant: Christopher Brian REID, (US)

(72) Inventors: Christopher Brian REID, Los Angeles, CA (US); Que T. Collins, Santa Monica, CA (US)

(21) Appl. No.: 16/134,723

(22) Filed: Sep. 18, 2018

Related U.S. Application Data
(63) Continuation of application No. PCT/US2017/033234, filed on May 18, 2017.
Application No. PCT/US2017/033234 is a continuation of application No. 16/039,134, filed on Jul. 18, 2018.
Application No. 16/039,134 is a division of application No. 15/241,308, filed on Aug. 19, 2016, now abandoned.
Application No. 15/241,308 is a division of application No. 13/815,664, filed on Mar. 14, 2013, now abandoned.
(60) Provisional application No. 62/390,081, filed on Mar. 18, 2016.
(60) Provisional application No. 62/390,438, filed on Mar. 29, 2016.

(51) Int. Cl. A61K 31/496; A61K 45/06; A61K 31/327; A61K 31/194; A61K 38/18; A61K 36/00; A61K 31/122; A61K 38/06; A61K 31/4164; A61K 31/375; A61K 31/355; A61K 31/12

(52) U.S. Cl. CPC A61K 31/496; A61K 45/06; A61K 31/327; A61K 31/194; A61K 38/1816; A61K 36/00; Y02A 50/389; A61K 38/063; A61K 31/4164; A61K 31/375; A61K 31/355; A61K 31/12; A61K 31/122

(57) ABSTRACT

A composition for reducing miR-3120 expression in a cell or tissue of a subject comprises at least one oil or extract selected from a group comprising an orange, frankincense and cannabis oil or extract. A method for reducing miR-3120 expression in a cell or tissue of a subject comprises administering to the subject a composition comprising at least one oil or extract selected from the group comprising an orange, frankincense and cannabis oil or extract.

### CROSS REFERENCE TO RELATED APPLICATIONS

This application is a continuation of PCT/US2017/033234, filed May 18, 2017, which claims priority to U.S. Provisional Patent App. No. 62/390,081, filed Mar. 18, 2016, and U.S. Provisional Patent App. No. 62/390,438, filed Mar. 29, 2016; and is a continuation of U.S. patent application Ser. No. 16/039,134, filed Jul. 18, 2018, which is a divisional of Ser. No. 15/241,308 filed Aug. 19, 2016, which is a divisional of U.S. patent application Ser. No. 13/815,664, filed Mar. 14, 2013, the disclosures of each of which are hereby incorporated by reference herein in their entireties.

### FIELD OF THE INVENTION

The invention relates to compositions and methods for treating or preventing diseases using natural extracts, their derivatives, or their components.

### BACKGROUND OF THE INVENTION

In industrialized countries, cancer and metabolic diseases represent major causes of morbidity and mortality. Recent studies have demonstrated numerous molecular targets for the prevention and/or treatment of cancer and psoriasis. One such target is microRNA-21 (miR21). As most of the targets of miR-21 are tumor suppressors, miR-21 is associated with a wide variety of cancers including that of breast, ovaries, cervix, colon, lung, liver, brain, esophagus, prostate, pancreas, and thyroid. Also in a genome-wide screen, it was found deregulation of microRNA expression in psoriasis skin. miR-21 is one of the microRNAs significantly up-regulated in psoriasis skin lesions. A 2014 meta-analysis of 36 studies evaluated circulating miR-21 as a biomarker of various carcinomas, finding it has potential as a tool for early diagnosis. miR-21 is one of the most frequently upregulated miRNAs in solid tumours, and its high levels were first described in B cell lymphomas. Overall, miR-21 is considered to be a typical ‘onco-miR’, which acts by inhibiting the expression of phosphatases, which limit the activity of signalling pathways such as AKT and MAPK. miR-21 can be transcriptionally activated by NF-κB and downregulate phosphatases PDCD4 and PTEN.

Synthetic drugs for treatment of a diseases associated with miR-21 expression are known in the prior art. But synthetic drug development is slow and costly, often running into the billions of dollars. While the benefits of various abundant and inexpensive natural products, including natural oils and extracts, have been postulated and speculated upon for centuries, there has been very little success in achieving national approval (e.g. FDA approval) of natural compounds so that they can be used to benefit animals and patients. As many natural products are often complex and heterogenous, appropriate technologies for rapidly and efficiently predicting efficacy and toxicity of such natural compounds has been sorely lacking. Thus, there has been a long held and still unmet need to apply advanced and costly methods, such as gene array, to determine such efficacy and toxicity and teach appropriate health, veterinary, and medical uses for an orange, frankincense, cannabis, etc. products in a cost effective and efficient manner.

### SUMMARY OF THE INVENTION

Virtually all patients could benefit from novel, efficacious drug compositions. The present invention provides novel orange, frankincense, cannabis and other natural oil and extract related compositions and inexpensive drug compositions for preventing and/or treating various conditions, diseases, and maladies especially metabolic diseases and disorders, cancer, psoriasis and improving health and well-being in general. The present invention also provides corresponding methods for producing such compositions and methods of preventing a condition, disorder or disease and treating a condition, disorder or disease in an animal or human, wherein the compositions, manufacture, or products of the present invention are provided to said animal or human.

In a first aspect, according to the present invention, the problem of the prior art is solved by a composition for reducing miR-21 expression in a cell or tissue of a subject comprising deuterium depleted water (DDW) and/or at least one oil or extract selected from the group comprising orange, frankincense, cannabis or other natural oil or extract or at least one substance or compound isolated from said at least one oil or extract. The composition may further comprise at least one carrier and/or at least one excipient, wherein said composition is used for treatment of a disease associated with miR-21 expression.

In a second aspect, this invention relates to a method for reducing mir-21 and other oncogene expression in a cell or tissue of a subject comprising administering to the subject a composition comprising deuterium depleted water (DDW) and/or at least one oil or extract selected from the group comprising orange, frankincense, cannabis or other natural oil or extract or at least one substance or compound isolated from said at least one oil or extract.

The composition may further comprise at least one carrier and/or at least one excipient, wherein the method is for treatment of a disease associated with mir-21 and/or other oncogene expression.

The method of suppressing miR21 can be seen as a method of treating and/or preventing cancer, psoriasis, and or any other disease associated with miR-21 and/or other oncogene expression.

The method may be accomplished by administering to the subject an appropriate amount of composition comprising deuterium depleted water (DDW) and/or at least one oil or extract selected from the group comprising orange, frankincense, cannabis or other natural oil or extract or at least one substance or compound isolated from said at least one oil or extract, which showing potent miR21 suppression in vitro and in vivo or at least one substance or compound with miR21 suppressive activity that are isolated from such extracts. At least one extract selected from the group comprising orange, frankincense, cannabis or other natural oil or extract or at least one substance or compound isolated from said at least one extract and other natural oils and/or extracts that we tested at 1:1000 and 1:5000 dilutions reduced miR21 expression between 17 fold and 3 fold in cultured cell lines. The extracts covered by this disclosure include ones chemically-modified in numerous ways, including but not limited to, for example, by exposure to acids, conjugation to other compounds, or incorporation into lipid carriers. Likewise the extracts or extracted responsible compounds may be combined with other agents in various forms and compositions and functional foods (see below).

Also the invention provides for a method of treating an animal or human patient comprising at least one extract selected from the group comprising orange, frankincense, cannabis, or other natural oil or extract or at least one substance or compound isolated from said at least one extract and other natural oils and/or extracts.

In some embodiments, the methods of the present invention provide for multiple at least one extract selected from the group comprising orange, frankincense, cannabis or other natural oil or extract or at least one substance or compound isolated from said at least one extract and other natural oils and/or extracts are used in combination so as to achieve additive or synergistic effects.

In a third aspect, this invention relates to a method of producing iPS cells that are less prone to malignant transformation due to suppression of miR-21 and other oncogene expression in said cells, wherein said suppression of miR-21 and other oncogene expression comprises cultivating iPS cells with deuterium depleted water and/or at least one oil or extract selected from the group comprising an orange, frankincense, cannabis or other natural oil or extract or at least one substance or compound isolated from said at least one oil or extract.

In addition to activating cell surface receptors, at least one extract selected from the group comprising orange, frankincense, cannabis or other natural oil or extract or at least one substance or compound isolated from said at least one extract and other natural oils and/or extracts are capable of readily penetrating cell membranes and exerting potent effects, including but not limited to, gene expression and epigenetic reprogramming (Jaenisch, 2003; Weinhold, 2006) of cells. Accordingly, cannabis and other natural oils and extracts are taught, in part, herein for reprogramming or converting cells from a first phenotype to a second phenotype e.g. a cancerous state to a non-cancerous state, or a less potent state to a more potent state. Some natural oils and extracts, may for example, be utilized to reprogram somatic, differentiated, or other non-pluripotent cells to a pluripotent state. Some natural oils and extracts, may for example, be utilized to reprogram somatic, differentiated, or other non-totipotent cells to a totipotent state with or without development of embryonic morphology, e.g. blastocysts. Accordingly, the present invention teaches, in part, methods and compositions for cell reprogramming. The appropriate at least one extract selected from the group comprising orange, frankincense, cannabis or other natural oil or extract or at least one substance or compound isolated from said at least one extract and other natural oils and/or extracts of the present invention may be used alone or combined with other nucleic acid(s), protein(s), or small molecule(s) known to those skilled in the art, or demonstrated in the future, to produce cell reprogramming. At least one extract selected from the group comprising orange, frankincense, cannabis or other natural oil or extract or at least one substance or compound isolated from said at least one extract and other natural oils and/or extracts of the present invention may, for example, be used to produce self-renewal in cells not displaying self-renewal. at least one extract selected from the group comprising orange, frankincense, cannabis or other natural oil or extract or at least one substance or compound isolated from said at least one extract and other natural oils and/or extracts of the present invention may be used to block or inhibit aberrant self-renewal, abnormal cell proliferation and other characteristics associated with cancerous, neoplastic or dysplastic cells.

Likewise, animals and patients suffering from various disorders and diseases can benefit from the effects exerted by at least one extract selected from the group comprising orange, frankincense, cannabis or other natural oil or extract or at least one substance or compound isolated from said at least one extract and other natural oils and/or extracts. Patients inflicted with various clustering chronic diseases are typically treated with multiple drugs having distinct mechanisms of action. Accordingly, patients with multiple conditions suffer from cumulative side effects of multiple drugs, as well as adverse effects drug-drug interactions. Various, highly-purified, at least one extract selected from the group comprising orange, frankincense, cannabis or other natural oil or extract or at least one substance or compound isolated from said at least one extract and other natural oils and/or extracts of the present invention, including many that are commercially available, are suitable for use, alone or in combination, to replace an approved drug or approved drugs during treatment of an animal or human patient, thereby reducing polypharmacy and adverse drug-drug interactions. Additionally, at least one extract selected from the group comprising orange, frankincense, cannabis or other natural oil or extract or at least one substance or compound isolated from said at least one extract of the invention may be used in combination with one or more approved drugs to provide benefit to an animal or human patient.

Accordingly, the present invention teaches, in part, compositions comprising at least one extract selected from the group comprising orange, frankincense, cannabis or other natural oil or extract or at least one substance or compound isolated from said at least one extract and other natural oils and/or extracts. Examples of disorders prevented or ameliorated by administration of the compositions of this invention include but are not limited to inflammatory diseases that may be, oncological, genetic, ischemic, infectious, neurological, hematological, ophthalmological, rheumatoid, orthopedic, neurological, hematological, kidney, vascular, dermatological, gynecological, obstetric, otherwise physical, psychological, or psychiatric. The present invention further relates to a method of identifying agents, compounds or drugs useful in preventing or treating CDCP related diseases and conditions as well as other disorders, diseases and conditions treatable or preventable by the same agents, compounds or drugs.

The present invention teaches an efficient method and process for screening and determining appropriate uses, as well as caveats (e.g. potential toxicities) related to the use of natural products, including at least one extract selected from the group comprising orange, frankincense, cannabis or other natural oil or extract or at least one substance or compound isolated from said at least one extract and other natural oils and/or extracts, wherein such appropriate uses and caveats are defined according to the patterns of gene expression modulation described herein.

In some embodiments, the compositions, manufacture, products, processes, methods, and/or methods of use, prevention and treatment of the present invention comprise at least one extract selected from the group comprising orange, frankincense, cannabis or other natural oil or extract or at least one substance or compound isolated from said at least one extract and other natural oils and/or extracts suitable for use in the methods of the present invention will be ones screened for potential toxicity utilizing toxicity predictive platforms such as the Cellular Dynamics iCell and MyCell predictive toxicity testing platform(s), the BioMAP® Predictive Tox Panel, or similar platform for assessing potential toxicity.

In some embodiments, the compositions, manufacture, products, processes, methods, and/or methods of use, prevention and treatment of the present invention are screened for desired activity and efficacy using cell-based assay systems such as are available from Affymetrix, Illumina, Qiagen, Genecopoeia, Thermofisher BioMap Systems, BioMap Diversity Plus, BioMAP Oncology Systems, and BioMap EC50 ELECT, as well as the Cellular Dynamics iCell and MyCell efficacy and predictive toxicity testing platforms, and wherein said compositions, manufacture, products, processes, methods, and/or methods of use, prevention and treatment modulate gene expression as described herein.

In some preferred embodiments, the compositions, manufacture, products, processes, methods, and/or methods of use, prevention and treatment of the present invention combine two or more extracts wherein a first extract counteracts, to some extent, at least one toxic effect of a second extract.

In some preferred embodiments, the compositions, manufacture, products, processes, methods, and/or methods of use, prevention and treatment of the present invention combine two or more extracts in ratios of about 1:1 to about 1:100 or 1:1 to about 1:1000.

In some preferred embodiments, the compositions, manufacture, products, processes, methods, and/or methods of use, prevention and treatment of the present invention comprise said extracts diluted at about 1:10 to about 1:1,000 or 1:100 to about 1:10,000.

In some preferred embodiments, the compositions, manufacture, products, processes, methods, and/or methods of use, prevention and treatment of the present invention comprise said extracts diluted at about 1:100 to about 1:10,000 or 1:1,000 to about 1:100,000.

In some preferred embodiments, the compositions, manufacture, products, processes, methods, and/or methods of use, prevention and treatment of the present invention comprise two or more said extracts.

It has been taught that virtually all medications have the potential to cause significant side effects. It is also generally understood, accepted and taught that the various chronic diseases that cluster in patients CDCP as well as other disorders diseases and conditions (ODDC) have distinct mechanisms of disease and are therefore appropriately treated or prevented by providing multiple drugs with distinct mechanisms of action. These teachings have led to the phenomenon known as a “polypharmacy”. Accordingly, patients requiring treatment for multiple conditions frequently suffer from the cumulative side effects of multiple drugs, as well as adverse drug-drug interactions related to polypharmacy. Polypharmacy is especially problematic in treatment of the elderly Najjar et al., (2007) concluded that, “polypharmacy continues to increase and is a known risk factor for important morbidity and mortality.”

Often, polypharmacy results in reduced efficacy of one or more drugs. In addition, a drug provided to treat one chronic condition may worsen another. For example, many antidiabetes medications produce weight gain, thereby scuttling efforts and counteracting medications aimed at reducing patient obesity. Thus, it is also medically desirable to provide single agents, compounds or drugs as monotherapies capable of preventing or treating multiple conditions, thereby avoiding polypharmacy. Likewise, combination therapies involving a reduced number of agents, compounds, or drugs are also desirable as still reducing the level and risks of polypharmacy.

Therefore, the present invention provides compositions, manufacture, products, processes, methods, and/or methods of use, prevention and treatment for reducing or eliminating polypharmacy, as well. Therefore, the present invention provides compositions, manufacture, products, processes, methods, and/or methods of use, prevention and treatment of diseases or disorders. Likewise, an important feature of the present invention is the combination of naturally-occurring compounds named herein with other naturally-occurring compounds or with synthetic compounds. Such combinations may generally produce reduced side-effects as compared to combinations involving multiple pharmaceutical drugs (polypharmacy).

Embodiments, agents, compounds or drugs of the present invention may replace an equal or larger number of approved drugs in treating a patient.

ODDC comprise all conditions and diseases known to those skilled in the medical art, as the compounds and compositions described herein relate to a common pathway of cellular injury, cellular dysfunction, cellular derangement, and inflammation. For example, the present invention also provides compositions for preventing and treating parasitic diseases.

The present invention relates to various agents, compounds and drugs named herein. The agents, compounds and drugs of the present invention comprise i. at least one extract selected from the group comprising orange, frankincense, cannabis or other natural oil or extract or at least one substance or compound isolated from said at least one extract and other natural oils and/or extracts, ii. FDA approved drugs and iii. non-FDA approved drugs. The present invention teaches the use of at least one extract selected from the group comprising orange, frankincense, cannabis or other natural oil or extract or at least one substance or compound isolated from said at least one extract and other natural oils and/or extracts, at least one agent, compound, or drug of the present invention, alone or in combination with each other and in combination with agents, compounds or drugs, to treat or prevent a large variety of disorders and conditions for which the use of said, at least one extract selected from the group comprising orange, frankincense, cannabis or other natural oil or extract or at least one substance or compound isolated from said at least one extract and other oils and/or extracts, said agents or, in particular, said combinations has not been previously described or has been dismissed by prior teachings.

Similarly, the present invention teaches compositions, manufacture, products, processes, methods, and/or methods of use, prevention and treatment comprising agents, compounds or drugs (including orange, frankincense and cannabis oils and/or extracts) for uses and delivery that have not been previously described or that have been dismissed by prior teachings.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

### Additional Definitions

By “effective amount” is meant the amount of a required to ameliorate the symptoms of a disease relative to an untreated patient. The effective amount of active at least one extract selected from the group comprising orange, frankincense, cannabis or other natural oil or extract or at least one substance or compound isolated from said at least one extract and other natural oils and/or extracts, at least one agent, compound used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

By “ameliorate” is meant decrease, suppress, attenuate, diminish, arrest, or stabilize the development or progression of a disease. As used herein, the meaning of “ameliorate” includes lessening an effect, or reducing damage, or minimizing the effect or impact of an action, activity, or function, and includes, for example lessening the deleterious effects of a disease or condition. By “agent” is meant any small molecule chemical compound, antibody, nucleic acid molecule, or polypeptide, or fragments thereof.

By “modulation” is meant a change (increase or decrease) or alteration in the expression or activity levels or activity of a gene or polypeptide as detected by standard art known methods such as those described herein. As used herein, an alteration includes a 5% change in expression or activity levels, preferably a 25% change, more preferably a 40% change, and most preferably a 50% or greater change in expression or activity levels.”

By “reduces” is meant a negative modulation of at least 5%, 25%, 50%, 75%, or 100%.

By “analog” is meant a molecule that is not identical, but has analogous functional or structural features. For example, a polypeptide analog retains the biological activity of a corresponding naturally-occurring polypeptide, while potentially having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid. Likewise, analog herein refers to those compounds structurally related to the compound, agent or drug in question and which retains characteristic biological properties of the compound, agent or drug.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

CDCP refers to the large number of serious chronic diseases such as cardiovascular disease, diabetes, obesity, hyperlipidemia, PCOS and hypertension that have been observed to cluster in patients, and includes disorders comprising metabolic syndrome or syndrome X. Such disorders are common in industrialized countries.

In non-industrialized countries, infectious and parasitic diseases similarly threaten not only the lives of individuals, but the economic viability of families, communities, and societies as a whole. For example, protozoal illnesses continue to account for significant morbidity and mortality, especially in the tropical world. Malaria, which is caused by the protozoa, plasmodium falciparum, plasmodium vivax, plasmodium ovale, is endemic in to 90 countries in Africa, Asia, Oceania, South America, and the Caribbean, infects approximately 300-500 million people and kills 2.5 million people every year. Most of whom report lack of access or funds to purchase expensive artemisinin-based combination therapies. In one aspect, the invention relates to the use of one or more of an orange, frankincense, or other natural oil or extract alone or in combination with approved drugs or other compounds and agents listed herein.

For example, the present invention covers the combination of an essential oil listed herein with one or more of a ginger extract, sesquiterpene, zerumbone, monoterpene, an artemisin class compound, a metronidazole class compound, an itraconazole class compound, a ciprofloxacin class compound, an approved drug, and a drug approved for treating a protozoal infection.

ODDC comprise all conditions and diseases known to those skilled in the medical art other than chronic diseases that cluster in patients (CDCP).

As named and used herein, an agent, compound or drug of the present invention refers to the agent, compound or drug its analogs, and its derivatives including those derivatives described herein (e.g. glutathione conjugates, n-acetylcysteine conjugates, biotinylated derivatives, fluorinated derivatives, and derivatives having an NO donor moiety).

“An orange, frankincense, cannabis or other natural oil or extract or at least one substance or compound isolated from said at least one extract and other natural oils and/or extracts” refers to orange, frankincense, cannabis or other natural oil or extract or at least one substance or compound isolated from said at least one extract and other natural oils and/or extracts, a derivative of orange, frankincense, cannabis or other natural oil or extract or at least one substance or compound isolated from said at least one extract and other natural oils and/or extracts, an added sesquiterpene, a derivative of an added sesquiterpene, or other practicable agent, compound named herein.

### BRIEF DESCRIPTION OF THE DRAWINGS

FIG. 1 is a general cross-section view of a right sided version of “L”-shaped solid buccal or sublingual dosage form according to one of the preferred embodiments of the present invention.

FIG. 2 is a side cross-section view of a left sided version of “L”-shaped solid buccal or sublingual dosage form according to another preferred embodiment of the present invention.

FIG. 3 is a front view of perforated “L”-shaped solid buccal or sublingual dosage form according to another preferred embodiment of the present invention.

FIG. 4 is a general right view of perforated “L”-shaped solid buccal or sublingual dosage form from FIG. 3.

FIG. 5 is a front view of fenestrated or dimpled “T”-shaped solid buccal or sublingual dosage form according to yet another preferred embodiment of the present invention.

FIG. 6 is a right view of fenestrated or dimpled “T”-shaped solid buccal or sublingual dosage form from FIG. 5.

FIG. 7 is a is a front view of fenestrated or dimpled flattened solid buccal or sublingual dosage form according to yet another preferred embodiment of the present invention.

FIGS. 8A-8D are tables and diagrams illustrated NCCIT apoptotic RNA degradation after 24 hours incubation with various combinations of natural oils.

FIG. 9 is a heat map showing gene expression in cannabis extract treated cells relative to control.

FIGS. 10-17 are illustrations of biological activity of dilute natural oil extracts in cultured NCCIT cancer and 3T3 cells.

FIG. 10 depict an illustration of biological activity of dilute (1:1000 and 1:100) clove extract in cultured NCCIT cancer after 48 hours (10× and 40× magnification) relative to control.

FIG. 11 depict an illustration of biological activity of dilute (1:1000 and 1:100) thyme extract in cultured NCCIT cancer after 48 hours (10× and 40× magnification) relative to control.

FIG. 12 depict an illustration of biological activity of dilute (1:1000 and 1:100) lemon extract in cultured NCCIT cancer after 48 hours (10× and 40× magnification) relative to control.

FIG. 13 depict an illustration of biological activity of dilute (1:5000) clove extract and dilute (1:5000) frankincense extract in cultured 3T3 cells after 1 week (10× and 40× magnification) relative to control.

FIG. 14 depict an illustration of biological activity of dilute (1:5000) ginger extract and dilute (1:5000) orange extract in cultured 3T3 cells after 1 week (10× and 40× magnification) relative to control.

FIG. 15 depict an illustration of biological activity of dilute (1:5000) lemon grass extract and dilute (1:5000) lavender extract in cultured 3T3 cells after 1 week (10× and 40× magnification) relative to control.

FIG. 16 depict an illustration of biological activity of dilute (1:5000) clove extract, dilute (1:5000) frankincense extract, dilute (1:5000) ginger extract, dilute (1:5000) orange extract, dilute (1:5000) lemon grass extract, dilute (1:10000) lavender extract in cultured 3T3 cells after 2 weeks (10× magnification) relative to control.

FIG. 17 depict an illustration of biological activity of dilute (1:5000) clove extract, dilute (1:5000) frankincense extract, dilute (1:5000) ginger extract, dilute (1:5000) orange extract, dilute (1:5000) lemon grass extract, dilute (1:10000) lavender extract in cultured 3T3 cells after 2 weeks (20× magnification) relative to control.

### DETAILED DESCRIPTION OF THE INVENTION

The current invention incorporates the teachings of the herein cited and/or referenced publications and patent applications in their entireties. US20140271923 and WO2008/150814 are incorporated herein in their entireties.

Before describing the present invention in detail, it is to be understood that this invention is not limited to the particular embodiments, techniques, active agents, and the like as such may vary. It is also to be understood that the terminology used herein is for describing particular embodiments only, and is not intended to be limiting.

In a first aspect, the present invention relates to a composition for reducing mir-21 and/or other oncogene expression in a cell or tissue of a subject comprising deuterium depleted water (DDW) and/or at least one oil or extract selected from the group comprising orange, frankincense, cannabis or other natural oil or extract or at least one substance or compound isolated from said at least one oil or extract and at least one carrier and/or at least one excipient, wherein said composition is used for treatment of a disease associated with miR-21 expression.

In a second aspect the present invention relates to a method for reducing mir-21 and/or other oncogene expression in a cell or tissue of a subject comprising administering to the subject a composition comprising deuterium depleted water (DDW) and/or at least one oil or extract selected from the group comprising orange, frankincense, cannabis or other natural oil or extract or at least one substance or compound isolated from said at least one oil or extract and at least one carrier and/or at least one excipient, wherein the method is for treatment of a disease associated with mir-21 and/or other oncogene expression.

The compositions, methods, and/or methods of use, manufacture, products, processes, prevention and treatment of the present invention can be mixed with suitable pharmaceutical carriers (vehicles) or excipients known to the art (e.g. Kumar et al., 1996; Akers et al., 2002; Strickley et al., 2004; Jacob et al., 2010; Siddiqui et al., 2010; Pilcer et al., 2010). Examples include water-soluble organic solvents, non-ionic surfactants, water-insoluble lipids, organic liquids/semi-solids, cyclodextrins and phospholipids. They may also include gelatin, lactic acid, stearic acid or salts or complexes thereof, starch, milk, sugar, certain types of clay, including magnesium or calcium stearate, talc, oils, gums, vegetable fats, lipids, or and glycols.

Examples of suitable pharmaceutical vehicles are also described in Remington's Pharmaceutical Sciences, Alfonso R. Gennaro ed., Mack Publishing Co. Easton, Pa., 19th ed., 1995, pp. 1447 to 1676, incorporated herein by reference.

In some embodiments, said composition of the present invention is administered in a dose of from about 0.01 mg/kg of the individual's body weight to about 500 mg/kg of the individual's body weight.

Other natural oils and extracts suitable for use in the compositions and methods of the present invention include commercially available ones, such as commercially available mango oil, etc.

Such oil extracts may be used alone or in combination, and/or in combination with approved drugs and/or other agents and compounds of the present invention.

Natural oils and extracts suitable for use in the methods of the present invention include, but are not limited to, commercially available natural oils and extracts in the classes represented by the following exemplars: Agarwood; Agarwood; Almond, Aloe Vera; Bitter; Amber Oil, Avocado, Fossilized; Amber Oil, Fossilized; Ambrette Seed Fine; Ambrette Seed; Amyris; Angelica Root; Angelica Seed; arborvitae; Armoise (Mugwort); Balsam of Peru Oil; Balsam of Peru Resin; Basil, Sweet ct Linalool; Basil, Sweet ct Linalool; Basil, Sweet ct Methyl Chavicol; Beeswax Absolute; Bergamot; Bergamot k; Bergamot; Bergamot; Black Cumin; Black Currant; Caraway; Cardamom; Carnation Absolute; Carnation Extract; Carrot Seed; Cassie Absolute; Cedarwood, Atlas; Cedarwood, Himalayan; Cedarwood, Texas; Cedarwood, Virginia; Celery Seed; Chamomile, Blue; Chamomile, Roman; Champaca; Cilantro; Cinnamon; Cinnamon Bark; Cistus Traditional; Citronella; Citronella Wild; Clary Sage Absolute; Clary Sage, Bulgaria; Clary Sage, Russia; Clary Sage, USA; Clove Bud; Clove; Cocao Absolute; Coconut; Coffee Bean; Coffee Bean Oil; Cognac, Green; Coriander Seed; Coriander Seed; Cucumber Hydrosol; Cumin Seed; Cypress Leaf; Cypress, Blue; Davana; Eucalyptus, Blue Gum; Eucalyptus, Blue Mallee; Eucalyptus, Lemon; Eucalyptus, Narrow Leaf; Eucalyptus, Narrow Leaf; Fennel, Sweet; Fennel, Sweet; Fenugreek; Fir Needle; Fir, Balsam; Fir, Balsam Absolute; Fir, Balsam Absolute; Fir, Douglas; Fir, Silver; Frankincense; Frankincense, Somalia; Frankincense Frereana; Frankincense, Oman; Frankincense, Oman Rare; Frankincense, Somalia; Galbanum; Geranium Absolute; Geranium, Egypt; Geranium, Rose; Geranium, South Africa; Ginger; Ginger; Ginger; Ginger Lily; Ginger, Fresh; Goji; Grapefruit, Pink; Grapefruit, Ruby Red; Grapefruit, White; Hay Absolute; Helichrysum, Albania; Helichrysum, Croatia; Hemp; Hyssop Decumbens; Immortelle Absolute; Jasmine Absolute, Egypt; Jasmine Absolute, Egypt; Jasmine Absolute; Jasmine Absolute; Jasmine; Jasmine Concrete; Jasmine Extract; Jasmine Sambac Absolute; Jasmine Sambac Absolute; Juniper Berry; Juniper Berry; Juniper Leaf/Berry, Nepal; Juniper Leaf/Branch; Kava Kava; Kava Kava; Labdanum Absolute, Clear; Laurel Leaf; Lavandin, Grosso; Lavender High Elevation; Lavender Wild; Lavender Absolute; Lavender Hydrosol; Lavender, Bulgaria; Lavender, France; Lavender, Maillette; Lemon; Lemon Tea Tree; Lemongrass; Lemongrass Wild; Lime Distilled; Lime Expressed; Lime Essence Oil; Lime, Distilled; Liquidambar (Styrax; Lotus Absolute, Pink; Lotus Absolute, White; Availability; Mandarin, Green; Mandarin, Red; Mandarin, Yellow; Mango’ Marjoram; Melaleuca; Melissa; Myrrh; Myrrh, Somalia; Myrrh, Somalia; Myrtle, Green; Nagarmotha (Cypriol; Neroli Extra; Neroli Extra; Neroli, Egypt; Neroli, Egypt; Neroli, France; Neroli, France; Neroli, Morocco; Niaouli; Oakmoss Absolute; Orange Wild; Orange Blossom Absolute; Orange Blossom Absolute Fine; Orange Blossom Extract; Orange Essence Oil; Orange, Bitter Green; Orange, Bitter Red; Orange, Blood; Orange, Wild; Orange, Sweet; Oregano, Turkey; Orris Butter (15 irones; Osmanthus Absolute; Palmarosa, Nepal Wild; Palmarosa, Sri Lanka; Palo Santo; Patchouli Double Distilled; Patchouli; Patchouli, Dark; Patchouli, Light; Patchouli, Sri Lanka; Pepper, Black; Peppercorn, Pink; Peppermint, Chocolate; Peppermint, France; Peppermint; Peppermint, USA; Petitgrain Absolute; Petitgrain Bigarade; Petitgrain sur Fleurs; Petitgrain, Mandarin; Petitgrain, Mandarin; Piper, aduncum; Piper, malacophyllum; Pomegranate Seed; Ravensara Wild; Rhododendron Leaf; Rosalina; Rose Absolute, Bulgaria; Rose Absolute, Bulgaria; Rose Absolute, Egypt; Rose Absolute, Egypt; Rose Absolute, Morocco; Rose Absolute, Morocco; Rose de Mai Absolute; Rose de Mai Concrete; Rose de Mai Extract; Rose Hip Seed; Rose Otto, Bulgaria; Rose Otto, Turkey; Rose Otto, White; Rosemary Antioxidant; Rosemary ct Cineole; Rosemary ct Cineole; Rosemary ct Verbenone; Sage; Sandalwood Rare; Sandalwood Absolute, New Caledonia; Sandalwood, Australian Premium; Sandalwood, New Caledonia; Sandalwood, New Caledonia Extra; Sandalwood, Royal Hawaiian; Sea Buckthorn; Seaweed Absolute; Spearmint; Spearmint, USA; Spikenard; Spikenard, Green Wild; Spruce, Black; St. John's Wort; Tagetes; Tamanu (Foraha Oil; Tangerine Murcott; Tansy, Blue; Tea Tree; Thyme ct Linalool; Tobacco Absolute; Tonka Bean Absolute; Tonka Bean Absolute 20; Tuberose Absolute; Tuberose Extract; Turmeric; Turmeric; Vanilla Absolute;

Vanilla Bourbon; Vanilla Bourbon; Vanilla Bourbon; Verbena; Vetiver Double Distilled; Vetiver, El Salvador; Vernonia, polyanthes; Vetiver, Haiti; Vetiver, Sri Lanka; Violet Leaf Absolute; Violet Leaf Absolute; Virola, surinamensis; Vitamin E Oil; White Sage; Wintergreen Wild; Yarrow, Blue; Ylang Ylang Absolute; Ylang Ylang Complete, Comoros; Ylang Ylang Extra; Ylang Ylang I; Ylang Ylang II; Ylang Ylang III; Ylang Ylang, Fine; and Yuzu. etc. Such oil extracts may be used alone or in combination, and/or in combination with approved drugs and/or other agents and compounds of the present invention.

In some embodiments, the compositions, manufacture, products, processes, methods, and/or methods of use, prevention and treatment of the present invention comprise a compound, agent or drug extracted from cloves, black pepper, red chili, cinnamon, and ginger.

Particularly valuable are platforms offering analysis of alternative splicing changes.

In some preferred embodiments, the compositions, manufacture, products, processes, methods, and/or methods of use, prevention and treatment of the present invention combine said extracts which act synergistically.

In one preferred embodiment of the present invention the subject is a mammal.

In one embodiment of the present invention the subject is a non-mammal animal.

Such compositions, manufacture, products, processes, methods, and/or methods of use, prevention and treatment can be administered to a subject animal such as mammals (rat, mouse, domestic animals or human) via various routes.

In one preferred embodiment of the present invention the disease is selected from the group comprising cancer and psoriasis.

Examples of cancer related diseases and conditions include prostate cancer, breast cancer, lung cancer, colorectal cancer, bladder cancer, uterine cancer, ovarian cancer, lymphoma, skin cancer, stomach cancer, liver cancer, wasting diseases, and other cancers.

In some embodiments, the compositions of the present invention are useful for preventing or treating diseases and conditions related to the Prostate such as prostate enlargement and prostate cancer.

In some embodiments, the present invention provides means or adjunctive means of treating cancer (e.g. multiple myeloma, colorectal cancer, leukemic cells, Acute lymphoblastic leukemia, Acute myeloid leukemia, Adrenocortical carcinoma, AIDS-related cancers, AIDS-related lymphoma, Anal cancer, Appendix cancer, Astrocytoma, childhood cerebellar or cerebral, Basal cell carcinoma, Bile duct cancer, extrahepatic, Bladder cancer, Bone cancer, Osteosarcoma/Malignant fibrous histiocytoma, Brainstem glioma, Brain tumor, Brain tumor, cerebellar astrocytoma, Brain tumor, cerebral astrocytoma/malignant glioma, Brain tumor, ependymoma, Brain tumor, medulloblastoma, Brain tumor, supratentorial primitive neuroectodermal tumors, Brain tumor, visual pathway and hypothalamic glioma, Breast cancer, Bronchial adenomas/carcinoids, Burkitt lymphoma, Carcinoid tumor, childhood, Carcinoid tumor, gastrointestinal, Carcinoma of unknown primary, Central nervous system lymphoma, primary, Cerebellar astrocytoma, childhood, Cerebral astrocytoma/Malignant glioma, childhood, Cervical cancer, Childhood cancers, Chronic lymphocytic leukemia, Chronic myelogenous leukemia, Chronic myeloproliferative disorders, Colon Cancer, Cutaneous T-cell lymphoma, Desmoplastic small round cell tumor, Endometrial cancer, Ependymoma, Esophageal cancer, Ewing's sarcoma in the Ewing family of tumors, Extracranial germ cell tumor, Childhood, Extragonadal Germ cell tumor, Extrahepatic bile duct cancer, Eye Cancer, Intraocular melanoma, Eye Cancer, Retinoblastoma, Gallbladder cancer, Gastric (Stomach) Cancer, Gastric (Stomach) Cancer, Childhood, Gastrointestinal Carcinoid Tumor, Gastrointestinal Stromal Tumor (GIST), Germ Cell Tumor, Extracranial, Childhood, Germ Cell Tumor, Extragonadal, Germ Cell Tumor, Ovarian, Gestational Trophoblastic Tumor, Glioma, Adult, Glioma, Childhood Brain Stem, Glioma, Childhood Cerebral Astrocytoma, Glioma, Childhood Visual Pathway and Hypothalamic, Gastric Carcinoid, Hairy cell leukemia, Head and neck cancer, Heart cancer, Hepatocellular (liver) cancer, Hodgkin lymphoma, Hypopharyngeal cancer, Hypothalamic and visual pathway glioma, childhood, Intraocular Melanoma, Islet Cell Carcinoma (Endocrine Pancreas), Kaposi sarcoma, Kidney cancer (renal cell cancer), Laryngeal Cancer, Leukemias, Leukemia, acute lymphoblastic (also called acute lymphocytic leukemia), Leukemia, acute myeloid (also called acute myelogenous leukemia), Leukemia, chronic lymphocytic (also called chronic lymphocytic leukemia), Leukemia, chronic myelogenous (also called chronic myeloid leukemia), Leukemia, hairy cell, Lip and Oral Cavity Cancer, Liver Cancer (Primary), Lung Cancer, Non-Small Cell, Lung Cancer, Small Cell, Lymphomas, Lymphoma, AIDS-related, Lymphoma, Burkitt, Lymphoma, cutaneous T-Cell, Lymphoma, Hodgkin, Lymphomas, Non-Hodgkin (an old classification of all lymphomas except Hodgkin's), Lymphoma, Primary Central Nervous System, Macroglobulinemia, Waldenstrom, Malignant Fibrous Histiocytoma of Bone/Osteosarcoma, Medulloblastoma, Childhood, Melanoma, Melanoma, Intraocular (Eye), Merkel Cell Carcinoma, Mesothelioma, Adult Malignant, Mesothelioma, Childhood, Metastatic Squamous Neck Cancer with Occult Primary, Mouth Cancer, Multiple Endocrine Neoplasia Syndrome, Childhood, Multiple Myeloma/Plasma Cell Neoplasm, Mycosis Fungoides, Myelodysplastic Syndromes, Myelodysplastic/Myeloproliferative Diseases, Myelogenous Leukemia, Chronic, Myeloid Leukemia, Adult Acute, Myeloid Leukemia, Childhood Acute, Myeloma, Multiple (Cancer of the Bone-Marrow), Myeloproliferative Disorders, Chronic, Nasal cavity and paranasal sinus cancer, Nasopharyngeal carcinoma, Neuroblastoma, Non-Hodgkin lymphoma, Non-small cell lung cancer, Oral Cancer, Oropharyngeal cancer, Osteosarcoma/malignant fibrous histiocytoma of bone, Ovarian cancer, Ovarian epithelial cancer (Surface epithelial-stromal tumor), Ovarian germ cell tumor, Ovarian low malignant potential tumor, pancreatic cancer, islet cell cancer, Paranasal sinus and nasal cavity cancer, Parathyroid cancer, Penile cancer, Pharyngeal cancer, Pheochromocytoma, Pineal astrocytoma, Pineal germinoma, Pineoblastoma and supratentorial primitive neuroectodermal tumors, childhood, Pituitary adenoma, Plasma cell neoplasia/Multiple myeloma, Pleuropulmonary blastoma, Primary central nervous system lymphoma, Prostate cancer, Rectal cancer, Renal cell carcinoma (kidney cancer), Renal pelvis and ureter, transitional cell cancer, Retinoblastoma, Rhabdomyosarcoma, Salivary gland cancer, Sarcoma, Ewing family of tumors, Sarcoma, Kaposi, Sarcoma, soft tissue, Sarcoma, uterine, Sezary syndrome, Skin cancer (nonmelanoma), Skin cancer (melanoma), Skin carcinoma, Merkel cell, Small cell lung cancer, Small intestine cancer, Soft tissue sarcoma, Squamous cell carcinoma-see Skin cancer (nonmelanoma), Squamous neck cancer with occult primary, metastatic, Stomach cancer, Supratentorial primitive neuroectodermal tumor, childhood, T-Cell lymphoma, cutaneous-see Mycosis Fungoides and Sezary syndrome, Testicular cancer, Throat cancer, Thymoma, childhood, Thymoma and Thymic carcinoma, Thyroid cancer, Thyroid cancer, childhood, Transitional cell cancer of the renal pelvis and ureter, Trophoblastic tumor, Ureter and renal pelvis, transitional cell cancer, Urethral cancer, Uterine cancer, endometrial, Uterine sarcoma, Vaginal cancer, Visual pathway and hypothalamic glioma, childhood, Vulvar cancer, Waldenstrom macroglobulinemia, Wilms tumor (kidney cancer), childhood, etc.) and other cancers, in vitro or in vivo, comprising the steps of: contacting said cells with an amount of agent(s), compound(s) or drug(s) of the present invention delivered by a composition effective to inhibit the proliferation of the cancer cells.

In some embodiments, the present invention provides the means of inducing apoptosis in cancer cells in vitro or in vivo, comprising the steps of: contacting said cells with an amount of at least one agent, compound or drug of the present invention delivered by a composition effective to induce apoptosis in the cancer cells.

In some embodiments, the present invention provides the means of increasing the cytotoxic effects of at least one chemotherapeutic agents against the cancer cells, comprising the steps of: contacting said cells with said at least one chemotherapeutic agent, compound or drug of the present invention delivered by a composition wherein said composition of the present invention increases the cytotoxic effects of said one or more chemotherapeutic agent against the cancer cells.

In some embodiments, at least one chemotherapeutic agent is selected from the group consisting of vincristine, BCNU, melphalan, cyclophosphamide, Adriamycin, prednisone, velcade, thalidomide, and dexamethasone.

In some embodiments, said cancer cells are CD138+ plasma cells.

In some embodiments, the present invention provides the means of treating multiple myeloma or other cancer in an individual, comprising the step of administering a therapeutically effective amount of a composition of the present invention to said individual.

In one preferred embodiment the extract according to the present invention is produced by CO₂ extraction, DMSO extraction, combination of CO₂ extraction and DMSO extraction, cold-press extraction and steam distillation extraction.

Methods for producing an orange, frankincense, cannabis and other extracts (e.g. natural oils, absolutes, and concretes, etc.) are well known to the art (e.g. Harborne, 1998. Phytochemical Methods A Guide to Modern Techniques of Plant Analysis; I. Walinga, J. J. van der Lee, V. J. G. Houba, W. van Vark, I. Novozamsky, 1995, Plant Analysis Manual; Elizabeth M. Williamson, David T. Okpako, Fred J. Evans, 1996, Selection, Preparation and Pharmacological Evaluation of Plant Material; U.S. Pat. No. 6,241,975 B1; Schnaubelt, K. (2002). Biology of Essential Oils. San Rafael, Calif.: Terra Linda Scent; Guenther, E. (1982). The Essential Oils. Melbourne, Fl: Krieger Publishing; Food and Agriculture Organization of the United Nations (1995). Basic Principles of Steam Distillation. Retrieved Aug. 18, 2005, from http://www.fao.org/docrep/V5350e/V5350e13.htm; Catty, S. (2001). Hydrosols: The Next Aromatherapy. Rochester, Vt.: Healing Arts Press; Burnett, C. (2014) Safety Assessment of Citrus-Derived Peel Oils as Used in Cosmetics, Cosmetic Ingredient Review, Personal Care Products Council; NTP. (2000), Lemon Oil, Lime Oil, National Toxicology Program, U.S. Department of Health & Human Services; Guba, R. (2002); The Modern Alchemy of Carbon Dioxide Extraction. International Journal of Aromatherapy 12 (3), 120-126; http://www.edenbotanicals.com/extraction-methods, CN102391911: Supercritical extraction method for orange peel essential oil; Parameters optimization of supercritical fluid-CO₂ extracts of frankincense using response surface methodology and its pharmacodynamics effects.”/Jing Zhou, Xing-miao Ma, Bi-Han Qiu, Jun-xia Chen, Lin Bian, Lin-mei Pan//Journal of Separation Science, Volume 36, Issue 2, January 2013, Pages 383-390) incorporated herein in their entireties.

### Sources of Orange, Frankincense and Cannabis (Natural) Oils and Extracts

Steam distillation is the most well-known technique for extracting natural oil from plants, however there are many other methods suitable for obtaining and concentrate the aromatic constituents of plant materials. The examples of methods of obtaining natural oils and extracts below are for illustrative purposes only are not limiting upon the invention.

### Natural Oils: Distillation & Expression

Natural oils are produced in the cells of plants. The oils may be concentrated through steam distillation (and sometimes hydro or water distillation or a combination thereof). Steam distillation involves bubbling steam through the plant material. As natural oils are typically immiscible in water and have a higher boiling point, the natural oils vaporize at lower temperatures. Other methods used to produce essential oils include dry or vacuum distillation, dry/destructive distillation, and expression (“cold press”). Expression involves obtaining oil by applying high mechanical pressure to the plant material.

### Concretes & Absolutes: Solvent Extraction

Concretes and Absolutes are highly concentrated aromatic materials extracted by first extracting the aromatic oil with a solvent, then removing the solvent to derive a semisolid to solid waxy substance called a concrete. Concretes are soluble in both carrier oil and alcohol, though often it is necessary to filter out any insoluble waxes and solid material. Aromatic oils may be extracted and separated from most of the plant waxes and nonaromatic material with ethyl alcohol. After the ethyl alcohol is removed, the remaining substance is called an absolute. Absolutes still contain some waxes and pigments, but are mostly comprised of the concentrated aromatic oil. In addition, they may contain a small percentage of alcohol (typically up to 2 or 3 percent).

### CO2 Extracts: Solvent Extraction

CO2 extracts, like natural oils contain many beneficial therapeutic properties, but are extracted using CO2 (carbon dioxide) gas under pressure at ambient temperature. The advantage of CO2 extraction is that the CO2 returns to its gaseous state by lowering its pressure, allowing the gas to quickly and completely dissipate. Depending on the pressure used, a “select” or “total” extract will result.

### Organic Extracts

Organic extraction is a gentle extraction process involving certified organic solvents. The resulting product, extracted without added heat, may preserve bioactivity and therapeutic value of delicate cannabis compounds.

### Resins & Other Types of “Oils”.

Aromatic essences may be collected from the resin that oozes out of the bark of trees after tapping. Likewise, in destructive distillation, a starting material (such as Benzoin resin) is super-heated and cooked until an oil substance is obtained from the solid starting material.

### Cannabis DMSO Extraction

Extraction A: In this process, 5 g of dried cannabis was placed in a conical tube with 50 ml and allowed to stand at room temperature overnight until the soluble matter dissolved. The mixture was then strained, the damp solid material pressed, and the combined liquids clarified by filtration to remove remaining solid plant materials to form a stock solution.

### Cannabis Oil Extraction Methods

Starting with dry cannabis

1. Place cannabis in 100% isopropanol (11b into 2 gal)
2. Crush/mash leaves into solvent

### 3. Filter

4. Set aside filtrate
5. Pour additional solvent over damp mashed leaves (just enough to cover)
6. Repeat steps 2 and 3
7. Combine with other filtrate
8. Discard plant material
9. Boil away solvent (do not go over 143° C.) in a well ventilated area

Using double boiler:

Fill the bottom of double boiler with water. Pour filtrate into the top pan. Turn burner on high and wait for top pot to boil. Once the liquid bubbles, immediately turn off burner. The boiling water will do the rest. Let the mixture in the top pan bubble for 15 to 20 minutes. Once the oil is nice and thick, it is complete. Remove it from the pan and scrape it out onto parchment paper when it cools.

10. Once cooled take up extract into syringe.

### Sample Alternative Cannabis Oil Extraction Methods.

Supercritical fluid with or without an organic solvent modifier such as methanol may be used as an extractant. Supercritical fluids are materials that are under sufficient pressure and heat that they are no longer distinctly liquid or gaseous; as a consequence, they have the penetrating power of gases and the solvating power of liquids.

Dried cannabis is decarboxylated by heating at 120° C. for 1 hour. Decarboxylated botanical raw material is packed into a single column and exposed to liquid CO2 under pressure. With the following parameters:

Pressure: 60 bar+/−10 bar. Temperature: 10° C.+/−5° C. Time: Approximately 8 hours. CO2 mass flow 1250 kg/hour+/−.20%. Following depressurization and venting off of the CO2 the crude extract is collected into sealed vessels. The crude BDS extract is held at −20° C.+/−.5° C. The extract is then mixed with ethanol “winterization” solution with the following parameters: extraction temp 36-44° C., ratio ethanol/product approximately 2:1, freezer temperature −25° C. to −15° C., time 48-54 hours.

The ethanol solution produced in the second extraction stage requires filtration (20 km) to remove the resulting precipitation. (About 6 hours).

The final stage of the manufacturing process is the removal of ethanol and any water that may be present by heating at 60° C. to give a vapor temperature of 40° C. under a vacuum of 172 mbar. The distillation under these conditions continues until there is little or no visible condensate. Reducing the vacuum further, in stages, down to approximately 50 mbar, completes water removal. On completion the extract is transferred into sealed stainless steel containers and stored in a freezer at −20° C.

In some embodiments, the compositions, manufacture, products, processes, methods, and/or methods of use, prevention and treatment of the present invention comprise compounds, agents or drugs extracted from ginger, cinnamon, black pepper, or cloves using ethanol or methanol extraction techniques.

In one preferred of the preferred embodiments composition further comprises at least one diluent, and/or at least one stabilizer, and/or at least one surfactant, and/or at least one salt or buffering agent.

In some embodiment, the surfactant is a nonionic surfactant (e.g. polysorbate or Tween 80).

In some embodiments, Tween 80, a polyethylene glycol or a polyoxyethylene polyoxypropylene glycol is included at approximately 0.001% (w/v) to about 10% (w/v).

In embodiments involving a stabilizer, the stabilizer may be any suitable stabilizer known to the art (e.g. Stella and Rajewski, 1997; Merisko-Liversidge and Liversidge, 2003; U.S. Pat. No. 5,376,359). The stabilizer, may for example, be an amino acid, such as for instance, glycine; or an oligosaccharide, such as for example, sucrose, tetralose, lactose or a dextran. The stabilizer may also be a sugar alcohol, such as mannitol or a combination the stabilizer types described above.

In some embodiment, a stabilizer or stabilizers constitute approximately 0.1% to about 10% weight for weight of the compound.

Examples of acceptable salts useful in the invention include, but are not limited salts formed with inorganic acids (e.g. those selected from the group consisting of hydrochloric, hydrobromic, sulfuric, phosphoric, nitric or equivalent), or salts formed with acids or organic acids (e.g. acetic, oxalic, tartaric, succinic, malic, fumaric, aleic, ascorbic, benzoic acid, tannic, alginic, polyglutamic, naphthalene sulfonic acid, naphthalene disulfonic acid and polygalacturonic).

Treatment, exposure, combining or complexing of the compounds, extracts, oils, agents, and/or drugs, etc. of the present invention “with acids” will frequently result in beneficial changes to bioavailability, pharmacokinetics, metabolism, toxicity and/or excretion of the products of said acid treatments, and/or their metabolites, as compared to the untreated, unexposed, uncombined, and uncomplexed, compounds, extracts, oils, agents, and/or drugs, etc. The same may often be true with respect to treatment with various chemical bases. Accordingly, such products of acid or base treatment, exposure, combining or complexing are covered by the various embodiments described herein. In some embodiments, the compositions, manufacture, products, processes, methods, and/or methods of use, prevention and treatment useful in the methods of the present invention contain one or more conventional additives.

Additives include a solubilizer (e.g. US20070021325; U.S. Pat. No. 6,669,964; WO2009126950; WO2009101263). Additives may comprise glycerol or an antioxidant such as for example, benzalkonium chloride, benzyl alcohol, chloretone or chlorobutanol. Additives may also include an anesthetic.

To reduce oxidation and spoilage, the pharmaceutical compositions, manufacture, products, processes, methods, and/or methods of use, prevention and treatment may be stored under nitrogen gas or argon gas in sealed vials.

In one preferred embodiment buffering agent is selected from the group comprising sodium biphosphate, potassium biphosphate, sodium bicarbonate, potassium bicarbonate, carboxylic acids and their salts.

The buffering agents of the present invention may be any salt or buffering agent. Examples include sodium chloride, potassium chloride, or sodium phosphate or potassium phosphate.

In some embodiments, the salt and/or buffering agent is useful in maintaining osmolality in a suitable range for administration of the composition to a human or an animal. The salt or buffering agent may preferably be present at isotonic concentration of about 150 mM to about 300 mM.

Examples buffers include sodium biphosphate, potassium biphosphate, sodium bicarbonate, potassium bicarbonate, carboxylic acids and their salts, such as, ascetic acid/sodium acetate and citric acid/potassium citrate.

The buffering agent will in some embodiments, maintain the pH of the composition in the range of about 5.5 to about 7.5.

In one preferred embodiment the composition further comprises at least one second active agent having therapeutic benefit.

In some embodiments, the composition and method further comprises an additional agent, drug or compound such as an FDA approved at least one agent, compound or drug or non-FDA approved at least one agent, compound or drug.

In some embodiments the invention also specifically covers the use of compounds, agents, and drugs specified or named herein (and their analogs), in conjunction with other anti-hypertensive agents, cardioprotectant agents, anti-obesity agents, fertility agents, glycemic control agents, anti-hyperlipidemic agents, anti-atherosclerotic agents, anti-cancer agents, anti-chemotherapeutic resistance agents, and other approved agents and drugs as part of combination therapies and medicinal compositions.

In some embodiments, the invention relates to novel compositions and delivery methods that increase the availability of various compounds, agents and drugs to the body, especially via the oral route, particularly when such drugs and compounds otherwise lack significant oral bioavailability. In some embodiments, the invention relates to a composition comprising deuterium depleted water (DDW) and/or at least one extract selected from the group comprising orange, frankincense, cannabis or other natural oil or extract or at least one substance or compound isolated from said at least one oil or extract and other natural oils and/or extracts and/or at least one other an equivalent effective amount of other agent, compound, or drug of the present invention.

Further, in some embodiments, the ratio of at least one extract selected from the group comprising orange, frankincense, cannabis or other natural oil or extract or at least one substance or compound isolated from said at least one oil or extract and other natural oils and/or extracts and other at least one agent, compound, or drug of the present invention to other active compounds or agents in the compositions, manufacture, products, processes, methods, and/or methods of use, prevention and treatment ranges from 1:10 to 10:1.

In some embodiments, the ratio of at least one extract selected from the group comprising orange, frankincense, cannabis or other natural oil or extract or at least one substance or compound isolated from said at least one extract and other natural oils and/or extracts and other at least one agent, compound, or drug of the present invention to glutathione is 1:1.

In some embodiments, the ratio of at least one extract selected from the group comprising orange, frankincense, cannabis or other natural oil or extract or at least one substance or compound isolated from said at least one extract and other natural oils and/or extracts and other at least one agent, compound, or drug of the present invention to glutathione is 1:4 to 1:10.

In some embodiments, the ratio of the at least one extract selected from the group comprising orange, frankincense, cannabis or other natural oil or extract or at least one substance or compound isolated from said at least one extract and other natural oils and/or extracts and/or at least one other agent, compound, or drug of the present invention to other active compounds or agents in the compositions, manufacture, products, processes, methods, and/or methods of use, prevention and treatment ranges from 1:50 to 50:1 based upon dry weight.

In some embodiments, at least one extract selected from the group comprising orange, frankincense, cannabis or other natural oil or extract or at least one substance or compound isolated from said at least one extract and other natural oils and/or extracts, agents, compounds, products, derivatives, structural variants, and/or drugs described herein are combined with zerumbone, a sesquiterpene, an agents, agents a compound, compounds, a drug, drugs, and/or their structural variants, etc., described in US20140271923.

In some embodiments, the compositions, manufacture, products, processes, methods, and/or methods of use, prevention and treatment of the present invention comprise at least one extract selected from the group comprising orange, frankincense, cannabis or other natural oil or extract or at least one substance or compound isolated from said at least one extract and other natural oils and/or extracts and/or at least one other agent, compound, or drug of the present invention, selected from a methoxyflavone (especially a dimethoxyflavone), n-acetylcysteine, glutathione, a glutathione precursor or a glutathione enhancing agent or a known intracellular glutathione promoting agent, folinic acid, folic acid, trimethylglycine, vitamin D, and medicinal iron.

In some embodiments, the composition comprises glutathione, especially reduced glutathione.

In some embodiments, this invention relates to compositions, manufacture, products, processes, methods, and/or methods of use, prevention and treatment for achieving clinical benefit related to an increase in intracellular glutathione.

In some embodiments, the compositions, manufacture, products, processes, methods, and/or methods of use, prevention and treatment comprising at least one extract selected from the group comprising orange, frankincense, cannabis or other natural oil or extract or at least one substance or compound isolated from said at least one extract and other natural oils and/or extracts and/or other at least one agent, compound, or drug of the present invention and an approved drug further comprise reduced glutathione.

In some embodiments, at least one extract selected from the group comprising orange, frankincense, cannabis or other natural oil or extract or at least one substance or compound isolated from said at least one extract and other natural oils and/or extracts and/or at least one other named agent, compound, or drug of the present invention and/or reduced glutathione is incorporated into the approved drug's composition along with the approved drug without otherwise altering the approved drug's composition.

In some embodiments, at least one extract selected from the group comprising orange, frankincense, cannabis or other natural oil or extract or at least one substance or compound isolated from said at least one extract and other natural oils and/or extracts and/or at least one other agent, compound, or drug of the present invention and/or reduced glutathione is incorporated into the approved drug's composition along with the approved drug while adjusting the concentration of at least one of the compositions active drug, stabilizer, buffer, vehicle, excipient, etc.

In some embodiments, reduced glutathione (in doses ranging from about 200 mg tp 2000 mg) is added to any of the compositions, manufacture, products, processes, methods, and/or methods of use, prevention and treatment described herein.

In some embodiments, the compositions, manufacture, products, processes, methods, and/or methods of use, prevention and treatment of the present invention comprise an analog of a compound, agent, or drug named herein.

In some embodiments, reduced glutathione is added to any of the compositions, manufacture, products, processes, methods, and/or methods of use, prevention and treatment described herein.

In some embodiments, a compound or a drug of the classes exemplified herein, are combined with glutathione or an antioxidant in a nanoemulsion, nanoparticles (e.g. WO/2010/013224), nanovault, nanofiber, nanotube or other nanostructure.

In some embodiments, the composition comprises at least one agent or compound extracted from ginger, such as, zingerone, a gingerol, or a shogaol.

In some embodiments, the composition comprises at least one an amino acid. In some embodiments, the composition comprises L-cysteine.

The present invention contemplates composition comprising agents, compounds, or drugs selected from the group consisting of zingerbene, agoraspirol, amorphine, anhydro-β-rotunol, aromadendrine, azulene, bisabolene, bisabolol, cadalene, cadinene, cadrina-1,4-diene, caryophyllene, cedrene, cedrol, cerapictol, ceratopicanol, clovene, copaene, cubebene, eudalene, eudesmol, farnesene, farnesol, as well as their derivatives and analogs.

The present invention also contemplates composition comprising agents, compounds, or drugs selected from the group consisting of germacrene, guaiazulene, guaiol, gurjunene, hexahydrohumulene, himachalene, hinesol, humulene, junipene, longifolene, lubiminol, khusimone, khusinol, khusimol, nootkatone, santalene, santalol, santanol, santonene, selinene, solavetivone, spatulenol, sterpurine, sulcatine, thujopsene, valerenol, vetispirene, vetivazulene, vetivene, vetiverol, vetivone, viridiflorine, and viridiflorol as well as their derivatives and analogs.

In some embodiments, the analogs/derivatives of the present inventions are produced through addition of a mono-phenyl ring, addition of a heterocycle, addition of a substituted amide, addition of an unsubstituted amide, addition of a carbonyl imidazole, addition of a CN functional group, addition of a CONH2 functional group, addition of a CONHNH2 functional group, addition of a CO-D-Glu(OAc)4 functional group, and/or addition of a ketone to one of the following: an approved drug (e.g. one named or described herein), an OTC drug, a sesquiterpene, a sesquiterpenoid, a sesquiterpene lactone (e.g. lactucin, lactuopicrin, 8-deoxylactucin, picriside A, crepidiaside A, jacquinellin, jacquinellin glycoside, chamissonolide, helenalin, alantolactone, dehydrocostus lactone, costunolide), a id, an ATF4 modulator, an FST1 modulator, an FST1 modulator, an NRF2 modulator, a KEAP1 modulator, a flavone, a flavonoid, quercetin, a shogaol (e.g. 6-shogaol), a gingerol (e.g. 6-gingerol), zingerol, kavalactone, sulforaphane, allyl-, butyl- and phenylethyl-isothiocyanate, chlorophyllin, alpha-lipoic acid, allicin, plumbagin, protandim, capsaicin, a capsaicinoid, piperine, asafetida, eugenol, piperlongumine, pellitorine, zingiberine, tBHQ, CDDO-lm, MC-LR, epigallocatechin-3-gallate, a compound found in wasabi, cafestol, xanthohumol, 5-O-caffeoylquinic acid, N-methylpyridinium, resveratrol, nootkatone, caffeic acid phenethyl ester, 3-O-Caffeoyl-1-methylquinic acid, silymarin, kahweol, garlic organosulfur compounds, lycopene, carnosol (rosemany), an avicin, oltipraz, CDDO, a neurite outgrowth promoting prostaglandin, vitamin D, a B vitamin, andrographolide, an amino acid, s-allylcysteine, Vitamin A, Vitamin C, Vitamin E, β carotene, trans-2-hexenal, cyclopentenone, ajoene, Dihydro-CDDO-trifluoroethyl amide, Hypochlorous acid, Fragrant unsaturated aldehydes (e.g. trans-cinnamaldehyde, safranal, 2,4-octadienal, citral, and trans-2,cis-6-nonadienal), 2-OHE, 4-OHE, bucillamine, acrolein, momordin, momordol, momordicin I, momordicin II, momordicosides, momordicin-28, momordicinin, momordicilin, momordenol, momorcharin, cucurbitacin B, charantin, charantosides, goyaglycosides, α-eleostearic acid, 15,16-dihydroxy-α-eleostearic acid, antirheumatic gold(I) compounds, an avicin, dithiolethione, an approved drug, an OTC drug, and/or a compound, agent or drug extracted from cloves, black pepper, red chili, cinnamon (e.g. cinnamic aldehyde), ginger, garlic, onion, fennel, bay leaves, nutmeg, saffron, coriander, an ATF4 modulator, an FST1 modulator, an NRF2 modulator or a KEAP1 modulator.

The sesquiterpenes or monoterpenes of the present invention may also represent a lactone compound, a ketolactone compound, an alcohol compound a ketone compound, an aldehyde compound, an ester compound, an ether compound, or a carboxylic acid compound. The present invention covers compositions, manufacture, products, processes, methods, and/or methods of use, prevention and treatment comprising agents, compounds and drugs of the present invention, their derivatives, analogs, and isomers. These derivatives, analogs, and isomers include, but are not limited to acetyl, acetate, phenylacetate, hydro, dihydro, formate, methyl ether, dimethylether, caprylate, valeriate, isovaleriate, alcohol, aldehyde, ketone, epoxide, lactone and cyclases derivatives.

In some embodiments, the compositions, manufacture, products, processes, methods, and/or methods of use, prevention and treatment of the present invention comprise agent, compounds, or drugs selected from curcumin, zingerone, a methoxyflavone, vitamin C, n-acetylcysteine, trimethylglycine, folinic acid, folic acid, an amino acid and/or reduced glutathione.

In some embodiments, the compositions, manufacture, products, processes, methods, and/or methods of use, prevention and treatment of the present invention comprise agent, compounds, or drugs selected from kavalactone, sulforaphane an isoselenocyanate compound of sulforaphane, alpha-lipoic acid, and/or allicin.

In further embodiments, the compositions, manufacture, products, processes, methods, and/or methods of use, prevention and treatment comprising at least one extract selected from the group comprising orange, frankincense, cannabis or other natural oil or extract or at least one substance or compound isolated from said at least one extract and other natural oils and/or extracts and/or other at least one agent, compound, or drug of the present invention and an approved drug further comprise a vitamin D.

In further embodiments, the compositions, manufacture, products, processes, methods, and/or methods of use, prevention and treatment comprising at least one extract selected from the group comprising orange, frankincense, cannabis or other natural oil or extract or at least one substance or compound isolated from said at least one extract and other natural oils and/or extracts and/or other at least one agent, compound, or drug of the present invention and an approved drug further comprise a vitamin B.

The present invention covers compositions comprising agents, compounds and drugs of the present invention, their derivatives, analogs, and isomers. These derivatives, analogs, and isomers include, but are not limited to acetyl, acetate, phenylacetate, hydro, dihydro, formate, methyl ether, dimethylether, caprylate, valeriate, isovaleriate, alcohol, aldehyde, ketone, epoxide, lactone and cyclases derivatives.

In some embodiments, the analogs/derivatives of the present inventions are produced through conjugation of an amino acid, protein, glutathione, LHRH, bovine serum albumin (BSA) or non-protein to an approved drug, an OTC drug, a sesquiterpene, a sesquiterpenoid, one or more natural oil or other natural extract(s) and/or other agent(s), compound(s), or drug(s) of the present invention, 8-hydroxy-alpha-humulene, glutathione, auraptene, ethacrynic acid, curcumin, a curcuminoid, hispolon, dehydroxyhispolon, methoxyhispolon, bisdemethylcurcumin, hispolon methyl ether, hydroxyhispolon, methoxyhispolon methyl ether, a triterpenoid, zingerone, reservatrol, vanillin, rosmarinic acid, a methoxyflavone, a sesquiperetene, n-acetylcysteine, trimethylglycine, folinic acid, folic acid, an amino acid, an ATF4 modulator, an FST1 modulator, an NRF2 modulator, a KEAP1 modulator, a flavone, a flavonoid, quercetin, a shogaol (e.g. 6-shogaol), a gingerol (e.g. 6-gingerol), zingerol, kavalactone, sulforaphane, allyl-, butyl- and phenylethyl-isothiocyanate, chlorophyllin, alpha-lipoic acid, allicin, plumbagin, protandim, capsaicin, a capsaicinoid, piperine, asafetida, eugenol, piperlongumine, pellitorine, zingiberine, tBHQ, CDDO-Im, MC-LR, epigallocatechin-3-gallate, a compound found in wasabi, cafestol, xanthohumol, 5-O-caffeoylquinic acid, N-methylpyridinium, resveratrol, nootkatone, caffeic acid phenethyl ester, 3-O-Caffeoyl-1-methylquinic acid, silymarin, kahweol, garlic organosulfur compounds, lycopene, carnosol (rosemany), an avicin, oltipraz, CDDO, a neurite outgrowth promoting prostaglandin, vitamin D, a B vitamin, andrographolide, an amino acid, s-allylcysteine, Vitamin A, Vitamin C, Vitamin E, β carotene, trans-2-hexenal, cyclopentenone, ajoene, Dihydro-CDDO-trifluoroethyl amide, Hypochlorous acid, Fragrant unsaturated aldehydes (e.g. trans-cinnamaldehyde, safranal, 2,4-octadienal, citral, and trans-2,cis-6-nonadienal), 2-OHE, 4-OHE, bucillamine, acrolein, antirheumatic gold(I) compounds, an avicin, dithiolethione, an approved drug, an OTC drug, and/or a compound, agent or drug extracted from cloves, black pepper, red chili, cinnamon (e.g. cinnamic aldehyde), ginger, garlic, onion, fennel, bay leaves, nutmeg, saffron, coriander, an ATF4 modulator, an FST1 modulator, an NRF2 modulator or a KEAP1 modulator.

In some embodiments, the analogs/derivatives of the present inventions are derived from at least one extract selected from the group comprising orange, frankincense, cannabis or other natural oil or extract or at least one substance or compound isolated from said at least one extract and other natural oils and/or extracts and/or at least one agent, compound, or drug of the present invention are a sesquiterpenoid, a sesquiterpene lactone (e.g. lactucin, lactuopicrin, 8-deoxylactucin, picriside A, crepidiaside A, jacquinellin, jacquinellin glycoside, chamissonolide, helenalin, alantolactone, dehydrocostus lactone, costunolide), a sesquiterpene sulfate, reduced glutathione, auraptene, ethacrynic acid, curcumin, a curcuminoid, hispolon, dehydroxyhispolon, methoxyhispolon, bisdemethylcurcumin, hispolon methyl ether, hydroxyhispolon, methoxyhispolon methyl ether, a triterpenoid (e.g. Betulinic acid), zingerone, reservatrol, vanillin, rosmarinic acid, a methoxyflavone, a sesquiperetene, n-acetylcysteine, trimethylglycine, folinic acid, folic acid, an amino acid, an FST1 modulator, NRF2 modulator, KEAP1 modulatorflavone, a flavonoid, quercetin, a shogaol (e.g. 6-shogaol), a gingerol (e.g. 6-gingerol), zingerol, kavalactone, sulforaphane, allyl-, butyl- and phenylethyl-isothiocyanate, chlorophyllin, alpha-lipoic acid, allicin, plumbagin, protandim, capsaicin, a capsaicinoid, piperine, asafetida, eugenol, piperlongumine, pellitorine, zingiberine, tBHQ, CDDO-Im, MC-LR, epigallocatechin-3-gallate, a compound found in wasabi, modihydrocapsaicin, cafestol, 16-O-methyl cafestol, xanthohumol, isoxanthuhumolol, 5-O-caffeoylquinic acid, N-methylpyridinium, resveratrol, nootkatone, caffeic acid phenethyl ester, 3-O-Caffeoyl-1-methylquinic acid, silymarin, kahweol, garlic organosulfur compounds, lycopene, carnosol (rosemany), an avicin, oltipraz, CDDO, a neurite outgrowth promoting prostaglandin, vitamin D, a B vitamin, andrographolide, an amino acid, s-allylcysteine, Vitamin A, Vitamin C, Vitamin E, β carotene, trans-2-hexenal, cyclopentenone, ajoene, Dihydro-CDDO-trifluoroethyl amide, Hypochlorous acid, Fragrant unsaturated aldehydes (e.g. trans-cinnamaldehyde, safranal, 2,4-octadienal, citral, and trans-2,cis-6-nonadienal), 2-OHE, 4-OHE, bucillamine, momordin, momordol, momordicin I, momordicin II, momordicosides, momordicin-28, momordicinin, momordicilin, momordenol, momorcharin, cucurbitacin B, charantin, charantosides, goyaglycosides, α-eleostearic acid, 15,16-dihydroxy-α-eleostearic acid, antirheumatic gold(I) compounds, an avicin, dithiolethione, an approved drug, an OTC drug, and/or a compound, agent or drug extracted from cloves, black pepper, red chili, ginger, garlic, onion, fennel, bay leaves, nutmeg, saffron coriander and cinnamon (e.g. cinnamic aldehyde).

In some embodiments, the composition of the present invention comprises agents, compounds or drugs named herein and is and are used to prevent or treat cancer metastasis.

In some embodiments, a sesquiterpene other than at least one natural oil or other natural extract is provided: a sesquiterpene lactone (e.g. lactucin, lactuopicrin, 8-deoxylactucin, picriside A, crepidiaside A, jacquinellin, jacquinellin glycoside, chamissonolide, helenalin, alantolactone, dehydrocostus lactone, costunolide), or a sesquiterpene lactone sulfate (Sessa et al., 2008).

In some embodiments, the composition and method of the present invention comprises agents, compounds or drugs of the various classes named herein (e.g. at least one extract selected from the group comprising orange, frankincense, cannabis or other natural oil or extract or at least one substance or compound isolated from said at least one extract and other natural oils and/or extracts, sesquiterpene, zingerone, oltipraz, sulfuraphane, etc.), anti-EGFR agents (e.g. EGFR antibody, cetuximab and panitumumab), anti-VEGF agents (e.g. VEGF antibody), and/or another approved drug to prevent or treat cancer metastasis.

In some embodiments, the composition and method of the present invention comprises agents, compounds or drugs named herein (e.g. ones that are glutathione-conjugated, biotinylated, fluorinated, containing an NO moiety, etc.), anti-EGFR agents (e.g. EGFR antibody, cetuximab, necitumumab and panitumumab), anti-VEGF agents (e.g. VEGF antibody), and/or another approved drug (e.g. an NSAID) to prevent or treat cancer metastasis.

In some embodiments, the present invention provides the means of increasing the cytotoxic effects of at least one chemotherapeutic agents against multiple myeloma or other cancer cells in an individual, comprising the steps of: administering to said individual said at least one chemotherapeutic agents and an agent, compound or drug of the present invention, wherein said composition of the present invention increases the cytotoxic effects of said one or more chemotherapeutic agents against multiple myeloma cells in said individual.

In some embodiments, at least one chemotherapeutic agents is selected from the group consisting of vincristine, BCNU, melphalan, cyclophosphamide, Adriamycin, prednisone velcade, thalidomide, and dexamethasone or other approved chemotherapeutic listed herein.

In some embodiments, the composition of may comprise at least one extract selected from the group comprising orange, frankincense, cannabis or other natural oil or extract or at least one substance or compound isolated from said at least one extract and other natural oils and/or extracts, at least one natural compound, at least one synthetic compound, and/or at least one approved drug, and are further defined by their effects on gene expression as described below.

When treating or preventing cancer, the compositions, manufacture, products, processes, methods, and/or methods of use, prevention and treatment will, in some preferred embodiments, modulate the expression of at least 2%, preferably at least 20%, and most preferably, >30% of genes selected from SPP1, Col6a2, INHBA, Col6a1, RP6-24A23.7, Gstm7, Nkd1, Nkd1, Pxdn, Sema5a, Sema5a, KANK4, TUBA1A, COL3A1, Inhbb, LIN28B, SPARC, FOXG1, Nef1, Miat, LDHB, S100A2, S100A2, S100A2, ZFP36L1, IFIT2, TMEM47, Tbx1, Pxdn, CCL5, CDH2, MMP1, S100A2, UBB, Map2, Worthington, Tbx1, CELF2, CDH1.

When treating or preventing cancer, the compositions, manufacture, products, processes, methods, and/or methods of use, prevention and treatment will, in some preferred embodiments, modulate the expression of at least 2%, preferably at least 20%, and most preferably, >30% of genes selected from SPP1, Col6a2, INHBA, Col6a1, RP6-24A23.7, Gstm7, Nkd1, Nkd1, Pxdn, Sema5a, Sema5a, KANK4, TUBA1A, COL3A1, Inhbb, LIN28B, SPARC, FOXG1, Nef1, Miat, LDHB, S100A2, S100A2, S100A2, ZFP36L1, IFIT2, TMEM47, Tbx1, Pxdn, CCL5, CDH2, MMP1, S100A2, UBB, Map2, Worthington, Tbx1, CELF2, CDH1, Cacna1c, Col6a2, JAM3, PSAT1, Slco4a1, UBB, S100A2, SALL1, HOXD10, MSN, IFIT2, Mgp, Cacna1c, CAV1, NEFL, NEFL, Miat, Nef1, AKR1B1, Foxa2, Foxa2, Pmaip1, Ramp3, CALD1, GSTP1, NEFL, DMD, VIM, Lin7a, ALDH1A1, AGPS, Rbms3, PNMAL1, BCAT1, PAX6, ZNF655, RSAD2, Chl1, SPINK7, FEZ1, CDH1, IFIT2, Cys1tr1, HMGA2, POU4F1, DKK1, UCHL1, DNER, SPINK6, CD52, CCL5, PDPN, CNRIP1, PDE5A, NRN1, Col6a1, Ccbe1, IFIH1, Foxd1, Ndst3, DLK1, HOXA9, Rbms3, IFIT2, RSAD2, Sprr1a, IFI27, HOXA9, OLR1, DEFA6, PTPRD, AGR2, Map2, RP11-834C11.4, TCF4, DZIP1, IGF2BP3, AKT3, AC116614.1, Inhbb, Aldh1a1, Kif1a, ERVMER34-1, TDO2, EMP1, IFIT3, S100A2, PRAC1, Slc14a1, NPTX2, CDKN2A, CCND2, Aff3, Myo5b, Ror1, Pmaip1, Mdfic, GBP1, OVALY, IFIT3, LCE3D, MALAT1, PRKCQ-AS1, WDR72, GAS1, HOXD13, Gstm7, Aldh1a2, Ror1, Lrrc32, Ror2, Basp1, IL13RA2, MAL2, IL1A, IFIH1, Slc14a1, IFIT3, ALDH1A2, SPAG16, RNF217, WBP5, IFI16, Gm13373, Ror2, Slco4a1, Ikzf2, Ccbe1, Lin7a, Atp6v0d2, CXCL10, COL1A1, EIF1AY, GPR82, SPINK13, IER3, Ugt2b34, DDX58, RGS1, INHBA, Bend7, ELAVL2, FSTL1, Fgfr1, SPOCK1, ST3GAL6, OSBPL3, FOXF2, CMPK2, Gramd1b, Mdfic, Chl1, Pax2, CMPK2, Mgp, Aldh1a1, GGH, B2M, LUM, BMP2, EDNRA, FABP4, MMP2, ITGA5, PPAPDC1A, Bend7, POU3F3, IL1RL1, CPVL, LUM, EPB41L3, IL1RL1, Foxd1, TACSTD2, DEFA5, Gas1, IFIT3, GPX2, Tfap2b, CCL5, GBP1, GBP1, HS3ST3B1, IFIT3, FN1, MDFIC, IL1RL1, ZNF521, AUTS2, SERPINB2, SLC44A5, Gfra1, Kif1a, SERPINB2, DLX2, Gng2, Aff3, Snhg18, Kcnmb4, Pdcd1lg2, Aldh1a2, Sel1l3, VIM, LUM, CDH11, SLC6A15, IL33, SERPINE1, HSPA1B, VWF, IL13RA2, SLC12A8, MMP1, ACTA2, F2RL1, FOXP3, CDC20B, SLC6A15, SPON1, ZEB1, Basp1, EMX2, Six3, IFI27, RP24-317M4.6, Sgcd, St3gal5, KCNJ16, Myo1b, Tmem200a, Fstl4, CMPK2, Ugt2b34, DDX58, MGP, CXCL8, HBA2, MAL2, GDF15, GPR34, GPNMB, SCG2, MMP1, F2RL1, ASXL3, TWIST1, BTG3, VCAN, DSC3, Six3, Ndst3, MFAP5, Cacng7, Cacng7, MSX2, RSAD2, IFIH1, A2M, COL5A1, HSPA1A, CYP24A1, DCBLD2, IL6ST, RAP2B, SFRP4, NCAM1, GOLIM4, NEFM, SAMD5, SLC1A3, SLC35F1, ZNF518B, SLC35G2, Add2, Pax2, St3gal5, Hivep3, Myo5b, IFNL2, HERC5, PDCD10, IL11, HS3ST3B1, PDCD10, ASCL1, CMBL, FGB, GPX8, CREB5, Gpnmb, DDR2, AGR3, IL1RL1, BCL11A, COL4A1, CXCL12, CCDC186, UCHL1, Adcy1, SCN3A, IFNL2, Tfap2b, Zbtb10, Clic5, Gm10419, LPCAT2, WNT5A, SCG2, PPAPDC1A, RP11-43F13.1, HNRNPLL, Mapk4, CDH11, GLUL, HAVCR1, ACKR3, NEFL, ZFYVE16, MPP1, SLITRK5, ZFPM2, TNFSF15, AKR1C3, SUSD5, SERPINB2, TP73-AS1, SDHAF3, GNAI1, SPX, Tacstd2, Mxra7, IFIT1, UCHL1, PARK2, MAGEA12, Wnt11, Tmem200a, Sel1l3, Arhgdib, IL6, C4orf26, TNFAIP3, TRIB1, BHLHE41, Mapk4, CLIC6, PAPD4, TNFSF4, FAT3, ANO5, TUB, WNT5A, FGF9, ADAMTS1, POSTN, CHI3L1, CYP1A1, MTAP, IQGAP2, Shc2, GGH, Pearl, MXD1, Aig1, SRPX, Gas1, CXCL11, SLC18A1, Plch1, Aspa, PAH, IFNL1, IFNB1, EGR4, IFNL1, IFIT1, IFIT1, IFIT1, ANO4, MME, PKIB, DDX3Y, HERC5, HSPA7, HTRA1, CHIT1, REG4, IFIT1, RSAD2, FGA, IGFBP5, NCF2, OAS2, FBN1, DAB2, TGFBI, Ifi205, Cxcl13, Arhgef16, SERPINE2, AKR1B10, B2M, CMYA5, BMP2, MALAT1, FBXL21, CXCR4, CMPK2, GPX2, NEAT1, SLC7A11, CDH2, ERVMER34-1, AKR1B10, Susd4, Ngf, OASL, CXCL11, MAGEA12, BCHE, AA414768, Evl, Snhg18, Pdcd1lg2, Wnt11, GBP1, TNFAIP3, IL1B, GABBR2, VIP, Cys1tr1, ATP6V0D2, IFFO1, INSM1, SUCO, Bmp7, Slc4a4, TOP2A, TOP2A, PPM1K, NEK2, Adcy1, Aig1, Chst1, Abca1, AA414768, Penk, AI504432, Slfn4, Adh7, Fstl4, FSTL1, XYLT1, TNFAIP6, CACHD1, Gm5127, MGP, POSTN, TSTD1, MAFF, MMP2, IFI44L, IFIT2, SPP1, ID2, AL133493.2, F2RL2, EREG, CDH1, TM4SF18, NOX4, GLUL, CACNA2D1, IFI44L, MX2, C5, PEG3, FAM65C, JMY, HEY1, GLB1L3, ALDH1L2, SH3BGRL, EBF3, FLRT2, FLRT2, TACSTD2, ASPM, PMEL, TFCP2, EIF2AK3, BIRC5, SPP1, PAH, Gata2, IFNB1, CXCL14, Klhl29, RP24-317M4.6, Susd4, Lrrc32, ZEB2, FOXJ1, Bean1, CLU, OASL, Wisp2, Olfm1, CH25H, DDX58, MAFB, CTGF, IFNL2, ENPP2, MYOF, EGR4, TRIM22, Arhgdib, Gata6, ISG15, CCDC80, CD109, FAM198B, Arg1, BMP2, DCSTAMP, FABP4, IL18, CXCL8, PRUNE2, ZCCHC12, KRT6A, IFI44L, ENSGALG00000005812, RP11-362K14.7, MMP3, ARSB, CARD16, GJC1, FAR2, CPNE8, KLHL28, ALDH2, DLX6, ZNF426, CYP24A1, NRXN3, FOXP2, HRASLS, PNMA2, NCF2, CENPF, HOXD9, Pearl, GLIPR1, TNFRSF11B, IL6, HMGA2, MMP3, CCNB1, Podxl, Pkhd1, SEZ6L, TRIM22, PNLDC1, Myo1b, XYLT1, ADAM19, MFAP5, LRRC17, Dnase1l3, Ly6i, FCER1G, GJA1, ABCB1, MAGEA12, NTS, NPPB, IGFBP7, MX1, ABCC2, Worthington, Rd, EPDR1, BNIP3L, PSG5, LRCH2, SH3GL2, POPDC3, CCDC8, NUDT11, SH3BGRL, ZNF717, KCNQ5, TBX18, ZIC1, PNPLA8, FGF4, Bmp7, Cbr1, Otud7a, Cpvl, AGR2, IFI44L, CGA, AMBN, CST1, TWIST1, IL6, Mctp2, Gmpr, S1pr3, UGT8, Lingo1, Kcnmb4, Nr2f1, Calca, F2RL2, EDNRA, MAF, RP3-428L16.2, PRSS12, SP8, HERC5, ZEB2, FOSB, Penk, Evl, Slfn4, Ubash3b, APCDD1L-AS1, Fgfr1, Wisp2, Gata6, ADAMTS6, CXCL8, EPHB1, PRRX1, CAV2, P3H2, GPR183, BST2, TMEM200A, MMP13, Adamdec1, Cxcl9, MMP12, RSPO3, SGK1, MT1F, NRK, CD274, SFTPB, TNFSF10, HEP21, NEAT1, PCDH10, OAS2, TNC, SP8, CHRM3, GLIPR1, TFCP2, AKAP12, PDE10A, COL11A1, MGP, SNCA, NID1, ZNF582-AS1, ANK2, HOXB8, LGALS1, FGF4, DLK1, GLIPR1, TNC, Cpvl, ISG15, ZC3HAV1, PADI4, CTC-444N24.11, SGMS2, Tmem100, CXCL10, Gata2, SERPINB5, TTC3, Gramd1b, AKR1B10, FCRL4, Mxra7, Scmh1, Gpr165, ISG15, OASL, DDX60.

When treating or preventing psoriasis the compositions will, in some preferred embodiments, modulate the expression of at least 2%, preferably at least 20%, and most preferably, >30% of genes selected from, IL36A, DEFB4A, SPRR2C, IL19, HSPD1P3, PI3, CLEC3A, SPRR2F, VNN3, S100A12, CXCL8, TCN1, HSPD1P2, CXCL11, TMPRSS11D, IL17A, SPRR2B, DEFB4B, SERPINB4, S100A7A, TMPRSS11A, LCE3A, TNIP3, S100A7A, SPRR3, KRT24, KRT6C, SPRR2A, S100A7A, S100A7A, S100A7A, S100A7A, S100A9, S100A7, S100A7A, S100A8, S100A9, S100A12, SERPINB3, S100A7A, LCE3B, TCN1, S100A12, IL17F, S100A12, CXCL1, AKR1B10, TCN1, CXCL9, HEPHL1, SPRR2C, S100A9, IDO1, TCN1, LCE3C, SPRR2C, TCN1, RSAD2, CXCL10, SPRR2C, SERPINB3, SPRR2C, CXCL13, S100A12, SERPINB3, SPRR2D, C12orf74, AKR1B10, S100A12, AKR1B10, CTC-490G23.2, S100A9, S100A9, SERPINB3, TCN1, IL22, S100A12, CCL8, SERPINB3, GDA, LTF, CLDN17, S100A12, TCN1, AL591704.7, IL20, IL36G, SPRR2C, SPRR3, TMPRSS11D, SPRR2C, SPRR2C, S100A12, S100A7A, IL36G, CASP5, SPRR2C, SERPINB3, IGFL1, TCN1, APOBEC3A, TCN1, C10orf99, NOS2, C10orf99, CXCL8, C10orf99, S100A12, LCE3D, SERPINB3, TCN1, RP11-398B16.2, SERPINB3, AKR1B10, VNN3, MMP12, S100A9, SERPINB3, TCN1, SPRR2C, KRT16, LINC01206, S100A12, RP4-529N6.2, IGFL1, LCE3D, RHCG, IL36G, S100A9, OAS2, S100A9, LCE3E, S100A9, HRNR, SERPINB3, CXCL6, LTF, ISG20, TCN1, HERC6, ZP4, CLCA3P, ISG20, SPRR2C, EPGN, CXCL13, S100A9, ADGRF1, RHCG, AKR1B1P1, S100A12, OASL, ISG15, SERPINB3, TCN1, KYNU, IL12B, MMP12, IL17C, IL19, SPRR2C, FADS2P1, AKR1B10, IL36A, IFI44L, ENKUR, PLEKHG7, S100A12, S100A9, CCL20, CXCL8, ABCG4, CTA-384D8.31, CXCL8, LCN2, GDA, MUC4, VNN3, VNN3, LCN2, GDA, VNN3, S100A12, HMGN2P23, CHAC1, RP11-350J20.12, KYNU, TCN1, TCN1, S100A12, CHAC1, RHCG, OASL, LTF, S100A9, LINC01269, KYNU, GDA, TCN1, SERPINB3, KRT16P2, SPRR2G, KYNU, IL36G, LCE3D, RARRES3, WARS, STAT1, IL26, IFIT3, ATP12A, HTR3B, KRT6A, OASL, HABP2, S100A8, S100A9, C10orf99, PLA2G4D, S100A12, HPSE, TRIM15, MMP1, GJB2, IL36G, LCN2, GDA, SPRR2C, CXCL8, KLK6, SPRR2C, ATP12A, CTA-384D8.35, IL36G, ATP12A, KRT16, SLC6A14, TCN1, MX1, IRF1, TNIP3, HIST1H4C, OR5H8, KRT16P3, ADAMDEC1, AC087762.1, AKR1B15, RGS1, IL36G, LCN2, AKR1B10, AKR1B10, S100A8, GDA, RHCG, RP11-430H10.2, RP11-1079J22.1, SOCS1, APOL1, OAS1, OAS3, SOST, LCN2, ATP12A, HTR3A, CXCL5, APOBEC3A, KYNU, TMPRSS11D, RP11-430H10.3, CCL7, CLEC6A, PRSS27, TMPRSS11D, HPSE, LTF, IL36G, CXCL8, LTF, KYNU, KYNU, AKR1B10, S100A9, LCN2, CCL20, CLLU1OS, HPSE, S100A8, KYNU, SERPINB13, AKR1B10, KYNU, RP11-526F3.1, ANKRD34B, AKR1B10, KYNU, S100A7A, SPRR2G, LCN2, ATP12A, CXCL8, TEX101, CHI3L2, LTF, HPSE, LCN2, KRT16, KRT16, CXCL8, LCN2, DDX58, GBP1, IFI35, IFIH1, TYMP, CTA-384D8.35, PRSS27, KLHDC7B, ATP12A, KRT16, DSC2, RP11-63P12.7, UGT1A7, AZGP1P2, KLK13, SERPINB3, TMPRSS11D, ATP12A, AKR1B10, KYNU, AKR1B10, HRH3, AC007163.3, ADAMDEC1, ADAMDEC1, RHCG, CXCL8, LCN2, RHCG, LCN2, IFI6, PLA1A, SELL, CHAT, SERPINB3, RND1, ZNF812, KRT16, CXCL8, IL36G, KLK13, UGT1A9, OSM, TRIM10, CXCL10, DYNAP, LGALS9B, KYNU, AKR1B10, CCL3L3, CTB-33018.1, IL21-AS1, AKR1B10, VCAN, LCN2, VNN3, RP11-502H18.2, ADAMDEC1, KYNU, IGFL1, CXCL13, KLK6, CXCL8, OASL, KRT16, LCN2, S100A12, IL36G, KRT16, FCN1, CHI3L2, TCN1, SERPINB3, LCE3D, HPSE, GDA, SPRR2C, MYH13, CNGB1, CXCL13, C10orf99, KLK6, IGFL1, SPRR2E, MIR31HG, RPL26P34, EGR4, MMP1, LINC01398, IFNG, S100A9, UGT1A8, AAK1, KLK6, HPSE, SPRR1A, SPRR1A, GZMB, IL26, SERPINB13, IL36G, IFIT1, LCE3D, C10orf99, KCNK10, IFNWP19, RP1-52J10.9, CXCL1, RHCG, KYNU, KYNU, KRT16, KYNU, SPRR2C, MX2, PLAC8, XAF1, LCN2, HYAL4, CHI3L2, PAPL, CXCL8, HPSE, SHD, FABP5, ZC3H12A, REN, SERPINB13, LINC00402, IFI27, NLRP7, AC004870.5, IRF7, LTF, DSC2, LINC01405, LRRC55, CD177, RHCG, CHI3L2, IGFL1, CLEC7A, SPRR2A, HPSE, CXCL13, IL21, KLK9, SERPINB13, TNIP3, S100A9, HPSE, KLK6, S100A12, LAMP3, RTP4, TCN1, KYNU, KLK6, TCN1, CXCL9, KYNU, SERPINB3, SERPINB3, EHBP1L1, ATP12A, SPRR1B, CD177, MTNR1A, S100A8, OASL, OAS2, KLK6, FABP5P1, IFNE, PRPF19, IGFL1, KYNU, ARSF, LINC01215, DSC2, KLK13, CXCL1, LCE3D, LCN2, CXCL1, CXCL13, IL36G, KLK6, C10orf99, C10orf99, CXCL8, LCN2, S100A12, S100A12, S100A12, SERPINB3, IFI44, SECTM1, CHI3L2, GDA, TMPRSS11D, S100A8, KYNU, KYNU, SPRR2C, KLK6, RHCG, CYP24A1, CXCL1, SPRR2A, KLK13, LCE3D, KRT6A, TYMP, FOSL1, AC092117.1, HYAL4, RP11-428L9.2, KLK13, IFI27, CTA-384D8.34, UGT1A10, SLC6A14, TMPRSS11D, RHCG, CTSC, AKR1B10, EREG, GZMB, CXCL9, HPSE, SPRR2C, GDA, IL36G, TNIP3, SPRR2A, SLC6A14, LCN2, SERPINB13, LCN2, SLAMF8, ACE2, TAP2, RGS1, LILRB2, SERPINB3, CLEC4A, TMPRSS11D, KLK6, AKR1B10, IL36G, ATP12A, TEX101.

In some embodiments, the composition comprises at least two agents, compounds, or drugs of the present invention or their analogs, derivatives, and isomers.

In some embodiments, the composition of the present invention comprises a methoxyflavone, a dimethoxyflavone, a trimethoxyflavone, or a tetramethoxyflavone.

In some embodiments, the composition of the present invention comprises a derivative of a methoxyflavone, of a dimethoxflavone, of a trimethoxyflavone, or of a tetramethoxyflavone.

In some embodiments, the composition of the present invention comprises a VEGF inhibitor.

In some embodiments, a D vitamin or a B vitamin is added to any of the compositions, manufacture, products, processes, methods, and/or methods of use, prevention and treatment described herein.

In some embodiments, an ATF4 modulator (see Roybal et al. 2005 and WO/2009/020601) and/or FST1 modulator is added to any of the compositions, manufacture, products, processes, methods, and/or methods of use, prevention and treatment described herein.

In some embodiments, an NRF2 and/or KEAP1 modulator is added to any of the compositions, manufacture, products, processes, methods, and/or methods of use, prevention and treatment described herein.

In some embodiments, the compositions, manufacture, products, processes, methods, and/or methods of use, prevention and treatment comprise an ATF4 modulator.

In some embodiments, the compositions, manufacture, products, processes, methods, and/or methods of use, prevention and treatment comprise an NRF2 and/or KEAP1 modulator.

In some embodiments, the compositions, manufacture, products, processes, methods, and/or methods of use, prevention and treatment of the present invention comprise at least two compounds and agents selected from agents, compounds and drugs of the present invention (e.g. at least one extract selected from the group comprising orange, frankincense, cannabis or other natural oil or extract) or at least one substance or compound isolated from said at least one extract and other natural oils and/or extracts, a sesquiterpene, a sesquiterpenoid, a sesquiterpene lactone (e.g. lactucin, lactuopicrin, 8-deoxylactucin, picriside A, crepidiaside A, jacquinellin, jacquinellin glycoside, chamissonolide, helenalin, alantolactone, dehydrocostus lactone, costunolide), a sesquiterpene sulfate, reduced glutathione, auraptene, ethacrynic acid, curcumin, a curcuminoid, hispolon, dehydroxyhispolon, methoxyhispolon, bisdemethylcurcumin, hispolon methyl ether, hydroxyhispolon, methoxyhispolon methyl ether, a triterpenoid (e.g. Betulinic acid), zingerone, reservatrol, vanillin, rosmarinic acid, a methoxyflavone, a sesquiperetene, n-acetylcysteine, trimethylglycine, folinic acid, folic acid, an amino acid, an FST1 modulator, NRF2 modulator, KEAP1 modulatorflavone, a flavonoid, quercetin, a shogaol (e.g. 6-shogaol), a gingerol (e.g. 6-gingerol), zingerol, kavalactone, sulforaphane, allyl-, butyl- and phenylethyl-isothiocyanate, chlorophyllin, alpha-lipoic acid, allicin, plumbagin, protandim, capsaicin, a capsaicinoid, piperine, asafetida, eugenol, piperlongumine, pellitorine, zingiberine, tBHQ, CDDO-Im, MC-LR, epigallocatechin-3-gallate, a compound found in wasabi, cafestol, xanthohumol, 5-O-caffeoylquinic acid, N-methylpyridinium, resevratrol, caffeic acid phenethyl ester, 3-O-Caffeoyl-1-methylquinic acid, silymarin, kahweol, garlic organosulfur compounds, lycopene, carnosol (rosemany), an avicin, oltipraz, CDDO, a neurite outgrowth promoting prostaglandin, vitamin D, a B vitamin, andrographolide, an amino acid, s-allylcysteine, Vitamin A, Vitamin C, Vitamin E, 3 carotene, trans-2-hexenal, cyclopentenone, ajoene, Dihydro-CDDO-trifluoroethyl amide, Hypochlorous acid, Fragrant unsaturated aldehydes (e.g. trans-cinnamaldehyde, safranal, 2,4-octadienal, citral, and trans-2,cis-6-nonadienal), 2-OHE, 4-OHE, bucillamine, acrolein, momordin, momordol, momordicin I, momordicin II, momordicosides, momordicin-28, momordicinin, momordicilin, momordenol, momorcharin, cucurbitacin B, charantin, charantosides, goyaglycosides, α-eleostearic acid, 15,16-dihydroxy-α-eleostearic acid, antirheumatic gold(I) compounds, an avicin, dithiolethione, an approved drug, and/or a compound, agent or drug extracted from bitter gourd, cloves, black pepper, red chili, ginger, garlic, onion, fennel, bay leaves, nutmeg, saffron coriander, cinnamon (e.g. cinnamic aldehyde), an ATF4 modulator, an FST1 modulator, an NRF2 modulator or a KEAP1 modulator.

In some embodiments, the compounds, agent, or drugs selected for inclusion in the compounds and compositions of the present invention are provided in doses of appropriate to achieve a plasma concentration of between 2 and 10 uM (xanthohumol, lycopene), of between 10 and 150 uM (at least one extract selected from the group comprising orange, frankincense, cannabis or other natural oil or extract or at least one substance or compound isolated from said at least one oil or extract and other natural oils and/or extracts), a sesquiterpene, a sesquiterpenoid, a sesquiterpene lactone (e.g. lactucin, lactuopicrin, 8-deoxylactucin, picriside A, crepidiaside A, jacquinellin, jacquinellin glycoside, chamissonolide, helenalin, alantolactone, dehydrocostus lactone, costunolide), a sesquiterpene sulfate, sulforaphane, curcumin, ethacrynic acid, epigallocatechin-3-gallate, resveratrol, nootkatone, piperine, cafestol, carnosol, cinnamaldehyde, quercetin, chalcone, chlorophyllin), and of between 100 and 200 uM (sulforaphane, s-allylcysteine, piperine; capsaicin, carnosol, cinnamaldehyde, and 3-O-Caffeoyl-1-methylquinic acid).

In some embodiments, the at least one compound, agent, or drug selected for inclusion in the compounds and compositions of the present invention are provided as conjugates (amino acid/protein conjugates, LHRH conjugates, bovine serum albumin (BSA)-conjugate, and non-protein conjugates) derived according to methods known to the art (e.g. WO/2010/033580; WO/2010/057503; WO/2010/013224; WO2007098504; Ploemen, et al., 1993; 1994; Awashti et al., 2000; Lo et al., 2007; Pinnen et al., 2007; Ehrlich et al., 2007; More and Vince, 2008; 2010; Cacciatore et al., 2010).

In some other embodiments, the at least one extract selected from the group comprising orange, frankincense, cannabis or other natural oil or extract or at least one substance or compound isolated from said at least one oil or extract and other natural oils and/or extracts and/or at least one other agent, compound, or drug of the present invention is coupled or conjugated to glutathione according methods effective to produce such coupling or conjugation (e.g. WO2007098504).

In some embodiments, compositions, manufacture, products, processes, methods, and/or methods of use, prevention and treatment comprising at least one extract selected from the group comprising orange, frankincense, cannabis or other natural oil or extract or at least one substance or compound isolated from said at least one oil or extract and other natural oils and/or extracts and/or at least one other agent, compound, or drug of the present invention further comprise zingerone.

In some embodiments, compositions, manufacture, products, processes, methods, and/or methods of use, prevention and treatment comprising at least one extract selected from the group comprising orange, frankincense, cannabis or other natural oil or extract or at least one substance or compound isolated from said at least one oil or extract and other natural oils and/or extracts and/or at least one other agent, compound, or drug of the present invention further comprise zingerone at doses of between 100 to 600 mg/kg.

In some embodiments, the agents, compounds and drugs of the present invention are biotinylated (e.g. U.S. Pat. Nos. 4,794,082; 5,521,319).

In some embodiments, the compositions, manufacture, products, processes, methods, and/or methods of use, prevention and treatment of the present invention provide at least one extract selected from the group comprising orange, frankincense, cannabis or other natural oil or extract or at least one substance or compound isolated from said at least one oil or extract and other natural oils and/or extracts and/or at least one other agent, compound, or drug of the present invention in combination with at least one of any of the agents, compounds, or drugs named herein.

In some embodiments, the sesquiterpene lactone(s) of the present invention is a pseudoguaianolide, a xanthanolide, an eudesmanolide, a germacranolide, an elemanolides, an ambrosanolide, a seco-ambrosanolide, a seco-eudesmanolide, a helenanolide, a eremophilanolide, a bakkenolide, an elemanolide, a cadinanolide, a chrymoranolide, a guaianolides and/or a pseudogual inolide.

Additional chemical definitions, chemical structures, and formulae for compounds and compound classes useful in the present invention, as well as exemplary compositions, are further described in patent applications WO/2005/065667 as well other the other patent applications and articles referenced herein. They are all incorporated herein in their entirety.

In some embodiments, the non-approved agent(s), compound(s) or drug(s) modulate enzymes in the NRF2 pathway.

In addition to being used as a monotherapy, the therapeutic methods of the present invention will also find use in combination therapies.

In some embodiments, the composition described herein comprises nationally approved agents, compounds or drugs listed herein and/or approved agents, compounds or drugs belonging to the drug classes represented by Abilify (aripiprazole), ABREVA (docosanol), Accolate, Accretropin (somatropin rDNA Original), Aciphex (rabeprazole sodium), Actemra (tocilizumab), Actiq, Activella (Estradiol/Norethindrone Acetate) Tablets, Actonel, ACTOplus met (pioglitazone hydrochloride and metformin hydrochloride), ACTOS, Acular (ketorolac tromethamine ophthalmic solution) 0.5%, Acular (ketorolac tromethamine ophthalmic solution) 0.5%, Acuvail (ketorolac tromethamine), Acyclovir Capsules, Adcirca (tadalafil), Adderall (mixed salts of a single-entity amphetamine), Adderall XR, Advicor (extended-release niacin/lovastatin), Afinitor (everolimus), Agenerase (amprenavir), Aggrenox, Agrylin (anagrelide HCL), Agrylin (anagrelide HCL), AK-Con-A (naphazoline ophthalmic), Akten (lidocaine hydrochloride), Alamast, Albenza (albendazole), Aldara (imiquimod), Aldurazyme (laronidase), Alesse (100 mcg levonorgestrel/20 mcg ethinyl estradiol tablets), Alimta (pemetrexed for injection), Alinia (nitazoxanide), Allegra (fexofenadine hydrochloride), Allegra-D, Alora, Aloxi (palonosetron), Alphagan (brimonidine), AlphaNine SD Coagulation Factor IX (Human), Alrex, Altabax (retapamulin), Altocor (lovastatin) Extended-Release Tablets, Alvesco (ciclesonide), Amaryl (Glimepiride), Amerge, Amevive (alefacept), Amitiza (lubiprostone), Amoxil (amoxicillin), Ampyra (dalfampridine), Amrix (cyclobenzaprine hydrochloride extended release), Androderm (Testosterone Transdermal System), AndroGel testosterone gel, AneuVysion Assay, Anexsia, Angiomax (bivalirudin), Antizol Injection, Anzemet, Anzemet, Aphthasol, Aplenzin (bupropion hydrobromide), Apokyn (apomorphine hydrochloride), Apthasol (Amlexanox), Aptivus (tipranavir), Aptivus (tipranavir), Arava, Aredia (pamidronate disodium for injection), Arestin (minocycline hydrochloride), Argatroban Injection, ARICEPT (donepezil hydrochloride), Arimidex (anastrozole), Arixtra, Aromasin Tablets, Arranon (nelarabine), Arthrotec, Arzerra (ofatumumab), Asacol (mesalamine), Astelin nasal spray, Astepro (azelastine hydrochloride nasal spray), Atacand (candesartan cilexetil), Atacand (candesartan cilexetil), Atacand (candesartan cilexetil), Atracurium Besylate Injection, Atridox, Atridox, Atrovent (ipratropium bromide), Atryn (antithrombin recombinant lyophilized powder for reconstitution), Augmentin (amoxicillin/clavulanate), Avandamet (rosiglitazone maleate and metformin HCl), Avandia (rosiglitazone maleate), Avastin (bevacizumab), Avastin (bevacizumab), Avelox I.V. (moxifloxacin hydrochloride), Avinza (morphine sulfate), Avita Gel, Avita Gel, Avonex (Interferon Beta 1-A), Axert (almotriptan malate) tablets, Axid AR (nizatidine, Axona (caprylidene), AzaSite (azithromycin), Azmacort (triamcinolone acetonide) Inhalation Aerosol, Azor (amlodipine besylate; olmesartan medoxomil), Azulfidine EN-tabs Tablets (sulfasalazine delayed release tablets, USP), Bactroban Cream, Bactroban Nasal 2% (mupirocin calcium ointment), Banzel (rufinamide), Baraclude (entecavir), Baycol (cerivastatin sodium), Bayer Extra Strength Asprin, BeneFIX (coagulation Factor IX (recombinant)), BeneFIX (coagulation Factor IX (recombinant)), Benicar, Benzamycin (erythromycin 3%-benzoyl peroxide 5% topical gel), Bepreve (bepotastine besilate ophthalmic solution), Berinert (C1 Esterase Inhibitor (Human)), Besivance (besifloxacin ophthalmic suspension), Betapace AF Tablet, Betaxon, Bextra, Bexxar, Biaxin XL (clarithromycin extended-release tablets), BiDil (isosorbide dinitrate/hydralazine hydrochloride), Boniva (ibandronate), Botox (onabotulinumtoxinA), Botox (onabotulinumtoxinA), Botox Cosmetic (botulinum toxin type A), Bravelle (urofollitropin for injection, purified), Breathe Right, Bromfenac, Brovana (arformoterol tartrate), BSS Sterile Irrigating Solution, Busulflex, Byetta (exenatide), Caduet (amlodipine/atorvastatin), Cafcit Injection, Cambia (diclofenac potassium for oral solution), Campath, Campostar, Campral (acamprosate calcium), Camptosar, Canasa (mesalamine), Cancidas, Captopril and hydrochlorotiazide, Captopril and hydrochlorotiazide, Carbaglu (carglumic acid), Carbatrol, Cardizem® (Diltiazem HCl for injection) Monvial®, Carrington patch, Caverject (alprostadil), Cayston (aztreonam for inhalation solution), CEA-Scan, Cedax (ceftibuten), Cefazolin and Dextrose USP, Ceftin (cefuroxime axetil), Celexa, CellCept, Cenestin, Cenestin, Cernevit, Cervarix [Human Papillomavirus Bivalent (Types 16 and 18) Vaccine, Recombinant, Cetrotide, Chantix (varenicline), Children's Advil (pediatric ibuprofen), Children's Motrin Cold, Chloraprep (chlorhexidine gluconate), Cialis (tadalafil), Cimetadine Hydrochloride Oral Solution 300 mg/5 ml, Cimetidine Hydrochloride Oral Solution, Cimetidine Hydrochloride Oral Solution, Cimzia (certolizumab pegol), Cimzia (certolizumab pegol), Cinryze (C1 Inhibitor (Human)), Cipro (ciprofloxacin HCl), Cipro (ciprofloxacin HCl), Cipro (ciprofloxacin) I.V. and Cipro (ciprofloxacin HCl) tablets, Clarinex, Clarithromycin (Biaxin), Claritin RediTabs (10 mg loratadine rapidly-disintegrating tablet), Claritin Syrup (loratadine), Claritin-D 24 Hour Extended Release Tablets (10 mg loratadine, 240 mg, pseudoephedrine sulfate), Clemastine fumarate syrup, Cleocin (clindamycin phosphate), Cleocin (clindamycin phosphate), Cleviprex (clevidipine), Climara, Clindamycin phosphate topical gel, Clindamycin Phosphate Topical Solution USP 1%, Clolar (clofarabine), Clomipramine hydrochloride, Clonazepam, Coartem (artemether/lumefantrine), Colazal (balsalazide disodium), Colcrys (colchicine), Combivir, Comtan, Concerta, Condylox Gel 0.5% (pokofilox), Confide, Copaxone, Corlopam, Corvert Injection (ibutilide fumarate injection), Cosopt, Covera-HS (verapamil), Crestor (rosuvastatin calcium), Crinone 8% (progesterone gel), Crixivan (Indinavir sulfate), Curosurf, Cuvposa (glycopyrrolate), Cycloset, bromocriptine mesylate, Cylert, Cymbalta (duloxetine), Dacogen (decitabine), Daptacel, Degarelix (degarelix for injection), DentiPatch (lidocaine transoral delivery system), Depakote (divalproex sodium), Depakote (divalproex sodium), Depakote ER (divalproex sodium), Dermagraft-TC, Desmopressin Acetate (DDAVP), Desmopressin Acetate (DDAVP), Desonate (desonide), Detrol (tolterodine tartrate), Detrol LA (tolterodine tartrate), Differin (adapalene gel) Gel, 0.1%, Diltiazem HCL, Extended-Release Capsules, Diovan (valsartan), Diovan (valsartan), Diovan HCT (valsartan), Ditropan XL (oxybutynin chloride), Ditropan XL (oxybutynin chloride), Doribax (doripenem), Dostinex Tablets (cabergoline tablets), Doxil (doxorubicin HCl liposome injection), Droxia; Dulera (mometasone furoate+formoterol fumarate dihydrate), DuoNeb (albuterol sulfate and ipratropium bromide), Durezol (difluprednate), dutasteride, Dynabac, DynaCirc CR, EDEX, Edluar (zolpidem tartrate), Effexor (venlafaxin HCL), Effexor XR (venlafaxin HCl), Efient (prasugrel), Egrifta (tesamorelin for injection), Elaprase (idursulfase), Elestrin (estradiol gel), Elidel, Eligard (leuprolide acetate), Elitek (rasburicase), ella (ulipristal acetate), Ellence, Elliotts B Solution (buffered intrathecal electrolyte/dextrose injection), Elmiron (pentosan polysulfate sodium), Eloxatin (oxaliplatin/5-fluorouracil/leucovorin), Embeda (morphine sulfate and naltrexone hydrochloride), Emend (aprepitant), Enbrel (etanercept), Entereg (alvimopan), Entocort EC (budesonide), Epivir (lamivudine), Epivir (lamivudine), Epogen, Eraxis (anidulafungin), Erbitux (cetuximab), Esclim, Estradiol tablets, Estradiol tablets, Estradiol Transdermal System, Estratab (0.3 mg), EstroGel (estradiol gel 0.06%), Estrostep (norethindrone acetate and ethinyl estradiol), Estrostep (norethindrone acetate and ethinyl estradiol), Estrostep (norethindrone acetate and ethinyl estradiol), Ethyol (amifostine), Ethyol (amifostine), Etodolac, Etodolac, Etodolac, Eulexin (flutamide), Evamist (estradiol), Evista (raloxifene hydrochloride), Evista (raloxifene hydrochloride), Evista (raloxifene hydrochloride), Evoxac, Exalgo (hydromorphone hydrochloride) extended release, Excedrin Migraine, Exelon (rivastigmine tartrate), Exelon (rivastigmine tartrate), Extavia (Interferon beta-I b), Extina (ketoconazole), Fabrazyme (agalsidase beta), Famvir (famciclovir), Famvir (famciclovir), Fanapt (iloperidone), Faslodex (fulvestrant), Femara (letrozole), Femara (letrozole), Femhrt Tablets, FemPatch, Femstat 3 (butoconazole nitrate 2%), FEMSTAT One, Fenofibrate, Feraheme (ferumoxytol), Feridex I.V., Ferrlecit, Fertinex (urofollitropin for injection, purified), Finacea (azelaic acid) Gel, 15%, Finevin, Flagyl ER, FLOMAX, Flonase Nasal Spray, Flovent Rotadisk, Floxin otic, Floxin Tablets (ofloxacin tablets), FluMist (Influenza Virus Vaccine), Fluzone Preservative-free, Focalin (dexmethylphenidate HCl), Follistim™ (follitropin beta for injection), Folotyn (pralatrexate injection), Foradil Aerolizer (formoterol fumarate inhalation powder), Forteo (teriparatide), Fortovase, Fosamax (alendronate sodium), Fosrenol, lanthanum carbonate, Fragmin, Frova (frovatriptan succinate), Fusilev (levoleucovorin), Fuzeon (enfuvirtide), Galzin (zinc acetate), Gardasil (quadrivalent human papillomavirus (types 6, 11, 16, 18), recombinant vaccine), Gastrocrom Oral Concentrate (cromolyn sodium), GastroMARK, Gelnique (oxybutynin chloride), Gemzar (gemcitabine HCL), Gemzar (gemcitabine HCL), Generic Transdermal Nicotine Patch, Genotropin (somatropin) injection, Genotropin (somatropin) lyophilized powder, Geodon (ziprasidone mesylate), Geref (sermorelin acetate for injection), Gilenya (fingolimod), Gleevec (imatinib mesylate), Gleevec (imatinib mesylate), Gliadel Wafer (polifeprosan 20 with carmustine implant), Glipizide Tablets, Glucagon, Glucagon, Glyburide Tablets, Glyburide Tablets, Glyburide Tablets, Glyset (miglitol), Gonal-F (follitropin alfa for injection), Halaven (eribulin mesylate), Havrix, Hectorol (Doxercalciferol) Injection, Hepsera (adefovir dipivoxil), Herceptin, Herceptin (trastuzumab), Hiberix (Haemophilus b Conjugate Vaccine; Tetanus Toxoid Conjugate), Humalog (insulin lispro), Humatrope (somatropin [rDNA origin] for injection), Humira (adalimumab), Hycamtin (topotecan hydrochloride), Hycamtin (topotecan hydrochloride), Lamin, Ilaris (canakinumab), Imagent (perflexane lipid microspheres), Imitrex (sumatriptan) injection and tablets, Imitrex (sumatriptan) nasal spray, Increlex (mecasermin), INFANRIX (Diphtheria and Tetanus Toxoids and Acellular Pertussis, Vaccine Adsorbed), Infasurf, INFERGEN (interferon alfacon-1), Inform HER-2/neu breast cancer test, Innohep (tinzaparin sodium) injectable, Inspra (eplerenone tablets), Integrilin, Intelence (etravirine), Interstim Continence Control Therapy, Intron A (Interferon alfa-2b, recombinant), Intron A (interferon alfa-2b, recombinant), Intron A (interferon alfa-2b, recombinant), Intuniv (guanfacine extended-release), Invanz, Invega (paliperidone), Invirase (saquinavir), Iontocaine, Iressa (gefitinib), Isentress (raltegravir), Istodax (romidepsin), IvyBlock, Ixempra (ixabepilone), Ixiaro (Japanese Encephalitis Vaccine, Inactivated, Adsorbed), Jalyn (dutasteride+tamsulosin), Januvia (sitagliptin phosphate), Jevtana (cabazitaxel), Kadian, Kalbitor (ecallantide), Kaletra Capsules and Oral Solution, Kapvay (clonidine hydrochloride), Keppra, Ketek (telithromycin), Ketoprofen, Kineret, Klaron (sodium sulfacet amide lotion) Lotion, 10%, Kogenate FS (Antihemophilic Factor Recombinant), Krystexxa (pegloticase), Kuvan (sapropterin dihydrochloride), Kytril (granisetron) solution, Kytril (granisetron) tablets, Lamictal (lamotrigine) Chewable Dispersible Tablets, Lamictal Chewable Dispersible Tablets, Lamisil (terbinafine hydrochloride) Dermagel, 1%, Lamisil (terbinafine hydrochloride) Solution, 1%, Lamisil (terbinafine hydrochloride) Tablets, Lamisil Solution, 1%, Lantus (insulin glargine [rDNA origin] injection), Lantus (insulin glargine [rDNA origin] injection), Latuda (lurasidone), Lescol (fluvastatin sodium), Lescol (fluvastatin sodium) capsules, Rx, Lescol XL (fluvastatin sodium) tablet, extended release, Letairis (ambrisentan), Leukine (sargramostim), Leukine (sargramostim), Levaquin, Levitra (vardenafil), Levo-T (levothyroxine sodium), Levoxyl, Lexapro (escitalopram oxalate), Lexiva (fosamprenavir calcium), Lexxel (enalapril maleate-felodipine ER), Lidoderm Patch (lidocainepatch 5%), Lithobid (Lithium Carbonate), Livalo (pitavastatin), Lodine (etodolac), Lodine XL (etodolac), Lodine XL (etodolac), Lotemax, Lotrisone (clotrimazole/betamethasone diproprionate) lotion, Lotronex (alosetron HCL) Tablets, Lovenox (enoxaparin sodium) Injection, Lovenox (enoxaparin sodium) Injection, Lovenox (enoxaparin sodium) Injection, Lucentis (ranibizumab), Lumigan (bimatoprost ophthalmic solution), Lunesta (eszopiclone), Lupron Depot (leuprolide acetate for depot suspension), Lupron Depot (leuprolide acetate for depot suspension), Lupron Depot (leuprolide acetate for depot suspension), Lusedra (fospropofol disodium), Lustra, LUVOX (fluvoxamine maleate), Luxiq (betamethasone valerate) Foam, Lyrica (pregabalin), Lysteda (tranexamic acid), Macugen (pegaptanib), Malarone (atovaquone; proguanil hydrochloride) Tablet, Marplan Tablets, Mavik (trandolapril), Maxalt, Mentax (1% butenafine HCl cream), Mentax (1% butenafine HCl cream), Mentax (1% butenafine HCl cream), Menveo (meningitis vaccine), MERIDIA, Merrem I.V. (meropenem), Mesnex, Metadate CD, Metaglip (glipizide/metformin HCl), Metaprotereol Sulfate Inhalation Solution, 5%, Metozolv ODT (metoclopramide hydrochloride), MetroLotion, Mevacor (lovastatin) tablets, Miacalcin (calcitonin-salmon) Nasal Spray, Micardis (telmisartan), Micardis HCT (telmisartan and hydrochlorothiazide), Microzide (hydrochlorothiazide), Migranal, Minoxidil Topical Solution 2% for Women, Miraluma test, Mirapex, Mircera (methoxy polyethylene glycol-epoetin beta), Mircette, Mirena (levonorgestrel-releasing intrauterine system), Mobic (meloxicam) Tablets, Monistat 3 (miconazole nitrate), Monistat 3 (miconazole nitrate), Monurol, Moxatag (amoxicillin), Mozobil (plerixafor injection), Multaq (dronedarone), Muse, Mylotarg (gemtuzumab ozogamicin), Myobloc, Myozyme (alglucosidase alfa), Naglazyme (galsulfase), Naltrexone Hydrochloride Tablets, Namenda (memantine HCl), Naprelan (naproxen sodium), Nasacort AQ (triamcinolone acetonide) Nasal Spray, Nasacort AQ (triamcinolone acetonide) Nasal Spray, NasalCrom Nasal Spray, Nascobal Gel (Cyanocobalamin, USP), Nasonex Nasal Spray, Natazia (estradiol valerate+dienogest), Natrecor (nesiritide), Neulasta, Neumega, Neupogen, Neupro (rotigotine), Neurontin (gabapentin), Neurontin (gabapentin) oral solution, Neurontin (gabapentin) oral solution, Nexavar (sorafenib), Nexium (esomeprazole magnesium), Niaspan, NicoDerm CQ, Nicorette (nicotine polacrilex), Nicotrol nasal spray, Nicotrol transdermal patch, Nitrostat (nitroglycerin) Tablets, Nolvadex, NORCO tablets (Hydrocodone Bitartrate/Acetaminophen 10 mg/325 mg), Norditropin (somatropin (rDNA origin) for injection), Noritate, Normiflo, Norvir (ritonavir), Norvir (ritonavir), Novantrone (mitoxantrone hydrochloride), NovoLog (insulin aspart), Novolog Mix 70/30, Novothyrox (levothyroxine sodium), Noxafil (posaconazole), Nplate (romiplostim), Nuedexta (dextromethorphan hydrobromide and quinidine sulfate), Nutropin (somatropin-rDNA origin), Nutropin (somatropin-rDNA origin), NuvaRing, Nuvigil (armodafinil), Ocuflox (ofloxacin opthalmic solution) 0.3%, OcuHist, Oleptro (trazodone hydrochloride), Omnicef, Onglyza (saxagliptin), Onsolis (fentanyl buccal), Oral Cytovene, Oravig (miconazole), Orencia (abatacept), Orencia (abatacept), Orfadin (nitisinone), Ortho Evra, Ortho Tri-Cyclen Tablets (norgestimate/ethinyl estradiol), Ortho-Prefest, OsmoCyte Pillow Wound Dressing, Ovidrel (gonadotropin, chorionic human recombinant), Oxycodone and Aspirin, Oxycodone with Acetaminophen 5 mg/325 mg, OxyContin (oxycodone HCl controlled-release), Oxytrol (oxybutynin transdermal system), Ozurdex (dexamethasone), Pancreaze (pancrelipase), Panretin Gel, Patanase (olopatadine hydrochloride), Paxil (paroxetine hydrochloride), Paxil CR (paroxetine hydrochloride), Paxil CR (paroxetine hydrochloride), Pediarix Vaccine, Peg-Intron (peginterferon alfa-2b), Pegasys (peginterferon alfa-2a), Pennsaid (diclofenac sodium topical solution), Pentoxifylline, Pepcid Complete, Periostat (doxycycline hyclate), Periostat (doxycycline hyclate), PhosLo, Photodynamic Therapy, Photofrin, Pindolol, Plavix (clopidogrel bisulfate), Plavix (clopidogrel bisulfate), Plenaxis (abarelix for injectable suspension), Posicor, Pradaxa (dabigatran etexilate mesylate), Pramipexole, Prandin, Pravachol (pravastatin sodium), Pravachol (pravastatin sodium), Precose (acarbose), Premarin (conjugated estrogens), Prempro, Prempro & Premphase (conjugated estrogens/medroxyprogesterone acetate tablets), PREVACID(R) (lansopraxole), PREVEN; Emergency Contraceptive Kit, Prevnar 13 (Pneumococcal 13-valent Conjugate Vaccine), Prevpac, Prevpac, Prezista (darunavir), Priftin, Prilosec (omeprazole), Prilosec (omeprazole), Prilosec (omeprazole), Prilosec (omeprazole)/Biaxin (clarithromycin) Combination Therapy, Prinivil or Zestril (Lisinopril), ProAmatine (midodrine), Procanbid (procainamide hydrochloride extended-release tablets), Prochloroperazine, Prochlorperazine, Prograf, Proleukin, Prolia (denosumab), Promacta (eltrombopag), Prometrium, Prometrium, Propecia, Proscar, Protonix (pantoprazole sodium) Delayed Release Tablets, Protonix (pantoprazole sodium) Delayed-Release Tablets, Protonix (pantoprazole sodium) Intravenous composition, Protopic (tacrolimus) ointment, Provenge (sipuleucel-T), Proventil HFA Inhalation Aerosol, Prozac Weekly (fluoxetine HCl), Pulmozyme (dornase alfa), Pulmozyme (dornase alfa), Quadramet (Samarium Sm 153 Lexidronam Injection), Quixin (levofloxacin), Qutenza (capsaicin), Qvar (beclomethasone dipropionate), Ranexa (ranolazine), Ranitidine Capsules, Ranitidine Tablets, Rapamune (sirolimus) oral solution, Rapamune (sirolimus) Tablets, Raplon, Raxar (grepafloxacin), Rebetol (ribavirin), REBETRON™ Combination Therapy, Rebif (interferon beta-1a), Reclast (zoledronic acid), Reclast (zoledronic acid), Redux (dexfenfluramine hydrochloride), Refludan, REGRANEX (becaplermin) Gel, Relenza, Relpax (eletriptan hydrobromide), Remeron (Mirtazapine), Remeron SolTab (mirtazapine), Remicade (infliximab), Remicade (infliximab), Reminyl (galantamine hydrobromide), Remodulin (treprostinil), Renagel (sevelamer hydrochloride), Renagel (sevelamer hydrochloride), RenaGelRenagel (sevelamer hydrochloride), Renova (tretinoin emollient cream), Renvela (sevelamer carbonate), ReoPro, REPRONEX (menotropins for injection, USP), Requip (ropinirole hydrochloride), Rescriptor Tablets (delavirdine mesylate tablets), Rescula (unoprostone isopropyl ophthalmic solution) 0.15%, RespiGam (Respiratory Syncitial Virus Immune Globulin Intravenous), Restasis (cyclosporine ophthalmic emulsion), Retavase (reteplase), Retin-A Micro (tretinoin gel) microsphere, 0.1%, Revlimid (lenalidomide), Reyataz (atazanavir sulfate), Rhinocort Aqua Nasal Spray, Rid Mousse, Rilutek (riluzole), Risperdal Oral composition, Ritalin LA (methylphenidate HCl), Rituxan, Rocephin, Rocephin, Rotarix (Rotavirus Vaccine, Live, Oral), Rotateq (rotavirus vaccine, live oral pentavalent), Rozerem (ramelteon), Rythmol, Sabril (vigabatrin), Saizen, Salagen Tablets, Samsca (tolvaptan), Sanctura (trospium chloride), Sancuso (granisetron), Saphris (asenapine), Savella (milnacipran hydrochloride), Sclerosol Intrapleural Aerosol, Seasonale, Lo Seasonale, Seasonique (ethinylestradiol+levonorgestrel), SecreFlo (secretin), Selegiline tablets, Self-examination breast pad, Selzentry (maraviroc), Sensipar (cinacalcet), Seprafilm, Serevent, Seroquel® (quetiapine fumarate) Tablets, Silenor (doxepin), Simponi (golimumab), Simulect, Singulair, Skelid (tiludronate disodium), Skin Exposure Reduction Paste Against Chemical Warfare Agents (SERPACWA), Soliris (eculizumab), Somatuline Depot (lanreotide acetate), Somavert (pegvisomant), Sonata, Spectracef, Spiriva HandiHaler (tiotropium bromide), SPORANOX (itraconazole), Sprix (ketorolac tromethamine), Sprycel (dasatinib), Stavzor (valproic acid delayed release), Stelara (ustekinumab), Strattera (atomoxetine HCl), Stromectol (ivermectin), Subutex/Suboxone (buprenorphine/naloxone), Sulfamylon, Supartz, Supprelin LA (histrelin acetate), Sustiva, Sutent (sunitinib), Symlin (pramlintide), Synagis, Synercid I.V., Synthroid (levothyroxine sodium), Synvisc, Synvisc-One (Hylan GF 20), Tamiflu capsule, Tarceva (erlotinib, OSI 774), Tasigna (nilotinib hydrochloride monohydrate), Tasmar, Tavist (clemastine fumarate), Tavist (clemastine fumarate), Taxol, Taxotere (Docetaxel), Tazorac topical gel, Teczem (enalapril maleate/diltiazem malate), Teflaro (ceftaroline fosamil), Tegretol (carbamazepine), Tegretol XR (carbamazepine), Tekamlo (aliskiren+amlodipine), Tekturna (aliskiren), Temodar, Tequin, Testim, Testoderm TTS CIII, Teveten (eprosartan mesylate plus hydrochlorothiazide), Teveten (eprosartan mesylate), Thalomid, Tiazac (diltiazem hydrochloride), Tiazac (diltiazem hydrochloride), Tiazac (diltiazem hydrochloride), Tikosyn Capsules, Tilade (nedocromil sodium), Tilade (nedocromil sodium), Tilade (nedocromil sodium), Timentin, Timentin, Tindamax, tinidazole, Tobi, Tolmetin Sodium, Topamax (topiramate), Topamax (topiramate), Toprol-XL (metoprolol succinate), Torisel (temsirolimus), Toviaz (fesoterodine fumarate), Tracleer (bosentan), Travatan (travoprost ophthalmic solution), Trazadone 150 mg, Treanda (bendamustine hydrochloride), Trelstar Depot (triptorelin pamoate), Trelstar LA (triptorelin pamoate), Tri-Nasal Spray (triamcinolone acetonide spray), Tribenzor (olmesartan medoxomil+amlodipine+hydrochlorothiazide), Tricor (fenofibrate), Tricor (fenofibrate), Trileptal (oxcarbazepine) Tablets, Trilipix (fenofibric acid), Tripedia (Diptheria and Tetanus Toxoids and Acellular Pertussis, Vaccine Absorbed), Trisenox (arsenic trioxide), Trivagizole 3 (clotrimazole) Vaginal Cream, Trivora-21 and Trivora-28, Trizivir (abacavir sulfate; lamivudine; zidovudine AZT) Tablet, Trovan, Twinrix, Tygacil (tigecycline), Tykerb (lapatinib), Tysabri (natalizumab), Tysabri (natalizumab), Tyvaso (treprostinil), Tyzeka (telbivudine), Uloric (febuxostat), Ultracet (acetaminophen and tramadol HCl), UltraJect, UroXatral (alfuzosin HCl extended-release tablets), Urso, UVADEX Sterile Solution, Valcyte (valganciclovir HCl), Valstar, Valtrex (valacyclovir HCl), Vancenase AQ 84 mcg Double Strength, Vanceril 84 mcg Double Strength (beclomethasone dipropionate, 84 mcg), Inhalation Aerosol, Vaprisol (conivaptan), Vectibix (panitumumab), Velcade (bortezomib), Veltin (clindamycin phosphate and tretinoin), Venofer (iron sucrose injection), Ventolin HFA (albuterol sulfate inhalation aerosol), Veramyst (fluticasone furoate), Verapamil, Verdeso (desonide), Veregen (kunecatechins), VERSED (midazolam HCl), Vesicare (solifenacin succinate), Vfend (voriconazole), Viadur (leuprolide acetate implant), Viagra, Vibativ (telavancin), Victoza (liraglutide), Vidaza (azacitidine), Videx (didanosine), Vimovo (naproxen+esomeprazole), Vimpat (lacosamide), Vioxx (rofecoxib), VIRACEPT (nelfinavir mesylate), Viramune (nevirapine), Viread (tenofovir disoproxil fumarate), Viread (tenofovir disoproxil fumarate), Viroptic, Visicol Tablet, Visipaque (iodixanol), Vistide (cidofovir), Vistide (cidofovir), Visudyne (verteporfin for injection), Vitrasert Implant, Vitravene Injection, Vivelle (estradiol transdermal system), Vivelle (estradiol transdermal system), Vivelle-Dot (estradiol transdermal system), Vivitrol (naltrexone for extended-release injectable suspension), Vivitrol (naltrexone for extended-release injectable suspension), Votrient (pazopanib), Vpriv (velaglucerase alfa for injection), Vyvanse (Lisdexamfetamine Dimesylate), Warfarin Sodium tablets, Welchol (colesevelam hydrochloride), Western blot confirmatory device, Wilate (von Willebrand Factor/Coagulation Factor VIII Complex (Human), Xeloda, Xeloda, Xenazine (tetrabenazine), Xenical/Orlistat Capsules, Xeomin (incobotulinumtoxinA), Xgeva (denosumab), Xiaflex (collagenase clostridium histolyticum), Xifaxan (rifaximin), Xifaxan (rifaximin), Xigris (drotrecogin alfa [activated]), Xolair (omalizumab), Xopenex, Xyrem (sodium oxybate), Xyzal (levocetirizine dihydrochloride), Yasmin (drospirenone/ethinyl estradiol), ZADITOR, Zagam (sparfloxacin) tablets, Zanaflex (tizanidine hydrochloride), Zantac 75 Efferdose, Zelnorm (tegaserod maleate) Tablets, Zelnorm (tegaserod maleate) Tablets, Zemaira (alpha1-proteinase inhibitor), Zemplar, Zenapax, Zenpep (pancrelipase), Zerit (stavudine), Zerit (stavudine), Zevalin (ibritumomab tiuxetan), Ziprasidone (ziprasidone hydrochloride), Zipsor (diclofenac potassium), Zirgan (ganciclovir ophthalmic gel), Zithromax (azithromycin), Zocor, Zofran, Zofran, Zoladex (10.8 mg goserelin acetate implant), Zoloft (sertraline HCl), Zoloft (sertraline HCl), Zoloft (sertraline HCl), Zometa (zoledronic acid), Zometa (zoledronic acid), Zomig (zolmitriptan), Zomig (zolmitriptan), Zonegran (zonisamide) Capsules, Zortress (everolimus), Zosyn (sterile piperacillin sodium/tazobactam sodium), Zuplenz (ondansetron oral soluble film), Zyban Sustained-Release Tablets, Zyclara (imiquimod), Zyflo (Zileuton), Zymaxid (gatifloxacin ophthalmic solution), Zyprexa, and Zyrtec (cetirizine HCl). In such instances, the approved drug(s) may typically be provided at or about the same dosage as usually prescribed for a particular indication, although lower or higher dosages may be desirable depending upon the clinical picture.

In some embodiments, the compositions or drug combinations of the present invention comprise one or more erythropoietin-like agent selected from erythropoietin, Darbepoetin (Aranesp), Epocept (Lupin pharma), Epogen, Epogin, Eprex, Procrit, NeoRecormon, Recormon, Methoxy polyethylene glycol-epoetin beta (Mircera), Dynepo, Epomax, Silapo (Stada), Retacrit, Epocept, EPOTrust, Erypro Safe, Repoitin, Vintor, Epofit, Erykine, Wepox, Espogen, ReliPoietin, Shanpoietin, Zyrop, or EPIAO (rHuEPO).

In some embodiments, one or more agents, compounds and drugs of the present invention are selected from the list comprising at least one extract selected from the group comprising orange, frankincense, cannabis or other natural oil or extract or at least one substance or compound isolated from said at least one oil or extract and other natural oils and/or extracts, sesquiterpene, a sesquiterpenoid, a sesquiterpene lactone (e.g. lactucin, lactuopicrin, 8-deoxylactucin, picriside A, crepidiaside A, jacquinellin, jacquinellin glycoside, chamissonolide, helenalin, alantolactone, dehydrocostus lactone, costunolide), a sesquiterpene sulfate, reduced glutathione, auraptene, ethacrynic acid, curcumin, a curcuminoid, hispolon, dehydroxyhispolon, methoxyhispolon, bisdemethylcurcumin, hispolon methyl ether, hydroxyhispolon, methoxyhispolon methyl ether, a triterpenoid (e.g. Betulinic acid), zingerone, reservatrol, vanillin, rosmarinic acid, a methoxyflavone, a sesquiperetene, n-acetylcysteine, trimethylglycine, folinic acid, folic acid, an amino acid, an FST1 modulator, NRF2 modulator, KEAP1 modulatorflavone, a flavonoid, quercetin, a shogaol (e.g. 6-shogaol), a gingerol (e.g. 6-gingerol), zingerol, kavalactone, sulforaphane, allyl-, butyl- and phenylethyl-isothiocyanate, chlorophyllin, alpha-lipoic acid, allicin, plumbagin, protandim, capsaicin, a capsaicinoid, piperine, asafetida, eugenol, piperlongumine, pellitorine, zingiberine, tBHQ, CDDO-lm, MC-LR, epigallocatechin-3-gallate, a compound found in wasabi, cafestol, xanthohumol, 5-O-caffeoylquinic acid, N-methylpyridinium, resveratrol, nootkatone, caffeic acid phenethyl ester, 3-O-Caffeoyl-1-methylquinic acid, silymarin, kahweol, garlic organosulfur compounds, lycopene, carnosol (rosemany), an avicin, oltipraz, CDDO, a neurite outgrowth promoting prostaglandin, vitamin D, a B vitamin, andrographolide, an amino acid, s-allylcysteine, Vitamin A, Vitamin C, Vitamin E, carotene, trans-2-hexenal, cyclopentenone, ajoene, Dihydro-CDDO-trifluoroethyl amide, Hypochlorous acid, Fragrant unsaturated aldehydes (e.g. trans-cinnamaldehyde, safranal, 2,4-octadienal, citral, and trans-2,cis-6-nonadienal), 2-OHE, 4-OHE, bucillamine, acrolein, momordin, momordol, momordicin I, momordicin II, momordicosides, momordicin-28, momordicinin, momordicilin, momordenol, momorcharin, cucurbitacin B, charantin, charantosides, goyaglycosides, α-eleostearic acid, 15,16-dihydroxy-α-eleostearic acid, antirheumatic gold(I) compounds, an avicin, dithiolethione, an approved drug, and/or a compound, agent or drug extracted from cloves, black pepper, red chili, ginger, garlic, onion, fennel, bay leaves, nutmeg, saffron coriander and cinnamon (e.g. cinnamic aldehyde) is combined with one or more approved drugs of the drug classes exemplified herein or listed above. In such instances, an accompanying approved agent(s), compound(s) or drug(s) may typically be provided at or about the same dosage as usually prescribed for a particular indication, although lower or higher dosages may be desirable depending upon the clinical picture.

Typically, the resulting novel combination will be useful in preventing or treating a condition, disease or disorder for which the approved drug is considered to be approved for use.

The agents, compounds, and drugs of the present invention (including their analogs and derivatives) may be administered before or after the other agent in intervals ranging from seconds to weeks. In embodiments where the other approved therapy and the agents, compounds, and drugs of the present invention are applied separately to the cell, one would generally ensure that a sufficient amount of time did not pass such that the agent and the agents, compounds, and drugs of the present invention would still be able to exert an advantageous, combined effect.

Approved therapies include drug therapies, immunotherapy, gene therapy, radiotherapy, chemotherapy, surgery, etc.

In one embodiment, daily doses of the compounds do not exceed 20 mg iron, 400 mcg of folic acid, 1600 mcg of folinic acid, 2000 mg of trimethylglycine, 3000 mg N-acetylcysteine, and the roughly bioequivalent dose of reduced glutathione, a glutathione precursor, or a known intracellular-glutathione promoting agent.

In some embodiments, the compound(s) or drug(s) of the present invention are administered with ascorbic acid.

In some embodiments, the compositions, manufacture, products, processes, methods, and/or methods of use, prevention and treatment of the present invention comprise 5,7 dimethoxyflavone and/or 6,3-dimethxoyflavone.

In some embodiments, the compositions, manufacture, products, processes, methods, and/or methods of use, prevention and treatment of the present invention comprise zingerone, a shogaol, and/or a gingerol.

In some embodiments, the compositions, manufacture, products, processes, methods, and/or methods of use, prevention and treatment of the present invention comprise a compound, agent, or drug extracted from ginger, curcumin, a methoxyflavone, vitamin C, n-acetylcysteine, trimethylglycine, folinic acid, folic acid and/or reduced glutathione.

In some embodiments, the compositions, manufacture, products, processes, methods, and/or methods of use, prevention and treatment of the present invention comprise an amino acid other than phenylalanine.

In some embodiments, combining at least one extract selected from the group comprising orange, frankincense, cannabis or other natural oil or extract or at least one substance or compound isolated from said at least one oil or extract and other natural oils and/or extracts and/or at least one other agent, compound, or drug of the present invention with an approved drug will allow the skilled clinician to reduce the amount of an approved drug required to achieve clinical benefits, while simultaneously reducing the risk or severity of side effects associated with the treatment or prevention protocol.

In some embodiments, combining at least one extract selected from the group comprising orange, frankincense, cannabis or other natural oil or extract or at least one substance or compound isolated from said at least one oil or extract and other natural oils and/or extracts and/or at least one other named agent, compound, or drug of the present invention with an approved drug will allow the skilled clinician to increase the amount of an approved drug provided to a patient to achieve greater benefit from that approved drug, while simultaneously reducing the risk or severity of side effects associated with the treatment or prevention protocol. With respect to the approved drugs provided within compositions, manufacture, products, processes, methods, and/or methods of use, prevention and treatment of the present invention, the amount that will be effective in the treatment of a particular disorder or condition disclosed herein will depend on the nature of the disorder or condition, and can be determined by clinical techniques and in vitro or in vivo assays.

The precise dose of a drug to be employed will also depend on the route of administration, and should be decided according to the judgment of the practitioner and each patient's circumstances. However, suitable dosage ranges for oral administration are generally about 0.001 mg to about 1000 mg of an approved drug of the invention, or a pharmaceutically acceptable salt or complex thereof, per kg body weight/day. A bioequivalent amount of the approved drug will typically be provided by routes other than the oral route, is such a route of delivery is selected.

In some embodiments, the dose of the at least one extract selected from the group comprising orange, frankincense, cannabis or other natural oil or extract or at least one substance or compound isolated from said at least one oil or extract and other natural oils and/or extracts and/or at least one other agent, compound, or drug of the present invention in such compositions varies from 0.5 mg/kg to 500 mg/kg.

In some embodiments, the dose of at least one extract selected from the group comprising orange, frankincense, cannabis or other natural oil or extract or at least one substance or compound isolated from said at least one oil or extract and other natural oils and/or extracts and/or at least one other agent, compound, or drug of the present invention in such compositions varies from 5 mg/kg to 200 mg/kg.

In part, the invention relates to a method for preventing chemotherapeutic resistance (including cancer chemotherapeutic resistance). The method comprises administering a composition comprising at least one extract selected from the group comprising orange, frankincense, cannabis or other natural oil or extract or at least one substance or compound isolated from said at least one oil or extract and other natural oils and/or extracts and/or at least other agent, compound, or drug of the present invention.

In part, the invention relates to a method for preventing chemotherapeutic resistance (including cancer chemotherapeutic resistance). The method comprises administering a composition comprising at least one agent, compound, or drug of the present invention (such as those named herein).

In some embodiments, the composition(s) and combination(s) of the present invention comprise metronidazole and itraconazole to treat a protozoal disease with synergistic efficacy.

In some embodiments, an additional agent(s), compound(s) or drug(s) of the present invention, as well as metronidazole, is combined with itraconazole to treat a protozoal disease.

In some embodiments, the composition(s) and combination(s) of the present invention comprise metronidazole, ciprofloxacin and itraconazole.

In some embodiments, the composition(s) and combination(s) of the present invention comprise metronidazole, ciprofloxacin and itraconazole.

In some embodiments, the composition(s) and combination(s) of the present invention comprise metronidazole, itraconazole and a non-approved drug named herein.

In some embodiments, the composition(s) and combination(s) of the present invention comprise metronidazole, itraconazole and a sesquiterpene.

In some embodiments, metronidazole, ciprofloxacin are combined with itraconazole to treat a protozoal disease with synergistic efficacy.

In some embodiments, the drug compositions for preventing or treating a protozoal disease comprise agents from the drug classes represented by metronidazole and itraconazole and/or additional agent(s), compounds(s), or drug(s) of the present invention.

In some embodiments, the composition or method for treating or preventing a parasitic illness comprises: one or more triazoles selected from Itraconazole, Fluconazole, Isavuconazole, Ravuconazole, Posaconazole, Voriconazole and Terconazole; one or more nitroimidazoles selected from Metronidazole, Tinidazole, Nitroimidazole, Azanidazole, Secnidazole, Ornidazole, Propenidazole, and Nimorazole; and one or more agents, compounds, or drugs described herein.

In further embodiments, the composition or method for treating or preventing a parasitic illness comprises one or more triazoles selected from Itraconazole, Fluconazole, Isavuconazole, Ravuconazole, Posaconazole, Voriconazole and Terconazole; one or more nitroimidazoles selected from Metronidazole, Tinidazole, Nitroimidazole, Azanidazole, Secnidazole, Ornidazole, Propenidazole, and Nimorazole; mefloquine, doxycycline, choroquine, hydroxychoroquine, Malarone, atovaquone, Proguanil (Malarone), an artemisinin-based compound, antimony, amphotericin, miltefosine, paromomycin, and one or more agents, compounds, or drugs described herein.

In one preferred embodiment the composition takes the form of solution, liquid, gel, suspension, emulsion, lotion, tablet, pill, pellet, capsule, powder, sustained-release formulation, suppository, emulsion, aerosol, spray, drop, nanoemulsion, buccal or sublingual form, a transdermal patch or other form suitable for use, such as cosmetic cream, body lotion, body milk, ointment or shampoo.

The present compositions, manufacture, products, processes, methods, and/or methods of use, prevention and treatment therefore, can take the form of solutions, liquids (e.g. WO2010106191), gels (e.g. WO2007126915), suspensions, emulsion, tablets, pills, pellets, capsules, capsules containing liquids (e.g. WO2010106191), powders (US20040162273), sustained-release suppositories, emulsions, aerosols, sprays (e.g. WO/2010/109482), drops, suspensions, nanoemulsions (e.g. WO2010070675), sublingual compositions (e.g. WO2009067536), a transdermal patch (e.g. U.S. Pat. Nos. 5,004,610; 5,342,623; 5,344,656; 5,364,630; 5,462,745; and 5,508,038; 5,077,104; 5,268,209; 4,908,027; 5,633,008; 4,839,174; 4,943,435; and 5,167,242) or any other form suitable for use.

Pharmaceutical compositions containing the at least one agent, compound, or drug of the present invention may be prepared in any form, such as oral dosage form (powder, tablet, capsule, soft capsule, aqueous medicine (e.g. U.S. Pat. No. 6,068,850), syrup, elixirs pill, powder, sachet, granule), or topical preparation (cream, ointment, lotion, gel, emulgel (e.g. WO2007129162), balm, patch, paste, spray solution, aerosol and the like), or injectable preparation (solution, suspension, emulsion).

The compositions, manufacture, products, processes, methods, and/or methods of use, prevention and treatment of the invention relate to preventing the effects of aging and cell death induced by UV radiation. The invention also relates to the use of these compositions as tan extenders.

The compositions of the invention can be in the form of cosmetic creams, gels, lotions, milks, emulsions and solutions, ointments, sprays, oils, body lotions, shampoos, lotions after-shave, deodorants, soaps, lip sticks protectors, sticks and pencils for makeup.

The compositions of the present invention may comprise flavorings (e.g. extract of ginger, mint, strawberry, vanilla, etc).

In some embodiments, the compositions, manufacture, products, processes, methods, and/or methods of use, prevention and treatment of the present invention are the product of mixing the compounds and drugs in their wet or liquid forms.

In some embodiments, the compositions, manufacture, products, processes, methods, and/or methods of use, prevention and treatment of the present invention are the product of mixing the compounds and drugs in their wet or liquid forms, and subsequently preparing solutions, suspensions, emulsion, tablets, pills, pellets, capsules containing liquids (e.g. WO2010106191), powders, sustained-release suppositories, emulsions, aerosols, sprays.

In some embodiments, the compositions, manufacture, products, processes, methods, and/or methods of use, prevention and treatment of the present invention are the product of mixing the compounds and drugs in their dry or solid forms.

In the form of a gel, the compositions, manufacture, products, processes, methods, and/or methods of use, prevention and treatment include suitable excipients such as cellulose esters or other gelling agents such as carbopol, guar gum, etc.

The compositions in pharmaceutical dosage forms may be used in the form of their pharmaceutically acceptable salts and complexes, and also may be used alone or in appropriate association, as well as in combination with other pharmaceutically active compounds.

According to the present invention a buccal or sublingual dosage form may be also be a “T”- or “L”-shaped solid dosage form. Perforated and fenestrated versions, as well as non-perforated and non fenestrated versions of this dosage form are also possible.

The dimpled or fenestrated dosage form may appear somewhat similarly when viewed from the lateral aspect, fenestrated or dimpled dosage forms have much the same appearance and the fenestrations or dimples may themselves be of various shapes. Perforated and fenestrated dosage forms permit flow of saliva through the dosage form, as well as around it, speeding dissolution.

In some embodiments, the posterior portion of the buccal dosage form will be smaller than the anterior portion, thereby better conforming to the shape of the mouth. Also, the anterior and posterior portions may, in some embodiments, be rounded or angles to conform more comfortably to the buccal cavity of the user.

In some embodiments, the portion of the “T” or “L” shaped dosage form abutting the cheek is rounded to produce a semilunar shape to the portion occupying the space between the gum(s) and cheek when viewed in the anterior-posterior plane, so as to approximate the curvature of the cheek. However, when viewed from anteriorly or posteriorly, the fenestrated dosage form will typically have a flattened appearance while the dimpled dosage form may be flat or semilunar. The fenestrated form may be associated with a perpendicular or nearly perpendicular bite surface member to achieve either an “L” or “T-dosage form”.

The dosage asymmetric forms may be considered and prepared as left sided and right sided versions to be selected according to user preference or as directed by a qualified physician or other clinician.

The dosage forms could be produced either using molds and mold technology known to the arts, or using extrusion methods known to those skilled in the art, wherein the extruded materials comprises the “T” or “L” shape that can be easily cut or chopped into appropriate lengths then, in some embodiments, stamped and further cut to produces ridges and the rounded or angled edges.

The exact dimensions of each portion of the “T”-shaped or “L” shaped dosage forms can be varied to the size of the user and the dose to be delivered, as can the proportion of drug/agent to other materials in the formulation. Examples of such “T”-shaped or “L” shaped dosage forms include, but are not limited to troches, gels, tablets, and other dosage form types amenable to such use.

In one preferred embodiment the composition takes the form of nanoparticles, nanovaults, nanotubes, nanofibers, etc. and/or liposomes, micelles, other lipid based carriers, etc.

In one preferred embodiment the composition is a gum, stable liquid, troche, tablet, capsule, gummy, sports drink, sublingual composition, condiment, nutritional drink, polyethylene glycol-coated preparation, suspension, syrup, soft gel, topical formulation, nanoemulsion, nanoparticle preparation, food mixture, powder mixture, topical gel, sunscreen, lozenge, cream, aqueous formulation, injectable formulation.

The present invention includes any compositions known to the art that is suitable for administration of the agents, drugs, and compositions useful in the methods of the present invention. Examples include tablets (U.S. Pat. No. 4,209,513), capsules (e.g. US 2010/0021535; U.S. Pat. No. 7,011,846), such as gelatin capsules (e.g. U.S. Pat. No. 5,698,155), pills, troches (e.g. U.S. Pat. No. 3,312,594), elixirs, suspensions, syrups (e.g. U.S. Pat. No. 6,790,837), wafers (e.g. Wen and Park, 2010), chewing gum (e.g. Chaudhary and Shahiwala, 2010; Semwal et al. 2010); U.S. Pat. No. 6,531,114; Surana et al, 2010), etc.

In specific some embodiments, the oral dose of an approved drug is about 0.01 mg to about 100 mg/kg body weight/day, and more preferably about 0.1 mg to about 75 mg/kg body weight/day, and more preferably about 0.5 mg to 5 mg/kg body weight/day.

In some embodiments, the compositions, manufacture, products, processes, methods, and/or methods of use, prevention and treatment of the present invention utilize a soft gel capsule (U.S. Pat. Nos. 2,780,355, 4,497,157, 4,777,048, 4,780,316, 5,037,698 and 5,376,381).

In some embodiments, the compositions, manufacture, products, processes, methods, and/or methods of use, prevention and treatment of the present invention are the product of mixing compounds, agents and drugs in their dry or solid forms and subsequently encapsulating those compounds, agents and drugs in a capsule for oral administration.

In some embodiments, the compositions, manufacture, products, processes, methods, and/or methods of use, prevention and treatment of the present invention are the product of mixing compounds, agents and drugs in their dry or solid forms and subsequently suspending those compounds, agents and drugs in a suspension.

In some embodiments, the compositions, manufacture, products, processes, methods, and/or methods of use, prevention and treatment of the present invention are the product of mixing compounds, agents and drugs in their dry or solid forms and subsequently preparing solutions, suspensions, emulsion, tablets, pills, pellets, capsules, capsules containing liquids, powders, sustained-release suppositories, emulsions, aerosols, sprays.

In some embodiments, the compositions, manufacture, products, processes, methods, and/or methods of use, prevention and treatment of the present invention are the product of mixing compounds, agents and drugs in their dry or solid forms, preparing tablets, pills, pellets, capsules, capsules, etc. and subsequently coating those compounds, agents and drugs with an enteric coating.

In some embodiments, at least one extract selected from the group comprising orange, frankincense, cannabis or other natural oil or extract or at least one substance or compound isolated from said at least one oil or extract and other natural oils and/or extracts and/or other at least one agent, compound, or drug of the present invention is provided in 250 mg, 500 mg, or 1000 mg doses at a frequency suitable to maintain a desirable plasma concentration.

In some embodiments, at least one extract selected from the group comprising orange, frankincense, cannabis or other natural oil or extract or at least one substance or compound isolated from said at least one oil or extract and other natural oils and/or extracts and/or other at least one agent, compound, or drug of the present invention is provided at a dosage suitable to achieve a plasma concentration of between 1 and 1000 uM.

In some embodiments, at least one extract selected from the group comprising orange, frankincense, cannabis or other natural oil or extract or at least one substance or compound isolated from said at least one oil or extract and other natural oils and/or extracts and/or other at least one agent, compound, or drug of the present invention is provided at a dosage suitable to achieve a plasma concentration of between 5 and 500 uM.

In some embodiments, at least one extract selected from the group comprising orange, frankincense, cannabis or other natural oil or extract or at least one substance or compound isolated from said at least one oil or extract and other natural oils and/or extracts and/or other at least one agent, compound, or drug of the present invention is provided at a dosage suitable to achieve a plasma concentration of between 15 and 100 uM.

In one preferred embodiment the composition is a functional food.

The compositions, manufacture, products, processes, methods, and/or methods of use, prevention and treatment described herein also allow for the production of a “functional health food” comprising the agent(s), compound(s), or drug(s) of the present invention for the prevention and improvement of a condition, disease, or disorder in a subject.

The term “a functional health food” defined herein is the functional food providing enhanced physical; psychological, physiological, or other functionality by adding the compositions, agent(s), compound(s), drug(s), analogs, derivatives, or compositions of the present invention to conventional food for the benefit of a human or mammal.

In one preferred embodiment the composition further comprises at least one of omega-3 fatty acids, olive oil, or other source of lipid.

In some embodiments, at least one agent, compound, or drug of the present invention further comprises at least one omega 3 fatty acids, olive oil, or other source of lipid.

In some embodiments, one or more agents, compounds, or drugs of the present invention is conveyed to the body in conjunction with omega-3 fatty acids.

In some embodiments, one or more agents, compounds, or drugs of the present invention is conveyed to the body in conjunction with omega-3 fatty acids together in a capsule.

It will be apparent to those skilled in the art that various modifications and variations can be made in the compositions, use and preparations of the present invention without departing from the spirit or scope of the invention.

In some embodiments, the agents, compounds, or drugs of the present invention are modified chemically using novel means as well as any means known to the art (e.g. Brandi et al., 2003; Kassouf et al., 2006; Chao et al., 2007; Cho et al., 2007; Weng et al., 2007; Lin et al., 2008; U.S. Pat. No. 6,974,801).

In one preferred embodiment the method comprises topical or systemic administration of said composition for the treatment of a disease associated with mir-21 and/or other oncogene expression in a subject in need thereof.

Such compositions, manufacture, products, processes, methods, and/or methods of use, prevention and treatment can be administered to a subject animal such as mammals (rat, mouse, domestic animals or human) via various routes. All modes of administration are contemplated (e.g. orally, rectally or by intravenous, intramuscular, subcutaneous, intra-cutaneous, intrathecal, epidural or intra-cerebroventricular injection).

In some embodiments, said method comprises orally, parenterally, sublingually or topically, or by various routes simultaneously administration of said composition or combination of the present invention With respect to the present invention, an agent, compound, or drug may, for example, be administered orally, parenterally (e.g. intravenously, intramuscularly, subcutaneously), intranasally, topically (e.g. WO/2009/153373; WO/2010/070675; WO2007126915), or transdermally (e.g. Cevc and Blume, 2001). Topical compositions include, for example, emulsions, gels, and sunscreens (e.g. WO2010129213; WO2007001484; WO2006099687). The CTFA Cosmetic Ingredient Handbook, Seventh Edition, 1997 and the Eighth Edition, 2000 (both incorporated by reference herein in their entirety) describe a wide variety of cosmetic and pharmaceutical ingredients suitable for use in the compositions of the present invention.

Examples of these functional classes disclosed in this reference include: absorbents, skin protectants, abrasives, anticaking agents, antifoaming agents, antioxidants, binders, biological additives, buffering agents, bulking agents, chelating agents, chemical additives, colorants, cosmetic astringents, cosmetic biocides, denaturants, drug astringents, external analgesics, film formers, fragrance components, humectants, opacifying agents, pH adjusters, plasticizers, SPF boosters, reducing agents, skin bleaching agents, skin-conditioning agents (emollient, humectants, miscellaneous, and occlusive), solvents, foam boosters, hydrotropes, solubilizing agents, suspending agents (nonsurfactant), sunscreen agents, ultraviolet light absorbers, waterproofing agents, and viscosity increasing agents (WO2010129213).

Other routes of administration include rectal administration, intrathecal administration, administration involving mucosal absorption, and administration in aerosolized form (e.g. U.S. Pat. Nos. 5,126,123; 5,544,646).

The present invention covers the administration of compositions, manufacture, products, processes, methods, and/or methods of use, prevention and treatment useful in the methods of the invention to an animal by sustained release. Such administration is selected when it is considered beneficial to achieve a certain level of the drug in a body compartment over a longer period of time (e.g. serum or plasma concentration).

In some embodiments, the compositions, manufacture, products, processes, methods, and/or methods of use, prevention and treatment of the present invention are suitable for oral administration including extended release compositions (e.g. Pouton, 2000; Prasad et al., 2003; WO/2010/137027; WO/2020/129337; WO/2010/127100; WO/2010/127191; WO/2010/119300; WO/2010/114801; WO/2010/103544). and controlled release compositions (WO 02/083106; U.S. Pat. Nos. 5,567,439; 6,838,094; 6,863,902; 6,905,708).

The present invention calls for the administration of an agent, compound, or drug to a human in an amount effective for achieving its benefit. Typical daily doses of compounds comprising the composition vary approximately in the range of 0.5 mg to 5000 mg. The effective amount of the compound to be administered can be readily determined by those skilled in the art, for example, through pre-clinical trials, clinical trials, and by methods known to scientists, physicians and clinicians.

The present invention covers in vivo methods for the administration of a compound, agent or drug to an animal. These methods may vary and are not limited to those described herein.

Within the pre-clinical and clinical period, any method known to the art may be employed for contacting a cell, organ or tissue with an agent, compound, or drug. Suitable methods include in vitro, ex vivo, or in vivo methods. In vitro methods include cultured samples. For example, a cell can be placed in medium and incubated with a compound, agent or drug under conditions suitable for assaying its activity (especially at least one extract selected from the group comprising orange, frankincense, cannabis or other natural oil or extract or at least one substance or compound isolated from said at least one oil or extract and other natural oils and/or extracts and/or at least one other agent, compound, or drug of the present invention-like activity). Appropriate incubation conditions may be readily determined by those skilled in the art.

An effective amount of a compound, agent or drug useful in the methods of the present invention, may be administered to an animal by known methods. The compound may be administered systemically or topical.

In one preferred embodiment the method comprises administration of said composition in the form of nanoparticles, nanovaults, or liposomes.

In some embodiments, the compounds, drugs or agents of the present invention may be administered to a cell in vitro, ex vivo, or in vivo utilizing nanoparticles (Martins et al., 2009; WO/2010/013224), Such delivery allows for improved absorption and/or pharmacokinetics of the compounds, drugs or medicinal compositions.

In some embodiments, the compounds, drugs or agents of the present invention may be administered to a cell utilizing liposomes, nanoparticles, nanocapsules, nanovaults, etc. (see Goldberg et al., 2007; Li et al., 2007; Martins et al., 2009; Hu et al., 2010; Huang et al., 2010).

In some embodiments, the agents, compounds, drugs of the present invention may be administered to a cell in vitro, ex vivo, or in vivo utilizing nanoparticles, liposomes (WO/2010/009186; WO/2009/141450; WO/2009/065065; WO/2004/069224; WO/1999/013865), nanocapsules, nanovaults.

In some embodiments, the compounds, drugs or medicinal compositions of the present invention may be administered to a cell utilizing liposomes, nanocapsules, nanovaults, nanosuspensions, etc. (see Sholer et al., 2001; Goldberg et al., 2007; Li et al., 2007; Hu et al.; 2010; Huang et al., 2010).

In some embodiments, the compounds, drugs or medicinal compositions of the present invention may be administered using nanovaults engineered to allow cell type specific targeting (Kickhoefer et al. ACS Nano 3, 27-36 (2009)).

In some embodiments, the compounds, drugs or medicinal compositions of the present invention may be administered using recombinant nanovaults.

In some embodiments, the compounds, agents, or drugs of the present invention are incorporated into nanoparticles allowing absorption of orally administered compositions, manufacture, products, processes, methods, and/or methods of use, prevention and treatment increasing bioavailability (especially oral bioavailability).

In some embodiments, a compound, agent or drug of the present invention is incorporated into nanoparticles, liposomes, and/or nanovaults allowing increased bioavailability of the compound, agent or drug.

In some embodiments, the compounds, agents, or drugs of the present invention are loaded into solid lipid nanoparticles by ultrasonic and high-pressure homogenization.

In some embodiments, compounds, agents and drugs of the present invention are loaded into solid lipid nanoparticles by ultrasonic and high-pressure homogenization along with Sodium Carboxymethyl Cellulose.

In some embodiments, the drugs and compounds of the present invention are 44 incorporated into engineered nanomaterials, nanoliposomes, nanoemulsions (e.g. WO2010070675), nanoparticles and nanofibers (Weiss et al., 006; 2007) for further incorporation into all manner of medicinal compositions and food items of all types, including, for example, milkshakes, muffins, hamburgers, fruit cocktails, granola, trail mix, vitamin drinks, sports drinks (U.S. Pat. Nos. 5,780,094; 4,981,687), nutritional supplements and energy drinks (U.S. Pat. No. 5,744,187; etc. (see Handbook of Functional Lipids; and “Food Nanotechnology, an overview” by Sekhon (2010), as well as Milk and Milk Products: Technology, Chemistry and Microbiology by Varnam and Sutherland (2001) for reviews).

In some embodiments, the compositions, manufacture, products, processes, methods, and/or methods of use, prevention and treatment of the present invention comprise a combination of compositions, manufacture, products, processes, methods, and/or methods of use, prevention and treatment named herein.

In some embodiments, the compounds, agents, or drugs of the present invention are loaded into solid lipid nanoparticles by ultrasonic and high-pressure homogenization.

In some embodiments, the compounds, agents, or drugs of the present invention are loaded into solid lipid nanoparticles (e.g. KR1020080014379; WO/2006/102768; WO/2000/006120).

In some embodiments, the compounds, agents, or drugs of the present invention are loaded into solid lipid nanoparticles by ultrasonic and high-pressure homogenization along with Sodium Carboxymethyl Cellulose (Hu et al., 2010).

In some embodiments, the compounds, agents, or drugs of the present invention are encapsulated into solid lipid nanoparticles (SLN) utilizing a double emulsion solvent evaporation (w/o/w) method (Li et al., 2010).

In some embodiments, the compound, agent or drug of the present invention may be delivered in the form of an aqueous solution (e.g. WO/2000/025,765), a lipid, or in a lyophilized form.

In some embodiments, the compositions, manufacture, products, processes, methods, and/or methods of use, prevention and treatment of the present invention are the product of mixing compounds, agents and drugs in their dry or solid forms and subsequently loading those compounds, agents and drugs into lipid.

In some embodiments, the compositions, manufacture, products, processes, methods, and/or methods of use, prevention and treatment of the present invention are the product of mixing compounds, agents and drugs in their dry or solid forms and subsequently loading those compounds, agents and drugs into liposomes (e.g. see Langer, 1990. Science 249:1527-1533; Treat et al, in Liposomes in the Therapy of Infectious Disease and Cancer, Lopez-Berestein and Fidler (eds.), Liss, N.Y., pp. 353-365 (1989); Lopez-Berestein, ibid., pp. 317-327).

In some embodiments, the compositions, manufacture, products, processes, methods, and/or methods of use, prevention and treatment of the present invention are the product of mixing compounds, agents and drugs in their dry or solid forms and subsequently loading those compounds, agents and drugs into solid lipid nanoparticles.

In some embodiments, the compositions, manufacture, products, processes, methods, and/or methods of use, prevention and treatment of the present invention are the product of mixing compounds, agents and drugs in their dry or solid forms followed by loading those compounds, agents and drugs into solid lipid nanoparticles and/or liposomes followed by drying or lyophilizing the mixture.

In some embodiments, the dried or lyophilized liposomes and/or solid lipid nanoparticles are encapsulated for oral administration.

In some embodiments, compounds, agents, or drugs of the present invention are PEGylated by Chemical conjugation with PEG.

In some embodiments, the compounds, agents, or drugs of the present invention are complexed with crystalline ascorbic acid in solid lipid nanoparticles.

In some embodiments, the agents, compounds, or drugs of the present invention are conjugated, coupled, linked or complexed with glutathione (GSH).

In some embodiments, wherein at least one extract selected from the group comprising orange, frankincense, cannabis or other natural oil or extract or at least one substance or compound isolated from said at least one oil or extract and other natural oils and/or extracts and/or at least one other agent, compound, or drug of the present invention is selected for use in the present invention, at least one extract selected from the group comprising orange, frankincense, cannabis or other natural oil or extract or at least one substance or compound isolated from said at least one oil or extract and other natural oils and/or extracts and/or at least one other agent, compound, or drug of the present invention is conjugated, coupled, linked or complexed with glutathione.

In some embodiments, the agents, compounds, or drugs of the present invention are conjugated, coupled, linked or complexed with a nitric oxide (NO)-donor moiety.

In one embodiment, the agents, compounds, or drugs of the present invention are conjugated, coupled, linked or complexed with a nitric oxide (NO)-donor moiety.

Relevant example methods for coupling or conjugating a nitric oxide moiety to an agent, compound, or drug named herein have previously been described (e.g. WO92/01668, WO 95/30641, WO 97/16405; U.S. Pat. No. 5,859,053; WO/2002/011706; WO2010118968).

In some embodiments, an nitric oxide (NO)-donor moiety is conjugated, coupled, linked or complexed with an approved drug named or described herein, at least one extract selected from the group comprising orange, frankincense, cannabis or other natural oil or extract or at least one substance or compound isolated from said at least one oil or extract and other natural oils and/or extracts and/or at least one other agent, compound, or drug of the present invention, glutathione, auraptene, ethacrynic acid, curcumin, a curcuminoid, hispolon, dehydroxyhispolon, methoxyhispolon, bisdemethylcurcumin, hispolon methyl ether, hydroxyhispolon, methoxyhispolon methyl ether, a triterpenoid, zingerone, reservatrol, vanillin, rosmarinic acid, a methoxyflavone, a sesquiperetene, n-acetylcysteine, trimethylglycine, folinic acid, folic acid, an amino acid, an ATF4 modulator, an FST1 modulator, an NRF2 modulator, a KEAP1 modulator, a flavone, a flavonoid, quercetin, a shogaol (e.g. 6-shogaol), a gingerol (e.g. 6-gingerol), zingerol, kavalactone, sulforaphane, allyl-, butyl- and phenylethyl-isothiocyanate, chlorophyllin, alpha-lipoic acid, allicin, plumbagin, protandim, capsaicin, a capsaicinoid, piperine, asafetida, eugenol, piperlongumine, pellitorine, zingiberine, tBHQ, CDDO-lm, MC-LR, epigallocatechin-3-gallate, a compound found in wasabi, cafestol, xanthohumol, 5-O-caffeoylquinic acid, N-methylpyridinium, resveratrol, nootkatone, caffeic acid phenethyl ester, 3-O-Caffeoyl-1-methylquinic acid, silymarin, kahweol, garlic organosulfur compounds, lycopene, carnosol (rosemany), an avicin, oltipraz, CDDO, a neurite outgrowth promoting prostaglandin, vitamin D, a B vitamin, andrographolide, an amino acid, s-allylcysteine, Vitamin A, Vitamin C, Vitamin E, β carotene, trans-2-hexenal, cyclopentenone, ajoene, Dihydro-CDDO-trifluoroethyl amide, Hypochlorous acid, Fragrant unsaturated aldehydes (e.g. trans-cinnamaldehyde, safranal, 2,4-octadienal, citral, and trans-2,cis-6-nonadienal), 2-OHE, 4-OHE, bucillamine, acrolein, momordin, momordol, momordicin I, momordicin II, momordicosides, momordicin-28, momordicinin, momordicilin, momordenol, momorcharin, cucurbitacin B, charantin, charantosides, goyaglycosides, α-eleostearic acid, 15,16-dihydroxy-α-eleostearic acid, antirheumatic gold(I) compounds, an avicin, dithiolethione, an approved drug, and/or a compound, agent or drug extracted from cloves, black pepper, red chili, cinnamon (e.g. cinnamic aldehyde), ginger, garlic, onion, fennel, bay leaves, nutmeg, saffron, coriander an ATF4 modulator, an FST1 modulator, an NRF2 modulator or a KEAP1 modulator.

In some embodiments, the agents, compounds, or drugs of the present invention covalently linked through an aromatic spacer to an NO-releasing moiety (e.g. —ONO2) (Del Soldato et al., 1999; Bratasz et al., 2006).

In some embodiments, wherein at least one extract selected from the group comprising orange, frankincense, cannabis or other natural oil or extract or at least one substance or compound isolated from said at least one oil or extract and other natural oils and/or extracts and/or at least one other agent, compound, or drug of the present invention is selected for use in the present invention, at least one extract selected from the group comprising orange, frankincense, cannabis or other natural oil or extract or at least one substance or compound isolated from said at least one oil or extract and other natural oils and/or extracts and/or at least one other agent, compound, or drug of the present invention is conjugated, coupled, linked or complexed with a nitric oxide-donor moiety.

In some embodiments, wherein oltipraz is selected for use in the present invention, the oltipraz is conjugated, coupled, linked or complexed with a nitric oxide-donor moiety.

In some embodiments, wherein at least one extract selected from the group comprising orange, frankincense, cannabis or other natural oil or extract or at least one substance or compound isolated from said at least one oil or extract and other natural oils and/or extracts and/or at least one other agent, compound, or drug of the present invention is selected for use in the present invention, the at least one extract selected from the group comprising orange, frankincense, cannabis or other natural oil or extract or at least one substance or compound isolated from said at least one oil or extract and other natural oils and/or extracts and/or at least one other agent, compound, or drug of the present invention is covalently linked through an aromatic spacer to an NO-releasing moiety (e.g. —ONO2) (Del Soldato et al., 1999; Bratasz et al., 2006).

In some embodiments, the agents, compounds, or drugs of the present invention are conjugated, coupled, linked or complexed with a nitric oxide-donor moiety as well as with glutathione.

In some embodiments, the agents, compounds and drugs of the present invention are biotinylated, fluorinated, or difluorinated.

In some embodiments, the compositions, manufacture, products, processes, methods, and/or methods of use, prevention and treatment of the present invention are used to incubate the cells of WO2008150814.

In some embodiments, the compositions, manufacture, products, processes, methods, and/or methods of use, prevention and treatment of the present invention are used in combination with the cells, vectors or methods of WO2008150814.

In one preferred embodiment the method comprises administration of said composition by targeted therapeutic approach.

In a third aspect the present invention relates to a method of producing iPS cells that are less prone to malignant transformation due to suppression of miR-21 and other oncogene expression in said cells, wherein said suppression of miR-21 and other oncogene expression comprises cultivating iPS cells with an orange, frankincense, cannabis and/or other natural oil extract.

Production and incubation of iPS cells in the presence of one or more of orange, frankincense, cannabis and/or other natural oil extract (1:100-1:50,000) can be achieved according to methods and cell culture conditions known to those skilled in the art, such as those exemplified by Takahashi et al., 2006; 2007; etc. WO20081508 A2; Yu et al., 2007; Kim et al., 2009; Wu et al., 2009; Rhee et al., 2011. Incubation of mammalian cells in the presence of one or more orange, frankincense, cannabis and/or other natural oil extract (1:100-1:50,000) is associated with 3 to 17 fold reductions in mir-21 and other oncogene expression that are associated with carcinogenesis. Accordingly, the Likewise, incubation of cells in orange, frankincense, cannabis and/or other natural oil extract (1:100-1:50,000) unexpectedly enhances the efficiency of pluripotency induction as mir-21 expression is reduced (see below).

In some embodiments, the production and incubation of iPS cells is further accomplished in the presence of medium comprising deuterium-depleted water (DDW).

In one embodiment, the extract comprises a cannabinoid.

In one embodiment, the extract consists of a cannabinoid.

In one embodiment, the extract comprises a limonene.

In one embodiment, the extract consists of a limonene.

In one embodiment, the extract comprises an alpha pinene.

In one embodiment, the extract consists of an alpha pinene.

In one embodiment, the extract comprises a terpene.

In one embodiment, the extract consists of a terpene.

In other embodiments, the extract comprises a compound described as constituting at least 5% of a natural oil extract.

The data below are Gene array data related to cannabis, frankincense, wild orange, and lavender extract treatment of 3T3 cells, and demonstrate reduction of miR21 in treated 3T3 cells (Tables 1-4).

TABLE 1
Selected Gene Array Data from Cannabis Oil-Treated 3T3 cells

| | Gene | | | fold_change_C |
|---|---|---|---|---|
| Probe Set ID | Symbol | 3T3-CON | 3T3-CO | OvsCon |
| TC17000728.hg.1 | MIR21 | 6.92532 | 2.806117 | −17.37815479 |
| TC01001501.hg.1 | MIR3120 | 9.395077 | 4.523763 | −29.26925257 |
| TC11000631.hg.1 | MALAT1 | 4.048334 | 3.318412 | −1.658549419 |
| TC06001484.hg.1 | MAS1L | 4.572498 | 3.685736 | −1.8490215 |
| TC02001922.hg.1 | RAB1A | 6.538365 | 5.603444 | −1.911785946 |
| TC03000969.hg.1 | SOX2 | 4.906857 | 5.655364 | 1.680053296 |

TABLE 2
Selected Gene Array Data from DMSO Cannabis Extract-Treated 3T3 cells

| Probe Set ID | Gene Symbol | 3T3-CON | 3T3-CE | fold_change_CEvsCon |
|---|---|---|---|---|
| TC05000673.hg.1 | MIR4461 | 4.550079 | 8.317367 | 13.616538 |
| TC14000273.hg.1 | RN7SL2 // RN7SL1 | 9.822859 | 11.98309 | 4.4698642 |
| TC02002861.hg.1 | SNORD20 | 3.25094 | 5.008501 | 3.3812601 |
| TC07001141.hg.1 | LOC100505921 | 3.008192 | 4.756217 | 3.3589842 |
| TC19000827.hg.1 | MIR526B | 2.541619 | 4.195579 | 3.1469625 |
| TC05000734.hg.1 | VTRNA1-1 | 2.956722 | 4.33672 | 2.6026801 |
| TC12000477.hg.1 | OR6C1 | 4.6803 | 6.010515 | 2.5144014 |
| TC05001540.hg.1 | MTRNR2L2 | 4.27511 | 5.570661 | 2.4547073 |
| TC02001930.hg.1 | MIR4778 | 3.115399 | 4.408515 | 2.4505677 |
| TC08000845.hg.1 | SCXA | 5.220664 | 6.447866 | 2.3411251 |
| TC07000964.hg.1 | MIR548F3 | 4.907326 | 6.132639 | 2.3380617 |
| TC09000367.hg.1 | FAM75D3 | 3.548059 | 4.684898 | 2.1989869 |
| TC09000783.hg.1 | MIR4669 | 7.98129 | 9.104883 | 2.1788894 |
| TC06000695.hg.1 | MIR4485 | 5.921429 | 7.039267 | 2.170215 |
| TC21000041.hg.1 | MIR99A | 3.830645 | 4.946096 | 2.1666273 |
| TC04000110.hg.1 | USP17 | 3.266303 | 4.284289 | 2.02509 |
| TC08000107.hg.1 | MIR3926-2 | 6.17768 | 7.192966 | 2.0213036 |
| TC19002637.hg.1 | SRRM5 | 3.109423 | 4.083926 | 1.9649642 |
| TC06004147.hg.1 | OR5V1 | 3.178946 | 4.150453 | 1.9608878 |
| TC01002426.hg.1 | SNORD99 | 6.905677 | 7.876818 | 1.9603904 |
| TC09000366.hg.1 | FAM75D4 | 3.529314 | 4.492832 | 1.9500593 |
| TC08001037.hg.1 | MIR320A | 5.41256 | 6.36142 | 1.9303467 |
| TC01001965.hg.1 | RPS7P5 | 3.104998 | 4.041234 | 1.9135293 |
| TC13000753.hg.1 | POU4F1 | 4.854694 | 5.788876 | 1.9108069 |
| TC15000631.hg.1 | MIR548H4 | 4.457307 | 5.325378 | 1.8252208 |
| TC09000365.hg.1 | FAM75D5 | 3.635556 | 4.487873 | 1.8053981 |
| TC08000416.hg.1 | MIR4470 | 3.801283 | 4.640168 | 1.7886672 |
| TC05001142.hg.1 | MIR4454 | 6.244821 | 7.060274 | 1.7598507 |
| TC07001913.hg.1 | LOC100129148 | 3.399925 | 4.177668 | 1.7144466 |
| TC17001325.hg.1 | MIR3184 | 4.246152 | 5.008083 | 1.6957588 |
| TC13000766.hg.1 | SLITRK1 | 3.968979 | 4.704122 | 1.6645625 |
| TCUn_gl000219000002.hg.1 | LOC283788 | 5.127021 | 5.852768 | 1.6537567 |
| TC10001632.hg.1 | CALHM1 | 4.721365 | 5.443615 | 1.649753 |
| TC01003789.hg.1 | ST13P19 | 3.707287 | 4.424474 | 1.6439735 |
| TC05000854.hg.1 | MIR1303 | 5.482394 | 6.198945 | 1.6432489 |
| TC07001078.hg.1 | MIR339 | 5.17849 | 5.890056 | 1.6375807 |
| TC04000096.hg.1 | USP17 | 3.936101 | 4.640936 | 1.6299582 |
| TC21000276.hg.1 | MIR3156-3 | 3.424891 | 4.123882 | 1.623369 |
| TC01001579.hg.1 | LOC284648 | 4.668182 | 5.356995 | 1.6119567 |
| TC01002656.hg.1 | KTI12 | 3.552426 | 4.23487 | 1.6048562 |
| TC12000416.hg.1 | LOC100509541 | 6.352322 | 7.029487 | 1.5989945 |
| TC17000305.hg.1 | MTRNR2L1 | 4.000413 | 4.675094 | 1.5962438 |
| TC15000072.hg.1 | SNORD116-30 | 5.225821 | 5.89973 | 1.5953898 |
| TC13000594.hg.1 | MIR320D1 | 3.970938 | 4.638028 | 1.5878669 |
| TC6_cox_hap2000247.hg.1 | OR5V1 | 3.496347 | 4.147997 | 1.5709639 |
| TC6_mann_hap4000211.hg.1 | OR5V1 | 3.496347 | 4.147997 | 1.5709639 |
| TC11001275.hg.1 | MIR483 | 7.054221 | 7.699982 | 1.5645644 |
| TC12000851.hg.1 | MIR4497 | 3.99744 | 4.622372 | 1.5421381 |
| TC01003249.hg.1 | LCE3D | 5.963001 | 6.579346 | 1.5329865 |
| TC12001163.hg.1 | APOBEC1 | 3.66153 | 4.277122 | 1.5321866 |
| TC20000047.hg.1 | FTLP3 | 5.549015 | 6.156843 | 1.5239631 |
| TC6_ssto_hap7000214.hg.1 | OR5V1 | 3.530279 | 4.12869 | 1.5140481 |
| TC11000376.hg.1 | LOC221122 | 4.744594 | 4.159111 | −1.500541 |
| TC01003971.hg.1 | LINC00582 | 4.905364 | 4.318703 | −1.501767 |
| TC0X000110.hg.1 | PRDX4 | 4.416517 | 3.829738 | −1.50189 |
| TC17001066.hg.1 | MED31 | 5.845514 | 5.256004 | −1.504736 |
| TC15001832.hg.1 | KIF7 | 4.556356 | 3.965207 | −1.506446 |
| TC0X000283.hg.1 | MIR362 | 4.42761 | 3.836059 | −1.506866 |
| TC16000684.hg.1 | LOC100289580 | 5.12906 | 4.537263 | −1.507123 |
| TC14002249.hg.1 | IGHV3-9 | 4.450374 | 3.858484 | −1.50722 |
| TC02000554.hg.1 | IGKV2D-26 | 5.496436 | 4.903982 | −1.507809 |
| TC11000059.hg.1 | MIR4686 | 6.619994 | 6.027097 | −1.508272 |
| TC06004094.hg.1 | C6orf164 | 4.014333 | 3.420784 | −1.508954 |
| TC06001090.hg.1 | BTF3P10 | 4.191702 | 3.596652 | −1.510525 |
| TC0X001503.hg.1 | MAGEA12 | 4.704114 | 4.105707 | −1.514044 |
| TC03000405.hg.1 | MAGI1-AS1 | 4.121046 | 3.521678 | −1.515053 |
| TC16000061.hg.1 | MIR4516 | 8.462434 | 7.863055 | −1.515064 |
| TC17001731.hg.1 | SUPT4H1 | 6.254887 | 5.655118 | −1.515474 |
| TC16001348.hg.1 | LOC100506183 | 5.117866 | 4.516862 | −1.516772 |
| TC0X001207.hg.1 | PCDH19 | 4.461317 | 3.860111 | −1.516984 |
| TC04000734.hg.1 | MIR4799 | 4.393453 | 3.787511 | −1.521972 |
| TC21000511.hg.1 | MYL6P1 | 8.352276 | 7.745141 | −1.523231 |
| TC17000375.hg.1 | MIR632 | 6.977433 | 6.370017 | −1.523528 |
| TC03002003.hg.1 | TMEM212-AS1 | 5.135472 | 4.527884 | −1.52371 |
| TC22000052.hg.1 | MIR185 | 6.054836 | 5.446895 | −1.524083 |
| TC07000478.hg.1 | SNORA14A | 5.49603 | 4.884725 | −1.52764 |
| TC09001578.hg.1 | PSMB7 | 5.115465 | 4.50301 | −1.528859 |
| TC07000863.hg.1 | MIR4468 | 6.037353 | 5.424823 | −1.528938 |
| TC1_gl000191_random000002.hg.1 | SRSF10 | 5.390841 | 4.778305 | −1.528944 |
| TC0X001279.hg.1 | CAPN6 | 4.562117 | 3.949294 | −1.529249 |
| TC03000120.hg.1 | HMGB1P5 | 6.440309 | 5.822442 | −1.534605 |
| TC0X000144.hg.1 | TAB3-AS1 | 4.920715 | 4.296206 | −1.541686 |
| TC22000477.hg.1 | MIR3198-1 | 5.457746 | 4.833069 | −1.541866 |
| TC19001662.hg.1 | MIR3191 | 6.206716 | 5.578877 | −1.545249 |
| TC10001227.hg.1 | FAM25E | 5.10046 | 4.47102 | −1.546964 |
| TC10001280.hg.1 | FAM25D | 5.10046 | 4.47102 | −1.546964 |
| TC02004977.hg.1 | IGKV2D-30 | 5.128536 | 4.498679 | −1.547412 |
| TC14002277.hg.1 | IGHV4-61 | 5.998764 | 5.368762 | −1.547567 |
| TCO1002332.hg.1 | MIR4253 | 5.177356 | 4.54161 | −1.553741 |
| TC22001447.hg.1 | IGLJ6 | 5.513013 | 4.876783 | −1.554262 |
| TC10000146.hg.1 | MIR4675 | 5.203658 | 4.566187 | −1.5556 |
| TC19001718.hg.1 | MIR150 | 5.015656 | 4.377628 | −1.556201 |
| TC05001181.hg.1 | ZNF622 | 4.341874 | 3.703545 | −1.556525 |
| TC02002739.hg.1 | MIR4776-2 | 4.715297 | 4.075414 | −1.558203 |
| TC03000900.hg.1 | MIR551B | 4.659123 | 4.018653 | −1.558837 |
| TC16000068.hg.1 | MIR3677 | 8.732668 | 8.091662 | −1.559416 |
| TC0Y000135.hg.1 | TTTY16 | 4.149169 | 3.505605 | −1.562184 |
| TC02000521.hg.1 | SNORD94 | 5.756224 | 5.102463 | −1.573264 |
| TC02000386.hg.1 | MEIS1 | 5.342184 | 4.686654 | −1.575195 |
| TC10001727.hg.1 | NKX1-2 | 6.178034 | 5.519317 | −1.578678 |
| TC08001490.hg.1 | LOC100506652 | 4.246839 | 3.587184 | −1.579705 |
| TC05000701.hg.1 | EGR1 | 6.076075 | 5.414505 | −1.581803 |
| TC02004947.hg.1 | IGKV2-29 | 4.542328 | 3.880454 | −1.582136 |
| TC06001129.hg.1 | SYNJ2-IT1 | 5.518583 | 4.854848 | −1.584179 |
| TC15001286.hg.1 | MIR1282 | 7.444377 | 6.775241 | −1.59012 |
| TC05000998.hg.1 | MIR1271 | 5.289784 | 4.620173 | −1.590644 |
| TC0X001426.hg.1 | MIR504 | 4.942764 | 4.266575 | −1.597913 |
| TC10001270.hg.1 | MIR4294 | 6.331278 | 5.653034 | −1.600191 |
| TC22001449.hg.1 | IGLC7 | 5.00244 | 4.320865 | −1.60389 |
| TC02005030.hg.1 | ASPRV1 | 4.401732 | 3.719292 | −1.604852 |
| TC14002247.hg.1 | IGHV3-7 | 4.448695 | 3.76616 | −1.604957 |
| TC11000031.hg.1 | SNORA52 | 6.992956 | 6.310286 | −1.605108 |
| TC02001959.hg.1 | MIR1285-2 | 5.849719 | 5.164115 | −1.608375 |
| TC20000049.hg.1 | LOC728228 | 5.366404 | 4.677855 | −1.611662 |
| TC12002060.hg.1 | TMEM229B | 5.986041 | 5.296348 | −1.61294 |
| TC15000130.hg.1 | LOC100507026 | 6.384228 | 5.690782 | −1.617142 |
| TC11000631.hg.1 | MALAT1 | 4.048334 | 3.352844 | −1.619434 |
| TC19001450.hg.1 | UPK1A-AS1 | 4.469384 | 3.766242 | −1.628047 |
| TC12002007.hg.1 | MIR4472-2 | 7.091542 | 6.370074 | −1.648859 |
| TC03000116.hg.1 | VENTXP7 | 5.061167 | 4.339243 | −1.64938 |
| TC0X000908.hg.1 | MIR23C | 4.656479 | 3.928347 | −1.656493 |
| TC14000418.hg.1 | SRSF5 | 5.784508 | 5.055527 | −1.657468 |
| TC01002803.hg.1 | FUBP1 | 5.0316 | 4.302098 | −1.658067 |
| TC04001565.hg.1 | PCDH18 | 4.138818 | 3.408705 | −1.658769 |
| TC15000670.hg.1 | LOXL1 | 6.682571 | 5.951731 | −1.659605 |
| TC14000919.hg.1 | SNORD8 | 5.6997 | 4.957218 | −1.673052 |
| TC03000436.hg.1 | MIR4444-1 | 7.254175 | 6.502887 | −1.683295 |
| TC17000367.hg.1 | MIR4725 | 8.839814 | 8.086047 | −1.68619 |
| TC03001845.hg.1 | HMGN2P25 | 6.340228 | 5.584965 | −1.687939 |
| TC13000800.hg.1 | LINC00361 | 6.131493 | 5.372622 | −1.692166 |
| TC02000536.hg.1 | MIR4435-1 | 4.229815 | 3.4665 | −1.697386 |
| TC13000192.hg.1 | PSME2P2 | 7.02319 | 6.25856 | −1.698934 |
| TC14000661.hg.1 | MIR370 | 5.515684 | 4.747818 | −1.702749 |
| TC09000199.hg.1 | LOC158376 | 5.082819 | 4.307443 | −1.711636 |
| TC01003491.hg.1 | DPT | 4.531039 | 3.75511 | −1.712292 |
| TC15001664.hg.1 | MIR631 | 5.664675 | 4.88813 | −1.713024 |
| TC11001820.hg.1 | OR5A2 | 5.302281 | 4.521126 | −1.718506 |
| TC01003990.hg.1 | SNORA14B | 5.419961 | 4.636399 | −1.721376 |
| TC12001942.hg.1 | MIR619 | 4.351392 | 3.566768 | −1.722643 |
| TC07001353.hg.1 | SNORA5C | 5.99114 | 5.194278 | −1.737318 |
| TC08000752.hg.1 | MIR1205 | 4.203527 | 3.404663 | −1.739731 |
| TC05001779.hg.1 | LOC100506102 | 4.522556 | 3.704961 | −1.762465 |
| TC21000151.hg.1 | MEMO1P1 | 5.212935 | 4.394856 | −1.763057 |
| TC0X000562.hg.1 | MIR1911 | 6.034199 | 5.211784 | −1.768364 |
| TC02001251.hg.1 | MIR4776-1 | 4.524966 | 3.691679 | −1.78174 |
| TC05000067.hg.1 | SNORD123 | 5.656614 | 4.821428 | −1.784087 |
| TC12000813.hg.1 | MIR3922 | 6.145116 | 5.308115 | −1.786333 |
| TC05000721.hg.1 | IGIP | 4.114312 | 3.266652 | −1.79958 |
| TC16002106.hg.1 | SLC7A5P1 | 6.685352 | 5.837394 | −1.799951 |
| TC11002391.hg.1 | SNORD14E | 6.725938 | 5.874277 | −1.804577 |
| TC17001609.hg.1 | MAPT-AS1 | 6.233504 | 5.378329 | −1.808978 |
| TC20000146.hg.1 | LOC100653248 // LOC100652902 | 6.592179 | 5.736722 | −1.809332 |
| TC04000007.hg.1 | MIR571 | 5.499165 | 4.638078 | −1.816406 |
| TC08001548.hg.1 | EXT1 | 7.248168 | 6.380135 | −1.825173 |
| TC13000052.hg.1 | MIR2276 | 5.436802 | 4.566241 | −1.828374 |
| TC15001577.hg.1 | SCARNA14 | 6.018013 | 5.139254 | −1.838793 |
| TC17_ctg5_hap1000006.hg.1 | MAPT-AS1 | 7.054317 | 6.168624 | −1.847652 |
| TC12001455.hg.1 | SNORA2B | 4.749049 | 3.8623 | −1.849005 |
| TC0X000201.hg.1 | CASK-AS1 | 6.63735 | 5.746821 | −1.853856 |
| TC0X001393.hg.1 | MIR503 | 5.655488 | 4.742947 | −1.882358 |
| TC10000755.hg.1 | MIR146B | 4.405058 | 3.487614 | −1.888766 |
| TC05001498.hg.1 | LOC100506390 | 5.657677 | 4.738353 | −1.891229 |
| TC18000522.hg.1 | SNORA37 | 6.159097 | 5.193272 | −1.95318 |
| TC02000923.hg.1 | ACVR2A | 5.802602 | 4.834877 | −1.955754 |
| TC05001097.hg.1 | MIR4456 | 4.292994 | 3.307941 | −1.979386 |
| TC0X000969.hg.1 | CASK | 6.003073 | 5.015 | −1.983534 |
| TC15001614.hg.1 | RPL29P30 | 4.310699 | 3.291327 | −2.027036 |
| TC20000447.hg.1 | MIR5095 | 6.098718 | 5.070028 | −2.040171 |
| TC09001705.hg.1 | MIR3689C | 4.72253 | 3.691184 | −2.04393 |
| TC04000160.hg.1 | MIR218-1 | 4.198035 | 3.163975 | −2.047779 |
| TC09001602.hg.1 | SNORA65 | 6.945372 | 5.908411 | −2.051901 |
| TC11001962.hg.1 | CD248 | 7.315282 | 6.259718 | −2.078531 |
| TC09000963.hg.1 | MIR31 | 4.515691 | 3.442785 | −2.103666 |
| TC07001599.hg.1 | MIR1285-1 | 8.055191 | 6.952641 | −2.147339 |
| TC01000109.hg.1 | HMGN2P17 | 6.237407 | 5.13103 | −2.153043 |
| TC20000581.hg.1 | MIR103B2 | 5.218163 | 4.08578 | −2.192205 |
| TC09000642.hg.1 | MIR181B2 | 5.001238 | 3.864694 | −2.198537 |
| TC20000045.hg.1 | MIR103A2 | 5.559469 | 4.244699 | −2.487627 |
| TC11001454.hg.1 | SNORD14B | 4.412351 | 3.09752 | −2.487732 |
| TC11002382.hg.1 | MIR125B1 | 4.025867 | 2.418391 | −3.047183 |
| TC01002776.hg.1 | MIR186 | 4.327394 | 2.684098 | −3.123787 |
| TC16001253.hg.1 | SNORD71 | 4.481426 | 2.82519 | −3.151931 |
| TC02000882.hg.1 | MIR128-1 | 4.627045 | 2.931888 | −3.238121 |
| TC09000457.hg.1 | MIRLET7F1 | 4.557094 | 2.663987 | −3.714343 |
| TC15001581.hg.1 | SNORD16 | 8.125998 | 6.053993 | −4.204706 |
| TC18000505.hg.1 | SNORD58A | 4.184764 | 2.077527 | −4.308653 |
| TC0X001430.hg.1 | MIR505 | 4.918149 | 2.605037 | −4.969539 |
| TC09001461.hg.1 | MIR32 | 4.260347 | 1.908035 | −5.106419 |
| TC01001501.hg.1 | MIR3120 | 9.395077 | 6.85801 | −5.804078 |
| TC01003533.hg.1 | SNORD78 | 5.392019 | 2.741608 | −6.278461 |
| TC17001801.hg.1 | MIR5047 | 7.933146 | 5.281707 | −6.282937 |
| TC17000728.hg.1 | MIR21 | 6.92532 | 3.855031 | −8.399416 |

TABLE 3
Selected Gene Array Data from Frankincense Extract-Treated 3T3 cells

| | | | | fold |
|---|---|---|---|---|
| | | | | change_Frvs |
| Probe Set ID | Gene Symbol | 3T3-CON | 3T3-Fr | Con |
| TC14000273.hg.1 | RN7SL2 // RN7SL1 | 9.822859 | 11.36809 | 2.918507954 |
| TC21000041.hg.1 | MIR99A | 3.830645 | 5.161523 | 2.515557209 |
| TC07001141.hg.1 | LOC100505921 | 3.008192 | 4.315206 | 2.474288968 |
| TC07000132.hg.1 | MIR1183 | 4.003375 | 5.243876 | 2.362805704 |
| TC15000631.hg.1 | MIR548H4 | 4.457307 | 5.660846 | 2.303039252 |
| TC13000829.hg.1 | GPR183 | 3.215075 | 4.392686 | 2.262018926 |
| TC13000501.hg.1 | LINC00415 | 7.914881 | 9.037157 | 2.176901303 |
| TC11002395.hg.1 | MIR4493 | 4.508823 | 5.589189 | 2.114572463 |
| TC09000159.hg.1 | SNORD121B | 5.19182 | 6.248574 | 2.080245791 |
| TC0X001456.hg.1 | MIR891A | 4.0286 | 5.083563 | 2.077664921 |
| TC08000107.hg.1 | MIR3926-2 | 6.17768 | 7.198474 | 2.02903535 |
| TC15001729.hg.1 | MIR4514 | 4.935244 | 5.9113 | 1.967080501 |
| TC08000416.hg.1 | MIR4470 | 3.801283 | 4.773881 | 1.962371246 |
| TC03001957.hg.1 | SCARNA7 | 3.739947 | 4.683506 | 1.923266919 |
| TC11001275.hg.1 | MIR483 | 7.054221 | 7.991772 | 1.915274266 |
| TC11000254.hg.1 | MIR4486 | 5.023749 | 5.956617 | 1.909067349 |
| TC14000653.hg.1 | MIR665 | 5.032068 | 5.947501 | 1.886135078 |
| TC03000673.hg.1 | MIR1280 | 8.704777 | 9.605453 | 1.866940566 |
| TC06000853.hg.1 | MIR587 | 4.409224 | 5.279844 | 1.82844851 |
| TC03000886.hg.1 | MIR720 | 3.483964 | 4.343086 | 1.813934045 |
| TC02002861.hg.1 | SNORD20 | 3.25094 | 4.106964 | 1.810043037 |
| TC06001974.hg.1 | LINC00577 | 4.980947 | 5.823207 | 1.792856476 |
| TC02002068.hg.1 | MIR4780 | 6.847927 | 7.680294 | 1.780604368 |
| TC06001484.hg.1 | MAS1L | 4.572498 | 5.387801 | 1.759667685 |
| TC6_apd_hap1000059.hg.1 | MAS1L | 4.572498 | 5.387801 | 1.759667685 |
| TC6_cox_hap2000121.hg.1 | MAS1L | 4.572498 | 5.387801 | 1.759667685 |
| TC6_dbb_hap3000110.hg.1 | MAS1L | 4.572498 | 5.387801 | 1.759667685 |
| TC6_mann_hap4000099.hg.1 | MAS1L | 4.572498 | 5.387801 | 1.759667685 |
| TC6_mcf_hap5000103.hg.1 | MAS1L | 4.572498 | 5.387801 | 1.759667685 |
| TC6_qbl_hap6000111.hg.1 | MAS1L | 4.572498 | 5.387801 | 1.759667685 |
| TC6_ssto_hap7000103.hg.1 | MAS1L | 4.572498 | 5.387801 | 1.759667685 |
| TC07000965.hg.1 | MIR548T | 4.072807 | 4.885757 | 1.756800049 |
| TC09001461.hg.1 | MIR32 | 4.260347 | 5.071271 | 1.754334678 |
| TC03000780.hg.1 | SLC9A9-AS2 | 3.955446 | 4.752753 | 1.73785414 |
| TC05001142.hg.1 | MIR4454 | 6.244821 | 7.024767 | 1.717066602 |
| TC07001289.hg.1 | MIR1200 | 3.965137 | 4.734403 | 1.704402414 |
| TC04000110.hg.1 | USP17 | 3.266303 | 4.034055 | 1.70261471 |
| TC11000137.hg.1 | OR2AG1 | 4.18035 | 4.921514 | 1.671523919 |
| TC0X000282.hg.1 | MIR500A | 5.220169 | 5.95955 | 1.66945939 |
| TC22000108.hg.1 | IGLV5-37 | 5.971971 | 6.699737 | 1.656072692 |
| TC20000961.hg.1 | MIR4756 | 4.678939 | 5.406463 | 1.655794923 |
| TC01001250.hg.1 | LELP1 | 6.186014 | 6.912663 | 1.654790981 |
| TC14002281.hg.1 | IGHV2-70 | 5.099775 | 5.82426 | 1.652310704 |
| TC05000673.hg.1 | MIR4461 | 4.550079 | 5.272623 | 1.650089182 |
| TC12000558.hg.1 | MIRLET7I | 4.339975 | 5.060487 | 1.647766709 |
| TC11000344.hg.1 | MIR3973 | 3.288022 | 4.008344 | 1.647549716 |
| TC6_apd_hap1000061.hg.1 | ZFP57 | 3.903519 | 4.617661 | 1.640507284 |
| TC09000783.hg.1 | MIR4669 | 7.98129 | 8.684109 | 1.627682149 |
| TC05001074.hg.1 | OR2V2 | 3.412985 | 4.103467 | 1.613822602 |
| TC14002269.hg.1 | IGHV1-45 | 4.557597 | 5.246749 | 1.612335527 |
| TC08000845.hg.1 | SCXA | 5.220664 | 5.908313 | 1.61065667 |
| TC03001210.hg.1 | EAF1-AS1 | 3.865999 | 4.552849 | 1.609764896 |
| TC14000725.hg.1 | MIR494 | 4.663588 | 5.348782 | 1.607918185 |
| TC10001640.hg.1 | MIR4482-1 | 3.457113 | 4.138968 | 1.604201092 |
| TC17000949.hg.1 | TEX19 | 5.270492 | 5.944767 | 1.595794634 |
| TC11001726.hg.1 | SNORD67 | 4.208288 | 4.880819 | 1.593866725 |
| TC09001709.hg.1 | MIR3689F | 6.685246 | 7.354944 | 1.590739942 |
| TC01003244.hg.1 | HRNR | 3.339468 | 4.008787 | 1.590322105 |
| TC07000964.hg.1 | MIR548F3 | 4.907326 | 5.574337 | 1.587779968 |
| TC01003249.hg.1 | LCE3D | 5.963001 | 6.627163 | 1.584647552 |
| TC01001244.hg.1 | IVL | 4.033257 | 4.694733 | 1.58170001 |
| TC21000210.hg.1 | LOC100506021 | 5.020618 | 5.679872 | 1.579265793 |
| TC14002229.hg.1 | IGHD2-15 | 5.663311 | 6.312611 | 1.568407015 |
| TC14001261.hg.1 | C14orf162 | 4.154953 | 4.794117 | 1.557426414 |
| TC14000722.hg.1 | MIR758 | 3.969651 | 4.608716 | 1.557319545 |
| TC16000667.hg.1 | LOC146513 | 3.543378 | 4.181916 | 1.556750778 |
| TC20000124.hg.1 | MIR3192 | 4.230597 | 4.863161 | 1.550317817 |
| TC07001913.hg.1 | LOC100129148 | 3.399925 | 4.02935 | 1.54694832 |
| TC02000372.hg.1 | LOC730184 | 4.53846 | 5.167092 | 1.546098249 |
| TC02001302.hg.1 | MIR26B | 5.441505 | 6.069453 | 1.545365398 |
| TC12000070.hg.1 | LOC100507560 | 4.011845 | 4.639619 | 1.545179026 |
| TC01003381.hg.1 | OR6N1 | 4.502001 | 5.128548 | 1.543865423 |
| TC02001459.hg.1 | MIR4269 | 5.502205 | 6.123354 | 1.538099676 |
| TC22000103.hg.1 | IGLV4-60 | 6.688641 | 7.306957 | 1.535082295 |
| TC12001163.hg.1 | APOBEC1 | 3.66153 | 4.275623 | 1.530595433 |
| TC09001534.hg.1 | MIR147A | 6.712748 | 7.320776 | 1.524174411 |
| TC11001012.hg.1 | LOC100506870 | 3.760885 | 4.367905 | 1.523109854 |
| TC16001112.hg.1 | LOC388276 | 4.302728 | 4.90918 | 1.522510312 |
| TC13000594.hg.1 | MIR320D1 | 3.970938 | 4.577223 | 1.522334084 |
| TC09000456.hg.1 | MIRLET7A1 | 5.336246 | 5.938571 | 1.518161215 |
| TC22000698.hg.1 | MB | 3.446457 | 4.044221 | 1.513369212 |
| TC09000367.hg.1 | FAM75D3 | 3.548059 | 4.144822 | 1.512319539 |
| TC02004944.hg.1 | IGKV2-24 | 5.720501 | 6.312888 | 1.507739303 |
| TC6_apd_hap1000014.hg.1 | OR2H2 | 3.588051 | 4.179769 | 1.507040303 |
| TC04000702.hg.1 | GUSBP5 | 3.58862 | 4.175904 | 1.502415654 |
| TC12000487.hg.1 | METTL7B | 3.747918 | 4.33377 | 1.500925116 |
| TC06001875.hg.1 | EEF1A1 | 6.101389 | 5.516092 | −1.500347826 |
| TC06000516.hg.1 | RPL10A | 5.605467 | 5.019469 | −1.501077017 |
| TC02000500.hg.1 | TMSB10 | 7.335292 | 6.749011 | −1.501371498 |
| TC02002704.hg.1 | MIR3130-1 | 4.866237 | 4.279479 | −1.50186798 |
| TC15001272.hg.1 | RPS3AP47 | 4.722627 | 4.133708 | −1.5041193 |
| TC10000412.hg.1 | DDX50 | 4.634588 | 4.045026 | −1.504789826 |
| TC15000259.hg.1 | SPRED1 | 4.726825 | 4.135536 | −1.506592236 |
| TC01001146.hg.1 | PPIAL4E | 5.324164 | 4.729788 | −1.509819411 |
| TC0X001174.hg.1 | MAGT1 | 4.901742 | 4.306714 | −1.510501901 |
| TC08001485.hg.1 | YWHAZ | 5.730916 | 5.13518 | −1.511243359 |
| TC04000748.hg.1 | SNORD73A | 5.169149 | 4.573295 | −1.511366971 |
| TC17001785.hg.1 | CCDC47 | 4.137696 | 3.541068 | −1.512178031 |
| TC20000779.hg.1 | EIF2S2 | 5.952834 | 5.356167 | −1.51221891 |
| TC02000521.hg.1 | SNORD94 | 5.756224 | 5.159173 | −1.512621468 |
| TC17001454.hg.1 | MIEN1 | 5.693154 | 5.095053 | −1.513722762 |
| TC17001036.hg.1 | PFN1 | 7.137118 | 6.537572 | −1.515239662 |
| TC17000553.hg.1 | MIR2117 | 4.759161 | 4.156869 | −1.518126489 |
| TC16002106.hg.1 | SLC7A5P1 | 6.685352 | 6.082582 | −1.518629565 |
| TC04000856.hg.1 | SAP30 | 4.16624 | 3.561524 | −1.520679372 |
| TC03001618.hg.1 | RPL24 | 4.976219 | 4.371339 | −1.520852247 |
| TC17001020.hg.1 | UBE2G1 | 4.754099 | 4.149082 | −1.520996676 |
| TC08001750.hg.1 | VPS28 | 6.033446 | 5.42819 | −1.521248668 |
| TC01001266.hg.1 | RPS27 | 6.896022 | 6.289976 | −1.522081911 |
| TC03001723.hg.1 | SNX4 | 4.103076 | 3.494904 | −1.524326552 |
| TC14000968.hg.1 | PSME2 | 6.049039 | 5.440576 | −1.524634048 |
| TC19000235.hg.1 | NFIX | 7.725858 | 7.114291 | −1.527917874 |
| TC20000847.hg.1 | LOC100505725 | 4.089451 | 3.477668 | −1.52814665 |
| TC19001922.hg.1 | CHMP2A | 5.92929 | 5.316396 | −1.529323909 |
| TC17001633.hg.1 | COPZ2 | 5.463604 | 4.850303 | −1.529755409 |
| TC19002630.hg.1 | PSENEN | 4.884567 | 4.270872 | −1.530173242 |
| TC17001542.hg.1 | HMGN2P42 | 5.727414 | 5.113628 | −1.530269763 |
| TC02005005.hg.1 | MOB4 | 4.416603 | 3.801963 | −1.531175871 |
| TC03000677.hg.1 | RAB7A | 6.0616 | 5.442779 | −1.535619728 |
| TC17001058.hg.1 | DERL2 | 5.055295 | 4.435922 | −1.536207395 |
| TC05000572.hg.1 | FTMT | 4.668657 | 4.048903 | −1.536613145 |
| TC17001800.hg.1 | DDX5 | 6.389706 | 5.768218 | −1.538461137 |
| TC0X000636.hg.1 | HPRT1 | 5.248297 | 4.626742 | −1.538532586 |
| TC22000125.hg.1 | IGLJ4 | 5.289522 | 4.66782 | −1.538689359 |
| TC03001845.hg.1 | HMGN2P25 | 6.340228 | 5.717078 | −1.540234482 |
| TC14000632.hg.1 | YY1 | 6.615057 | 5.991838 | −1.540308148 |
| TC11000631.hg.1 | MALAT1 | 4.048334 | 3.423007 | −1.542560421 |
| TC09000916.hg.1 | NFIB | 6.509025 | 5.883199 | −1.543094055 |
| TC16001019.hg.1 | YPEL3 | 6.655644 | 6.028966 | −1.544005616 |
| TC09001578.hg.1 | PSMB7 | 5.115465 | 4.488542 | −1.544267843 |
| TC04001163.hg.1 | RAC1P2 | 5.788292 | 5.158435 | −1.547411606 |
| TC18000088.hg.1 | SNRPD1 | 4.338849 | 3.703486 | −1.553328538 |
| TC0Y000109.hg.1 | VAMP7 | 4.653505 | 4.017261 | −1.554277387 |
| TC0X000606.hg.1 | STAG2 | 4.685826 | 4.047481 | −1.556542534 |
| TC05000561.hg.1 | MIR1244-1 | 6.104167 | 5.463463 | −1.559089772 |
| TC12001190.hg.1 | MIR1244-1 | 6.104167 | 5.463463 | −1.559089772 |
| TC01006391.hg.1 | HNRNPU | 5.973352 | 5.331461 | −1.560373065 |
| TC15001068.hg.1 | MIR4508 | 5.64237 | 4.99978 | −1.561129264 |
| TC05001320.hg.1 | PAIP1 | 5.026083 | 4.381352 | −1.563447744 |
| TC13000466.hg.1 | RPS12P23 | 4.246922 | 3.601424 | −1.564279162 |
| TC06000530.hg.1 | SRSF3 | 4.710351 | 4.063598 | −1.56564052 |
| TC03001495.hg.1 | ARF4 | 4.639943 | 3.992484 | −1.566406873 |
| TC09000134.hg.1 | DNAJA1 | 5.092569 | 4.445023 | −1.566501336 |
| TC04000902.hg.1 | SLC25A4 | 5.337005 | 4.686033 | −1.570225762 |
| TC04000237.hg.1 | UBE2K | 4.979033 | 4.327894 | −1.570407535 |
| TC01000859.hg.1 | MTF2 | 4.577747 | 3.923407 | −1.573895767 |
| TC15000335.hg.1 | PDIA3 | 4.691738 | 4.037266 | −1.574039778 |
| TC02000315.hg.1 | SPTBN1 | 5.51998 | 4.863128 | −1.576638599 |
| TC05000247.hg.1 | GPBP1 | 4.646715 | 3.984981 | −1.581982894 |
| TC02001062.hg.1 | HNRNPA3 | 8.386349 | 7.723068 | −1.583680162 |
| TC0X000210.hg.1 | RPL19P20 | 5.790275 | 5.12652 | −1.584200568 |
| TC04001323.hg.1 | CNOT6L | 4.980573 | 4.315133 | −1.586051921 |
| TC19000964.hg.1 | RPS5 | 7.030276 | 6.364357 | −1.586578606 |
| TC19001774.hg.1 | ETFB | 6.086036 | 5.417975 | −1.588935982 |
| TC17001722.hg.1 | VEZF1 | 6.87086 | 6.20272 | −1.589022992 |
| TC19000500.hg.1 | LOC644189 | 4.403179 | 3.733371 | −1.590861235 |
| TC15000609.hg.1 | RAB11A | 5.790462 | 5.120288 | −1.591264875 |
| TC01003278.hg.1 | GATAD2B | 7.003942 | 6.333628 | −1.591419299 |
| TC17001698.hg.1 | TOBI | 4.663505 | 3.993052 | −1.591572636 |
| TC04001086.hg.1 | DHX15 | 4.867498 | 4.191006 | −1.598248788 |
| TC01001291.hg.1 | HMGN2P18 | 4.960291 | 4.282629 | −1.599545465 |
| TC19001078.hg.1 | SNRPEP4 | 5.592162 | 4.913364 | −1.600805468 |
| TC01003293.hg.1 | UBE2Q1 | 6.290261 | 5.611365 | −1.600914212 |
| TC16001087.hg.1 | DNAJA2 | 5.370364 | 4.689581 | −1.603009527 |
| TC15000659.hg.1 | MIR630 | 6.422822 | 5.741395 | −1.603725249 |
| TC03001176.hg.1 | SNORA7A | 6.05602 | 5.374066 | −1.604311179 |
| TC15001286.hg.1 | MIR1282 | 7.444377 | 6.760858 | −1.60605244 |
| TC6_ssto_hap7000088.hg.1 | RPS18 | 8.924051 | 8.238903 | −1.607866917 |
| TC06000883.hg.1 | AMD1 | 4.695722 | 4.006057 | −1.612908951 |
| TC0X000212.hg.1 | KDM6A | 4.759834 | 4.06695 | −1.616511757 |
| TC13000573.hg.1 | CSNK1A1L | 5.519701 | 4.825323 | −1.618186622 |
| TC16000875.hg.1 | GSPT1 | 4.817438 | 4.119778 | −1.621872039 |
| TC02001616.hg.1 | LAPTM4A | 4.558009 | 3.856361 | −1.626361535 |
| TC07000128.hg.1 | RPL23P8 | 4.852993 | 4.148611 | −1.629446517 |
| TC0X000199.hg.1 | DDX3X | 5.134817 | 4.428263 | −1.631901522 |
| TC19001687.hg.1 | RPL18 | 7.727967 | 7.021253 | −1.632082516 |
| TC12000435.hg.1 | PFDN5 | 5.225925 | 4.517718 | −1.633772381 |
| TC03000832.hg.1 | MBNL1 | 5.664012 | 4.955076 | −1.634598142 |
| TC17000375.hg.1 | MIR632 | 6.977433 | 6.268409 | −1.634697851 |
| TC03000816.hg.1 | SELT | 4.244185 | 3.534144 | −1.635850606 |
| TC08000667.hg.1 | ENY2 | 4.509495 | 3.797819 | −1.637705559 |
| TC05000659.hg.1 | UBE2B | 4.53978 | 3.825863 | −1.640251454 |
| TC10000755.hg.1 | MIR146B | 4.405058 | 3.689862 | −1.64170624 |
| TC08000424.hg.1 | YTHDF3 | 6.250556 | 5.534941 | −1.642183107 |
| TC16000759.hg.1 | MRPS34 | 5.674678 | 4.958537 | −1.642781949 |
| TC17001772.hg.1 | MED13 | 4.645951 | 3.927115 | −1.645853586 |
| TC09001588.hg.1 | PPP6C | 4.001479 | 3.279346 | −1.649619166 |
| TC01000299.hg.1 | RPL11 | 5.335133 | 4.609773 | −1.653313141 |
| TC19000735.hg.1 | RPS11 | 6.486194 | 5.759977 | −1.654295546 |
| TC02002682.hg.1 | SUMO1 | 4.827639 | 4.09363 | −1.663254573 |
| TC0X000792.hg.1 | VAMP7 | 4.431813 | 3.695829 | −1.66553307 |
| TC12001609.hg.1 | ATP5B | 5.213948 | 4.477503 | −1.666065361 |
| TC19000360.hg.1 | UBA52 | 6.698521 | 5.961961 | −1.666198172 |
| TC06000697.hg.1 | PTP4A1 | 4.446811 | 3.702919 | −1.674687603 |
| TC03000965.hg.1 | FXR1 | 5.426085 | 4.680681 | −1.676443661 |
| TC11000059.hg.1 | MIR4686 | 6.619994 | 5.87333 | −1.677908448 |
| TC09001284.hg.1 | MIR7-1 | 5.424988 | 4.675774 | −1.680876816 |
| TC15001549.hg.1 | PPIB | 6.120805 | 5.367199 | −1.686001711 |
| TC17001081.hg.1 | GABARAP | 7.154794 | 6.398185 | −1.689514814 |
| TC15001634.hg.1 | NPTN | 7.241183 | 6.483265 | −1.691048456 |
| TC06001281.hg.1 | RANBP9 | 5.250926 | 4.491182 | −1.693190149 |
| TC15000670.hg.1 | LOXL1 | 6.682571 | 5.920768 | −1.695608378 |
| TC06001165.hg.1 | QKI | 6.577658 | 5.811338 | −1.700925556 |
| TC09001543.hg.1 | RAB14 | 6.098108 | 5.33127 | −1.701536383 |
| TC17001066.hg.1 | MED31 | 5.845514 | 5.076862 | −1.703677188 |
| TC03001766.hg.1 | CNBP | 5.623683 | 4.854891 | −1.703842522 |
| TC11000309.hg.1 | EIF3M | 4.800894 | 4.031566 | −1.704475663 |
| TC08000410.hg.1 | RAB2A | 4.290187 | 3.517171 | −1.708838433 |
| TC19001203.hg.1 | WDR83OS | 4.822986 | 4.049321 | −1.709607332 |
| TC05000166.hg.1 | NIPBL | 5.134871 | 4.358963 | −1.712267375 |
| TC17000466.hg.1 | PSMB3 | 5.77254 | 4.992606 | −1.71705232 |
| TC06000937.hg.1 | ASF1A | 4.218333 | 3.436528 | −1.719280572 |
| TC09000687.hg.1 | TMSB4XP4 | 6.356374 | 5.574488 | −1.719377104 |
| TC17000052.hg.1 | PSMB6 | 5.817444 | 5.031171 | −1.724613406 |
| TC10001410.hg.1 | PPP3CB | 4.742918 | 3.950762 | −1.731660367 |
| TC01001857.hg.1 | H3F3A | 5.32069 | 4.526773 | −1.733775378 |
| TC15001743.hg.1 | RPS17 | 6.552969 | 5.757362 | −1.735807545 |
| TC15001754.hg.1 | RPS17 | 6.552969 | 5.757362 | −1.735807545 |
| TC02000970.hg.1 | PSMD14 | 4.980259 | 4.183622 | −1.737047253 |
| TC14000605.hg.1 | PAPOLA | 4.691407 | 3.892722 | −1.739514856 |
| TC0X000911.hg.1 | RPS6KA3 | 4.413763 | 3.614691 | −1.73998154 |
| TC02002647.hg.1 | SF3B1 | 5.311529 | 4.50944 | −1.74362404 |
| TC01001331.hg.1 | CYCSP52 | 4.445886 | 3.643714 | −1.743724356 |
| TC11002240.hg.1 | DCUN1D5 | 6.132967 | 5.329648 | −1.745111237 |
| TC20000047.hg.1 | FTLP3 | 5.549015 | 4.744746 | −1.746260754 |
| TC0X001207.hg.1 | PCDH19 | 4.461317 | 3.655943 | −1.747598775 |
| TC05000067.hg.1 | SNORD123 | 5.656614 | 4.848636 | −1.750755961 |
| TC17001882.hg.1 | H3F3B | 6.070302 | 5.251262 | −1.764231645 |
| TC05001367.hg.1 | LOC100653008 // LOC100652914 | 6.173776 | 5.353742 | −1.765447598 |
| TC07001891.hg.1 | LUZP6 | 6.043021 | 5.222705 | −1.76579272 |
| TC05000658.hg.1 | MIR3661 | 7.279772 | 6.458873 | −1.766506429 |
| TC02002826.hg.1 | CUL3 | 4.125062 | 3.297724 | −1.774408277 |
| TC11003469.hg.1 | STT3A | 4.779898 | 3.951407 | −1.775826948 |
| TC11001417.hg.1 | EIF4G2 | 8.009993 | 7.181435 | −1.775909421 |
| TC15001652.hg.1 | COX5A | 4.423629 | 3.583231 | −1.790544036 |
| TC17_ctg5_hap1000006.hg.1 | MAPT-AS1 | 7.054317 | 6.210587 | −1.794684195 |
| TC17001078.hg.1 | MIR324 | 4.103482 | 3.237063 | −1.823131974 |
| TC05001181.hg.1 | ZNF622 | 4.341874 | 3.469276 | −1.830957114 |
| TC03000120.hg.1 | HMGB1P5 | 6.440309 | 5.567283 | −1.83150038 |
| TC16001133.hg.1 | NUDT21 | 5.474231 | 4.595953 | −1.838179941 |
| TC01001149.hg.1 | PPIAL4D | 5.34361 | 4.463593 | −1.840396987 |
| TC01003149.hg.1 | PPIAL4D | 5.34361 | 4.463593 | −1.840396987 |
| TC06001090.hg.1 | BTF3P10 | 4.191702 | 3.298536 | −1.857247392 |
| TC07001823.hg.1 | WASL | 5.404926 | 4.506792 | −1.86365395 |
| TC15001577.hg.1 | SCARNA14 | 6.018013 | 5.117529 | −1.866692122 |
| TC19000587.hg.1 | RPS19 | 7.001619 | 6.100407 | −1.867634313 |
| TC08001458.hg.1 | RPL30 | 5.733222 | 4.828846 | −1.87173475 |
| TC11001423.hg.1 | CSNK2A1P | 5.287188 | 4.379592 | −1.875917004 |
| TC0X000706.hg.1 | FMRI-IT1 | 4.278802 | 3.370976 | −1.876216093 |
| TC19001521.hg.1 | RPS16 | 6.226418 | 5.315427 | −1.880336675 |
| TC12003242.hg.1 | PCBP2 | 8.785668 | 7.873739 | −1.881559615 |
| TC10000623.hg.1 | PTEN | 5.781565 | 4.862614 | −1.890740015 |
| TC17001609.hg.1 | MAPT-AS1 | 6.233504 | 5.314217 | −1.891180415 |
| TC17000024.hg.1 | PAFAH1B1 | 5.534348 | 4.611858 | −1.895383784 |
| TC04001578.hg.1 | PPP1R14BP3 | 6.060392 | 5.135296 | −1.898810589 |
| TC14000418.hg.1 | SRSF5 | 5.784508 | 4.847188 | −1.914967623 |
| TC12001455.hg.1 | SNORA2B | 4.749049 | 3.8101 | −1.917131104 |
| TC01000979.hg.1 | RAP1A | 5.679533 | 4.736298 | −1.922835041 |
| TC14002294.hg.1 | PSMA6 | 4.752563 | 3.799373 | −1.936149024 |
| TC11002353.hg.1 | DDX6 | 5.48931 | 4.526114 | −1.949624114 |
| TC07000478.hg.1 | SNORA14A | 5.49603 | 4.531966 | −1.950797461 |
| TC02001644.hg.1 | SF3B14 | 4.388421 | 3.423838 | −1.951499374 |
| TC02000923.hg.1 | ACVR2A | 5.802602 | 4.834877 | −1.955754114 |
| TC0X000110.hg.1 | PRDX4 | 4.416517 | 3.448743 | −1.955820541 |
| TC17000816.hg.1 | LOC124685 | 5.628424 | 4.656113 | −1.961980904 |
| TC03001976.hg.1 | PDCD10 | 5.540936 | 4.563399 | −1.969100846 |
| TC15001473.hg.1 | CNOT6LP1 | 4.487798 | 3.505769 | −1.975241427 |
| TC02002884.hg.1 | DNAJB3 | 4.410896 | 3.4228 | −1.983565448 |
| TC15001685.hg.1 | ETFA | 5.367002 | 4.371588 | −1.993652548 |
| TC12000601.hg.1 | RAP1B | 4.480546 | 3.463821 | −2.023320689 |
| TC11001962.hg.1 | CD248 | 7.315282 | 6.281682 | −2.047126138 |
| TC01003015.hg.1 | CSDE1 | 7.2094 | 6.153724 | −2.078691986 |
| TC17001731.hg.1 | SUPT4H1 | 6.254887 | 5.19046 | −2.09133909 |
| TC17000473.hg.1 | RPL19 | 7.462895 | 6.392712 | −2.099699689 |
| TC0X000969.hg.1 | CASK | 6.003073 | 4.930348 | −2.103402579 |
| TC15000130.hg.1 | LOC100507026 | 6.384228 | 5.308319 | −2.108049874 |
| TC03001759.hg.1 | FTH1P4 | 5.648375 | 4.568739 | −2.113502765 |
| TC20000885.hg.1 | RPS2P7 | 5.867107 | 4.779521 | −2.125181413 |
| TC19001208.hg.1 | C19orf43 | 6.846635 | 5.742829 | −2.149209321 |
| TC10000164.hg.1 | YWHAZP3 | 5.566947 | 4.443683 | −2.17839262 |
| TC21000511.hg.1 | MYL6P1 | 8.352276 | 7.223123 | −2.187302869 |
| TC07001347.hg.1 | PURB | 5.12075 | 3.986145 | −2.195584409 |
| TC21000151.hg.1 | MEMO1P1 | 5.212935 | 4.063756 | −2.217876449 |
| TC01003990.hg.1 | SNORA14B | 5.419961 | 4.261175 | −2.232694716 |
| TC20000146.hg.1 | LOC100653248 // LOC100652902 | 6.592179 | 5.428381 | −2.240464706 |
| TC02000882.hg.1 | MIR128-1 | 4.627045 | 3.451471 | −2.258827343 |
| TC05000721.hg.1 | IGIP | 4.114312 | 2.917461 | −2.292387604 |
| TC01002776.hg.1 | MIR186 | 4.327394 | 3.129814 | −2.29354625 |
| TC15001342.hg.1 | COPS2 | 4.840778 | 3.620209 | −2.330386098 |
| TC08001548.hg.1 | EXT1 | 7.248168 | 6.01527 | −2.350386476 |
| TC16001148.hg.1 | CSNK2A2 | 6.4688 | 5.226803 | −2.365257082 |
| TC18000505.hg.1 | SNORD58A | 4.184764 | 2.938612 | −2.372078904 |
| TC01001501.hg.1 | MIR3120 | 9.395077 | 8.113411 | −2.431195652 |
| TC11003475.hg.1 | RPS13 | 5.796555 | 4.514335 | −2.432129419 |
| TC02001922.hg.1 | RAB1A | 6.538365 | 5.254374 | −2.435116846 |
| TC02000332.hg.1 | EIF3FP3 | 5.682837 | 4.370251 | −2.483863684 |
| TC0X000201.hg.1 | CASK-AS1 | 6.63735 | 5.254511 | −2.607810431 |
| TC11002391.hg.1 | SNORD14E | 6.725938 | 5.330383 | −2.630897423 |
| TC13000192.hg.1 | PSME2P2 | 7.02319 | 5.579358 | −2.720424896 |
| TC09001602.hg.1 | SNORA65 | 6.945372 | 5.456001 | −2.807665372 |
| TC03000436.hg.1 | MIR4444-1 | 7.254175 | 5.756509 | −2.823854979 |
| TC07001599.hg.1 | MIR1285-1 | 8.055191 | 6.509108 | −2.920232021 |
| TC14000798.hg.1 | SNORA28 | 4.250294 | 2.674725 | −2.980530218 |
| TC09001589.hg.1 | HSPA5 | 6.197709 | 4.587939 | −3.052031812 |
| TC17000728.hg.1 | MIR21 | 6.92532 | 5.307908 | −3.068241412 |
| TC16001253.hg.1 | SNORD71 | 4.481426 | 2.730186 | −3.366477914 |
| TC05001498.hg.1 | LOC100506390 | 5.657677 | 3.8859 | −3.414742994 |
| TC17001801.hg.1 | MIR5047 | 7.933146 | 6.146515 | −3.450082833 |
| TC09000457.hg.1 | MIRLET7F1 | 4.557094 | 2.727695 | −3.553889932 |
| TC11001454.hg.1 | SNORD14B | 4.412351 | 2.454423 | −3.885036088 |
| TC07001462.hg.1 | SKP1P1 | 5.473197 | 3.441776 | −4.088073118 |
| TC01003533.hg.1 | SNORD78 | 5.392019 | 3.329841 | −4.176162934 |
| TC15001581.hg.1 | SNORD16 | 8.125998 | 5.9578 | −4.494616425 |

TABLE 4
Selected Gene Array Data from Orange Extract-Treated 3T3 cells

| | | | | fold |
|---|---|---|---|---|
| | | | | change_Orvs |
| Probe Set ID | Gene Symbol | 3T3-CON | 3T3-Or | Con |
| TC05000673.hg.1 | MIR4461 | 4.550079 | 7.845676 | 9.819142 |
| TC05001540.hg.1 | MTRNR2L2 | 4.27511 | 6.642415 | 5.159764 |
| TC14000273.hg.1 | RN7SL2 // RN7SL1 | 9.822859 | 11.77166 | 3.860536 |
| TC06000695.hg.1 | MIR4485 | 5.921429 | 7.580209 | 3.157494 |
| TC15000054.hg.1 | SNORD116-8 | 2.675926 | 4.289651 | 3.06041 |
| TC02002887.hg.1 | MSL3P1 | 4.056669 | 5.581986 | 2.8785 |
| TC20000974.hg.1 | MTRNR2L3 | 2.985364 | 4.461706 | 2.782423 |
| TC01003789.hg.1 | ST13P19 | 3.707287 | 5.167145 | 2.750813 |
| TC11001411.hg.1 | MTRNR2L8 | 5.25792 | 6.700375 | 2.71783 |
| TC08001037.hg.1 | MIR320A | 5.41256 | 6.850599 | 2.709523 |
| TC01001965.hg.1 | RPS7P5 | 3.104998 | 4.527943 | 2.681323 |
| TC11001334.hg.1 | OR51B4 | 3.000185 | 4.390987 | 2.622244 |
| TC11001487.hg.1 | NAV2-AS1 | 3.757996 | 5.108868 | 2.550662 |
| TC09000827.hg.1 | MIR4479 | 5.591455 | 6.908024 | 2.490731 |
| TC05000976.hg.1 | MIR4634 | 5.571525 | 6.85874 | 2.440565 |
| TC05000854.hg.1 | MIR1303 | 5.482394 | 6.754982 | 2.415946 |
| TC09000368.hg.1 | FAM75D1 | 3.154871 | 4.408372 | 2.384193 |
| TC09000043.hg.1 | MIR4665 | 5.081307 | 6.293707 | 2.317228 |
| TC22001440.hg.1 | IGLV7-43 | 4.387714 | 5.566163 | 2.263333 |
| TC19001654.hg.1 | MIR320E | 5.138947 | 6.288947 | 2.219139 |
| TC02004944.hg.1 | IGKV2-24 | 5.720501 | 6.848382 | 2.185375 |
| TC12000477.hg.1 | OR6C1 | 4.6803 | 5.804807 | 2.18027 |
| TC19000587.hg.1 | RPS19 | 7.001619 | 8.124441 | 2.177725 |
| TC22000779.hg.1 | MIR659 | 3.162537 | 4.281244 | 2.171523 |
| TC12000851.hg.1 | MIR4497 | 3.99744 | 5.112935 | 2.166693 |
| TC17000775.hg.1 | SNORD104 | 4.399087 | 5.514315 | 2.166292 |
| TC19000185.hg.1 | MIR638 | 4.886649 | 5.981811 | 2.136371 |
| TC19000717.hg.1 | FTL | 5.380933 | 6.471504 | 2.129583 |
| TC19000829.hg.1 | MIR525 | 3.076784 | 4.163461 | 2.123843 |
| TC02000414.hg.1 | PCBP1 | 5.609785 | 6.686197 | 2.108785 |
| TC19001651.hg.1 | GNG8 | 5.092001 | 6.161027 | 2.098016 |
| TC05000561.hg.1 | MIR1244-1 | 6.104167 | 7.172461 | 2.096952 |
| TC12001190.hg.1 | MIR1244-1 | 6.104167 | 7.172461 | 2.096952 |
| TC16001086.hg.1 | C16orf87 | 5.375694 | 6.433058 | 2.081126 |
| TC02000332.hg.1 | EIF3FP3 | 5.682837 | 6.724731 | 2.058929 |
| TC09000365.hg.1 | FAM75D5 | 3.635556 | 4.655344 | 2.027621 |
| TC11001012.hg.1 | LOC100506870 | 3.760885 | 4.780482 | 2.027353 |
| TC21000007.hg.1 | MIR3687 | 5.041083 | 6.051366 | 2.014306 |
| TC07001299.hg.1 | TRGV4 | 3.333298 | 4.338046 | 2.006593 |
| TC22000103.hg.1 | IGLV4-60 | 6.688641 | 7.688278 | 1.999497 |
| TC15001272.hg.1 | RPS3AP47 | 4.722627 | 5.703018 | 1.973 |
| TC17001081.hg.1 | GABARAP | 7.154794 | 8.134536 | 1.972113 |
| TC02001930.hg.1 | MIR4778 | 3.115399 | 4.093756 | 1.97022 |
| TC03000969.hg.1 | SOX2 | 4.906857 | 5.884963 | 1.969878 |
| TC01000826.hg.1 | SH3GLB1 | 3.565946 | 4.543586 | 1.969241 |
| TC06000414.hg.1 | MIR219-1 | 3.099403 | 4.074826 | 1.966218 |
| TC6_cox_hap2000104.hg.1 | MIR219-1 | 3.099403 | 4.074826 | 1.966218 |
| TC6_dbb_hap3000096.hg.1 | MIR219-1 | 3.099403 | 4.074826 | 1.966218 |
| TC6_mann_hap4000088.hg.1 | MIR219-1 | 3.099403 | 4.074826 | 1.966218 |
| TC6_mcf_hap5000089.hg.1 | MIR219-1 | 3.099403 | 4.074826 | 1.966218 |
| TC6_qbl_hap6000095.hg.1 | MIR219-1 | 3.099403 | 4.074826 | 1.966218 |
| TC01001950.hg.1 | GPR137B | 3.945215 | 4.919755 | 1.965015 |
| TC14001098.hg.1 | RPS29 | 4.337722 | 5.311153 | 1.963505 |
| TC0X001192.hg.1 | MIR361 | 3.155392 | 4.121763 | 1.953919 |
| TC06004064.hg.1 | HIST1H3H | 3.228592 | 4.186237 | 1.942137 |
| TC06002154.hg.1 | LOC100507406 | 3.947572 | 4.899403 | 1.934326 |
| TC11000600.hg.1 | MIR1237 | 6.69723 | 7.634903 | 1.915436 |
| TC16000360.hg.1 | MIR762 | 4.220536 | 5.154593 | 1.910641 |
| TC12000416.hg.1 | LOC100509541 | 6.352322 | 7.281645 | 1.904382 |
| TC06001436.hg.1 | TOB2P1 | 3.472974 | 4.398901 | 1.899905 |
| TC04000298.hg.1 | MIR4449 | 6.257283 | 7.181866 | 1.898136 |
| TC09001602.hg.1 | SNORA65 | 6.945372 | 7.850095 | 1.872185 |
| TC17001339.hg.1 | MIR4733 | 3.561949 | 4.466373 | 1.871797 |
| TC06000732.hg.1 | OOEP-AS1 | 4.690115 | 5.592884 | 1.869651 |
| TC12000425.hg.1 | EIF4B | 5.376654 | 6.279009 | 1.869115 |
| TC19000039.hg.1 | RPS15 | 5.434027 | 6.330772 | 1.861861 |
| TC09000367.hg.1 | FAM75D3 | 3.548059 | 4.4447 | 1.861726 |
| TC20000047.hg.1 | FTLP3 | 5.549015 | 6.444175 | 1.859816 |
| TC03000519.hg.1 | PCNP | 4.632959 | 5.52191 | 1.851829 |
| TC06002254.hg.1 | MIR4466 | 8.022274 | 8.9067 | 1.84603 |
| TC02000555.hg.1 | IGKV2D-24 | 5.855568 | 6.739562 | 1.845477 |
| TC07000653.hg.1 | MIR4285 | 5.6988 | 6.572588 | 1.832468 |
| TC06001480.hg.1 | OR2W1 | 3.149997 | 4.020931 | 1.828847 |
| TC6_apd_hap1000055.hg.1 | OR2W1 | 3.149997 | 4.020931 | 1.828847 |
| TC6_cox_hap2000117.hg.1 | OR2W1 | 3.149997 | 4.020931 | 1.828847 |
| TC6_dbb_hap3000105.hg.1 | OR2W1 | 3.149997 | 4.020931 | 1.828847 |
| TC6_mann_hap4000095.hg.1 | OR2W1 | 3.149997 | 4.020931 | 1.828847 |
| TC6_mcf_hap5000099.hg.1 | OR2W1 | 3.149997 | 4.020931 | 1.828847 |
| TC6_qbl_hap6000108.hg.1 | OR2W1 | 3.149997 | 4.020931 | 1.828847 |
| TC6_ssto_hap7000099.hg.1 | OR2W1 | 3.149997 | 4.020931 | 1.828847 |
| TC17001768.hg.1 | NACA2 | 3.760291 | 4.627501 | 1.824132 |
| TC19000104.hg.1 | RPL36 | 7.14966 | 8.01296 | 1.819195 |
| TC12002103.hg.1 | RPL22P19 | 3.885564 | 4.744356 | 1.813519 |
| TC07001716.hg.1 | RPL19P12 | 3.550223 | 4.404374 | 1.807695 |
| TC16000069.hg.1 | MIR940 | 6.147295 | 6.998251 | 1.803696 |
| TC09000366.hg.1 | FAM75D4 | 3.529314 | 4.376192 | 1.798605 |
| TC07001028.hg.1 | LOC100128822 | 4.588253 | 5.431568 | 1.794168 |
| TC14000449.hg.1 | MIR4505 | 4.379337 | 5.221066 | 1.792197 |
| TC0X000026.hg.1 | MIR4767 | 7.175657 | 8.013459 | 1.787325 |
| TC17001731.hg.1 | SUPT4H1 | 6.254887 | 7.091312 | 1.78562 |
| TC16000785.hg.1 | MIR3178 | 6.064056 | 6.899166 | 1.783993 |
| TC20000489.hg.1 | RPS21 | 5.798862 | 6.628416 | 1.777136 |
| TC13000747.hg.1 | MIR3665 | 5.83964 | 6.668992 | 1.776887 |
| TC17001950.hg.1 | MIR4740 | 5.839115 | 6.666828 | 1.77487 |
| TC0X000787.hg.1 | F8A2 | 4.632463 | 5.457766 | 1.771907 |
| TC22001456.hg.1 | MIF | 6.284064 | 7.104576 | 1.766033 |
| TC03000243.hg.1 | TMEM42 | 3.243719 | 4.059777 | 1.760589 |
| TC01003764.hg.1 | LOC100653154 // LOC100652798 | 4.221741 | 5.03622 | 1.758663 |
| TC19001462.hg.1 | POLR2I | 4.65386 | 5.468328 | 1.75865 |
| TC14000544.hg.1 | LOC400236 | 3.955805 | 4.766711 | 1.754313 |
| TC18000133.hg.1 | RNF138 | 3.485646 | 4.293253 | 1.750306 |
| TC10001776.hg.1 | NKX6-2 | 4.973963 | 5.781083 | 1.749715 |
| TC17000514.hg.1 | EIF1 | 5.555455 | 6.355236 | 1.740837 |
| TC22000629.hg.1 | C22orf31 | 3.421077 | 4.216178 | 1.735199 |
| TC08001481.hg.1 | PABPC1 | 7.377176 | 8.171358 | 1.734094 |
| TC06000547.hg.1 | LOC100505550 | 4.568107 | 5.357632 | 1.728505 |
| TC11000803.hg.1 | TPBGL | 4.113005 | 4.898604 | 1.723808 |
| TC17000939.hg.1 | NPB | 4.647816 | 5.425736 | 1.714657 |
| TC19000066.hg.1 | S1PR4 | 5.627247 | 6.404182 | 1.713487 |
| TC19001054.hg.1 | EEF2 | 6.144917 | 6.921491 | 1.713058 |
| TC6_mcf_hap5000153.hg.1 | ZBTB12 | 4.585016 | 5.359829 | 1.710968 |
| TC16000051.hg.1 | SNHG9 | 5.48661 | 6.25807 | 1.706996 |
| TC15000639.hg.1 | RPLP1 | 4.781231 | 5.552156 | 1.706363 |
| TC07000128.hg.1 | RPL23P8 | 4.852993 | 5.623571 | 1.705953 |
| TC04001319.hg.1 | CCNI | 4.806638 | 5.5755 | 1.703925 |
| TC17000987.hg.1 | MIR212 | 4.92356 | 5.686556 | 1.697011 |
| TC03001104.hg.1 | LOC152217 | 3.831018 | 4.592961 | 1.695773 |
| TC10001661.hg.1 | RPL13AP6 | 5.855871 | 6.614902 | 1.692354 |
| TC06000996.hg.1 | RPS12 | 6.971004 | 7.724992 | 1.686448 |
| TC03000016.hg.1 | ARL8B | 3.302924 | 4.056353 | 1.685795 |
| TC11000170.hg.1 | IPO7 | 4.15205 | 4.903461 | 1.683438 |
| TC02002360.hg.1 | MIR663B | 5.723829 | 6.47522 | 1.683415 |
| TC22000633.hg.1 | RASL10A | 4.886625 | 5.638011 | 1.683409 |
| TC20000449.hg.1 | RBM38 | 4.680871 | 5.43119 | 1.682165 |
| TC19001057.hg.1 | ZBTB7A | 5.749765 | 6.494245 | 1.67537 |
| TC12000355.hg.1 | LOC100506125 | 4.124137 | 4.867512 | 1.674088 |
| TC02002190.hg.1 | RPL22P11 | 3.291282 | 4.033565 | 1.672821 |
| TC14001262.hg.1 | LOC100289511 | 4.554801 | 5.2932 | 1.668323 |
| TC01002656.hg.1 | KTI12 | 3.552426 | 4.290461 | 1.667903 |
| TC09001136.hg.1 | LOC100132439 // FAM27E3 | 3.837921 | 4.570834 | 1.661991 |
| TC02000378.hg.1 | ACTR2 | 3.976848 | 4.709688 | 1.661907 |
| TC05001230.hg.1 | MIR4279 | 6.146451 | 6.879255 | 1.661866 |
| TC12001752.hg.1 | NAP1L1 | 4.161489 | 4.892557 | 1.659867 |
| TC19000415.hg.1 | PLEKHF1 | 6.881925 | 7.610463 | 1.656959 |
| TC11000209.hg.1 | FAR1-IT1 | 3.67644 | 4.40398 | 1.655813 |
| TC08001469.hg.1 | MIR599 | 3.742795 | 4.46982 | 1.655222 |
| TC06000223.hg.1 | HIST1H2AG | 4.759904 | 5.484085 | 1.651963 |
| TC20000360.hg.1 | ZSWIM1 | 5.064005 | 5.78636 | 1.649873 |
| TC08001714.hg.1 | RHPN1-AS1 | 5.159625 | 5.879391 | 1.646915 |
| TC22000107.hg.1 | IGLV5-48 | 6.879433 | 7.597331 | 1.644784 |
| TC15001068.hg.1 | MIR4508 | 5.64237 | 6.36005 | 1.644535 |
| TC17000305.hg.1 | MTRNR2L1 | 4.000413 | 4.718067 | 1.644506 |
| TC10000383.hg.1 | ADO | 3.948715 | 4.663918 | 1.641714 |
| TC14002269.hg.1 | IGHV1-45 | 4.557597 | 5.271059 | 1.639734 |
| TC20000151.hg.1 | SSTR4 | 5.670147 | 6.383241 | 1.639316 |
| TC01002430.hg.1 | TMEM200B | 4.429708 | 5.142499 | 1.638972 |
| TC05000713.hg.1 | UBE2D2 | 5.10337 | 5.815283 | 1.637975 |
| TC16000886.hg.1 | ABCC6P2 | 4.204072 | 4.915456 | 1.637374 |
| TC20000495.hg.1 | NTSR1 | 4.542494 | 5.252423 | 1.635724 |
| TC09000697.hg.1 | SET | 5.822623 | 6.531628 | 1.634676 |
| TC0X000317.hg.1 | VTRNA3-1P | 4.655123 | 5.363026 | 1.633428 |
| TC17001428.hg.1 | MIR4734 | 6.254826 | 6.960719 | 1.631154 |
| TC21000266.hg.1 | RPL23AP4 | 5.290962 | 5.99508 | 1.629148 |
| TC0X000781.hg.1 | F8A2 | 4.590324 | 5.294365 | 1.629061 |
| TC05000736.hg.1 | VTRNA1-3 | 4.112596 | 4.816422 | 1.628819 |
| TC07000381.hg.1 | LOC649395 | 4.814817 | 5.517858 | 1.627933 |
| TC09000719.hg.1 | LOC100506190 // LOC100128077 | 5.650448 | 6.351682 | 1.625895 |
| TC19000271.hg.1 | MIR1470 | 5.338841 | 6.039804 | 1.62559 |
| TC13000632.hg.1 | SNORA31 | 4.158487 | 4.856488 | 1.622255 |
| TC09000661.hg.1 | ZBTB34 | 3.814536 | 4.512244 | 1.621926 |
| TC11001275.hg.1 | MIR483 | 7.054221 | 7.750942 | 1.620817 |
| TC13000766.hg.1 | SLITRK1 | 3.968979 | 4.662112 | 1.616791 |
| TC08000009.hg.1 | MIR596 | 6.468017 | 7.15636 | 1.611432 |
| TC17000972.hg.1 | YWHAE | 4.717672 | 5.405631 | 1.611003 |
| TC19001396.hg.1 | CEBPA | 5.887416 | 6.57298 | 1.608331 |
| TC19000964.hg.1 | RPS5 | 7.030276 | 7.715388 | 1.607827 |
| TC22000108.hg.1 | IGLV5-37 | 5.971971 | 6.656939 | 1.607666 |
| TC06000615.hg.1 | HSP90AB1 | 4.611507 | 5.29622 | 1.607382 |
| TC07000914.hg.1 | MTRNR2L6 | 4.668907 | 5.353436 | 1.607177 |
| TC11000030.hg.1 | RPLP2 | 6.320051 | 7.003219 | 1.605662 |
| TC19000344.hg.1 | RPL18A | 6.513666 | 7.195398 | 1.604064 |
| TC11000315.hg.1 | LOC338739 | 3.430565 | 4.111756 | 1.603463 |
| TC19001755.hg.1 | SNORD88B | 5.457413 | 6.138579 | 1.603435 |
| TC6_qbl_hap6000168.hg.1 | ZBTB12 | 3.796685 | 4.474918 | 1.600179 |
| TC05001164.hg.1 | DAP | 4.489797 | 5.167287 | 1.599355 |
| TC11003483.hg.1 | EEF1G | 7.28125 | 7.955442 | 1.595703 |
| TCUn_gl000219000002.hg.1 | LOC283788 | 5.127021 | 5.801057 | 1.59553 |
| TC15000835.hg.1 | LINC00052 | 3.450961 | 4.122829 | 1.593134 |
| TC18000053.hg.1 | SLC35G4 | 5.304691 | 5.976038 | 1.592559 |
| TC14001029.hg.1 | CFL2 | 4.566075 | 5.236341 | 1.591366 |
| TC08000416.hg.1 | MIR4470 | 3.801283 | 4.468447 | 1.587948 |
| TC02001394.hg.1 | PTMA | 6.542615 | 7.20974 | 1.587905 |
| TC09000824.hg.1 | C9orf172 | 5.236991 | 5.903757 | 1.58751 |
| TC6_apd_hap1000075.hg.1 | PPP1R18 | 5.92658 | 6.59316 | 1.587306 |
| TC6_dbb_hap3000128.hg.1 | PPP1R18 | 5.92658 | 6.59316 | 1.587306 |
| TC16002033.hg.1 | HBM | 4.926095 | 5.591447 | 1.585955 |
| TC12001183.hg.1 | A2ML1-AS2 | 5.072242 | 5.737494 | 1.585845 |
| TC11002390.hg.1 | HSPA8 | 3.973984 | 4.638262 | 1.584775 |
| TC03000423.hg.1 | GPR27 | 4.295853 | 4.95906 | 1.583599 |
| TC19001279.hg.1 | INSL3 | 5.188319 | 5.851189 | 1.583229 |
| TC08002629.hg.1 | RNF139 | 3.779168 | 4.440573 | 1.581622 |
| TC09000772.hg.1 | SNORD24 | 4.988654 | 5.647532 | 1.578854 |
| TC02000938.hg.1 | MIR4773-1 | 3.889886 | 4.545768 | 1.575579 |
| TC19001142.hg.1 | OR7G1 | 5.158237 | 5.808935 | 1.569928 |
| TC08000601.hg.1 | C8orf47 | 4.071659 | 4.720987 | 1.568437 |
| TC06001556.hg.1 | C4B-AS1 | 4.037448 | 4.686595 | 1.568241 |
| TC06001554.hg.1 | C4A-AS1 | 4.037448 | 4.686595 | 1.568241 |
| TC15001652.hg.1 | COX5A | 4.423629 | 5.072124 | 1.567532 |
| TC04000935.hg.1 | FRG1 | 4.323487 | 4.968333 | 1.563572 |
| TC0X001458.hg.1 | CXorf51A | 4.253369 | 4.896274 | 1.56147 |
| TC11002408.hg.1 | OR8B8 | 3.571435 | 4.213524 | 1.560587 |
| TC21000041.hg.1 | MIR99A | 3.830645 | 4.472729 | 1.560582 |
| TC05001115.hg.1 | MRPL36 | 6.430285 | 7.071369 | 1.5595 |
| TC11000646.hg.1 | CCDC85B | 4.318505 | 4.958952 | 1.558812 |
| TC6_mann_hap4000117.hg.1 | PPP1R18 | 5.860495 | 6.497839 | 1.555463 |
| TC6_mcf_hap5000118.hg.1 | PPP1R18 | 5.860495 | 6.497839 | 1.555463 |
| TC20000393.hg.1 | RNF114 | 4.393321 | 5.030416 | 1.555194 |
| TC12002099.hg.1 | UBC | 6.351896 | 6.985017 | 1.550916 |
| TC0X001554.hg.1 | F8A2 | 4.94487 | 5.5777 | 1.550604 |
| TC09000156.hg.1 | UBE2R2 | 6.119015 | 6.750616 | 1.549283 |
| TC01001510.hg.1 | PRDX6 | 3.534401 | 4.165977 | 1.549256 |
| TC08001099.hg.1 | DUSP4 | 4.625811 | 5.256572 | 1.548382 |
| TC11000225.hg.1 | OR7E14P | 4.929025 | 5.55874 | 1.547259 |
| TC19000735.hg.1 | RPS11 | 6.486194 | 7.115271 | 1.546575 |
| TC06000501.hg.1 | HMGA1 | 6.081682 | 6.709767 | 1.545512 |
| TC01004065.hg.1 | C1orf229 | 5.555701 | 6.182549 | 1.544188 |
| TC15000581.hg.1 | RAB8B | 3.522107 | 4.148733 | 1.54395 |
| TC11001213.hg.1 | LOC100507510 | 3.457521 | 4.084034 | 1.543829 |
| TC01003618.hg.1 | ARPC5 | 4.542419 | 5.168602 | 1.543476 |
| TC08001458.hg.1 | RPL30 | 5.733222 | 6.358747 | 1.542772 |
| TC02001211.hg.1 | GCSHP3 | 4.784435 | 5.404894 | 1.537364 |
| TC02002952.hg.1 | BOK-AS1 | 6.035021 | 6.655033 | 1.536888 |
| TC04001419.hg.1 | H2AFZ | 5.186314 | 5.806018 | 1.53656 |
| TC09000643.hg.1 | LOC100505607 | 4.415424 | 5.033965 | 1.535322 |
| TC02001686.hg.1 | FTH1P3 | 5.396425 | 6.013774 | 1.534054 |
| TC02002536.hg.1 | OLA1 | 5.18556 | 5.80267 | 1.5338 |
| TC09000416.hg.1 | MIR3153 | 4.094689 | 4.709519 | 1.531378 |
| TC11001063.hg.1 | ATP5L | 3.55317 | 4.166617 | 1.52991 |
| TC05003396.hg.1 | KIF2A | 3.757178 | 4.369742 | 1.528974 |
| TC14001296.hg.1 | PNMA1 | 4.207286 | 4.819842 | 1.528966 |
| TC19001027.hg.1 | LINGO3 | 5.231982 | 5.844098 | 1.528499 |
| TC20000399.hg.1 | CEBPB | 5.205296 | 5.817341 | 1.528424 |
| TC12001227.hg.1 | CSDA | 4.653245 | 5.265069 | 1.52819 |
| TC03000808.hg.1 | WWTR1-AS1 | 4.801518 | 5.413045 | 1.527876 |
| TC17001894.hg.1 | F0XJ1 | 4.251142 | 4.861246 | 1.526369 |
| TC10001427.hg.1 | DUPD1 | 5.543242 | 6.153162 | 1.526175 |
| TC11000806.hg.1 | RPS3 | 5.154682 | 5.762587 | 1.524044 |
| TC17001116.hg.1 | RPL26 | 4.81932 | 5.426471 | 1.523248 |
| TC0X001276.hg.1 | GNG5P2 | 4.371895 | 4.978842 | 1.523033 |
| TC05000372.hg.1 | F2RL1 | 5.632197 | 6.238837 | 1.522709 |
| TC16000670.hg.1 | FOXC2 | 5.092462 | 5.699029 | 1.522632 |
| TC13000073.hg.1 | SNORA27 | 3.790621 | 4.396334 | 1.521731 |
| TC03001189.hg.1 | HDAC11-AS1 | 3.808107 | 4.413559 | 1.521455 |
| TC03001238.hg.1 | UBE2E1-AS1 | 4.991354 | 5.595057 | 1.519612 |
| TC01000507.hg.1 | TMCO2 | 4.300678 | 4.904234 | 1.519457 |
| TC07000131.hg.1 | SP4 | 4.53939 | 5.142725 | 1.519224 |
| TC12001920.hg.1 | LOC100287944 | 4.239803 | 4.842271 | 1.518312 |
| TC18000493.hg.1 | IER3IP1 | 4.691775 | 5.294092 | 1.518153 |
| TC09000432.hg.1 | MIR3910-1 | 4.552649 | 5.153717 | 1.516839 |
| TC17001846.hg.1 | FAM104A | 4.287311 | 4.888024 | 1.516466 |
| TC05002126.hg.1 | ZNF354A | 4.589365 | 5.18961 | 1.515974 |
| TC19000531.hg.1 | EIF3K | 5.614336 | 6.214451 | 1.515837 |
| TC21000006.hg.1 | MIR3648 | 6.347371 | 6.947324 | 1.515667 |
| TC09001601.hg.1 | RPL12 | 5.268414 | 5.868079 | 1.515365 |
| TC13000631.hg.1 | TPT1 | 4.11669 | 4.715332 | 1.514291 |
| TC14001019.hg.1 | ARHGAP5-AS1 | 5.776831 | 6.374678 | 1.513456 |
| TC6_cox_hap2000243.hg.1 | CSNK2B | 5.419956 | 6.017608 | 1.513252 |
| TC6_cox_hap2000139.hg.1 | PPP1R18 | 5.935643 | 6.532912 | 1.51285 |
| TC0X000370.hg.1 | IGBP1 | 3.989222 | 4.585773 | 1.512097 |
| TC6_ssto_hap7000196.hg.1 | CSNK2B | 5.280958 | 5.876986 | 1.511549 |
| TC11001829.hg.1 | PATL1 | 4.946288 | 5.541584 | 1.510783 |
| TC13000213.hg.1 | INTS6-AS1 | 3.631888 | 4.227146 | 1.510743 |
| TC17000187.hg.1 | UBB | 5.454652 | 6.049006 | 1.509796 |
| TC10001632.hg.1 | CALHM1 | 4.721365 | 5.313964 | 1.507961 |
| TC06001002.hg.1 | TBPL1 | 5.14214 | 5.73453 | 1.507742 |
| TC12001144.hg.1 | SCARNA11 | 4.895072 | 5.485725 | 1.505928 |
| TC06001228.hg.1 | TUBB2B | 4.066791 | 4.656343 | 1.504779 |
| TC06001343.hg.1 | HIST1H2AB | 4.007389 | 4.596445 | 1.504262 |
| TC19002663.hg.1 | ZNF548 | 4.474639 | 5.063693 | 1.50426 |
| TC19000833.hg.1 | MIR518B | 3.695711 | 4.284378 | 1.503857 |
| TC02002427.hg.1 | MIR4773-2 | 3.987904 | 4.576196 | 1.503466 |
| TC19000197.hg.1 | SWSAP1 | 3.640721 | 4.22871 | 1.50315 |
| TC19000278.hg.1 | OR10H5 | 3.572731 | 4.160657 | 1.503084 |
| TC06001471.hg.1 | LOC401242 | 4.529268 | 5.11659 | 1.502455 |
| TC6_apd_hap1000052.hg.1 | LOC401242 | 4.529268 | 5.11659 | 1.502455 |
| TC6_dbb_hap3000103.hg.1 | LOC401242 | 4.529268 | 5.11659 | 1.502455 |
| TC6_mann_hap4000092.hg.1 | LOC401242 | 4.529268 | 5.11659 | 1.502455 |
| TC6_qbl_hap6000105.hg.1 | LOC401242 | 4.529268 | 5.11659 | 1.502455 |
| TC17001942.hg.1 | MIR657 | 5.494045 | 6.080672 | 1.501732 |
| TC17000464.hg.1 | CISD3 | 6.278322 | 6.863792 | 1.500528 |
| TC19000689.hg.1 | SNAR-A3 | 5.900589 | 6.485788 | 1.500246 |
| TC19000207.hg.1 | ZNF491 | 4.057921 | 3.471099 | −1.50193 |
| TC02001251.hg.1 | MIR4776-1 | 4.524966 | 3.93813 | −1.50195 |
| TC06000803.hg.1 | LOC100507024 | 4.969172 | 4.38221 | −1.50208 |
| TC01002332.hg.1 | MIR4253 | 5.177356 | 4.587452 | −1.50515 |
| TC09000468.hg.1 | MIR2278 | 4.89871 | 4.308197 | −1.50578 |
| TC06000937.hg.1 | ASF1A | 4.218333 | 3.626203 | −1.50747 |
| TC16000061.hg.1 | MIR4516 | 8.462434 | 7.870275 | −1.5075 |
| TC14002261.hg.1 | LOC100653245 | 6.089497 | 5.497311 | −1.50753 |
| TC12000059.hg.1 | LOC100507511 | 4.240054 | 3.645181 | −1.51034 |
| TC02004938.hg.1 | IGKV1-5 | 5.825066 | 5.229452 | −1.51112 |
| TC0Y000195.hg.1 | TTTY6B // TTTY6 | 4.049266 | 3.450491 | −1.51443 |
| TC17001648.hg.1 | MIR196A1 | 4.593892 | 3.994325 | −1.51526 |
| TC15000936.hg.1 | NR2F2 | 5.527656 | 4.92533 | −1.51816 |
| TC01002516.hg.1 | LOC728431 | 5.23946 | 4.635322 | −1.52007 |
| TC07000559.hg.1 | COL1A2 | 5.547641 | 4.939847 | −1.52393 |
| TC10001766.hg.1 | MIR378C | 5.133039 | 4.524848 | −1.52435 |
| TC11000282.hg.1 | MIR610 | 4.703804 | 4.095564 | −1.5244 |
| TC02004947.hg.1 | IGKV2-29 | 4.542328 | 3.933996 | −1.5245 |
| TC22000870.hg.1 | LOC100506695 // PHF21B | 5.146883 | 4.536935 | −1.5262 |
| TC0X001174.hg.1 | MAGT1 | 4.901742 | 4.290744 | −1.52732 |
| TC07001347.hg.1 | PURB | 5.12075 | 4.508843 | −1.52828 |
| TC0Y000075.hg.1 | TTTY6 | 4.025229 | 3.408903 | −1.53297 |
| TC07000766.hg.1 | MIR593 | 6.914992 | 6.29827 | −1.53339 |
| TC15001664.hg.1 | MIR631 | 5.664675 | 5.047149 | −1.53424 |
| TC08001284.hg.1 | LOC100505676 | 5.661897 | 5.042713 | −1.53601 |
| TC01000179.hg.1 | PRAMEF20 | 4.364079 | 3.742257 | −1.53882 |
| TC16002058.hg.1 | HPR | 4.026166 | 3.403859 | −1.53933 |
| TC11001804.hg.1 | OR10Q1 | 5.161554 | 4.538935 | −1.53967 |
| TC17001800.hg.1 | DDX5 | 6.389706 | 5.763003 | −1.54403 |
| TC07000907.hg.1 | TRBV5-1 | 4.034843 | 3.408021 | −1.54416 |
| TC17000535.hg.1 | VPS25 | 5.173048 | 4.544486 | −1.54602 |
| TC07000478.hg.1 | SNORA14A | 5.49603 | 4.866938 | −1.54659 |
| TC09000915.hg.1 | FLJ41200 | 4.488877 | 3.858426 | −1.54805 |
| TC13000052.hg.1 | MIR2276 | 5.436802 | 4.805642 | −1.54881 |
| TC12001082.hg.1 | KDM5A | 4.785029 | 4.15174 | −1.5511 |
| TC01002820.hg.1 | C1orf180 | 4.323572 | 3.690222 | −1.55116 |
| TC08001490.hg.1 | LOC100506652 | 4.246839 | 3.613326 | −1.55134 |
| TC22000862.hg.1 | SCUBE1 | 4.143461 | 3.508445 | −1.55295 |
| TC14002284.hg.1 | IGHV3-74 | 4.728541 | 4.092136 | −1.55445 |
| TC17000394.hg.1 | FNDC8 | 4.798313 | 4.161288 | −1.55512 |
| TC10000696.hg.1 | ZDHHC16 | 5.249322 | 4.612228 | −1.55519 |
| TC01004068.hg.1 | MIR3916 | 5.491997 | 4.854693 | −1.55542 |
| TC07001574.hg.1 | LOC100505932 | 4.225003 | 3.58538 | −1.55792 |
| TC03000224.hg.1 | HHATL-AS1 | 4.899507 | 4.259366 | −1.55848 |
| TC17001582.hg.1 | GPATCH8 | 6.013046 | 5.372785 | −1.55861 |
| TC16000570.hg.1 | NFAT5 | 4.900795 | 4.260095 | −1.55909 |
| TC12000813.hg.1 | MIR3922 | 6.145116 | 5.503224 | −1.56037 |
| TC06001791.hg.1 | DEFB113 | 4.012628 | 3.369208 | −1.56203 |
| TC03000264.hg.1 | NRADDP | 5.875864 | 5.23064 | −1.56398 |
| TC11000856.hg.1 | PCF11 | 4.585786 | 3.93862 | −1.56609 |
| TC0X001393.hg.1 | MIR503 | 5.655488 | 5.008254 | −1.56616 |
| TC12000376.hg.1 | PRPF40B | 4.453186 | 3.80541 | −1.56675 |
| TC17001609.hg.1 | MAPT-AS1 | 6.233504 | 5.583941 | −1.56869 |
| TC01002585.hg.1 | SZT2-AS1 | 5.369052 | 4.719324 | −1.56887 |
| TC11000638.hg.1 | MIR4489 | 7.796024 | 7.143852 | −1.57153 |
| TC04001473.hg.1 | MIR297 | 4.068296 | 3.413018 | −1.57492 |
| TC01001582.hg.1 | DHX9 | 4.293216 | 3.637929 | −1.57493 |
| TC02000315.hg.1 | SPTBN1 | 5.51998 | 4.864585 | −1.57505 |
| TC02002612.hg.1 | COL5A2 | 5.085433 | 4.430009 | −1.57508 |
| TC10000081.hg.1 | CELF2 | 5.820017 | 5.163046 | −1.57677 |
| TC13000763.hg.1 | SPRY2 | 5.285451 | 4.625376 | −1.58016 |
| TC20000597.hg.1 | LOC149837 | 4.456602 | 3.796487 | −1.58021 |
| TC09001704.hg.1 | MIR3689A | 5.023717 | 4.361292 | −1.58274 |
| TC03001395.hg.1 | MIR425 | 4.649437 | 3.984418 | −1.58559 |
| TC04000734.hg.1 | MIR4799 | 4.393453 | 3.727887 | −1.58619 |
| TC02002693.hg.1 | INO80D | 5.353393 | 4.685347 | −1.58892 |
| TC17000764.hg.1 | TANC2 | 5.377177 | 4.709125 | −1.58893 |
| TC0X000144.hg.1 | TAB3-AS1 | 4.920715 | 4.249573 | −1.59233 |
| TC17_ctg5_hap1000020.hg.1 | SPPL2C | 5.366756 | 4.692031 | −1.59629 |
| TC08000756.hg.1 | MIR1208 | 4.570964 | 3.891839 | −1.60117 |
| TC07000507.hg.1 | MIR548M | 4.041341 | 3.359273 | −1.60444 |
| TC19000780.hg.1 | CEACAM18 | 4.940919 | 4.257203 | −1.60627 |
| TC17000032.hg.1 | OR3A3 | 4.77773 | 4.093811 | −1.6065 |
| TC19001610.hg.1 | MIR4531 | 6.148597 | 5.459963 | −1.61176 |
| TC0X001342.hg.1 | THOC2 | 4.532162 | 3.843507 | −1.61178 |
| TC07001353.hg.1 | SNORA5C | 5.99114 | 5.302406 | −1.61187 |
| TC0X001500.hg.1 | MIR105-2 | 4.161591 | 3.471833 | −1.61301 |
| TC06000566.hg.1 | TREML5P | 4.915217 | 4.223903 | −1.61475 |
| TC09000456.hg.1 | MIRLET7A1 | 5.336246 | 4.643147 | −1.61675 |
| TC17000133.hg.1 | MIR3676 | 5.667037 | 4.972875 | −1.61794 |
| TC09001310.hg.1 | MIR4289 | 5.687288 | 4.992702 | −1.61842 |
| TC11002300.hg.1 | LOC100288346 | 5.114156 | 4.41808 | −1.62009 |
| TC13000717.hg.1 | PCDH9 | 4.073261 | 3.374372 | −1.62325 |
| TC01002803.hg.1 | FUBP1 | 5.0316 | 4.332179 | −1.62385 |
| TC09001561.hg.1 | OR1B1 | 5.330008 | 4.626914 | −1.62799 |
| TC17_ctg5_hap1000002.hg.1 | ARL17A | 4.982797 | 4.277744 | −1.6302 |
| TC0X001279.hg.1 | CAPN6 | 4.562117 | 3.855837 | −1.63159 |
| TC14001529.hg.1 | CINP | 4.243579 | 3.537285 | −1.63161 |
| TC12001942.hg.1 | MIR619 | 4.351392 | 3.64419 | −1.63263 |
| TC01002151.hg.1 | MIR4252 | 8.620723 | 7.912738 | −1.63352 |
| TC16001267.hg.1 | GLG1 | 4.556839 | 3.848842 | −1.63353 |
| TC02000875.hg.1 | MIR3679 | 5.038662 | 4.329994 | −1.63429 |
| TC01006377.hg.1 | SPRR2A | 4.15328 | 3.442726 | −1.63643 |
| TC0X001082.hg.1 | XAGE-4 | 4.087066 | 3.37427 | −1.63898 |
| TC01003762.hg.1 | C1orf147 | 5.635265 | 4.915102 | −1.64737 |
| TC01000266.hg.1 | LOC100506824 | 4.844906 | 4.121374 | −1.65122 |
| TC11003469.hg.1 | STT3A | 4.779898 | 4.055869 | −1.65179 |
| TC11001556.hg.1 | C11orf55 | 4.342326 | 3.616428 | −1.65393 |
| TC11000059.hg.1 | MIR4686 | 6.619994 | 5.89194 | −1.6564 |
| TC17000729.hg.1 | RPS6KB1 | 5.20421 | 4.475781 | −1.65683 |
| TC19000244.hg.1 | MIR181C | 4.38344 | 3.652814 | −1.65936 |
| TC02002647.hg.1 | SF3B1 | 5.311529 | 4.580748 | −1.65954 |
| TC10001251.hg.1 | GDF2 | 5.373111 | 4.641478 | −1.66052 |
| TC09001708.hg.1 | MIR3689D2 | 4.907048 | 4.175082 | −1.6609 |
| TC14000109.hg.1 | TRAV8-7 | 4.064114 | 3.328292 | −1.66535 |
| TC17000787.hg.1 | MIR634 | 6.098329 | 5.36239 | −1.66548 |
| TC16001053.hg.1 | PYDC1 | 5.860507 | 5.124064 | −1.66606 |
| TC09001614.hg.1 | MIR4672 | 5.860966 | 5.123324 | −1.66745 |
| TC09001709.hg.1 | MIR3689F | 6.685246 | 5.947473 | −1.6676 |
| TC01001027.hg.1 | MIR942 | 4.465426 | 3.725989 | −1.66952 |
| TC01002222.hg.1 | LOC645359 | 4.467091 | 3.725579 | −1.67193 |
| TC14000133.hg.1 | MMP14 | 5.698151 | 4.953106 | −1.67603 |
| TC02001959.hg.1 | MIR1285-2 | 5.849719 | 5.104124 | −1.67667 |
| TC16000823.hg.1 | CREBBP | 5.927039 | 5.176127 | −1.68286 |
| TC08000501.hg.1 | ZFHX4 | 4.949039 | 4.197508 | −1.68358 |
| TC02002927.hg.1 | MIR2467 | 5.251611 | 4.498095 | −1.6859 |
| TC17000001.hg.1 | LOC100506388 | 4.638553 | 3.881319 | −1.69025 |
| TC09000395.hg.1 | CDC20P1 | 4.820848 | 4.063584 | −1.69028 |
| TC02000926.hg.1 | EPC2 | 5.195574 | 4.434676 | −1.69455 |
| TC15000670.hg.1 | LOXL1 | 6.682571 | 5.921185 | −1.69512 |
| TC17000315.hg.1 | NLK | 5.451988 | 4.689928 | −1.69591 |
| TC17000925.hg.1 | MIR3065 | 5.678864 | 4.915815 | −1.69707 |
| TC16001154.hg.1 | CNOT1 | 4.9466 | 4.183294 | −1.69738 |
| TC12001455.hg.1 | SNORA2B | 4.749049 | 3.984152 | −1.69925 |
| TC05001581.hg.1 | MIR3660 | 4.472722 | 3.706095 | −1.70129 |
| TC19000235.hg.1 | NFIX | 7.725858 | 6.958988 | −1.70157 |
| TC09001589.hg.1 | HSPA5 | 6.197709 | 5.427835 | −1.70512 |
| TC01006403.hg.1 | KCNC4 | 4.584051 | 3.81015 | −1.70989 |
| TC03001383.hg.1 | MIR711 | 4.68726 | 3.912838 | −1.7105 |
| TC01000966.hg.1 | PROK1 | 5.546373 | 4.770395 | −1.71235 |
| TC21000130.hg.1 | SON | 4.591744 | 3.813155 | −1.71545 |
| TC11000561.hg.1 | GNG3 | 5.813546 | 5.032182 | −1.71876 |
| TC20000527.hg.1 | LOC100505815 | 4.418628 | 3.63587 | −1.72042 |
| TC05000871.hg.1 | C5orf52 | 4.77241 | 3.988854 | −1.72137 |
| TC12000012.hg.1 | WNK1 | 5.732969 | 4.94862 | −1.72231 |
| TC13000443.hg.1 | ANKRD20A9P | 4.342265 | 3.554048 | −1.72694 |
| TC05001097.hg.1 | MIR4456 | 4.292994 | 3.503296 | −1.72871 |
| TC11000133.hg.1 | ILK | 6.064645 | 5.274012 | −1.72983 |
| TC17001772.hg.1 | MED13 | 4.645951 | 3.847612 | −1.7391 |
| TC05000998.hg.1 | MIR1271 | 5.289784 | 4.48793 | −1.74334 |
| TC05000166.hg.1 | NIPBL | 5.134871 | 4.332732 | −1.74368 |
| TC10001762.hg.1 | MIR4297 | 7.987936 | 7.183937 | −1.74593 |
| TC17001724.hg.1 | SRSF1 | 4.897762 | 4.093619 | −1.74611 |
| TC11000631.hg.1 | MALAT1 | 4.048334 | 3.238661 | −1.75281 |
| TC12000348.hg.1 | H1FNT | 5.028391 | 4.217154 | −1.75472 |
| TC0X000212.hg.1 | KDM6A | 4.759834 | 3.948452 | −1.75489 |
| TC0X000556.hg.1 | SNORA35 | 4.651062 | 3.839049 | −1.75566 |
| TC12001091.hg.1 | MIR3649 | 4.90997 | 4.097091 | −1.75671 |
| TC1gl000191_random000002.hg.1 | SRSF10 | 5.390841 | 4.57509 | −1.76021 |
| TC15000813.hg.1 | DNM1P41 | 4.93222 | 4.108804 | −1.76959 |
| TC06001120.hg.1 | MIR1202 | 7.153215 | 6.325544 | −1.77482 |
| TC02001459.hg.1 | MIR4269 | 5.502205 | 4.672372 | −1.77748 |
| TC22001447.hg.1 | IGLJ6 | 5.513013 | 4.678329 | −1.78347 |
| TC11002353.hg.1 | DDX6 | 5.48931 | 4.650812 | −1.78819 |
| TC15001211.hg.1 | MEIS2 | 6.348711 | 5.506589 | −1.79268 |
| TC12002060.hg.1 | TMEM229B | 5.986041 | 5.141541 | −1.79564 |
| TC09001707.hg.1 | MIR3689B | 6.11372 | 5.268592 | −1.79642 |
| TC08000424.hg.1 | YTHDF3 | 6.250556 | 5.402244 | −1.80039 |
| TC01003491.hg.1 | DPT | 4.531039 | 3.681082 | −1.80245 |
| TC02001659.hg.1 | MIR1301 | 6.639485 | 5.787857 | −1.80454 |
| TC17000859.hg.1 | C17orf110 | 4.50396 | 3.646028 | −1.81244 |
| TC02001105.hg.1 | COL3A1 | 5.84789 | 4.984619 | −1.81916 |
| TC11000172.hg.1 | SNORA23 | 5.561623 | 4.697825 | −1.81982 |
| TC10000820.hg.1 | TCF7L2 | 6.150637 | 5.272392 | −1.83814 |
| TC22001449.hg.1 | IGLC7 | 5.00244 | 4.124182 | −1.83815 |
| TC07000817.hg.1 | LOC100506860 | 4.648329 | 3.765656 | −1.84379 |
| TC04001565.hg.1 | PCDH18 | 4.138818 | 3.254888 | −1.8454 |
| TC16001008.hg.1 | C16orf54 | 5.131753 | 4.247796 | −1.84543 |
| TC20000195.hg.1 | MIR3193 | 4.529956 | 3.641586 | −1.85108 |
| TC02001585.hg.1 | MIR4262 | 5.294123 | 4.399172 | −1.85955 |
| TC03000673.hg.1 | MIR1280 | 8.704777 | 7.809206 | −1.86035 |
| TC15000270.hg.1 | THBS1 | 5.229958 | 4.332397 | −1.86291 |
| TC02000386.hg.1 | MEIS1 | 5.342184 | 4.4412 | −1.86734 |
| TC01001077.hg.1 | LOC730256 // LOC642441 | 5.491196 | 4.58 | −1.8806 |
| TC06001686.hg.1 | MIR4462 | 4.464953 | 3.55092 | −1.88431 |
| TC14001287.hg.1 | LOC100653305 // LOC100652952 | 4.503014 | 3.586151 | −1.88801 |
| TC02000536.hg.1 | MIR4435-1 | 4.229815 | 3.309362 | −1.89271 |
| TC09001534.hg.1 | MIR147A | 6.712748 | 5.78512 | −1.90215 |
| TC17001159.hg.1 | MIR4731 | 6.981643 | 6.052115 | −1.90465 |
| TC14000919.hg.1 | SNORD8 | 5.6997 | 4.76953 | −1.9055 |
| TC02000404.hg.1 | MIR3126 | 4.564993 | 3.62425 | −1.91952 |
| TC02001416.hg.1 | SCARNA5 | 4.451994 | 3.503804 | −1.92945 |
| TC11000398.hg.1 | MIR3160-2 | 5.542532 | 4.591221 | −1.93363 |
| TC09001073.hg.1 | MIR4540 | 5.43216 | 4.464309 | −1.95592 |
| TC10001270.hg.1 | MIR4294 | 6.331278 | 5.359011 | −1.96192 |
| TC0X000282.hg.1 | MIR500A | 5.220169 | 4.245045 | −1.96581 |
| TC07001219.hg.1 | HOXA2 | 4.913581 | 3.925485 | −1.98357 |
| TC09000159.hg.1 | SNORD121B | 5.19182 | 4.201111 | −1.98716 |
| TC02002943.hg.1 | LOC728208 | 5.064889 | 4.06994 | −1.99301 |
| TC11000031.hg.1 | SNORA52 | 6.992956 | 5.993691 | −1.99898 |
| TC04001578.hg.1 | PPP1R14BP3 | 6.060392 | 5.056371 | −2.00558 |
| TC14000418.hg.1 | SRSF5 | 5.784508 | 4.772988 | −2.01603 |
| TC01003471.hg.1 | MIR921 | 4.654395 | 3.641995 | −2.01726 |
| TC0X000388.hg.1 | OGT | 4.624113 | 3.611639 | −2.01737 |
| TC10000164.hg.1 | YWHAZP3 | 5.566947 | 4.550217 | −2.02333 |
| TC12001854.hg.1 | MIR4303 | 5.22711 | 4.209801 | −2.02414 |
| TC03000814.hg.1 | TSC22D2 | 7.058623 | 6.039749 | −2.02634 |
| TC13000424.hg.1 | DCUN1D2-AS2 | 5.295826 | 4.275666 | −2.02814 |
| TC09001074.hg.1 | MIR4476 | 5.342408 | 4.319373 | −2.03219 |
| TC10000146.hg.1 | MIR4675 | 5.203658 | 4.178368 | −2.03537 |
| TC07000714.hg.1 | MIR3666 | 4.011671 | 2.983393 | −2.03959 |
| TC0X000201.hg.1 | CASK-AS1 | 6.63735 | 5.602173 | −2.04937 |
| TC09000642.hg.1 | MIR181B2 | 5.001238 | 3.938373 | −2.08908 |
| TC01003990.hg.1 | SNORA14B | 5.419961 | 4.356504 | −2.08993 |
| TC04000007.hg.1 | MIR571 | 5.499165 | 4.420955 | −2.11141 |
| TC19000151.hg.1 | HNRNPM | 7.013203 | 5.932759 | −2.11469 |
| TC20000477.hg.1 | MIR646 | 5.780217 | 4.695702 | −2.12066 |
| TC10001145.hg.1 | MIR604 | 4.563907 | 3.476836 | −2.12442 |
| TC03000832.hg.1 | MBNL1 | 5.664012 | 4.574232 | −2.12842 |
| TC11000769.hg.1 | MIR4692 | 6.829966 | 5.735733 | −2.135 |
| TC04000160.hg.1 | MIR218-1 | 4.198035 | 3.082331 | −2.16701 |
| TC10001508.hg.1 | MIR107 | 4.175469 | 3.058265 | −2.16926 |
| TC14000661.hg.1 | MIR370 | 5.515684 | 4.395719 | −2.17342 |
| TC0X001207.hg.1 | PCDH19 | 4.461317 | 3.339015 | −2.17694 |
| TC07001221.hg.1 | HOXA5 | 6.061344 | 4.921702 | −2.20326 |
| TC04000360.hg.1 | MIR1269A | 6.424496 | 5.284814 | −2.20332 |
| TC11001721.hg.1 | MIR3160-1 | 5.83736 | 4.687241 | −2.21932 |
| TC18000522.hg.1 | SNORA37 | 6.159097 | 5.002299 | −2.22962 |
| TC0X000969.hg.1 | CASK | 6.003073 | 4.841478 | −2.23705 |
| TC05000709.hg.1 | SNORA74A | 4.324363 | 3.161723 | −2.23867 |
| TC03001423.hg.1 | LUST | 4.709347 | 3.53739 | −2.25317 |
| TC08001005.hg.1 | MIR383 | 6.073693 | 4.895367 | −2.26314 |
| TC05000701.hg.1 | EGR1 | 6.076075 | 4.896471 | −2.26515 |
| TC02000882.hg.1 | MIR128-1 | 4.627045 | 3.432765 | −2.28831 |
| TC13000501.hg.1 | LINC00415 | 7.914881 | 6.717649 | −2.29299 |
| TC13000800.hg.1 | LINC00361 | 6.131493 | 4.929757 | −2.30016 |
| TC07000132.hg.1 | MIR1183 | 4.003375 | 2.800602 | −2.30182 |
| TC01002776.hg.1 | MIR186 | 4.327394 | 3.114914 | −2.31736 |
| TC13000320.hg.1 | MIR622 | 5.938068 | 4.705079 | −2.35053 |
| TC20000405.hg.1 | MIR645 | 5.223802 | 3.983041 | −2.36323 |
| TC04000467.hg.1 | MIR4451 | 5.594742 | 4.331674 | −2.40006 |
| TC22000477.hg.1 | MIR3198-1 | 5.457746 | 4.193546 | −2.40194 |
| TC11002382.hg.1 | MIR125B1 | 4.025867 | 2.750099 | −2.42128 |
| TC07000965.hg.1 | MIR548T | 4.072807 | 2.786881 | −2.43839 |
| TC15001577.hg.1 | SCARNA14 | 6.018013 | 4.722064 | −2.45538 |
| TC09000916.hg.1 | NFIB | 6.509025 | 5.199226 | −2.47907 |
| TC07001599.hg.1 | MIR1285-1 | 8.055191 | 6.742648 | −2.48379 |
| TC08000259.hg.1 | SNORD13 | 5.253273 | 3.923758 | −2.51318 |
| TC12002007.hg.1 | MIR4472-2 | 7.091542 | 5.735283 | −2.5602 |
| TC09000963.hg.1 | MIR31 | 4.515691 | 3.121112 | −2.62912 |
| TC05000721.hg.1 | IGIP | 4.114312 | 2.674805 | −2.71228 |
| TC20000447.hg.1 | MIR5095 | 6.098718 | 4.639575 | −2.74945 |
| TC03000436.hg.1 | MIR4444-1 | 7.254175 | 5.790649 | −2.75782 |
| TC11002391.hg.1 | SNORD14E | 6.725938 | 5.241825 | −2.79745 |
| TC08001548.hg.1 | EXT1 | 7.248168 | 5.724334 | −2.87554 |
| TC02000923.hg.1 | ACVR2A | 5.802602 | 4.26583 | −2.90145 |
| TC0X000283.hg.1 | MIR362 | 4.42761 | 2.858813 | −2.96657 |
| TC06000824.hg.1 | MIR2113 | 5.084683 | 3.468632 | −3.06535 |
| TC09001461.hg.1 | MIR32 | 4.260347 | 2.641848 | −3.07055 |
| TC11001454.hg.1 | SNORD14B | 4.412351 | 2.706433 | −3.26236 |
| TC09000457.hg.1 | MIRLET7F1 | 4.557094 | 2.845714 | −3.27474 |
| TC11001962.hg.1 | CD248 | 7.315282 | 5.380726 | −3.8226 |
| TC18000505.hg.1 | SNORD58A | 4.184764 | 2.159425 | −4.07088 |
| TC10000755.hg.1 | MIR146B | 4.405058 | 2.37747 | −4.07723 |
| TC20000581.hg.1 | MIR103B2 | 5.218163 | 3.097698 | −4.34834 |
| TC05000067.hg.1 | SNORD123 | 5.656614 | 3.479808 | −4.52151 |
| TC20000045.hg.1 | MIR103A2 | 5.559469 | 3.352126 | −4.61824 |
| TC0X000908.hg.1 | MIR23C | 4.656479 | 2.44546 | −4.63002 |
| TC11001820.hg.1 | OR5A2 | 5.302281 | 3.075891 | −4.67962 |
| TC0X001430.hg.1 | MIR505 | 4.918149 | 2.637829 | −4.85786 |
| TC14000798.hg.1 | SNORA28 | 4.250294 | 1.939967 | −4.95995 |
| TC14002223.hg.1 | IGHD2-21 | 7.056242 | 4.692148 | −5.14829 |
| TC16001253.hg.1 | SNORD71 | 4.481426 | 2.039632 | −5.43317 |
| TC17001801.hg.1 | MIR5047 | 7.933146 | 5.433694 | −5.65471 |
| TC01003533.hg.1 | SNORD78 | 5.392019 | 2.789227 | −6.07461 |
| TC15001581.hg.1 | SNORD16 | 8.125998 | 5.015806 | −8.63497 |
| TC17000728.hg.1 | MIR21 | 6.92532 | 3.543194 | −10.4261 |
| TC01001501.hg.1 | MIR3120 | 9.395077 | 5.338768 | −16.6368 |

TABLE 5
Selected Gene Array Data from Lavender Extract-Treated 3T3 cells

| | | | | fold |
|---|---|---|---|---|
| | | | | change_LVvs |
| Probe Set ID | Gene Symbol | 3T3-CON | 3T3-LV | Con |
| TC22001440.hg.1 | IGLV7-43 | 4.387714 | 5.137207 | 1.681202 |
| TC12000070.hg.1 | LOC100507560 | 4.011845 | 4.756153 | 1.675171 |
| TC01002656.hg.1 | KTI12 | 3.552426 | 4.283055 | 1.659362 |
| TC6_dbb_hap3000114.hg.1 | IFITM4P | 4.406431 | 5.118879 | 1.638582 |
| TC6_mann_hap4000103.hg.1 | IFITM4P | 4.406431 | 5.118879 | 1.638582 |
| TC6_qbl_hap6000115.hg.1 | IFITM4P | 4.406431 | 5.118879 | 1.638582 |
| TC6_ssto_hap7000106.hg.1 | IFITM4P | 4.693115 | 5.401864 | 1.634386 |
| TC02002528.hg.1 | LOC100287375 | 3.338941 | 4.032633 | 1.617417 |
| TC6_cox_hap2000125.hg.1 | IFITM4P | 5.132969 | 5.814871 | 1.604253 |
| TC06001436.hg.1 | TOB2P1 | 3.472974 | 4.102049 | 1.546573 |
| TC15001577.hg.1 | SCARNA14 | 6.018013 | 6.642136 | 1.541274 |
| TC09000643.hg.1 | LOC100505607 | 4.415424 | 5.013746 | 1.513955 |
| TC06000695.hg.1 | MIR4485 | 5.921429 | 5.315357 | −1.52211 |
| TC20000581.hg.1 | MIR103B2 | 5.218163 | 4.611418 | −1.52282 |
| TC0X001430.hg.1 | MIR505 | 4.918149 | 4.300916 | −1.53393 |
| TC01006376.hg.1 | SPRR2F | 4.327412 | 3.702894 | −1.5417 |
| TC07000964.hg.1 | MIR548F3 | 4.907326 | 4.271287 | −1.55406 |
| TC11000631.hg.1 | MALAT1 | 4.048334 | 3.361494 | −1.60975 |
| TC05001367.hg.1 | LOC100653008 // LOC100652914 | 6.173776 | 5.471752 | −1.62679 |
| TC05000921.hg.1 | MIR103B1 | 4.393016 | 3.602158 | −1.7301 |
| TC09001561.hg.1 | OR1B1 | 5.330008 | 4.535229 | −1.73481 |
| TC05000673.hg.1 | MIR4461 | 4.550079 | 3.619755 | −1.9057 |

In some embodiments the present invention relates to compositions applicable in the treatment of patients with multiple diseases, disorders or conditions. It has commonly been taught that virtually all medications pose significant, adverse, side-effects. Moreover, patients requiring treatment for multiple conditions are typically treated with multiple agents, compounds or drugs, and, as a result, frequently suffer injury due to drug-drug interactions.

It is medically desirable to provide compositions comprising synergistic combinations of at least one extract selected from the group comprising orange, frankincense, cannabis or other natural oil or extract or at least one substance or compound isolated from said at least one oil or extract and other natural oils and/or extracts which are capable of treating multiple conditions, yet demonstrate reduced tendency to drug-drug interactions as compared to the combinations of approved drugs commonly used to prevent or treat the same group of diseases, disorders and conditions.

It is a proposition of this invention that it is desirable to provide deuterium depleted water (DDW) and/or at least one extract selected from the group comprising orange, frankincense, cannabis or other natural oil or extract or at least one substance or compound isolated from said at least one oil or extract and other natural oils and/or extracts and/or other FDA approved and/or non-FDA agent(s), compound(s), or drug(s) of the present invention in the context of a large number of diseases and conditions.

It is a further proposition of this invention that it is desirable to provide deuterium depleted water (DDW) and/or at least one extract selected from the group comprising orange, frankincense, cannabis or other natural oil or extract or at least one substance or compound isolated from said at least one oil or extract and other natural oils and/or extracts and/or other FDA approved and/or non-FDA agent(s), compound(s), or drug(s) of the present invention to prevent a large number of diseases and conditions.

In some embodiments, the present invention relates to compounds, drugs, and agents, or compositions, manufacture, products, processes, methods, and/or methods of use, prevention and treatment that improve drug action, e.g. reduce chemotherapeutic resistance including, but not limited to, cancer chemotherapeutic resistance, chemotherapeutic resistance to anti-hypertensive agents, cardioprotectant agents, chemotherapeutic resistance to anti-obesity agents, fertility agents, chemotherapeutic resistance to glycemic control agents, chemotherapeutic resistance to anti-hyperlipidemic agents, chemotherapeutic resistance to an anti-atherosclerotic agent, etc.

For example, a composition (e.g. comprising at least one extract selected from the group comprising orange, frankincense, cannabis or other natural oil or extract or at least one substance or compound isolated from said at least one oil or extract and other natural oils and/or extracts that reduces hyperlipidemia may be one that appropriately modulates the amount or activity of a hyperlipidemia related gene (e.g. SREBP-1c or other hyperlipidemia related gene listed herein).

In some embodiments the novel compositions and delivery methods likewise provide for increased palatability, especially via the oral route, particularly when such drugs and compounds otherwise are unpalatable.

The invention also specifically covers the use of compounds, agents, and drugs specified or named herein (and their analogs), in conjunction with other anti-hypertensive agents, cardioprotectant agents, anti-obesity agents, fertility agents, glycemic control agents, anti-hyperlipidemic agents, anti-atherosclerotic agents, anti-cancer agents, anti-chemotherapeutic resistance agents, and other approved agents and drugs as part of combination therapies and medicinal compositions.

In some embodiments, the present invention relates to a method of identifying agents, compounds or drugs useful in preventing or treating CDCP related diseases and conditions as well as other disorders diseases and conditions treatable or preventable.

The present invention further relates to a method of identifying agents, compounds or drugs useful in preventing or treating CDCP related diseases and conditions as well as other disorders diseases and conditions treatable or preventable by the same agents, compounds or drugs.

The method may comprise administering a candidate agent, compound, or drug to an animal or contacting cells in vitro or in vivo with a candidate agent, compound, or drug and assaying the activity of the VEGF promoter in response.

Various methods known to those skilled in the art for assaying promoter activity may be utilized including, RT-PCR, Western blot, and the use of recombinant reporter constructs such as those comprising luciferase or fluorescent protein operably linked to the VEGF promoter. The VEGF promoter of the invention may be one deriving from multiple species, but is preferably a vertebrate promoter, and preferably a mammalian promoter, and preferably a human VEGF promoter.

Similarly, the activity or amounts of proteins regulated by VEGF may be assayed as another less direct means of assaying VEGF promoter activity. Suitable cells for conducting the assay(s) include those of mammals, e.g., laboratory animals, such as mice, rats, and other rodents as well as primates, etc. In one embodiment, the cell is a human cell.

Determining whether a compound reduces VEGF activity may include contacting the cell expressing VEGF with the agent, compound or drug. The term “contacting” refers to directly or indirectly bringing the cell and the compound together in physical proximity. The contacting may be performed in vitro or in vivo. For example, the cell may be contacted by delivering the agent, compound or drug to the cell through known techniques, such as microinjection, injecting the compound into the bloodstream of a mammal, and incubating the cell in a medium that includes the compound.

Also, determining whether an agent, compound, or drug reduces VEGF activity may further comprise measuring the level of VEGF activity in the cell. The level of VEGF may be measured by any method known in the art, including for example, immunohistochemistry, PCR analysis, RT-PCR, Northern blot, Western blot, ELISA assays, GFP reporter expression, luciferase reporter assays, etc. Accordingly, the level of VEGF activity may be assessed by measuring the level of induction of a reporter gene that is operably linked to the VEGF promoter or fused to the VEGF gene.

The level of VEGF activity may also be assessed by detecting the level of activity of a gene that is regulated by VEGF.

In some embodiments, cells that express VEGF in response to a known agent will be induced to express VEGF through exposure to that known agent and the level of VEGF activity measured in the cell in the presence of the candidate agent, compound or drug. Accordingly, the candidate's ability to reduce VEGF is measured in relation to the level of VEGF activity in the cell contacted with the known inducing agent.

In another aspect, the invention relates to a method for reducing VEGF activity in a cell in a human or animal in need thereof. The method includes administering to the human or animal an effective amount of an agent, compound or drug that inhibits VEGF activity (e.g. VEGF promoter activity) and that is named herein.

In some embodiments, the compositions, manufacture, products, processes, methods, and/or methods of use, prevention and treatment of the present invention comprise an agent, compound or drug that blocks VEGF promoter activation.

In some embodiments, two or more agents are selected for use in combination from the list including a sequiterpene (e.g. one or more natural oil extract(s)), glutathione, zingerone, curcumin or derivative, a flavone, flavonoid, a gingerol, a shogaol, an ATF4 modulator, an FST1 modulator, an NRF2 modulator, a KEAP1 modulator, a VEGF inhibitor, homocysteine, vitamin C, n-acetylcysteine, trimethylglycine, folinic acid, folic acid, reduced glutathione, an amino acid, an OTC drug, and an approved drug.

It has been taught that ATF4 activity is required to increase intracellular glutathione. While not bound by theory, the applicants believe, agents, compounds, or drugs increasing intracellular glutathione, while simultaneously inhibiting ATF4 are desirable for inclusion in the present invention, including those listed and described herein.

Finally, the invention relates to compositions, manufacture, products, processes, methods, and/or methods of use, prevention and treatment capable of extending the life span of a cell, tissue, organ, or organism (especially a human). If nothing else, cancerous cell behavior demonstrates that cellular immortality and cell death are gene expression and epigenetically determined phenomena.

The present invention provides for compositions, manufacture, products, processes, methods, and/or methods of use, prevention and treatment that can modulate re-program and/or re-set the “expiry date” inherent to all living things, thereby extending lifespan.

In part, the invention relates to a method for preventing or treating a CDCP or ODDC disease or condition. The method comprises administering a therapy, composition comprising at least one extract selected from the group comprising orange, frankincense, cannabis or other natural oil or extract or at least one substance or compound isolated from said at least one oil or extract and other natural oils and/or extracts and/or at least one other agent, compound, or drug of the present invention.

In part, the invention relates to methods for administering a compound, agent or drug with anti-hypertensive, anti-obesity, glycemic control, anti-hyperlipidemic, anti-atherosclerotic, and/or an anti-chemotherapeutic resistance properties to an animal, an invertebrate, a vertebrate, an insect, fish, amphibian, bird, mammal or to a human in need thereof. The method comprises administering a composition comprising deuterium depleted water (DDW) and/or at least one extract selected from the group comprising orange, frankincense, cannabis or other natural oil or extract or at least one substance or compound isolated from said at least one oil or extract and other natural oils and/or extracts and/or at least one other agent, compound, or drug of the present invention. Non-limiting examples of other disorders (herein termed “other disorders”, or ODDC) preventable or ameliorated by administration of the compositions, manufacture, products, processes, methods, and/or methods of use, prevention and treatment described herein include, but are not limited to inflammatory diseases, oncological diseases, genetic diseases, ischemic diseases, infectious diseases, neurological diseases, hematological diseases, kidney diseases, vascular diseases, dermatological diseases, opthamological diseases, rheumatoid diseases, orthopedic diseases, gynecological diseases, obstetric diseases, pediatric diseases, etc. Additional non-limiting examples include sepsis, contrast-induced nephropathy, chronic kidney disease, pulmonary fibrosis, hypoxic conditions, chemical-induced lung injury, respiratory distress disorder, anon gap acidosis, nephritis, lupus, interstitial lung disease, graft dysfunction, hepatitis, acute kidney injury, noise-induced hearing injuries, poison ingestion, retinopathy, neurotoxicity, cancer-induced injury such as ototoxicity, respiratory infections, autism, conditions involving vasospasm, and conditions considered treatable by provision of n-acetylcysteine, injectable reduced glutathione, or a known intracellular glutathione enhancing agent.

It should be understood, however, that the present invention, with respect to all of its aspects, covers the use of another compound, agent, or drugs specified herein in combination with at least one extract selected from the group comprising orange, frankincense, cannabis or other natural oil or extract or at least one substance or compound isolated from said at least one oil or extract and other natural oils and/or extracts or in place of at least one extract selected from the group comprising orange, frankincense, cannabis or other natural oil or extract or at least one substance or compound isolated from said at least one oil or extract and other natural oils and/or extracts so long as the ultimate composition is novel and effective.

In some embodiments, the agents, compounds, or drugs of the present invention comprise an allicin and an amino acid(s).

In some embodiments, the compositions, manufacture, products, processes, methods, and/or methods of use, prevention and treatment of the present invention will be ones useful for preventing or treating conditions and diseases related to the depletion of glutathione or to insufficient glutathione.

In some embodiments, the agents, compounds or drugs of the present invention are incorporated into compositions, manufacture, products, processes, methods, and/or methods of use, prevention and treatment for reducing polypharmacy.

In one embodiment, the compositions of the present invention reduce the risk of drug-drug interaction in a patient.

In some embodiments, the agents, compounds or drugs of the present invention are incorporated into compositions, manufacture, products, processes, methods, and/or methods of use, prevention and treatment for treating or preventing signs and symptoms of CDCP and ODDC.

In some embodiments, the compositions, manufacture, products, processes, methods, and/or methods of use, prevention and treatment of the present invention are useful for preventing or treating a Neurodegenerative disease or condition. Examples of such neurodegenerative diseases include Parkinson disease, Alzheimer disease, Multiple Sclerosis, Schizophrenia, Dementia, and Huntington's disease.

In some embodiments, the compositions, manufacture, products, processes, methods, and/or methods of use, prevention and treatment of the present invention are useful for preventing or treating a mental illness.

In some embodiments, the compositions, manufacture, products, processes, methods, and/or methods of use, prevention and treatment of the present invention are useful for preventing or treating diseases or conditions related to Aging. Examples of aging related diseases include Arthritis, Diabetes, Osteoarthritis, Cataracts, Macular Degeneration and Prostate enlargement. Many other aging related diseases represent a manifestation of decreased cellular telomerase and they likewise are considered preventable or treatable with the compositions, manufacture, products, processes, methods, and/or methods of use, prevention and treatment described herein. In some embodiments, the compositions, manufacture, products, processes, methods, and/or methods of use, prevention and treatment of the present invention are useful for preventing or treating diseases or conditions related to Liver Dysfunction. Examples of such conditions and diseases include Toxic Hepatitis, Viral Hepatitis (A, B, and C), Chronic Hepatitis, Acute alcoholic Hepatitis, Alcoholic Hepatic fibrosis, Hepatic toxin exposure, and Cirrhosis.

In some embodiments, the compositions, manufacture, products, processes, methods, and/or methods of use, prevention and treatment of the present invention are useful for preventing or treating diseases or conditions related to Lung dysfunction. Examples of such diseases and conditions include Asthma, Emphysema, Pneumonia, Bronchitis (chronic and acute), Cystic fibrosis, Pulmonary fibrosis, Chronic obstructive pulmonary disease (COPD), Adult respiratory distress syndrome (ARDS).

In some embodiments, the compositions, manufacture, products, processes, methods, and/or methods of use, prevention and treatment of the present invention are useful for preventing or treating diseases or conditions related to the Cardiovascular System. Examples of such diseases and conditions include Ischemia, Atherosclerosis & its consequences, Heart failure, Heart Attack, Reperfusion injury, Kidney failure, High blood pressure, Stroke, Impaired circulation, vasculitis, and various viral and non-viral carditis.

In some embodiments, the compositions, manufacture, products, processes, methods, and/or methods of use, prevention and treatment of the present invention are useful for preventing or treating diseases or conditions related to the Digestive System.

Examples of conditions and diseases related to the Digestive System include inflammatory bowel disease, Ulcerative colitis, Crohn's disease, Gastritis, Stomach cancer, Pancreatitis, Peptic ulcer disease.

In some embodiments, the compositions, manufacture, products, processes, methods, and/or methods of use, prevention and treatment of the present invention are useful for preventing or treating diseases or conditions related to Kidney Failure & Dialysis, Examples of such diseases and conditions include Kidney failure, Renal toxicity, and Injury related to dialysis.

In some embodiments, the compositions, manufacture, products, processes, methods, and/or methods of use, prevention and treatment of the present invention are useful for treating a condition involving the skin, especially allergic conditions and conditions related to immune dysfunction.

In some embodiments, the compositions, manufacture, products, processes, methods, and/or methods of use, prevention and treatment of the present invention are useful for treating Infectious diseases.

In some embodiments, the compositions, manufacture, products, processes, methods, and/or methods of use, prevention and treatment of the present invention are utilized as anti-infectives (e.g. antibiotics, anti-microbials, anti-fungals, and antivirals, anti-helminthics, etc.)

In some embodiments, the compositions, manufacture, products, processes, methods, and/or methods of use, prevention and treatment of the present invention are useful for preventing or treating Immune System related diseases and conditions. Such diseases and conditions include viral infection, HIV and AIDS, Toxic Hepatitis & cirrhosis, Viral hepatitis (type A, B, & C), Herpes virus infection, Common Cold, various Bacterial infections, Chronic fatigue syndrome, and autoimmune dysfunction.

In some embodiments, the compositions, manufacture, products, processes, methods, and/or methods of use, prevention and treatment of the present invention are useful for preventing or treating Skin Disorders. Examples of such diseases and conditions include Pruritus, Psoriases, Eczema, SLE (lupus), Vasculitis, Polymyositis, Mycosis fungoides, Scleroderma Pemphigoid, Atopic dermatitis, Contact dermatitis, Sebborrheic dermatitis, Dermatitis herpetiformis, Acne conglobata, Acne vulgaris, Vitiligo, Alopecia areata, and UV radiation skin damage.

The invention also provides compositions, manufacture, products, processes, methods, and/or methods of use, prevention and treatment (including but not limited to oral and topical compositions) for promoting hair growth.

In some embodiments, the compositions, manufacture, products, processes, methods, and/or methods of use, prevention and treatment of the present invention are useful for preventing or treating diseases and conditions related to the Eye, Ear, Nose, Throat & Teeth. Such conditions and diseases include Cataract, Glaucoma, Macular degeneration, Hearing loss, Ear infection, Sinusitis, Periodontal (gum) disease, and upper respiratory tract disease.

In some embodiments, the compositions, manufacture, products, processes, methods, and/or methods of use, prevention and treatment of the present invention are useful for preventing or treating diseases and conditions related to the Pregnancy, Lactation & Childbirth. Examples of such disorders include Pre-eclampsia, Eclampsia Hypertension, and Diabetes.

In some embodiments, the compositions, manufacture, products, processes, methods, and/or methods of use, prevention and treatment of the present invention are useful for treating neurological disorders such as schizophrenia, multiple sclerosis, epilepsy, seizures, depression and bipolar disorder.

In some embodiments, the compositions, manufacture, products, processes, methods, and/or methods of use, prevention and treatment of the present invention are useful for treating fragile X syndrome.

In some embodiments, the compositions, manufacture, products, processes, methods, and/or methods of use, prevention and treatment of the present invention are useful for preventing or treating injuries and conditions related to Exercise & Athletic Performance. Such conditions and diseases may, for example, occur in the context of over training (e.g. Over-Training Syndrome) & the related cellular stress.

In some embodiments, the compositions, manufacture, products, processes, methods, and/or methods of use, prevention and treatment of the present invention are useful for treating a newborn.

In some embodiments, the compositions, manufacture, products, processes, methods, and/or methods of use, prevention and treatment of the present invention are useful for treating a child.

In some embodiments, the compositions, manufacture, products, processes, methods, and/or methods of use, prevention and treatment of the present invention are useful for treating an adult human.

In some embodiments, the compositions, manufacture, products, processes, methods, and/or methods of use, prevention and treatment of the present invention are useful for preventing or treating diseases and conditions related to hormonal influences such as loss of hair and fertility.

In some embodiments, the compositions, manufacture, products, processes, methods, and/or methods of use, prevention and treatment of the present invention are useful for preventing or treating diseases and conditions related to toxic exposures.

In some embodiments, the compositions, manufacture, products, processes, methods, and/or methods of use, prevention and treatment of the present invention are useful for increasing telomerase activity in a cell when such an increase is desirable or preventing or treating diseases and conditions related to reduced or insufficient telomerase activity.

In some embodiments, the compositions, manufacture, products, processes, methods, and/or methods of use, prevention and treatment of the present invention are useful for the alleviation of pain, inhibition of platelet aggregation, lowering of fever and for prevention of cardiovascular disorders with reduced toxicity and/or reduced polypharmacy.

In some embodiments, the compositions, manufacture, products, processes, methods, and/or methods of use, prevention and treatment of the present invention are useful for vasorelaxant, antianginal, anti-inflammatory, analgesic and anti-thrombotic activity with lower gastrointestinal toxicity as compared to aspirin.

In one embodiment, chronic use of the compositions, manufacture, products, processes, methods, and/or methods of use, prevention and treatment of the present invention extend the lifespan of a cell, a tissue, an organ or an organism.

In one embodiment, chronic use of the compositions, manufacture, products, processes, methods, and/or methods of use, prevention and treatment of the present invention extend the lifespan of a human.

In one embodiment, the compositions, manufacture, products, processes, methods, and/or methods of use, prevention and treatment of the present invention are utilized as anti-infectives (e.g. antibiotics, anti-microbials, anti-fungals, and antivirals, anti-protozoals, anti-helminthics, etc.).

Furthermore, the compositions, manufacture, products, processes, methods, and/or methods of use, prevention and treatment of the present invention may, in some embodiments, also be beneficial in critical surgical patients, patients in intensive care settings, patients receivinghemodialysis.

In another part, the invention relates to compositions, manufacture, products, processes, methods, and/or methods of use, prevention and treatment for reducing an animal's body fat, increasing energy expenditure, and increasing oxygen consumption. Such activity represents organismal responses that may be assayed as a means of identifying compounds, drugs, and medicinal compositions suitable for preventing or treating CDCP, diseases related to CDCP, and/or chemotherapeutic resistance. Likewise, these organismal responses may be assayed to measure the efficacy of such compounds, drugs, and medicinal compositions.

In a further part, the invention relates to compositions, manufacture, products, processes, methods, and/or methods of use, prevention and treatment for increasing lipolysis, increasing expression of uncoupling protein 2 (UCP2) and beta-oxidation genes, decreasing expression of lipogenic genes in white adipose tissue, thereby increasing utilization and decreasing synthesis of fatty acids. Such activity represents organismal responses that may be assayed as a means of identifying compounds, drugs, and medicinal compositions suitable for preventing or treating CDCP, diseases related to CDCP, and/or chemotherapeutic resistance. Likewise, these organismal responses may be assayed to measure the efficacy of such compounds, drugs, and medicinal compositions.

In a further part, the invention relates to compositions, manufacture, products, processes, methods, and/or methods of use, prevention and treatment for increasing UCP 1, 2 and 3 expression in brown adipose tissue (BAT), thereby increasing thermogenesis. Such activity represents organismal responses that may be assayed as a means of identifying compounds, drugs, and medicinal compositions suitable for preventing or treating CDCP, diseases related to CDCP, and for chemotherapeutic resistance. Likewise, these organismal responses may be assayed to measure the efficacy of such compounds, drugs, and medicinal compositions.

In a further part, the invention relates to compositions, manufacture, products, processes, methods, and/or methods of use, prevention and treatment for improving ovulatory function (and thus fertility) in a female in need of such improvement, regularizing her menstrual cycle, and reducing hirsutism. Such activity represents organismal responses that may be assayed as a means of identifying compounds, drugs, and medicinal compositions suitable for preventing or treating CDCP, diseases related to CDCP, and/or chemotherapeutic resistance. Likewise, these organismal responses may be assayed to measure the efficacy of such compounds, drugs, and medicinal compositions.

In a further part, the invention relates to compositions, manufacture, products, processes, methods, and/or methods of use, prevention and treatment for lowering levels of circulating carbohydrate, preventing or treating age-related obesity, preventing or treating diet-related obesity, and preventing or treating steatosis. Such activity represents organismal responses that may be assayed as a means of identifying compounds, drugs, and medicinal compositions suitable for preventing or treating CDCP, diseases related to CDCP, and/or chemotherapeutic resistance. Likewise, these organismal responses may be assayed to measure the efficacy of such compounds, drugs, and medicinal compositions.

In a further part, the invention relates to compositions, manufacture, products, processes, methods, and/or methods of use, prevention and treatment for preventing or treating chronic hyperglycemia, and preventing or treating diet-induced diabetes. Such activity represents organismal responses that may be assayed as a means of identifying compounds, drugs, and medicinal compositions suitable for preventing or treating CDCP, diseases related to CDCP, and/or chemotherapeutic resistance. Likewise, these organismal responses may be assayed to measure the efficacy of such compounds, drugs, and medicinal compositions.

In a further part, the invention relates to compositions, manufacture, products, processes, methods, and/or methods of use, prevention and treatment for preventing or treating chemotherapeutic resistance in a cell, tumor, or cancer cell. Such activity represents organismal responses that may be assayed as a means of identifying compounds, drugs, and medicinal compositions suitable for preventing or treating CDCP, diseases related to CDCP, and/or chemotherapeutic resistance. Likewise, these organismal responses may be assayed to measure the efficacy of such compounds, drugs, and medicinal compositions.

In one aspect, the invention relates to compositions, manufacture, products, processes, methods, and/or methods of use, prevention and treatment with anti-hypertensive agent, cardioprotectant agent, anti-obesity agent, glycemic control agent, anti-hyperlipidemic agent, an anti-atherosclerotic agent, and/or an agent preventing or treating chemotherapeutic resistance.

In some embodiments, the compositions, manufacture, products, processes, methods, and/or methods of use, prevention and treatment of the present invention are utilized to counteract a high fat diet.

In some embodiments, the compositions, manufacture, products, processes, methods, and/or methods of use, prevention and treatment of the present invention are utilized to counteract a diet of excessive calories.

In some embodiments, at least one extract selected from the group comprising orange, frankincense, cannabis or other natural oil or extract or at least one substance or compound isolated from said at least one oil or extract and other natural oils and/or extracts and/or at least one other agent, compound, or drug of the present invention, with or without reduced glutathione, is provided to reduce resistance to an approved drug, including, but not limited to anti-cancer drugs, glycemic control drugs, anthypertensie drugs, lipid reducing drugs, etc.

In some embodiments, at least one extract selected from the group comprising orange, frankincense, cannabis or other natural oil or extract or at least one substance or compound isolated from said at least one oil or extract and other natural oils and/or extracts and/or at least one other agent, compound, or drug of the present invention is provided (with or without reduced glutathione) to reduce resistance to an FDA over-the-counter (OTC) drug.

Vitamin C may be provided whenever reduced glutathione is selected for inclusion in the compositions, manufacture, products, processes, methods, and/or methods of use, prevention and treatment of the present invention in an amount to 0.5% w/v as needed.

It should be understood that “at least one other natural oil and/or extract as well as FDA approved drugs, and non-FDA approved drugs, as used herein, refers to the naturally or synthetically obtained agent, compound or drug, as well as its analogs and derivatives e.g. as described in the examples below.

It should also be understood that the present invention covers the combination of at least one extract selected from the group comprising orange, frankincense, cannabis or other natural oil or extract or at least one substance or compound isolated from said at least one oil or extract and other natural oils and/or extracts, FDA approved drugs, and non-FDA approved drugs (e.g. at least one extract selected from the group comprising orange, frankincense, cannabis or other natural oil or extract or at least one substance or compound isolated from said at least one oil or extract and other natural oils and/or extracts, its analogs, or its derivatives) with other at least one agent, compound, or drug of the present invention.

It should also be understood that the present invention covers all compositions, manufacture, products, processes, methods, and/or methods of use, prevention and treatment wherein at least one extract selected from the group comprising orange, frankincense, cannabis or other natural oil or extract or at least one substance or compound isolated from said at least one oil or extract and other natural oils and/or extracts, its analogs, or its derivatives are replaced in those compositions, manufacture, products, processes, methods, and/or methods of use, prevention and treatment with other at least one agent, compound, or drug of the present invention.

In other embodiments, at least one extract selected from the group comprising orange, frankincense, cannabis or other natural oil or extract or at least one substance or compound isolated from said at least one oil or extract and other natural oils and/or extracts, and/or at least one other agent, compound, or drug of the present invention with or without reduced glutathione, is used in combination with approved drugs to provide enhanced anti-microbial action versus a targeted pathogen.

In other embodiments, at least one extract selected from the group comprising orange, frankincense, cannabis or other natural oil or extract or at least one substance or compound isolated from said at least one oil or extract and other natural oils and/or extracts, and/or at least one other agent, compound, or drug of the present invention is used (with or without reduced glutathione) in combination with OTC drugs to provide enhanced anti-microbial action versus a targeted pathogen.

Such compositions, manufacture, products, processes, methods, and/or methods of use, prevention and treatment may comprise biological molecules and small molecules as well as inorganic and organic compounds.

The terms “composition” refer to a substance, e.g., a compound, cell, etc., that limits cellular dysfunction and/or maintains normal function by preventing or treating the consequences of CDCP or disorders related to CDCP.

Disease related activity (including signs and symptoms of disease) is considered reduced according to the invention if it is reduced at least about 10%, preferably, at least about 20%, more preferably at least about 30%, even more preferably at least about 40%, and most preferably at least about 50% or more than in the absence of the compositions, manufacture, products, processes, methods, and/or methods of use, prevention and treatment. Optimally, at least about 70%, more optimally at least about 85%, and most optimally 100% of the symptoms or signs of CDCP, or a disease related to CDCP, are reduced in vitro, ex vivo, or in vivo.

Typically, the act of determining whether a composition modulates disease or a condition related activity at the tissue, organ or organismal level further includes measuring the parameters in a patient by which the disease or condition is defined. However, the present invention also covers a method for determining whether a composition modulates disease or a condition wherein at least one extract selected from the group comprising orange, frankincense, cannabis or other natural oil or extract or at least one substance or compound isolated from said at least one oil or extract and other natural oils and/or extracts are applied to a target cell or infectious organism (be it prokaryotic, eukaryotic, vertebrate, invertebrate, protozoal, helminthic, avian, mammalian, reptilian, piscean, or a virally-infected cell) and subsequent gene expression and protein expression changes in said target cell or infectious organism is assayed to determine whether the affected signal transduction pathways are ones that are known or can to be shown to be signal transduction pathways correlated with modulating disease or a condition.

For example, a composition (e.g. comprising at least one extract selected from the group comprising orange, frankincense, cannabis or other natural oil or extract or at least one substance or compound isolated from said at least one oil or extract and other natural oils and/or extracts) that promotes stem cell renewal may be one that increases the amount or activity of c-myc and/or KLF4.

For example, a composition (e.g. comprising at least one extract selected from the group comprising orange, frankincense, cannabis or other natural oil or extract or at least one substance or compound isolated from said at least one oil or extract and other natural oils and/or extracts) that may be used as a chemotherapeutic may be one that reduces the expression of a PRR+ Numb isoform or increase the amount or activity of p53 and pro-apoptotic genes, and/or other genes such as those described in Anticancer Genes edited by Grimm (2014).

Likewise, the compositions, manufacture, products, processes, methods, and/or methods of use, prevention and treatment of the invention may modulate one or more of the following: tissue inflammation or swelling; pro-atherogenic cytokine production by endothelial cells, endothelial dysfunction, an invasion of blood vessel walls by monocytes, conversion of monocytes/macrophages to foam cells, smooth muscle proliferation, smooth muscle migration from tunica media to intima, plaque initiation, plaque progression, and plaque rupture; production of adipokines (e.g. TNF-alpha, IL-6, leptin, plasminogen activator inhibitor-1 (PAI-1), angiotensinogen, resistin, and C-reactive protein (CRP) by fat cells; an increase in plasma cholesterol, an increase in plasma LDL, an increase in plasma triacylglycerols, a decrease in plasma HDL; an increase in blood glucose, an increase in fasting blood glucose, glucose intolerance, hyperinsulinemia, insulin resistance, HbA1, a dependence upon exogenous insulin; systolic and/or diastolic blood pressure, an angiotensin II, microalbuminuria; or cellular resistance to a chemotherapeutic agent.

In various embodiments, the composition for preventing or treating diseases related to CDCP, such as cardiovascular disease, diabetes, obesity, PCOS, steatosis, hyperlipidemia, and hypertension, as well as chemotherapeutic resistance, comprises one or more compounds and drugs selected from those named herein.

Anti-atherosclerotic activity refers to a composition's ability to induce a beneficial effect on blood vessels in vitro, ex vivo, or in vivo administration of the composition. Such beneficial effects include, but are not limited to preventing or reducing the likelihood of at least one of the following events: pro-atherogenic cytokine production by endothelial cells, endothelial dysfunction, an invasion of blood vessel walls by monocytes, conversion of monocytes/macrophages to foam cells, lipid oxidation, smooth muscle proliferation, smooth muscle migration from tunica media to intima, plaque initiation, plaque progression, and plaque rupture.

Anti-obesity activity refers to a composition's ability to induce a beneficial effect regarding excess weight gain upon in vitro, ex vivo, or in vivo administration of the composition. Such beneficial effects include, but are not limited to preventing or reducing the likelihood of one or more of the following events: production of adipokines (e.g. TNF-alpha, IL-6, leptin, plasminogen activator inhibitor-1 (PAI-1), angiotensinogen, resistin, and -reactive protein (CRP)) by fat cells.

Anti-hyperlipidemic activity refers to a composition's ability to induce a beneficial effect on lipid levels upon in vitro, ex vivo, or in vivo administration of the composition. Such beneficial effects include, but are not limited to preventing or reducing the likelihood of one or more of the following events: an increase in plasma cholesterol, an increase in plasma LDL, an increase in plasma triacylglycerols, and a decrease in plasma HDL.

For example, a composition (e.g. comprising at least one extract selected from the group comprising orange, frankincense, cannabis or other natural oil or extract or at least one substance or compound isolated from said at least one oil or extract and other natural oils and/or extracts) that reduces hyperlipidemia and/or atherosclerosis may be one that appropriately modulates the amount or activity of a hypertension related gene (AZ Fernandez, 2010; Masuyama and Hiramatsu, 2012; Khalil, 2014; ERS Cheyou, 2014)

Glycemic control refers to a composition's ability to induce a beneficial effect on glucose levels, insulin levels, glucose tolerance, and/or insulin tolerance upon in vitro, ex vivo, or in vivo administration of the composition. Such beneficial effects include, but are not limited to preventing or reducing the likelihood of one or more of the following events: an increase in random blood glucose, an increase in fasting blood glucose, glucose intolerance, hyperinsulinemia, insulin resistance, an increase in HbA1, an increased, an increased dependence upon exogenous insulin.

For example, a composition (e.g. comprising at least one extract selected from the group comprising orange, frankincense, cannabis or other natural oil or extract or at least one substance or compound isolated from said at least one oil or extract and other natural oils and/or extracts) that provides from improved glycemic control may be one that appropriately modulates the amount or activity of a glycemia related gene (e.g. V. Lyssenko, 2008; El-Osta, 2008; McCarthy, 2010; AL Siebel, 2010; Keating and El-Osta, 2013; HZ Huri, 2014; Rajasekar, 2015).

Anti-hypertensive activity refers to a composition's ability to induce a beneficial effect upon in vitro, ex vivo, or in vivo administration of the composition. Such beneficial effects include but are not limited to preventing or reducing the likelihood of one or more of the following events: an increase in systolic and/or diastolic blood pressure, an increase in angiotensin II, and an increase in microalbuminuria.

For example, a composition (e.g. comprising at least one extract selected from the group comprising orange, frankincense, cannabis or other natural oil or extract or at least one substance or compound isolated from said at least one oil or extract and other natural oils and/or extracts) that reduces hypertension may be one that appropriately modulates the amount or activity of a hypertension related gene (R M Millis, 2011; G H Kim, 2011; S. Friso 2015; Y P C Chang, 2007; H J Dai, 2013, E. Larsson, 2013; Marlene, 2012).

Anti-chemotherapeutic resistance activity refers to a composition's ability to induce a beneficial effect upon in vitro, ex vivo, or in vivo administration of the composition. Such beneficial effects include, but are not limited to preventing or reducing the likelihood of at least one of the following events: cellular resistance to a chemotherapeutic agent as demonstrated by increased or persistent dysfunction in spite of the application of an otherwise effective chemotherapeutic agent.

Anti-CDCP activity refers activity refers to a composition's ability to induce a beneficial effect upon in vitro, ex vivo, or in vivo administration of the composition. Such beneficial effects include, but are not limited to preventing or reducing the likelihood of at least one of the following events: pro-atherogenic cytokine production by endothelial cells, endothelial dysfunction, an invasion of blood vessel walls by monocytes, conversion of monocytes/macrophages to foam cells, smooth muscle proliferation, smooth muscle migration from tunica media to intima, plaque initiation, plaque progression, and plaque rupture; production of adipokines (e.g. TNF-alpha, IL-6, leptin, plasminogen activator inhibitor-1 (PAI-1), angiotensinogen, resistin, and C-reactive protein (CRP)) by fat cells; an increase in plasma cholesterol, an increase in plasma LDL, an increase in plasma triacylglycerols, a decrease in plasma HDL; an increase in blood glucose, an increase in fasting blood glucose, glucose intolerance, hyperinsulinemia, insulin resistance, an increase in HbA1, an increased dependence upon exogenous insulin; an increase in systolic and/or diastolic blood pressure, an increase in angiotensin II, an increase in microalbuminuria; or cellular resistance to a chemotherapeutic agent.

In some embodiments, the invention relates to a method for preventing or treating a variety of diseases and conditions including chronic diseases related to CDCP, such as cardiovascular disease, diabetes, obesity, PCOS, steatosis, hyperlipidemia, and hypertension and other disorders and conditions. The method comprises administering a composition comprising at least one at least one extract selected from the group comprising orange, frankincense, cannabis or other natural oil or extract or at least one substance or compound isolated from said at least one oil or extract and other natural oils and/or extracts and/or at least one other agent, compound, or drug of the present invention.

In a some embodiments, the compositions, manufacture, products, processes, methods, and/or methods of use, prevention and treatment further comprise another natural agent, compound, or drug such as a flavone or flavonoid (e.g. see USDA Database for the Flavonoid Content of Selected Foods), especially a 6,3-dimethoxyflavone or a 5,7-dimethoxyflavone.

When the compositions, manufacture, products, processes, methods, and/or methods of use, prevention and treatment, comprises curcumin, in some preferred embodiments the compositions, manufacture, products, processes, methods, and/or methods of use, prevention and treatment will modulate the expression of at least 2%, preferably at least 20%, and most preferably, >30% of genes selected from Lcn2, Reg3a, S100a8, Reg2, Lrg1, Clca4b, S100a9, Saa3, Reg3d, Reg3g, Hp, Spp1, Pla2g2a, Socs3, Ighm, Serpina3n, Dbp, Zbtb3, Ankra2, Cxcl13, Klk1b5, Speer5-ps1, Zbtb3, Ankra2, Speer5-ps1, Ctla2a, Reg3b, Chil3, Timp1, AV099323, 1700110K17Rik, AV099323, 1700110K17Rik, Retnlb, Ereg, Lbp, H1fnt, Ces1g, Fut2, Cxcl3, Plet1, C3, Igf1, Gm26744, Wfdc17, H1fnt, Rbp1, Uck2, Srp72, Ighg1, Wnt6, Enpep, Mptx1, Bmper, Gda, Gda, Stk32a, Cyp2d26, Coasy, Rrm2, Rrm2, Cxcl1, Igfbp4, Fpr2, Mettl7a1, Ly6e, Muc4, Crispld2, Crispld2, Disp1, Coasy, Ttc25, Ptgs2, Asns, Nr1d2, Ctgf, Ins2, Tc2n, Aasdhppt, Kdm4b, Abcb1a, Hist1h2bc, Lox, Fabp2, Tfrc, Erap1, Itga5, Ddit3, Tmem173, Arg1, Csf2rb, Wnt6, Tifa, Vcam1.

In a preferred embodiment, the compositions, manufacture, products, processes, methods, and/or methods of may comprise at least one extract selected from the group comprising orange, frankincense, cannabis or other natural oil or extract or at least one substance or compound isolated from said at least one oil or extract and other natural oils and/or extracts, at least one natural compound, at least one synthetic compound, and/or at least one approved drug, and are further defined by their effects on gene expression as described below.

When treating or preventing obesity, the compositions, manufacture, products, processes, methods, and/or methods of use, prevention and treatment will, in some preferred embodiments, modulate the expression of at least 2%, preferably at least 20%, and most preferably, >30% of genes selected from H19, Cyp3a16, Cpe, Efr3b, Zranb3, Fgf13, Rnf128, Ctsk, Sox4, Cadm1, Cadm1, Shisa9, Ankrd1, Ankrd1, Cd177, Nrcam, Fat3, Cap1, Btbd11, Fabp4, Igf2bp2, Itga6, Adcy3, Cdk18, Ociad2, Apbb2, Gpr176, Igf2bp2, Plxdc1, Sel1l3, NPR3, Scd1, Mgp, Gpnmb, Sox4, C77080, Syngr1, Rnf128, Npas4, S1pr5, Myl6b, Cpe, Cspg4, Ctsk, Itga6, Cda, Pgm5, Prlr, Cyfip2, Vsig4, Igf2r, Akr1b8, Csrp3, Slc45a3, Atp6v0d2, Eya4, Cx3cr1, Sorl1, Myo10, Mybph, Gpnmb, Aif1l, Trem3, Nxpe4, Chil3, Angpt2, Efr3b, Slc37a2, Mmp8, Hspa1b, Mfsd4, Cd51, Dlg3, Gdpd1, 8430408G22Rik, Atp6v0d2, Atp6v0d2, Actn1, Tpm2, Actn1, Plxdc1, Apoc2, Bcar3, Igf2, Itgb5, Fgf13, S100a9, ITGB2, Gpnmb, Dpy1913, Nceh1, Rasa1, Stab2, Fpr1, Eya4, Wfs1, Hpgds, Fcgr4, Btbd11, Ldlrad3, Abcb4, Tph2, Gpr137b, AC128618.1, NRP2, PHLDA2, Galnt15, Hspa1b, Hspa1b, Havcr2, Osbpl3, Arhgap19, Ppap2a, Fblim1, Nrip2, Pld3, Ahnak2, Ahnak2, Cd22, Abi3, Slc44a2, Hp, Ptprk, Decr1, Aldoa, Mmp12, Aldoa, Agpat1, Ly6d, Ndrg3, Gsn, Nceh1, Sh3bgrl2, Htr2b, Gdpd5, Wee1, Tmem206, Plxna2, Plxna2, Slc6a8, Zranb3, Ccsap, Fabp4, Tmem38b, Mfge8, Slc37a2, Enpp1, Slc12a2, Cmbl, Lgals3 bp, Scd2, Tpd5211, LOC100912195, HLA-DOA, SLAMF8, KIAA0930, JPH2, Acp5, Gyg, Cdca71, Tspan4, Nek6, Cd93, Ddc, Galnt12, Lipa, Akr1b10, Hgsnat, Myo1e, Slc27a1, Fbxo32, Zfyve9, Cd72, Gusb, Adgre4, Cd109, Pitpnc1, Afp, Akr1b8, E2f2, Serpine1, Uhrf1bp1l, Bckdk, Gpx7, Aqp7, Gpnmb, HLA-DMB, VMO1, Fcrl1, Cpd, Snx27, Neat1, Pde6c, Pxmp4, Pcp411, Dnmt1, Cebpa, Mvb12b, Atp13a2, Gnptab, Adgrl2, Cd72, Pparg, Pgap1, Dennd2a, Gm6483, Camk2d, Cd9, Aprt, Gngt2, Cldn34c1, Fcgr4, Hebp1, Fzd7, Hadha, Dip2c, Ceacam1, Fam149a, CHI3L1, S100a8, Mybph, Rrad, CTHRC1, Gna12, Col18a1, Col18a1, Rassf8, Jun, Osbpl3, Fbxo5, Rasa4, Lrp1, Fam132a, Map4k3, Nisch, Pkp2, Ttc7b, Wrb, Gm15513, Vma21, Apbb2, Igf2r, Gm4980, Pomk, Pitpnm1, Nus1, Slc39a11, Polr3k, Crtap, Scamp5, Herc3, D8Ertd82e, Smug1, Boll, Ctsd, Gpd1, Ccng1, Slc18b1, Fcgr2b, Crot, Atp1b4, Nudt7, Cc19, Il7r, Vipas39, Sgol2a, Acot2, Ankrd13c, Cd320, Mmp14, Igf2r, Cdk16, Slc52a2, Hfe2, Cyfip2, Crbn, Knop1, Cdkn2c, Ctsb, Marveld2, Galns, Clec4a2, Hk3, Syne1, Fgf13, Slc7a11, Clec4n, Plagl2, Plagl2, S1pr2, Prc1, Prc1, 4933438K21Rik, Gpr176, Ankrd26, Slc17a5, Dock4, Acer3, Sel1l3, Itgb5, Cdk18, Ttc30a1, Stx3, Tmem106a, Cipc, C6, Cdk20, Wwc1, Tor3a, IL1RN, CTGF, Lipf, Slc5a7, Atp1b1, Spp1, BCL6, Grina, Cndp2, Rab3il1, Soat1, Tmem43, Cyp2d9, Bcl6, Hsd17b 11, Tor3a, Tor3a, Ralgapa2, Hipk2, Sbsn, Idh3a, Rassf3, Cc128, Jakmip1, Ttyh2, Dhx32, Abcc5, Oxa1l, Camsap3, Lasp1, Specc1, Fam63a, Mtmr10, Mpzl1, Adipor1, Galc, Jag1, Nt5dc2, Mroh1, D8Ertd82e, Dgat2, Aprt, Tmem37, Endod1, Galnt7, Ms4a7, Mapre2, Polrmt, Msrb1, Myo10, Fam134b, Dennd4c, Fat3, Cd93, Lcmt2, Sorl1, Gpr137b, Rnf144a, Itgav, Itgav, Ogfrl1, Mfsd12, Abcg2, Wsb2, Uap1l1, Tbc1d23, Fam83f, Fcgr1, Arhgef7, HSD11B1, MMP9, TNMD, Pnpla3, Fabp5, Plet1, Krt79, Igfbp3, Itgax, SLC38A1, Tcirg1, Glg1, Stard3n1, Slc12a7, Guf1, Idh3g, Vipas39, Arl8b, Galc, Rcan1, Arl2, Bcat2, Alpl, Bloc1s6, Sigmar1, Fundc2, Airn, Slc35a4, Rfk, Armcx4, Ptgds, Slc16a1, Fgf21, Fam107b, Lipa, Prnp, Ankrd50, Csrp2 bp, Csrp2 bp, Fen1, Ckap4, Kctd12b, Mcm6, Arap3, Psen2, Ddx28, Tpd52, Mxd4, Gpt2, Kif22, Stau2, Hexb, Fignl1, Mxi1, Tmem65, Tmem38b, Tmem38b, Ctsl, Dock4, Cyp4v3, Golph31, Gnpnat1, Plxnb2, Lpcat2, 5430427019Rik, Tor4a, Fam214a, Mfap31, Msmo1, Nr1h3, Gclm, Airn, Ccnf, Pwwp2b, Il13ra1, Il13ra1, Pdpr, Mms19, Tmpo, Acot1, 2310022B05Rik, Cers6, Slc25a30, Fuca2, Atp6v1b2, Bmp1, Aida, N6amt2, N6amt2, Tfrc, Nav2, Sdc1, Gid4, Cyp8b1, Cs, Cs, Lpcat3.

When treating or preventing dermatitis, the compositions, manufacture, products, processes, methods, and/or methods of use, prevention and treatment will, in some preferred embodiments, modulate the expression of at least 2%, preferably at least 20%, and most preferably, >30% of genes selected from S100A9, AKR1B10, KRT16, LTF, MMP12, C10orf99, S100A8, S100A7A, COL6A6, DSC2, RGS1, CEP55, RRM2, NCAPG, NCAPG, HAS3, SERPINB3, IFI27, CCNB1, CCL18, KIF20A, NDC80, CKS2, CCL26, TYMP, KIF14, AURKA, ALYREF, BUB1, NEK2, CDC20, PRR11, HMMR, DLGAP5, KIF18B, WNT5A, MUC7, IQGAP3, GTSE1, PRKY, TTK, KRT6A, SCO2, MKI67, IGFL1, PI15, CCNA2, CASC5, NUF2, UHRF1, KYNU, KIF23, IRF7, LMNB1, SERPINB13, HNRNPH1, EHF, IL7R, KLHDC7B, CDK1, TOP2A, SELE, ASPM, TCN1, UBE2C, GJB2, LCE3D, CCR7, OAS1, DEPDC1B, MMP1, STAT1, CCL22, H2AFX, SOCS3, TMPRSS4, SNHG3, FOXM1, ZG16B, CDH3, SPC25, AURKB, KIAA0101, FANCI, ANLN, ZWINT, LINC01215, ZC3H12A, HS3ST3A1, GALNT6, ODF3B, E2F7, CDC25B, NUSAP1, BIRC5, CXCR4, ZC3HAV1, KIF4A, BIRC3, APOBEC3A, MIAT, NELL2, CLEC7A, CTSC, CRISP3, COL4A4, DEPDC1, CENPF, TK1, PTTG1, SGOL2, CXCL1, OAS2, OAS3, LAMP3, SLC7A5, KIF18A, DUOXA1, KIF11, DUOX1, EPSTI1, HJURP, SELO, S100A2, NAPSB, IFI6, NABP1, MX1, SAMSN1, BUB1B, SERPINB9, CH25H, ECT2, CTD-2228K2.7, GPR171, MELK, KIF15, MPZL2, CHEK1, XAF1, ISG15, C9orf84, CTLA4, CDCA3, TMC5, CCNB2, ADAM8, SLC16A3, HERC6, DSG3, NAA15, ALDH4A1, S100A12, KIF2C, E2F8, WHSC1, HIVEP3, CDKN3, CDKN3, TYMS, TAPBP, COL27A1, CDCA2, LMNB2, CENPE, RP11-303E16.2, LTB4R, COL6A5, F12, IL411, FAM83D, CDC7, TNC, TROAP, CD274, CENPA, CDC6, TPX2, MCM10, MYO10, VSNL1, GLS, AOC1, ZBED6CL, NRTN, CARHSP1, NUP210, SAMD9, RGS14, TTC39A, PARP12, RALGPS2, PRC1, NETO2, NOD2, ALDH16A1, GZMB, FSCN1, INO80D, JAK3, SLC4A11, MAD2L1, SLC16A14, WDR4, PARP9, MMP7, ACAP3, STIL, CD47, CDK5R1, FANCD2, OASL, PLAU, IL4R, SLC38A5, TNFSF10, TRIB2, HELLS, HIPK3, CCL2, KCNK6, UBA6, PBK, FOXE1, AKIRIN2, C6orf141, ARRDC1, ZBED2, STAT3, TACC3, APOL6, TRIM10, PNP, PEX13, MTFR2, MFHAS1, TPBG, CTC-251116.1, LCK, GEN1, CCND2, SMC4, IL26, CENPK, PLAT, TIMELESS, CARD10, NUP50, CCDC88C, KIAA1217, FBXO5, COTL1, ARHGAP11B, GINS3, NFASC, SLAMF8, ESPL1, SGK1, ITK, MAP3K14, IL27RA, TNPO1, ACAP1, SERPINB1, CAPN1, SECTM1, GINS1, DTL, RACGAP1, CCNE2, COPG1, IFI44, PGF, GBP1, SLAMF1, RP11-52612.5, SHMT2, ZC3H12D, SH3PXD2A-AS1, ITGAL, DENND4A, IDH3A, CCNF, ATAD2, HOMER1, PRRG4, MIB2, BAIAP2, PARP14, PRSS27, TNIP2, GLB1L3, TRPM1, IL12RB2, SPAG5, VPS9D1, ARNTL2, MCM5, STYK1, NAA50, POLR3G, LTB, GMFB, LINC00518, SHCBP1, KCNJ15, ST14, PSPH, SLC7A1, TSTA3, MAP4K4, E2F3, PPP2R2C, TRIP13, BCL3, RP11-295G20.2, SOCS1, FAM110C, DENND2D, MIR17HG, PPP2R3B, MICALL2, ANP32E, PSRC1, R3HDM4, C3orf62, RNF213, RNF213, CYP7B1, CDT1, LRP8, ZNF341, GNLY, GNLY, RAD52, NDRG4, TRMU, LGALS2, AKAP8L, PACSIN3, CD1B, CDCA5, MBNL2, WDR34, DDX58, SERPINE1, ESRP2, UBE2T, NFKB2, PKP3, EPHA2, AP1S3, NBEAL2, RASGRP1, RCC1, ABCA7, TUBGCP6, GJB6, E2F2, RC3H1, HS3ST3B1, CCDC137, CIITA, FBXO46, GPX2, STIP1, CTA-384D8.35, CTB-89H12.4, F5, RAB31, COMTD1, PAOX, FGD2, LARP4, CDC25C, DOCK10, CEPT1, THOC6, PECAM1, PVT1, CENPW, HYLS1, CDK10, FBXL6, ATG16L2, BRCA2, BRIP1, CC2D1A, SPCS3, ZBTB1, IRF1, ALDH3A1, CCL17, CDC42EP1, STKll, CISH, DOT1L, MICALL1, PLA2G3, GABRP, WDR76, SNRNP40, MICB, HN1, PARPBP, TNFAIP8L1, HGS, SFN, MAB21L3, SCAND1, SCAND1, FEN1, MARS, PIGR, C9orf142, FOXK2, FASTKD1, KCNN4, CDC45, BAX, FOSL2, POLQ, NCAPH, GPR68, IFIH1, RELB, ORC6, PPP1R10, SLC47A2, DHRS13, TMEM184A, ERAP1, S1PR5, ALS2CL, TEX9, ODF2, RTP4, ADAM19, IER5L, DNER, CYP27C1, LPAR5, ZWILCH, ZDBF2, TOB2, TMEM102, TC2N, SLC16A1, RNF145, CBLC, PIF1, LRRC45, SKA3, EME1, TNFRSF21, ZNF557, DDX39A, PHF19, RAB29, PYCARD, ATP2A3, MFI2, PUS10, RGS12, LURAP1L, SBNO1, ST8SIA4, MDFI, NEIL3, ENO1, AMMECR1, AMMECR1, SLAMF7, TMC6, CLEC10A, SYMPK, ZAP70, CYLD, LZTS1, TLR4, SLC31A2, PRG4, HINT3, RP11-467L13.7, RP11-326K13.4, RP11-757F18.5, RP11-757F18.5, UNC5B-AS1, MBP, TRAMIL1, ANKDD1A, LARP6, CREBRF, HHEX, SYT8, GUCY1A2, SHC3, SCD5, and LUM.

When treating or preventing alopecia, the compositions, manufacture, products, processes, methods, and/or methods of use, prevention and treatment will, in some preferred embodiments, modulate the expression of at least 2%, preferably at least 20%, and most preferably, >30% of genes selected from Cxcl9, Gzma, Cxcl1l, XIST, Cxcl10, Iigp1, Cc15, TSIX, Ms4a4b, Oasl2, Igtp, Ifi44, Gzmb, 2610528A11Rik, Gbp2b, Irgm2, HBB, Irgm1, Cd8a, CXCL10, Cxcr6, Clec7a, Isg15, Gbp3, Gbp7, CXCL9, Rtp4, Bst2, Zbp1, Ccl1, Cd274, Ddx60, Cyp7b1, Slfn4, Stat1, Herc6, Irf1, Itgae, Parp14, Mlkl, Trac, AW112010, Dhx58, Samhd1, Tnfsf10, Trim30a, Psmb9, Slfn2, Apol7a, Usp18, Tap2, AU020206, Klrc1, Vcam1, Uba7, Klrd1, Tap1, Xaf1, Ifi441, Psmb10, Tmem173, Dtx31, Lck, Irf8, Ifng, Psmb8, Stat2, Il18 bp, Nkg7, Ikzf3, Tnfaip2, Stat2, Zmynd15, Pglyrp1, 113ra2, Ly75, Apol8, Samd91, Serpinb9, Egln3, Rnf213, Spn, Rassf10, Lipg, Nlrc5, Nfkbie, Oas2, GZMA, Cxcl16, Tapbp, Bcl3, B4galnt1, Exoc314, RGS18, Eif2ak2, Cotl1, Batf2, SRGN, LYZ, CD1E, CD8A, Slfn10-ps, Lrrc4, Atp8b4, Cd3d, Icam1, Ndufa412, Irf9, Il2rb, IFI44, Laptm5, Cp, Ifi35, CD1B, STAT1, ST8SIA4, C920025E04Rik, GIMAP2, ADAMDEC1, SPP1, IFI44L, Ifih1, Ddx58, Ddx58, CHURC1, COL6A6, Gch1, Ms4a6b, Trim21, Car9, Bak1, GZMB, CLEC12A, GPR65, Trim25, Icos, Tapbpl, Erap1, Erap1, Irg1, Casp4, Mgarp, Parp10, Ifitm3, Cx3cl1, CCL5, H2-DMb1, GPR171, UGT8, HLA-F, EVI2B, Itgb7, Tlr3, Kcnn4, H2-K1, Ly6a, PRRX1, GYG2, IGF1, IRF8, MS4A4A, FCER1G, APOL6, SAMSN1, EPSTI1, PTPRC, Trim12c, Nfkb2, Tmem51, Psme1, Itgb8, TLR3, TLR3, SLC19A3, Gimap8, COL12A1, PDGFRL, MICB, SSBP2, AGTR1, IFIT2, FPR3, PPAP2B, EVI2A, EPHA3, CCNG2, RSAD2, CD48, IKZF1, IL15, Trim34a, BC064078, PDE4B, NR2F2, HLA-DRA, CCDC178, SAMD9L, CD28, PRF1, Oas1b, TSLP, CTSS, CTSS, GLIPR1, OGN, LCP2, LCP2, C1S, IFIT3, IL7, STMN2, PRKAR2B, CD2, GALNT7, HNMT, SLC16A7, CD33, Arhgap8, Tspo, Rbm43, Pfkl, CD1C, DACT1, HLA-DPA1, IGDCC4, Igf2bp2, ZFP14, XAF1, IL2RG, 1-Mar, JAK3, IL2RB, CD84, CD53, LCP1, RARRES3, IRF1, CCR2, FCGR2B, GBP4, FOXD1, FST, TNFSF13B, OAS2, AHR, ATP8B4, PSMB8-AS1, Stx11, Map3k14, Gm12185, Pitpnm1, N4BP2L1, PARP9, IL21R, P2RY13, PRKCB, DTX3L, DAB2, SMAD4, F13A1, CSF2RB, NAV3, MNDA, ITGAX, RAB30, ADGRE2, BTN3A3, BTN3A3, ITGAL, AOX1, DOCK10, POSTN, CMA1, BTN3A1, PRSS21, PSMB9, PSMB8, FCER1A, CTSW, KIF13A, CLEC4A, DCLK1, NAALAD2, TRIM22, PLEK, HGF, CCDC149, Nudt21, APOBEC3G, MPEG1, SLFN11, B2M, PRKACB, AGPAT9, HCP5, RP11-159D12.2, LPAR4, MDM2, TCP11L2, MUM1L1, TESC, TMEM132A, RP11-315F22.1, ERVW-1, FBN2, FAXC, ADAM23, WIPI1, PHKA2, RMND5A, EZR, LRRC15, EDN2, SLPI, PROCR, SEC14L2, ADCK1, CCHCR1, SORCS2, WDR72, RP11-1069G10.2, HHIP, Dirc2, FCHSD1, RP11-172H24.4, CBX2, Clorf105, SERINC2, UNC5B, ODC1, GALNT2, S100P, Hnmt, INPP4B, BAMBI, FRMPD1, NETO2, PCDH7, TJP1, AIM1L, KRT74, UNC5B-AS1, FHOD3, BMP7, RCAN1, NPC1, FLNB, TBC1D30, RUNDC3A, SLC45A2, ARL4C, TDH, CTD-3247F14.2, MYH14, MYH14, EFHD1, SLC1A6, LSMEM1, RAB3B, ATG4B, TRPM6, PINLYP, CABYR, USP2, DMBT1, LYPD6, KLHDC8A, WDR66, SLC10A4, Tmem246, CREB5, CREB5, PLEKHG4B, TTTY10, CYFIP2, KIF5C, FOXN1, KLK12, KLK12, BMP2, SOHLH2, MREG, HSPA14, KRT16, ATG9B, SPTBN5, KIF21A, PADI1, ELF5, CYP1B1-AS1, PLA2G2F, RP11-209D14.2, ATP8A2, SIGLEC10, JMY, Gls, PARM1, NLGN4Y, ERP27, CST4, WEE1, LINC00868, GLB1L2, Adh1, RFX2, DLX3, EHD3, EHD3, HSPB3, TRIM9, SMTNL2, PKP1, SLCO5A1, DLX4, CSNK1E, OVOL1, TTTY15, BEST3, DNAJC6, SLC7A8, RIMS2, Gpm6a, SP6, Sema3e, Slc38a3, SERPINA1, CNTNAP2, LEF1, CRNN, NCS1, PPP2R1B, ZNF503-AS1, SERPINA1, IL1F10, SH3GL3, OTUB2, LINGO1-AS2, SLN, HOXC13, PRKY, MSX2, KRT36, BNC2, CSDC2, ZFY-AS1, PSORS1C2, CAPN8, ABCA4, KRTAP19-3, WNK4, AC003958.2, RNASEH2CP1, KRT75, CAPN12, RNF182, FAM49A, KRT72, SLC7A11, ZFY, Wfdc3, KRT73, Sncg, COMP, KRT32, FGF18, KRTAP5-8, Adcy1, ZAR1, S100A3, PADI3, CST1, UTY, AC106876.2, RP11-115H13.1, PIRT, KRT82, KRT40, CHAC1, KRTAP10-11, KRT81, KRTAP7-1, KRT83, TTTY14, USP9Y, TXLNGY, DDX3Y, KDM5D, andEIF1AY.

When treating or preventing eczema, the compositions, manufacture, products, processes, methods, and/or methods of use, prevention and treatment will, in some preferred embodiments, modulate the expression of at least 2%, preferably at least 20%, and most preferably, >30% of genes selected from S100A9, AKR1B10, KRT16, MMP12, LTF, C10orf99, S100A8, S100A7A, COL6A6, DSC2, RGS1, CEP55, RRM2, SERPINB3, HAS3, NCAPG, NCAPG, IFI27, CCNB1, NDC80, KIF20A, CCL18, AURKA, TYMP, CCL26, KIF14, ALYREF, CKS2, BUB1, NEK2, CDC20, PRR11, IQGAP3, HMMR, DLGAP5, KIF18B, MUC7, WNT5A, KRT6A, TTK, PRKY, CASC5, NUF2, GTSE1, SCO2, MKI67, IGFL1, PI15, CCNA2, UHRF1, KYNU, KIF23, IRF7, LMNB1, SERPINB13, HNRNPH1, EHF, IL7R, KLHDC7B, CDK1, TOP2A, SELE, ASPM, CDH3, SPC25, TCN1, UBE2C, GJB2, LCE3D, CCR7, OAS1, DEPDC1B, MMP1, STAT1, CCL22, H2AFX, SOCS3, TMPRSS4, SNHG3, FOXM1, ZG16B, KIAA0101, AURKB, FANCI, ANLN, ZWINT, LINC01215, ZC3H12A, HS3ST3A1, GALNT6, ODF3B, E2F7, CDC25B, NUSAP1, BIRC5, CXCR4, ZC3HAV1, KIF4A, BIRC3, SLC7A5, APOBEC3A, MIAT, NELL2, CLEC7A, CTSC, CRISP3, COL4A4, DEPDC1, CENPF, TK1, PTTG1, SGOL2, CXCL1, OAS2, OAS3, LAMP3, SAMSN1, MX1, BUB1B, S100A2, NAPSB, IFI6, NABP1, SERPINB9, CH25H, ECT2, CTD-2228K2.7, GPR171, MELK, KIF15, MPZL2, CHEK1, XAF1, ISG15, C9orf84, CTLA4, CDCA3, TMC5, CCNB2, ADAM8, SLC16A3, HERC6, DSG3, KIF18A, DUOXA1, KIF11, DUOX1, EPSTI1, HJURP, SELO, NAA15, ALDH4A1, VSNL1, MYO10, S100A12, KIF2C, E2F8, WHSC1, HIVEP3, CDKN3, CDKN3, TYMS, TAPBP, COL27A1, CDCA2, LMNB2, CENPE, RP11-303E16.2, LTB4R, COL6A5, F12, IL411, FAM83D, CDC7, TNC, TROAP, CD274, CENPA, CDC6, TPX2, MCM10, UBA6, NRTN, ZBED6CL, AOC1, FOXE1, PBK, GLS, CARHSP1, NUP210, SAMD9, RGS14, TTC39A, PARP12, RALGPS2, PRC1, NETO2, NOD2, ALDH16A1, GZMB, FSCN1, INO80D, JAK3, SLC4A11, MAD2L1, SLC16A14, WDR4, PARP9, MMP7, ACAP3, STIL, CD47, CDK5R1, FANCD2, OASL, PLAU, IL4R, SLC38A5, TNFSF10, TRIB2, HELLS, HIPK3, CCL2, KCNK6, PEX13, PNP, TRIM10, APOL6, TACC3, MTFR2, COPG1, CCNE2, RACGAP1, DTL, AKIRIN2, C6orf141, ARRDC1, ZBED2, STAT3, MFHAS1, TPBG, CTC-251116.1, LCK, GEN1, CCND2, SMC4, IL26, CENPK, PLAT, TIMELESS, CARDO1, NUP50, CCDC88C, KIAA1217, FBXO5, COTL1, ARHGAP11B, GINS3, NFASC, SLAMF8, ESPL1, SGK1, ITK, MAP3K14, IL27RA, TNPO1, ACAP1, SERPINB1, CAPN1, SECTM1, GINS1, SH3PXD2A-AS1, IDH3A, DENND4A, ITGAL, CCNF, ATAD2, PKP3, RCC1, RASGRP1, NBEAL2, AP1S3, EPHA2, IFI44, PGF, GBP1, SLAMF1, RP11-52612.5, SHMT2, ZC3H12D, HOMER1, PRRG4, MIB2, BAIAP2, PARP14, PRSS27, TNIP2, GLB1L3, TRPM1, IL12RB2, SPAG5, VPS9D1, ARNTL2, MCM5, STYK1, NAA50, POLR3G, LTB, GMFB, LINC00518, SHCBP1, KCNJ15, ST14, PSPH, SLC7A1, TSTA3, MAP4K4, E2F3, PPP2R2C, TRIP13, BCL3, RP11-295G20.2, SOCS1, FAM110C, DENND2D, MIR17HG, PPP2R3B, MICALL2, ANP32E, PSRC1, R3HDM4, C3orf62, RNF213, RNF213, CYP7B1, CDT1, LRP8, ZNF341, GNLY, GNLY, RAD52, NDRG4, TRMU, LGALS2, AKAP8L, PACSIN3, CD1B, CDCA5, MBNL2, WDR34, DDX58, SERPINE1, ESRP2, UBE2T, NFKB2, ABCA7, TUBGCP6, GJB6, E2F2, CTA-384D8.35, CTB-89H12.4, F5, RAB31, COMTD1, PAOX, FGD2, LARP4, CDC25C, ZDBF2, TOB2, TMEM102, TC2N, SLC16A1, RNF145, CBLC, PIF1, RC3H1, HS3ST3B1, CCDC137, CIITA, FBXO46, GPX2, STIP1, DOCK10, CEPT1, THOC6, PECAM1, PVT1, CENPW, HYLS1, CDK10, FBXL6, ATG16L2, BRCA2, BRIP1, CC2D1A, SPCS3, ZBTB1, IRF1, ALDH3A1, CCL17, CDC42EP1, STK11, CISH, DOT1L, MICALL1, PLA2G3, GABRP, WDR76, SNRNP40, MICB, HN1, PARPBP, TNFAIP8L1, HGS, SFN, MAB21L3, SCAND1, SCAND1, FEN1, MARS, PIGR, C9orf142, FOXK2, FASTKD1, KCNN4, CDC45, BAX, FOSL2, POLQ, NCAPH, GPR68, IFIH1, RELB, ORC6, PPP1R10, SLC47A2, DHRS13, TMEM184A, ERAP1, S1PR5, ALS2CL, TEX9, ODF2, RTP4, ADAM19, IER5L, DNER, CYP27C1, LPAR5, ZWILCH, LRRC45, SKA3, EME1, TNFRSF21, ZNF557, DDX39A, PHF19, RAB29, PYCARD, ATP2A3, MFI2, PUS10, RGS12, LURAP1L, SBNO1, ST8SIA4, MDFI, NEIL3, ENO1, AMMECR1, AMMECR1, SLAMF7, TMC6, CLEC10A, SYMPK, ZAP70, CYLD, LZTS1.

When treating or preventing diabetes, the compositions, manufacture, products, processes, methods, and/or methods of use, prevention and treatment will, in some preferred embodiments, modulate the expression of at least 2%, preferably at least 20%, and most preferably, >30% of genes selected from Adipoq, Cfd, Galc, Hmgcs2, Gsta3, Car3, Akr1d1, Usp2, Reg3g, Reg3g, Slc7a13, Sult2a1, Cfhr1, Lap3, Xaf1, NPPA, Myh7, AC128618.1, Cpb2, Ankrd1, Ankrd1, Cd302, Ugt2b15, Ugt2b15, Hmgcs2, Acsm3, NPPA, Pdk4, Mcm6, EIF1AY, Steap4, Mlc1, Cyp1a1, Tubb5, Malat1, Gsta3, Nppa, Abcc3, Pdcd4, Aldh1a1, Mrps18c, Cp, Trh, Ubn1, Cacybp, SFRP4, Atp1a2, Hmgcs2, Cyp2e1, Prlr, Prlr, Pdk4, Aplnr, DSC1, Cpsf6, LOC100151767, Slbp, Sowahc, Amy1a, Penk, Rbm3, Adi1, Ankhd1, Gigyf2, DSC1, Fmr1, Adh7, Glyatl2, Ndfip2, Arid1b, Cfh, Myl6b, Cxcl9, Ppp1cb, Zcchc11, Pgpep1, Mmp13, Csrp3, Pik3ca, Mybph, Por, Psmc6, Dcun1d5, NEB, Upk1b, Vsnl1, Vsnl1, FRZB, Cbr2, Wisp1, Lpin2, Igfbp2, Dimt1, Herc2, G0s2, Zfp354a, Dhx9, Sc5d, NEB, Nnat, MYL4, Tnfaip8, Dleu7, Per2, Pfkfb1, Cyp2cl2, Srp54a, Gclm, Maff, Tnc, Hamp, SNORA14A, AC097374.4, FRZB, SLN, Col3a1, Idi1, Nef1, Mtmr7, Acta2, Brix1, Egr1, Sh3yl1, Myc, Usp33, P2ry1, AABR07044412.1, Cpeb2, Myh7, Penk, Ahctf1, Lurap1l, Pcp411, Cc112, Kif1b, Postn, U8, UTY, Alas2, Ankrd1, Mgl2, Eif1a, Yars2, Ssfa2, Memo1, Yipf4, Ehhadh, Lrif1, Wac, Hmgcr, Fgf13, S100a9, Acot2, ENPP2, Slitrk6, Tph2, Rgs1, Hamp, Calcb, Tnfrsf11b, Atf3, Fmod, Nabp1, Bcl6, Ptprg, Inmt, Rec8, Tpm2, PRELP, HAPLN1, ASB14, Acyp2, Rbms1, Acsl4, Enc1, Ncbp2, Zfp131, Rbm3, AC128962.1, RGD1565641, Utrn, Decr1, AC128618.1, AC128618.1, Myh7, Etnppl, Gm6484, Per2, Wasl, Ube2d2, Colec11, Sdf211, Ralgapa1, Mmp12, Lyve1, Cry1, Cnot7, Abcb1a, Cuedc1, Vdr, Klf6, Supt16, Rapgef6, Nfkbiz, Zfp429, Fbxo30, Tardbp, Zfp455, Ccnd2, Cyr61, Ak4, Sucnr1, Fmo5, U8, USP9Y, KLHL38, KLHL38, SFRP4, BAGE2, Pnrc1, Egln3, Eif2a, Sumf1, Hspb1, Lcor, Cidea, Klhl9, Tpd5211, LOC100912195, Sult1a1, Decr1, Penk, Angptl4, FMOD, IGSF10, Cndp1, Cml2, St8sia4, Fosb, Rasd1, Dnajb1, Dnajb1, Atrx, Scd2, Kitl, Akap9, COL14A1, KIAA1107, FAXDC2, RPS4Y1, P2RY14, MT-TS1, MT-TS1, Serpinh1, Zfp420, 2810002D19Rik, Srsf7, Myh11, Txnl4a, Arsk, Plscr1, Aldh1a1, Lama3, Pcmtd2, Smc2, Gpm6a, Miox, Cnih4, LOC100362409, Enc1, Wee1, Tsku, Nup153, Slc25a16, Cyp26b1, Jun, Egr1, Ppp1r1a, Col27a1, Reg3b, Hspa1b, Mat1a, Trh, HSPA2, RNU6-905P, MT-TS1, MT-TS1, NPR3, UGT2B10, SVEP1, P2RY14, FBXO40, Ccr5, Dpys, Antxr1, Tcf21, 3110045C21Rik, Cpsf6, Rgs19, Myct1, Slc7a2, Tfec, Asph, Gcnt2, Txnl1, Nmrk1, Afmid, Tmem38b, Sall1, Cd51, Fam195a, Fn3krp, Ankhd1, Dars, Ubxn8, Aass, Pyy, Gadd45g, Oxct1, LOC498705, Rgs4, C3, Yam1, Aqp7, Gpnmb, Txnip, Acot2, Decr1, Pdk4, Gpm6a, Elovl6, Cry1, Lmo7, Btnl9, Ces1d, Neat1, Iglc2.

When treating or preventing acne, the compositions, manufacture, products, processes, methods, and/or methods of use, prevention and treatment will, in some preferred embodiments, modulate the expression of at least 2%, preferably at least 20%, and most preferably, >30% of genes selected from LCN2, SLC12A8, TMPRSS4, AT3G05170, and KIAA0125.

When treating or preventing hyperlipidemia and/or atherosclerosis, the compositions, manufacture, products, processes, methods, and/or methods of use, prevention and treatment will, in some preferred embodiments, modulate the expression of at least 2%, preferably at least 20%, and most preferably, >30% of genes selected from Defb1, Pdk4, Fam107a, Tff3, Sult1e1, Slc25a4, HMOX1, Psat1, Lepr, Anxa13, Igdcc4, Nat8, Rasd2, Rcan2, Qpct, P2ry14, Nupr1, Ptgfr, UPP1, Timp4, MAFF, MAFF, Vldlr, Rab27a, HMOX1, CD55, Tomm401, HSPA1B, BAG3, UPP1, Pcp411, Acot1, Atp1a2, Serpina7, Cela3b, Gadd45b, Mthfd11, Nucb2, SLC7A11, DNAJB1, Prss8, Elovl7, Sgk1, Atp10d, SLC7A11, ASNS, Txnip, SQSTM1, TRIB3, Abcc4, SQSTM1, UPP1, Gstm3, Osbpl3, S100P, HSPA1B, JAG1, DUSP1, Usp18, Pnliprp1, Ddr1, Emp2, Dram1, PPP1R15A, PLIN2, TRIB3, 5330417C22Rik, ASNS, Nrg4, HMOX1, Cd36, Atp1b1, AKAP12, Prelid2, Ifi44, Tfrc, JAG1, Hsd17b11, ANXA1, DUSP5, CEBPB, Rbm39, Lgals4, Hk2, Slc13a2, VEGFA, Cox7a1, SQSTM1, NPC1, CEBPB, DNAJB9, HSPH1, CD55, KRT16, ZNF165, CLU, Abcg1, Cpne8, Gstm2, Atp6v0e2, Zmynd12, Gsn, Igfbp1, Ptpre, MAFF, CXCL3, EMP1, BAG3, SFN, CLU, Lpl, Plcb1, Acsl4, Rapgef5, Slc44a3, Dnajc12, Lyve1, Tuba8, Rpa1, Rpa1, Adora1, FAM129A, Gadd45g, ADM, SERPINH1, DNAJB9, S100P, Akr1c19, 1700019G17Rik, Cys1, 1700113H08Rik, Tacc2, Gmds, Arl2 bp, Chic1, Rpap3, FAM129A, Spp1, Setd8, GTPBP2, GRB10, ATF3, DNAJB1, ANXA1, S100P, Bex1, Zbtb16, Ppp1r3b, Baiap211, Nrbp2, Hspa2, Mpeg1, Megf9, Aifm3, P4ha2, Npdc1, Bicc1, Chchd6, Rsad2, Il1rn, Plin4, VEGFA, KLF4, GCLM, DUSP5, JAG1, Esrrg, Marco, Tenm2, Zfp618, Igfbp2, Pex11a, Ttll1, Grhl1, Gda, E330009J07Rik, Tnik, Ttpal, Bmp7, Itpr2, Cyfip2, Spc25, Pbx3, Bace1, Atp11a, Scy12, GRB10, GCLM, CCNG2, SERPINE1, PPP1R15A, AKR1C3, ABCC2, PLAUR, Dcaf1211, Lcn2, Slc8a3, Tgm2, Tgm2, Pdgfc, Ppm1l, BC048546, Ptgr1, Crat, Rab3d, Tbc1d4, LAMB3, Anxa2, Luc712, Stat1, Btg2, Cdkn1a, Bhlhb9, ASNS, CASP4, ASNS, GCNT3, PLIN2, HSPA4L, ATF3, NPC1, TXNRD1, DNAJB4, ADM, MARS, TAX1BP1, STK17A, NCF2, BLVRB, Tmem63a, Car14, Fus, Ugdh, Kynu, Fst, Dnaja4, Ogt, Trib2, Adck4, Ppic, Slc27a1, Plin5, Por, Gstm1, STC2, Tspan8, Tmem71, FAM13A, RIT1, PLIN2, NQO2, SULT1C2, PLIN2, TRIB3, CXCL3, DUSP5, GFPT1, ZFP36L1, ME1, SERPINE1, CEBPB, LDLR, IFRD1, CD55, AKAP12, HSPH1, SLCO3A1, RIOK3, TNFRSF10B, CLU, Me2, Dopey2, Comtd1, Entpd2, Col4a5, Mettl20, Ctse, BC023829, Akr1b7, Myom3, Lrp11, Setd7, Ifit1, Lrrc24, Tfdp2, Ctgf, Nudt19, Ppargc1a, Ppargc1a, Cyp39a1, CEBPG, ALAS1, GPRC5A, CEBPG, KLF4, TXNRD1, FLNB, CLIP1, FLNB, UGDH, SERPINE1, GADD45B, AKR1C3, STK17A, AKR1C3, CHIC2, Rabep2, Wnk4, Serpinb6a, 2010204K13Rik, ASNS, SLC7A11, NDRG1, TPBG, Zfhx4, Lix1l, Trpm6, Immt, Ywhag, Tmem191c, Ctps, Fmo5, Slc39a6, Pla2g16, Rcan3, 6-Sep, Tia1, Ankrd10, Dtymk, Lbp, Ifit2, Nudt5, Slc22a3, Vill, Pygb, Ppm1m, Pde7b, Glg1, Nhp2, Nhp2, Bcl2111, Mcm6, Osbpl11, Tkt, Bzw2, Cpm, Plce1, Trmt10a, Pigq, Acot2, Slc2a4, Acacb, Isg15, Sorcs2, Mcm4, Msrb3, Slc25a51, Cp, Gria3, Plekhb1, Lhx6, Fzd6, A530020G20Rik, Glb1, Rbbp8, Bri3 bp, Diap2, Aco2, Aco2, Rab11fip2, Vcam1, Raph1, Serinc2, Slc16a10, Cacna1b, Cystm1, Tmem218, Srrm1, Celf2, Hjurp, Id1, Prkdc, Kif2a, Abcc3, Bmp2, Oplah, Oxct1, Fos, Pspcl, IDH1, FLNC, P4HA2, NQO2, P4HA2, ATF3, ANGPTL4, SLC3A2, IFRD1, SFN, AKAP12, KRT81, KRT16, PSMD2, UBFD1, TNFRSF10B, ASPH, SLC1A4, RIOK3, PSMD12, Pik3r4, Ddhd2, ASNS, Gyg, Slc35g1, Fgf1, Pkd2, Cyp4a14, Dnajc1, Nmt2, Eid1, Krt8, Med121, Rbm5, Crtap, 4933431E20Rik, Mtpn, Irf7, Dclre1a, Ren1, Enc1, Dip2a, Ddit4, Reep5, Mir22hg, Ank3, 4632404H12Rik, D19Bwg1357e, Tubb2a, Nudt16, Tspan3, Lysmd2, Ybx3, Uck2, Impa2, Zwint, Tshz2, Rsbn1, Lgalsl, Pnisr, Notch1, Ehhadh, Sulf2, Dixdc1, Sestd1, Ofd1, Tmed6, Oasl2, Pfn2, Pdss1, Dfna5, Ctsb, Nisch, Ube2d3, Med1, Zfp207, ITPR1, SULT1C2, TAF7, SARS, PSMC4, GTPBP2, DNTTIP2, MSMO1, PLIN2, LAMB1, PSAP, DNAJB4, SPAG9, ULK1, YARS, EMP1, DNAJA1, ANGPTL4, AFF1, MARS, WARS, GARS, NGFR, CCPG1, ZFP36, SEL1L, ASPH, CTH, ZNF165, PLAUR, ANXA1, BLVRB, Gpd1l, and A930033H14Rik.

When treating or preventing an inflammatory or autoimmune condition, disorder or disease (e.g. inflammatory bowel disease) the compositions, manufacture, products, processes, methods, and/or methods of use, prevention and treatment will, in some preferred embodiments, modulate the expression of at least 2%, preferably at least 20%, and most preferably, >30% of genes selected from DUOX2, REG1B, S100A8, VNN1, DUOX2, SLC6A14, MMP1, MMP3, CXCL5, SLC6A14, HLA-DRA, CXCL8, SLC6A14, ALDOB, REG3A, REG1B, KYNU, CXCL1, REG1A, S100A8, REG3A, HLA-DRA, REG3A, PCSK1, KYNU, S100A8, REG1A, REG1A, ANXA1, HLA-DPA1, TCN1, SLC6A14, DUOX2, CXCL6, CHI3L1, CHI3L1, MMP12, CXCL1, CXCL2, REG3A, CXCL1, KYNU, PTGS2, MMP10, REG4, DUOXA2, DEFA6, GJA1, REG1B, MMP1, DEFA6, REG1B, CXCL3, VNN1, TNIP3, CXCL1, MMP3, CXCL5, REG1B, DUOX2, CXCL13, IDO1, SERPINB7, KYNU, ANXA1, LYZ, CXCL11, REG3A, DEFA5, SOCS3, HLA-DQB1, BCL2A1, DUOXA2, HLA-DRB1, SERPINB7, TNIP3, CXCL8, SLC6A14, LYZ, DUOXA2, MMP3, HLA-DPA1, VNN1, HLA-DRB1, CXCL1, DUOX2, TNC, INHBA, TCN1, REG4, CLDN2, HLA-DPB1, RBPMS, DEFA5, MMP12, CXCR4, DEFA5, IGLV1-47, CXCL5, CXCL2, TCN1, CEMIP, TAGAP, KLK10, FAM26F, TIMP1, HLA-DMA, CXCL6, LCN2, FCGR3B, CXCL3, FAM26F, CSF2RB, LYZ, SRGN, IL1B, IGKV3-20, REG4, IGKV4-1, CXCL8, SERPINB5, KCND3, IDO1, POU2AF1, HLA-DQA1, SERPINB5, VNN1, CXCL2, C2CD4A, IL1RN, LYZ, CDH3, PI15, DUSP4, HLA-DRA, HLA-DQB1, TGFBI, CCL20, COL15A1, PDE4B, C2CD4A, LCN2, SAMSN1, MMP1, MS4A1, LY96, MNDA, BST2, REG4, CXCL5, TCN1, PRAC1, CD74, KRT6B, DUSP4, FDCSP, TIMP1, TFF1, GJA1, CYP4X1, SLC28A3, CD74, IGKV1OR2-108, CXCL2, DMBT1, CXCL11, DEFA5, CYR61, RGS2, WNT5A, LPCAT1, TGFBI, HLA-DRA, C2CD4A, DMBT1, CARD6, ANXA10, SLCO1B3, CFI, LPCAT1, TRIM22, MXRA5, SERPINB3, IGFBP5, SERPINB3, FCRL5, IL11, RGS1, SPP1, PTPRC, PROK2, TNFAIP6, SELL, SERPINB5, CD55, IL7R, COL1A2, CTSE, C2CD4A, IGHV3-21, ALDOB, ALDOB, NPTX2, KCND3, GPX8, CXCL3, HLA-DMA, IRAK3, DUOXA2, SLC2A3, HLA-DPB1, CD55, DEFA5, C11orf96, CTHRC1, COL6A3, BASP1, HLA-DQB1, IGLV3-10, MMP7, MMP7, PDZKIIP1, FRAS1, CXCL2, BACE2, NEXN, HLA-DMB, SLC6A20, CPS1, HLA-DQB1, KLHL5, TRIB2, VNN1, SDR16C5, REG1A, IGHV4-34, CD274, S100A9, OSMR, NAMPT, C4BPB, CHI3L1, HLA-DMA, KLK10, CXCL13, LUM, CFI, C4BPB, CFI, PDZKIIP1, MMP3, CXCL3, PI15, ALDOB, PLAU, PLAU, GJA1, IGLV3-25, MXRA5, HLA-DPA1, HLA-DPA1, IGHM, MMP9, PFKFB3, TIMP1, COL12A1, ALOX5, HLA-DMA, IFI16, RBPMS, SOCS3, CXCL11, KRT6B, C4BPA, CCL20, SLC28A3, ALDH1A2, PMP22, IFI6, DSG3, TRIM22, TNIP3, EVI2B, MZB1, LAMP3, SLAMF7, IL1RN, RGCC, TMEM200A, DMBT1, SULT1C2, DDIT4, TRIM29, CXCL11, MTCL1, ELOVL5, TMPRSS3, TMPRSS3, ADM, S100A12, SLC6A14, LCN2, KCND3, VCAM1, IGHV3-23, TGM2, BCL2A1, FKBP11, EGR3, TMEM45A, CXCL3, CLDN2, LPCAT1, RGS2, CD74, IGLV1-47, PCSK1, RBPMS, WARS, ALDOB, S100P, TRIB2, SFTPA2, SLITRK6, ADGRG7, TM4SF20, SLC6A14, IL1RN, MMP10, CXCL5, SOCS3, IL13RA2, TNFRSF17, ROBO1, PLEK, QKI, CYTIP, LPCAT1, ACSL4, PCSK1, TMC5, CXCR4, IFIT3, GBP1, COL3A1, COL3A1, CDH11, C4BPA, DUSP4, TRIB2, COL5A2, SLC7A11, CDH3, FAM26F, MMP10, GPR183, HLA-DPA1, AIM2, TGFBI, SPINK4, ZFPM2, TMEM200A, PROK2, DMBT1, KYNU, IFI16, ADGRG7, IL33, CXCL10, PDZK1IP1, PDZK1IP1, TRIM22, IGLV3-19, S100A12, SPINK4, CXCL6, PITX1, RAB31, ART3, SERPINB9, LCP2, ELOVL5, HCLS1, SELE, NCF2, CHST15, TGM2, DEFA6, TMEM45A, FOXQ1, ANO6, BLVRA, BLVRA, RP11-693N9.2, MXRA5, BCL2A1, MS4A1, FOXQ1, DSE, LILRB1, COL1A1, COL4A1, NR4A2, GBP1, DOC2B, GREM1, TMEM158, DMBT1, RNF183, DCN, NID1, GAS1, PLEKHS1, NXF3, C4BPA, SI, CDH11, CHRM3, CYP4X1, RARRES3, GABRP, LPL, SEC24D, IFI44, ANKRD36BP2, CCL20, CEMIP, KCND3, CIS, SDR16C5, CD74, NOS2, MGP, CLDN1, NOS2, COL15A1, SDR16C5, PECAM1, CXCL8, PTGS2, MMP1, CD55, OLFM4, IDO1, LPL, LPL, MSN, DAPP1, TNFSF13B, ALOX5, INSC, WISP1, IL1B, CSF2RB, LOXL2, RFTN1, AZGP1, CXCL9, SLC6A6, STEAP4, DSG3, POU2AF1, DOCK8, CD44, VSIG1, FCGR3B, S100A8, RARRES3, TRNP1, CARD6, S100P, PSMB9, PLEKHS1, INSIG1, SACS, RP1-93H18.6, CFI, PCSK9, SEPP1, GALNT6, IFI6, ME1, ME1, ALDH1A2, IDO1, BNIP3, KRT6B, ALDH1A2, CD53, PTGS2, CXCR2, KLHL6, FSTL1, SERPINA1, AC104699.1, IL6, SPARC, IFI16, and PFKFB3.

When treating or preventing asthma, the compositions, manufacture, products, processes, methods, and/or methods of use, prevention and treatment will, in some preferred embodiments, modulate the expression of at least 2%, preferably at least 20%, and most preferably, >30% of genes selected from CST1, CST1, POSTN, ITLN1, CPA3, FETUB, CLCA1, POSTN, DPP4, CST2, SLC5A5, SERPINB2, CLC, SLC9B2, ITLN1, CDH26, TFF3, CA2, SH2D1B, CST2, ANO1, CLCA1, NTRK2, GCNT4, GCNT3, FAM177B, CPA3, CCL26, FETUB, SERPINB4, PTHLH, CEACAM5, CD200R1, TMEM45A, CD274, CD44, CLCA1, CD36, DNAJC12, HRH4, PRODH, SH2D1B, VSIG1, IL1RL1, TNC, PHLDB2, HPGDS, TCN1, CCBL1, GAPT, MS4A2, ADRA2A, SPDEF, CDH26, FGFBP1, CMYA5, CSTA, SLC6A14, RAET1L, TMEM71, FOXA3, NTRK2, SLC22A16, ADGRE1, ALOX15, EPHA4, SLC7A1, TFF1, FKBP5, CEACAM5, NOS2, NOS2, CD274, TPRXL, AKR1B10, TFCP2L1, CXCL14, CPA3, LINC01559, SERPINF1, DPYSL3, DHX35, CMYA5, GCNT3, SLC39A8, TSPAN8, CTSC, SLC9B2, TMEM211, CD109, HDC, FAM83D, PXDN, IL1R2, MT1G, SEC14L3, C2CD4B, TMEM74B, C3, PTGIR, HCRT, MTNR1A, NOX1, ALDH3A1, GBP4, GBP2, MUC5B, XIST, SULT1E1, VNN2, TMEM45A, MUC5B, FHOD3, GPR156, IK, EDIL3, SELL, SCGB1A1, ADTRP, SCGB3A1, GBP5, LTF, S100A9, CSH1, DUOXA2, IFI44L, ST8SIA4, C7orf26, PTGIR, DUOX2, HGD, HGD, and CSH1.

When treating or preventing allergy, the compositions, manufacture, products, processes, methods, and/or methods of use, prevention and treatment will, in some preferred embodiments, modulate the expression of at least 2%, preferably at least 20%, and most preferably, >30% of genes selected from IFI27, CCL8, APOBEC3A, NEXN, RSAD2, RSAD2, S100A9, IFITM1, KIAA0101, IFI44L, CYR61, IFIT1, IFIT2, CCL18, CTGF, RRM2, TOP2A, TNFSF10, TNFSF10, SERPING1, PLS3, AKR1B10, KRT16, ISG20, IFIT3, P2RY12, MS4A4A, HESX1, NGFRAP1, LGMN, MEST, CXCL10, CD163, FSTL1, CCL2, LTF, MMP12, CDK1, BEX1, HERC5, C10orf99, S100A8, OASL, PBK, RGS4, S100A7A, LGALS3BP, HIST1H2AE, PLOD2, NR2F2, ISG15, CCL24, IFITM3, DTL, CALD1, ANLN, PFN2, EFEMP1, COL1A1, CD80, PRC1, P2RY13, MX1, TPX2, RP4-781L3.1, C3AR1, COL6A6, FERMT2, ABCA1, NDC80, ASPM, DKK3, YAP1, GMPR, CMPK2, OAS1, IFITM2, CXCL11, TMEM200A, PRSS23, LINC00487, RNASE1, DSC2, RGS1, CEP55, TYMS, GCH1, CCL23, CCL26, IFI6, COL4A2, TFPI2, RRM2, RNF213, AIM2, WWTR1, VCAM1, HIST1H2AG, MELK, GPX8, SPARC, DDX58, IL411, FAM83D, CDKN3, SERPINB3, CCNB1, IFI27, NCAPG, NCAPG, HAS3, C12orf75, KIF20A, MYH10, NUF2, NNMT, IFI44, MX2, IL6, CD14, ZBP1, PRTFDC1, NDC80, CCL18, DLGAP5, CKS2, LAMB1, HMMR, NID1, LOXL1, CLDN11, FPR2, OAS2, OAS3, TPD52, ALYREF, AURKA, KIF14, TYMP, CCL26, CCL4, MARCKS, NEK2, CDC20, PTX3, HIST1H2BC, EPSTI1, EPSTI1, AJUBA, AXL, RP11-701P16.5, SIGLEC1, SP140, LIMCH1, BUB1, BCL2A1, WNT5A, KIF18B, DLGAP5, HMMR, IQGAP3, PRR11, RARRES3, LYSMD2, STAT1, CAV1, IGF2BP3, NT5E, UBE2C, HERC6, CD48, TSPAN6, MUC7, HIST1H3D, CCNB1, CCNA2, PI15, IGFL1, MKI67, SCO2, GTSE1, NUF2, CASC5, PRKY, KRT6A, UHRF1, TTK, FRMD3, AMOTL2, KIF20A, CCNE2, HIST1H2BD, DLG5, NFIB, CCL13, FOXG1, CXCL2, CD300A, HSD11B1, TTK, PDCD1LG2, PLSCR1, PLSCR1, ASPM, FZD6, ENPP2, JAKMIP2, AC004988.1, WBP5, GBP4, CTSL, GOS2, MMP12, MAD2L1, RTP4, HELZ2, SMARCA1, IFI35, RND3, KIF23, IRF7, LMNB1, SERPINB13, HNRNPH1, EHF, IL7R, KYNU, KLHDC7B, CDK1, TOP2A, SELE, DEPDC1, SAMD9, HSH2D, RHOBTB3, IFIT5, KIF11, GJC1, SPC25, ADGRG6, CTSLP8, VAMP5, BUB1B, CCNA2, OSMR, ARMCX1, HIST1H2AC, FPR1, SOD2, CTLA4, LY6E, GJB2, LCE3D, CCR7, OAS1, DEPDC1B, MMP1, STAT1, CCL22, H2AFX, SOCS3, SPC25, TCN1, UBE2C, TMPRSS4, SNHG3, FOXM1, ZG16B, CDH3, BIRC5, CXCR4, ZC3HAV1, KIF4A, BIRC3, MNDA, KIF2C, BRIP1, MATN2, HIST1H1C, ACVRL1, DDX60L, KIF23, GBP1, GBP1, FN1, SOCS3, PLA2G16, SNX7, DDAH1, APOL3, ZWINT, HOXC6, GGH, ID1, TRIMS, CENPK, CSRP2, HIST2H2BE, IRF7, MAFB, CFI, COL4A1, KIAA0101, FANCI, ANLN, ZWINT, LINC01215, ZC3H12A, HS3ST3A1, GALNT6, ODF3B, E2F7, AURKB, CDC25B, NUSAP1, IL15, YES1, BUB1, TAGLN, SECTM1, NCAPG, CDC6, IFIH1, MCAM, HIST1H2BH, IL7R, SIDT2, FANCI, EREG, SLAMF1, BIRC5, ODF3B, E2F7, PARP9, MYO1G, KLHDC7B, HOXB7, SAMD9L, SAMD9L, MT2A, CDC20, BATF2, PTPN14, CEP55, SAT1, FOXM1, ENPP4, SPATA13, HIST1H2BI, CDKN2A, PPM1K, PAWR, CLEC7A, CTSC, CRISP3, COL4A4, DEPDC1, CENPF, TK1, PTTG1, SGOL2, APOBEC3A, MIAT, NELL2, CXCL1, OAS2, OAS3, LAMP3, SLC7A5, BUB1B, SERPINB9, CH25H, ECT2, CTD-2228K2.7, GPR171, MELK, KIF15, S100A2, NAPSB, IFI6, NABP1, MPZL2, CHEK1, XAF1, ISG15, C9orf84, CTLA4, CDCA3, TMC5, CCNB2, ADAM8, SLC16A3, HERC6, DSG3, KIF18A, DUOXA1, KIF11, DUOX1, EPSTI1, HJURP, SELO, ANKRD29, CDCA7, SLC2A10, ANTXR1, ZNF367, CENPA, RAD51AP1, MAML2, NXN, CIS, UBE2L6, DDX60, IL10, TNFAIP6, SLAMF7, AC010518.2, TNIK, IL15RA, CENPF, COL5A2, MYBL1, AURKB, TMEM140, MASTL, PLEKHA7, C2, TGFBII1, SP110, MEIS2, FAM20A, LILRB2, FXYD6, LILRA5, SAMSN1, MX1, S100A12, KIF2C, E2F8, WHSC1, HIVEP3, CDKN3, CDKN3, TYMS, TAPBP, COL27A1, CDCA2, LMNB2, VSNL1, MYO10, CENPE, RP 11-303E16.2, LTB4R, COL6A5, F12, IL411, FAM83D, CDC7, TNC, TROAP, CD274, CENPA, CDC6, TPX2, MCM10, SERPINB9, MS4A6E, RGL1, BTN3A3, NCKAP1, MME, ANKDD1A, CD274, CD274, KIF4A, MCM10, PTK2, RARRES1, IGFBP4, TEAD1, SLFN5, XAF1, NEK2, FHL2, CD2AP, SGCE, RABGAP1L, DCBLD2, CDC45, FAM26F, FAM26F, HIST1H2BE, MT1X, DYSF, PTTG1, C1QB, SELL, ALDH4A1, NAA15, UBA6, CARHSP1, NUP210, SAMD9, RGS14, TTC39A, PARP12, RALGPS2, PRC1, NETO2, NOD2, ALDH16A1, GZMB, FSCN1, INO80D, JAK3, SLC4A11, MAD2L1, FOXE1, PBK, GLS, SLC16A14, WDR4, PARP9, MMP7, ACAP3, STIL, CD47, CDK5R1, FANCD2, OASL, PLAU, IL4R, SLC38A5, TNFSF10, TRIB2, HELLS, HIPK3, CCL2, KCNK6, MT1E, FCRLB, EGFR, ACP2, FOSB, HEG1, LARP6, CKS2, PTPRC, SAMD4A, MIR5188, CYSLTR1, CYSLTR1, PLAC8, PHLDB2, PML, PML, PML, OBSL1, EPDR1, SPATS2L, PNPT1, LOXL2, FCGR3B, RRAS2, CNN3, NAP1L5, CMKLR1, RHEBL1, ADAM8, PLK2, SYT11, ATP10A, ACSM5, WLS, DSP, HIVEP2, AOC1, CARD16, MT1H, GJA1, NRTN, ZBED6CL, TACC3, TCN2, GIMAP6, GIMAP6, CASC5, SLC25A48, TK1, TM4SF1, HIST1H3A, MTFR2, PEX13, PNP, TRIM10, APOL6, HDX, THBS1, GIMAP4, HES1, SLC38A1, TRIM14, GTSE1, AIF1, AIF1, ENAH, TLR3, KIF14, FBXO5, MS4A6A, JAK3, CD59, CD59, TYMP, NR1H3, PKIG, C1R, MOV10, BTG3, BTG3, TPBG, CTSV, IL1RN, TRIM6, OTOF, DSG2, RP11-810P12.7, ZNF618, MERTK, GULP1, DUSP1, UHRF1, CCNB2, NUSAP1, NUSAP1, CD72, GLUL, STMN1, THBD, C19orf66, C19orf66, RHBDF2, FAM46A, SASH1, CDCA3, NAMPT, ICAM2, SPTLC2, RHBDD2, TUSC3, MFHAS1, TPBG, CTC-251116.1, LCK, GEN1, CCND2, SMC4, IL26, CENPK, PLAT, TIMELESS, CARDO1, NUP50, CCDC88C, KIAA1217, FBXO5, COTL1, ARHGAP11B, GINS3, NFASC, COPG1, CCNE2, RACGAP1, DTL, AKIRIN2, C6orf141, ARRDC1, ZBED2, STAT3, SLAMF8, ESPL1, SGK1, ITK, MAP3K14, IL27RA, TNPO1, ACAP1, SERPINB1, CAPN1, SECTM1, GINS1, ATAD2, HOMER1, PRRG4, MIB2, BAIAP2, PARP14, PRSS27, TNIP2, GLB1L3, TRPM1, IL12RB2, SPAG5, VPS9D1, ARNTL2, MCM5, STYK1, NAA50, POLR3G, LTB, GMFB, LINC00518, SHCBP1, KCNJ15, ST14, PSPH, SLC7A1, TSTA3, MAP4K4, E2F3, PPP2R2C, TRIP13, BCL3, RP11-295G20.2, SOCS1, FAM110C, DENND2D, MIR17HG, PPP2R3B, MICALL2, PKP3, RCC1, RASGRP1, NBEAL2, AP1S3, EPHA2, IFI44, PGF, GBP1, SLAMF, RP11-52612.5, SHMT2, ZC3H12D, ANP32E, PSRC1, R3HDM4, C3orf62, RNF213, RNF213, CYP7B1, CDT1, LRP8, ZNF341, GNLY, GNLY, RAD52, NDRG4, TRMU, LGALS2, AKAP8L, PACSIN3, CD1B, CDCA5, MBNL2, WDR34, DDX58, SERPINE1, ESRP2, UBE2T, NFKB2, ABCA7, PARP14, GNG12, FBN1, UCHL1, BEX2, LRRC17, ADGRE2, TANC1, PSMB9, TAP1, RP11-489E7.4, TRIM69, CSRNP1, RP5-884M6.1, STMN3, TMEM173, HDGFRP3, TGFA, PTPRK, NLRC5, TFAP2A, GINS2, PNKD, MT1G, BLZF1, ROBO1, CHEK1, CHEK1, CDKN2C, BACE2, MFI2, CDCA5, OCIAD2, SLCO2B1, SLCO2B1, SLCO2B1, INHBA, PDLIM1, CAV2, PRR16, NR4A2, KDELC1, CAP2, NMI, SOCS2, SH3PXD2A-AS1, CCL5, NOD2, GIMAP8, KIF18B, FAM229B, HSPA1B, CCNF, IDH3A, DENND4A, ITGAL, TUBGCP6, GJB6, E2F2, GBP2, C1QC, MKI67, XRN1, LAPTM4B, APOL1, TXNIP, CLEC4G, PARP10, CENPU, SLIT2, FLOT1, PXDN, PERP, OIP5, USP11, ST3GAL6, EIF2AK2, HIST1H4B, SUCNR1, C5orf56, S100A2, C1QA, MAP2K6, TJP1, GADD45B, AC079305.10, ERC2, FPR3, DENND2D, FCER1G, MCOLN2, AMIGO2, PPA2, BARD1, CASP1, CASP1, IL6ST, PVRL2, NRP2, NRP2, DHX58, UBE2T, RP11-504A18.1, HIST1HIE, MS4A7, CKB, SLFN13, SLC12A9, ETNK1, ETNK1, STAT4, ADAM19, SIX1, AKAP7, S100A16, SLC8A1, SEMA6B, SOX9, KIF18A, WDR34, GBP3, PILRA, MAST4, PTPN2, HTRA1, LY6K, HK3, C9orf91, BST2, FAM64A, UNC93B1, FAT1, RAI14, SFRP1, RRAS, STC2, POLE2, AURKA, and ARAP2.

When treating or preventing arthritis, the compositions, manufacture, products, processes, methods, and/or methods of use, prevention and treatment will, in some preferred embodiments, modulate the expression of at least 2%, preferably at least 20%, and most preferably, >30% of genes selected from SPP1, FN1, SPP1, FN1, GPNMB, GPNMB, OLR1, CXCL10, LAMP3, CCL2, CTSL, LAMP3, RNASE1, MALAT1, CXCL9, Crabp2, MYOF, OLR1, MAF, CCL8, RNASE1, Medag, C1qtnf3, SLC39A8, LHFPL2, C1QB, MARCO, FSCN1, CCR5, APOC1, EMP1, Cc12, TACSTD2, FPR3, TACSTD2, TGM2, DPYSL2, AXL, IQGAP1, MYOF, MMP9, CD9, CADM1, RSAD2, DOCK4, Fndc1, Serpina1, GSN, IDO1, EMP1, CADM1, GBP1, MRC1, CHD9, ENPP2, APOC1, GZMK, F3, THRAP3, ATRX, FBP1, TUBB2A, CCR5, MAF, GSN, CD9, LILRB4, Postn, OSBPL8, ANXA4, CSTB, MAFF, Inhba, MARCO, CCND1, ANXA4, FSCN1, CD1C, AXL, LMNA, CXCL13, CXCR6, DUSP4, DUSP4, PLTP, GZMA, SYNJ2BP, Rgs5, SON, RIF1, Spon1, Igf1, IFNG, KLF13, PRDX1, Lbp, PRRC2C, PLEKHA2, IFI44L, CTSL, CCL18, PHLDA2, SLC16A3, SLC16A3, ALCAM, ENPP2, CCL2, MRC1, LGMN, STAT1, Aspn, Aqp1, ANXA2, Csrp2, SMC3, NR1H3, CSTB, RALBP1, ANXA2, P2RY14, P2RY14, PRDX1, CTNNA1, C1QB, RGS1, ALCAM, FGL2, MTHFD2, CD58, CTNNA1, MALAT1, BOD1L1, PTPRC, C3AR1, APOBEC3G, IQGAP1, TXN, RIN2, LMNA, CXCR6, LGMN, EPB41L3, DPYSL2, LILRB4, VSIG4, ACOT13, LAG3, CEP350, EZH1, BCL2A1, Medag, SLC8A1, LTN1, FBP1, ENG, Nbl1, Lrrc17, TUBB2A, ALOX5AP, APOBEC3G, STAT1, PLTP, CYFIP1, PLSCR1, FCER1A, LHFPL2, ATP2B4, PEA15, CAPG, VSIG4, RGL1, ACOT13, SERPING1, IL32, WSB2, CD163, WSB2, TGFBI, GZMB, Col4a1, Prss23, Cd44, LIMS1, THAP6, IKZF3, ZEB1, CTD-2017D11.1, TYMP, CD59, RGS1, Fabp4, Cdh13, Akr1b7, Cd93, Ctsc, Arsi, Rgs4, Npas2, IL1RN, TXN, SLCO2B1, ZMIZ1, CYFIP1, DOCK4, CD59, KYNU, GPR137B, PEA15, FRMD4B, MX1, LDLRAD4, TRIM14, LDLRAD4, ACP2, EGR2, CCND1, MMP12, CXCL13, EGR2, HPS5, BASP1, Tnc, Wisp2, AHCYL1, Olfm1l, S1pr1, P4ha3, Tnfrsf12a, PHLDA2, Tfpi2, AABR07051947.1, Mgst1, Col1a1, SPAG9, HECTD1, ABLIM1, CLIP1, ARHGAP18, EIF4G1, OTUD4, KIAA1551, SLC39A8, ATP10D, RNASE6, FLT1, OPTN, TUBA1B, GBE1, S100A11, IQGAP1, SLC31A1, GBE1, NAB1, ALDH2, ARPC5, DBI, ENG, SEL1L3, PSMD14, ACP5, MTHFD2, FGL2, FRMD4B, CTSZ, RALA, FCGR2B, CAPG, GRAMD4, FPR3, TGM2, CD163, HPRT1, ADCY7, CSF2RA, Tnfaip6, Enpp3, Arnt1, Plscr1, Emcn, Arnt1, Gucy1b3, Gucy1b3, Kitlg, Cthrc1, HELB, CCL18, Panx3, Abracl, Lgalsl, Tagln, TAOK1, KMT2C, SLC16A7, SART3, HIST1H4C, RASA1, RBPJ, CD2, PSMD14, CPNE3, RB1, S100A10, PTPN13, S100A11, SKAP2, S100A10, ALOX5AP, YWHAH, TUBA1B, ICAM1, PFKP, PCNA, EGR3, PRDX4, RALBP1, TRDC, HOPX, TMED10, PCMT1, TAP 1, GSTO1, CHSY1, PCMT1, AQP9, CREBL2, ATP1B3, CRIM1, FCGR2B, TRIM14, RNF13, DUSP3, DYNLL1, PLAU, DUSP3, TIMP1, ANXA5, CD96, CD58, ANXA5, CD63, LGALS3, NMI, IFIT3, CSF2RA, BAZ1A, KCTD12, Tppp3, Col12a1, Ccdc109b, Ifitm3, Sfrp4, Crispld2, Lamb1, Serpinf1, CHD4, HLA-DPA1, Cc17, Tgfa, Rnf144b, Kdr, Gap43, Cxcr4, Gpm6b, Ifit2, NFAT5, PCNP, CLK4, CCDC88A, RBM39, RSAD2, IFI44, CSF2RB, DBI, GBP1, CST7, CSF1R, RAP1GDS1, ANXA1, ACSL1, GLUL, TNFSF10, CPVL, ATP6V1A, PSEN1, ALAS1, PRDX3, TGFBI, VAMP8, PRKAR1A, IL2RB, VDR, CPNE3, MMP12, PLXNC1, CTSZ, LY75, PDLIM5, CTSH, ACTR3, LMNB1, CD81, CCR2, MMP9, IRF7, IDO1, FLT3, SFXN3, ISG15, COPB2, THBD, OAS2, SFXN3, HCK, ATP6V1D, SMC1A, HLA-DPB1, XCL2, FAM127A, BHLHE40, LGALS3, ACTR3, CREBL2, PDXK, FAM127A, KCTD12, AHCYL1, HSD17B4, LPXN, MARCKS, HPRT1, CLIC2, PAPSS1, Fam198b, Depdc1, Mmp13, Mmp12, Rab32, Ptges, Pxdn, Marcks, Crip1, Aqp3, PHF3, TRAF3IP3, ZNF567, SLFN5, Mme, Fam134b, Prrx2, Cis, Chsy1, CEP295, INO80D, SCAF11, SCAF11, SF1, G2E3, TRIM14, CCDC186, LIMA1, CNTRL, FCRL5, ZNF814, ACTR2, CCT5, PPP2CB, PRKAR1A, NR4A3, NR4A3, ACADM, GPR137B, HLA-DPA1, CCT5, SLCO2B1, PPT1, CRYZ, CSF1R, PSMA5, SDS, CRYZ, VAT1, PGK1, P2RX4, LPXN, IFI35, IFI16, IL2RG, RTN1, AHNAK, OPTN, TYMP, NR1H3, RECQL, MEF2A, MSR1, PSMB9, GLB1, CD1E, and PAM.

When treating or preventing hypertension, the compositions, manufacture, products, processes, methods, and/or methods of use, prevention and treatment will, in some preferred embodiments, modulate the expression of at least 2%, preferably at least 20%, and most preferably, >30% of genes selected from Akr1c3, Thbs4, RALGPS2, Slc5a12, Thbs4, Postn, Havcr1, Slc5a12, Spp1, Loxl1, Timp1, ZNF711, Comt, Slc5a2, Slc16a1, Lcn2, NEAT1, Col3a1, Sfrp4, Slc7a7, Bgn, Postn, Spp1, Fgf7, Me1, Scp2, Akip1, Emp1, Col1a1, Slc34a3, Slc6a6, Nppa, Akip1, Pla2g2a, NPIPB5, Gpnmb, Mab2113, Adamts1, Sgk1, MGAT4A, XPO1, Nppa, MALAT1, MERTK, RAB11FIP3, Cwc25, Csrp2, Sorbs2, Sorbs2, Maoa, C1qb, Serpine2, Pfkp, Thbs4, ADGRL3, SYTL2, ANKRD10-IT1, DAB2, Col1a1, Timp1, LOC299282, Slc4a4, Slc2a2, Apoe, Timp1, Nppa, Ctss, Bgn, Idh1, Jund, Myl1, Anxa1, Thy1, Maoa, Mgp, Loxl1, MEG3, Fga, Cp, CDK6, CLASP2, Nppa, Aebp1, PRKD3, Cyp2a3, Cyp2a3, Neurod1, Akip1, Cav1, Serping1, Nedd4, Emp1, Dab2, Nr4a1, Pkia, Postn, SMARCA2, PKM, Tmem176b, C1s, Vnn1, Spp1, Epb4113, Ptgds, Angptl4, Lcp1, TM2D1, Ccnd3, Slc6a6, Aldh6a1, Eef2, Clu, Mgp, Coro1a, Cebpd, LYRM9, MGEA5, UAP1L1, DUSP4, FBXO9, ZNF75D, CRIPAK, NIPSNAP3B, SPG7, ZNF514, Cc12, Canx, Lgals3, Serpine1, Pla2g2a, Pla2g2a, Crygd, Rhoa, Pja2, Rhoa, Thy1, Rps271, Lamc1, Sod1, Maoa, Cav1, RT1-Da, Apoe, Acta1, Akr1c14, Akr1c14, Pdyn, Pfkp, Creg1, RP4-717123.3, MDH1, ZMAT3, ZMAT3, and ZMAT3.

When treating or preventing polycystic ovarian syndrome (PCOS), the compositions, manufacture, products, processes, methods, and/or methods of use, prevention and treatment will, in some preferred embodiments, modulate the expression of at least 2%, preferably at least 20%, and most preferably, >30% of genes selected from TSPAN8, CKS2, LXN, HNRNPA2B1, MALAT1, SERPING1, GATA6, UQCRB, METTL7A, UQCRB, SLIRP, TM4SF1, PRPS2, DDX6, MRFAPiL1, PRRC2C, GTF2H5, RPS27L, PSMA4, ATP5E, SNRPD1, CYB5A, PDCD5, FIS1, SAR1A, APP, TXN, COX7B, HNMT, PLAGL1, HSPB11, SRSF7, EDF1, DCN, HMGB2, PDGFRA, HAT1, EIF4G3, NDUFS4, TBC1D4, MED13, ATP6V1D, LAMA2, SKP1, TCF4, CISD1, ENY2, NCBP2, SRSF3, DAD1, LRRC17, GMNN, PNRC2, LSM1, HSD17B4, SNRPD3, MYCBP2, AFF1, EHD1, EHD1, REPIN1, KCTD5, ABHD12, ABHD12, MYLK, MAZ, UBE2J1, CXXC5, JUND, ACTN1, HSPB6, TP53113, RHOJ, MTHFD1L, MTHFD1L, CRIP2, MTHFD2, ATP2B4, FAF1, TEAD2, FLYWCH1, CHAC1, ARHGEF2, CDV3, MBD6, SPRED1, JDP2, SPTBN1, AP2A1, CREM, PDLIM7, PDLIM7, SLC6A9, ECI1, C1QTNF1, VPS26B, GNA12, SESN2, GLT8D2, AP1M1, LIX1L, CCNYL1, MPZL1, MPZL1, PAWR, GLIPR2, LOX, TP53, TPP1, SNTB2, PARVA, ACTG2, PVR, DPP9, 11-Sep, CCDC6, LURAP1L, TM9SF4, SGK223, CYTH3, SLC7A5, DDHD1, CREB3L1, 11-Sep, BCAT1, MSRB3, MAPKAP1, INHBE, EZR, SULF2, NID1, AKIRIN2, MCL1, ZBTB45, HSPG2, AKT1S1, PXN, PCK2, GNS, TGFB2, SCARB2, TNFRSF10B, TMEM30A, CRNDE, SCD, VEGFA, F2RL1, MTHFD2, TM6SF1, EPPK1, RPS23, SURF4, TRIB3, TLE4, LPP, TOX2, AJUBA, SLC3A2, WDR1, CTBP1, UNC5B, LOXL2, IL6ST, EHD2, GPT2, SEC61A1, COL1A1, CLIC4, PHGDH, SLC1A4, PSAT1, ASNS, COL6A1, ARHGDIA, FAM129A, FN1, and THBS1.

When treating or preventing Alzheimer's disease, the compositions, manufacture, products, processes, methods, and/or methods of use, prevention and treatment will, in some preferred embodiments, modulate the expression of at least 2%, preferably at least 20%, and most preferably, >30% of genes selected from TRIM13, LINC01094, MS4A4A, IGF1R, INO80D, DNAJC3, ADAMTS2, F13A1, GPR155, WNK1, ITPRIPL2, DAPK1-IT1, PRELP, ZFP36L1, LTF, RNASE6, SOX9, SPEN, HIPK1, CSRNP3, TTBK2, RGPD5, GPATCH2L, CXCL12, ZNF500, TULP4, NFAT5, HIF3A, DAB2, FLCN, TNRC6B, MAML1, SLC5A3, RBMS3, SOX5, SRGAP1, FTX, NTRK3, FAM13A, HIVEP3, KMT2A, KDM5A, IRF2BP2, TM2D1, BMPR1B, MTSS1L, BTBD7, NAV1, MAP7, pk, ARHGAP31, S100A4, RP11-119D9.1, FTX, JAKMIP2, SGK494, ELK4, YME1L1, TNPO1, RIMBP2, DICER1, CBX5, STAG2, MSI2, PITPNB, BICD1, DDIT4L, MACF1, YY1, ZNF618, KDM4B, RSBN1, ADORA3, STAG3L1, ZFP90, GTF2H2C, ZBTB21, PTPN2, FBXO32, HMBOX1, GNA13, FOXC1, KANK2, RP11-10J21.2, RP11-690121.2, RP11-138A9.1, CTD-3051D23.4, PCBP2, TOB2, VTI1A, NOTCH3, NSD1, FGFR1OP2, MALAT1, ZNF785, ZNF785, KCNK12, RHOBTB3, SKI, SKI, POGZ, GPR146, RYBP, LMO4, SNX19, NKTR, ARID1B, ZBTB20, TBL1X, LPP, SYNE1, RSF1, RBMX, FDFT1, TGFBR3, PKM, SYNPO2, ORAI2, FZD7, CEP350, DTNA, PPFIBP1, TNS1, RP11-319G9.3, HLA-DQA1, TREM2, MLLT10, TLR5, FAM101B, RP11-386J22.3, RP11-617F23.2, NR2F2, NR2F2, FIGN, ZNF160, ZNF148, CABLES1, POLR1B, PGK1, TRIO, ALKBH6, DPY19L1, DZIP3, RIC3, GFM2, SNCA, PRKAR1A, TMEM9B, ANKRD29, PPFIA2, GOT1, FAM162A, CCNT2, PDE8A, GM2A, ERICH3, CADPS2, CDH10, GRM5, DZIP1, SHANK2, KALRN, FABP7, LRP1B, PTN, R3HDM1, KIAA11l09, GOLT1B, UGGT2, TRIT1, METTL3, CBR1, CA14, OXCT1, UBA5, BCAT1, BCAT1, PITHD1, UBLCP1, TUBGCP6, NUP85, PSPC1, SNRNP70, ELOVL6, RP4-555D20.2, SS18L1, ABAT, SRSF2, CCBL2, ATP6V1E1, PEX1, SLC16A6, NDRG4, SNX10, IDI1, IDI1, SEC61A2, PPP1R3E, GSTA4, KIAA1468, GUF1, ZRANB2, ARL1, CHRDL1, QTRT1, CYP26B1, FAM65B, PPP3CA, SACS, ITM2A, PKP4, SCN3A, FEZF2, MAP3K9, SLIT2, PTPRN2, CLDN10, TMEM126B, NRCAM, SRP54, GDA, GPHN, OCIAD1, EXTL2, FAM188A, B4GALT6, COL9A3, RTCA, PRMT8, LIG4, C10orf32, PART1, FADS3, DNM3, ATP6AP2, ZWILCH, HSD17B3, 7-Sep, DNAJC10, GOPC, RP1-39G22.7, ZFHX4-AS1, MIR22HG, UBE2T, TRO, TRO, UBXN4, PTP4A1, STS, GALC, ATP6V1G2, PCSK2, RALYL, FRY, EFR3A, RCAN2, AMFR, RB1CC1, SCN1A, NETO2, PRNP, YTHDC2, LAPTM4B, RBM25, PRPS1, GHITM, CMTM5, NIPAL2, SLC25A46, KCNAB1, MIR7-3HG, KPNA5, SCD5, ACP1, CAPRIN2, TFRC, NAV3, COPS8, PCP4, MAT2A, NAPG, LMBR1, RP11-545I5.3, TIMM23B, UNC80, NDFIP1, ABCD3, EML6, ELOVL2, RP3-428L16.2, VSNL1, PREPL, ACTR2, SGIP1, NNT, ABCC8, CORO6, ACSL6, SLC25A27, SLC25A27, PPM1E, PLCB1, KIAA0368, PRKAR2B, BEX4, SPHKAP, RAB6A, GNAS, STAT1, SERPINI1, ZNRD1-AS1, B3GALNT1, SLC39A6, GPCPD1, PAFAH1B1, ZNF767P, TTC3, TRAPPC11, OGT, PGRMC1, SCD, MDH1, THAP9-AS1, LINC01105, CACNA2D3, UNC13C, SATB2, CHML, CACNG3, TRIM36, CRYM, INTU, NUDT21, MYSM1, DDAH1, C9orf72, ITFG1, TARBP1, CNTN1, GLS, SCOC, RGS7, NEFH, PSMA5, TMTC4, VAMP1, C8orf46, RAB3B, TMEM30A, DCUN1D4, MEG3, HSPA13, ARGLU1, SCG3, MOXD1, ETV1, SYNGR3, RFK, ZNF204P, ANO3, VSNL1, ENPP5, HMGCS1, GDAP1, GAD1, SERPINI1, SRSF5, RPH3A, NECAB1, SCG2, NRXN1, PLK2, RGS4, NEFM, RXFP1, NAP1L5, TRPC1, NPY, SLC4A4, CIRBP, PRKCB, CNR1, SNAP25, COL5A2, COL5A2, RP3-525N10.2, GAS5, PCSK1, and SST.

When treating or preventing inflammation in the context of one or more conditions or disorders or diseases, the compositions, manufacture, products, processes, methods, and/or methods of use, prevention and treatment will, in some preferred embodiments, modulate the expression of at least 2%, preferably at least 20%, and most preferably, >30% of genes selected from CXCL9, CXCL13, ANKRD22, CXCL10, SLAMF7, CXCL11, GBP5, SERPINA1, HLA-DPA1, HLA-DRA, HLA-DQB1, FAM26F, CD74, HLA-DPB1, BCL2A1, CCL8, CHI3L1, SUCNR1, RP1-93H18.6, SLAMF8, CD2, GZMB, FCGBP, CD72, CCL4, HLA-DRB1, LYZ, CLEC5A, RGS1, CD8A, MARCO, CCR2, HLA-DOA, APOBEC3A, GZMA, SOD2, KLHDC7B, LILRB2, ADAMDEC1, CCL5, CCL5, CXCL8, FGL2, LAG3, HLA-DMA, TRAC, CD38, PTPRC, GBP1, GPR84, CD52, LILRB1, IL21R, CYTIP, HLA-DRB6, CLECL1, IFNG, ITGB2, CD3D, FPR3, Clorf162, CCR5, STAT1, DOCK10, IGHM, ZBED2, STAT1, PSMB9, LGALS2, AIM2, SIGLEC10, CCL2, LCK, GCH1, ITGAL, CD48, RARRES3, GPR65, TRAF3IP3, TNFAIP6, IL1RN, FBP1, RTN1, CTSS, CLEC4E, EVI2B, GZMK, IL411, TLR8, HEMGN, CECR1, GPR171, CA1, HLA-DMB, CYBB, TLR2, SAMSN1, TFEC, CLEC7A, PTGER4, NR4A2, GYPA, FBXO6, RUNX3, CTSH, VMO1, APOBEC3G, CTLA4, RGS1, C1QB, RARRES1, HLA-DPB2, LYPD5, GYPB, PTPN22, TNFSF13B, FYB, ITK, TAP1, MS4A7, PRF1, ACSL1, ACSL1, KCNJ10, 1-Mar, CD27, CD86, CD83, CD40, CCL18, RNASE6, LRRK2, CORO1A, NKG7, SLC7A10, SP140, GPR18, CCL7, AHSP, CD3E, BCL11B, TLR1, STAMBPL1, PILRA, CD300A, IL2RG, IL12RB1, TNFRSF9, CD300LF, LPXN, OAS1, GBP2, LILRB4, AQP9, GPRIN3, RAC2, PLEK, PLEK, CD37, GYPA, SNX20, LAPTM5, LYN, LYN, IGHD, SMCO4, THEMIS2, LAT2, IGLV3-10, PRKCB, PARVG, CLECL1, LST1, GCNT1, C1QA, TAGAP, CXCL16, IGSF21, CST7, GBP1, LCP2, LCP2, RASSF4, ADGRE2, IFI44L, CD53, PIK3AP1, MNDA, SIRPB1, TYROBP, PYCARD, C1S, ADGRE1, GBP4, PRKCQ, HLA-G, ARHGAP30, FCGR3B, HAVCR2, QPCT, GRIN3A, MS4A6A, DOCK2, IL13RA1, HCST, PLA2G2D, POU2F2, IRF1, C1QC, IL24, EPSTI1, BTBD16, SECTM1, CLEC12A, TRIM22, SH2D2A, FGR, WIPF1, HCK, NR4A2, NLRC5, CTSC, RHAG, PLXDC2, VNN2, FABP4, CIITA, ICOS, SLA, ALOX5AP, PLAUR, XK, CD247, BATF2, CTSB, IKZF3, HK3, AC005281.2, IL17RA, DAPP1, DAPP1, RSAD2, SCPEP1, SNX10, LAP3, TBXAS1, LILRA2, LAIR1, TSPAN33, IRF8, ARHGAP9, ARHGAP9, CYBA, MYH11, ECHDC2, pk, CEP68, ADGRL3, SAMD5, PDE3A, UACA, VANGL2, DST, PDE8B, RP1-152L7.5, TMEM100, FREM2, CDH11, KCNJ8, MAST4, RP3-368A4.6, NDNF, RP11-887P2.5, AGTR1, AC020571.3, GJC1, SYTL2, LPAR1, P2RY1, PPM1L, ENAH, PDZD2, FGFR3, CPE, SLC4A4, EFNA5, GUCY1A2, EPHA3, FAM84A, IDO2, FAT3, DOK6, EML6, ADAMTS9-AS2, OFD1, SOX6, SYNPO2, RP11-30506.3, TMEM56, PCSK5, PRSS35, PRDM6, EGFEM1P, ATP1A2, ZDBF2, COL8A1, PCSK5, MTUS1, ACTG2, ADGRL3, MIR143HG, C1QTNF7, PDK4, ST6GAL2, PARM1, EDIL3, ROR1, PTH2R, SCN7A, MEG3, BBOX1, TSPAN8, and LINC00551.

When treating or preventing fibrosis in the context of one or more conditions or disorders or diseases, the compositions, manufacture, products, processes, methods, and/or methods of use, prevention and treatment will, in some preferred embodiments, modulate the expression of at least 2%, preferably at least 20%, and most preferably, >30% of genes selected from MMP1, DNER, FOXG1, EIF1AY, MME, SFRP1, IGFBP5, ESM1, SERPINB2, TMEM200A, ADGRL4, FDCSP, PID1, MMP2, CXCL8, ANPEP, NRP2, LAMA4, SLC39A8, HS3ST3A1, ITGA2, COLEC12, ITGA6, SERPINE2, CLGN, KRTAP2-3, FHOD3, VEPH1, TACSTD2, SOX9, PKIA, B4GALT6, DUSP4, STEAP1, STC1, DOCK4, RAB27B, SPESP1, ITGA4, LPXN, PCOLCE2, SOX7, CXCL1, LAMA1, CXCL5, CXCL2, MAN1A1, DSG2, DSG2, CRISPLD1, TFAP2A, ALDH1A3, PERP, NTM, KRT81, SLC26A4, B3GNT5, PERP, BCL2A1, IFI27, LY96, GPC6, SSTR1, SULF1, CNTN3, RORA, SRPX, MMP3, CHRDL1, KRT14, GNG2, KCTD4, BDKRB2, ST6GALNAC5, CLMP, IL1B, TRHDE, FOXF1, EVI2A, SEMA3C, PPAPDC1A, ETV1, CDCP1, RP11-11N9.4, LTF, TNFRSF21, TSTD1, DPYD, SLC10A4, PTGS2, DMBT1, KCNK1, TMEM154, C14orf132, HLA-DPA1, UCHL1, NRN1, MOXD1, C4orf32, ROBO1, IRX3, COL13A1, SLC16A2, MSI2, CAMK4, DDX43, MIR31HG, GPR39, DUSP6, ARHGAP22, NFE2L3, TGFBR2, FAP, GCA, NOVA1, FBN2, CPED1, MOCOS, CHST11, MCC, ENPP2, AZGP1, TCN1, IL6, CNIH3, PBX1, FAM167A, ADTRP, FZD8, FAM109B, SLC6A15, CELF2, CCDC68, IFI44L, SYT14, NOX4, XYLT1, KRT34, FOXF2, LRCH2, ADAMTS3, CDH4, PLAU, PLAU, FAM20C, GLT8D2, CARD6, CCL2, ALX1, MX1, TSPAN13, HPCAL1, HPCAL1, SERPINF1, MAST4, FAM155A, PRDM1, SNAP25, CLCA4, TF, UGT8, CXCL3, MIR4458HG, MYO1D, PAX8-AS1, ABI3BP, MLF1, GOS2, CFH, WISP1, STEAP2, TEK, KCNJ8, HAS2, HAS2, CPNE8, HERC5, STK26, TMEM45A, PAG1, PLAT, FGF2, TPBG, IRX2, PSG5, CTC-231011.1, OSBPL6, TNFRSF19, PODXL, ANTXR2, CA12, OTX2, PKIB, BHLHE41, DRAM1, CDO1, INPP4B, KRTAP1-5, GSAP, GSAP, OGFRL1, FOXE1, MTSS1, ELOVL4, BCHE, GFPT2, SLC2A13, PLA2G4A, IFI44, STEAP1B, LINC01551, DNAH14, PRKG1, IGKV3-20, NFE2L3, HIST1H1C, FCRL5, TRIM2, NHSL1, NT5E, LXN, TGM2, PDE4B, NT5DC1, ZNF697, PPP1R3B, PTX3, TMTC1, PTPN13, DLX1, HERC6, TTLL7, FMNL2, AOX1, TNFAIP6, CTSB, CXCL5, NRIP3, LYPD6, NPY1R, CRNDE, KANSL1-AS1, SLFN5, TMEM155, TNIK, IFITM1, FLI1, KIAA1217, RARRES1, ZNF395, TMEM150C, HIST1H2AC, MCTP1, NMNAT2, LTBP2, PLAUR, SCD5, ANXA10, ENPP1, KIAA1462, LINC00960, LINC00960, SOX7, ANKRD36BP2, KYNU, CLCA2, PLAT, TCF21, CA12, MECOM, KCTD12, LPAR3, GCNT2, OAS2, PSG9, SLITRK5, CTSK, EPHB2, CA13, SVEP1, IL1R1, RASEF, DDX60, KYNU, LY6K, GPNMB, STC1, NTNG1, EDNRB, RGS5, CSTA, IGLV1-47, IGKV1OR2-108, DPP4, GALNT12, ENPP5, SMAGP, TAF4B, SH3D19, HMGN5, IRAK2, BEX2, SMOC1, TOR4A, GRB14, KIAA1549L, KIAA1549L, CDON, FUT8, FUT8, NRP1, CHI3L1, PRKAR2B, LRRC17, GSTO2, TMTC4, COL4A4, FAM169A, LRRC8C, SLC14A1, NFKBIA, OAS1, PABPC4L, SLC22A4, PDE7B, TLE1, APOL6, PTHLH, LIN7A, SEMA4B, EFNA5, TMEFF2, BMPR1B, RGS2, KLHL24, C12orf56, ZADH2, RAC2, PLEK2, SCN9A, FAM13A, APLP2, ITPR1, ARHGAP20, TSHZ1, SLCO3A1, CPPED1, HMOX1, HIST1H2BD, AMPD3, IL1A, DNAJC12, MPP4, MCOLN3, LPP, ZFYVE16, TOP2A, TEX15, LINC00973, HLA-DPB1, CIS, CPM, ATP2B1, GPRASP1, NLRP3, JAM2, AIG1, BEND7, SERPINB7, FAM117B, ERC1, TGFBR3, SERPINA1, OLFML1, CHST2, SCG5, SLC18B1, EVI2B, EPHX4, PTPRB, CXCL12, PTGS1, CPD, CPD, MYEF2, HDAC9, SOBP, HACD4, SUSD5, CLDN1, OASL, SERPINB13, NEFM, PDGFC, TBC1D8B, ARAP2, PGF, PLSCR4, CD47, RRAD, TLDC1, SMPDL3A, F5, SSH2, MAP9, TIGD2, KCNQ5, ZNF585B, IGLL3P, IGFBP3, CTC-444N24.11, CCL19, ANKRD10-IT1, RNF213, PAX3, S100A9, LINC00342, IGKV4-1, NUSAP1, DSG3, TBX3, TBX3, ITPR2, CPE, MX2, NFASC, VANGL2, C5orf30, ARHGEF3, SAT1, GALNT6, SEMA7A, TMOD2, TMEM106B, SPTLC3, GBP2, C1RL, IFIT1, GAS6, GAS6, PNMAL1, EMB, ANGPTL2, ID1, DDX58, C5orf63, ANGPT1, CASP1, KCNJ2, TNFAIP3, VEGFA, VEGFA, GYPC, EREG, SDC1, SDC1, KITLG, PCDH9, IL20RB, SERPINB8, CD109, FAM171B, EMP1, TFDP2, SPON1, CEP68, CD274, CCDC71L, PSG3, PSG3, HECW2, TOX2, SLC35D1, EPHA4, SRPX2, LYN, DENND1B, DENND1B, TNFSF15, ZFPM2, TEX9, CHEK1, MOK, HEBP2, TWIST2, DAZAP2, RNF135, ACAD8, PCNXL2, APCDD1L-AS1, CASP4, SMAD1, PAK1, FAM13C, LMO4, LMO4, ANXA3, TNFAIP2, BACE2, HCLS1, KCNMA1, and SLC25A27.

When inducing pluripotency, the compositions, manufacture, products, processes, methods, and/or methods of use, prevention and treatment will, in some preferred embodiments, modulate the expression of at least 2%, preferably at least 20%, and most preferably, >30% of genes selected from Oct4, Sox2, Nanog, Rps9, Rps9, Usp18, Nr0b1, Rpgrip1, Rps9, Cxcl5, Gm7325, Fgf4, Mmp13, STC1, NPPB, 4930447C04Rik, Zfp42, Gm7325, Pbx1, Ang, Pou4f2, Rps9, NEFL, Meis2, AK4, Ikzf2, Pi4k2b, BNIP3, CASQ2, Zfp42, Pou4f2, Rps9, Saa3, Fgf4, Cdc42ep2, Dppa2, Gdap10, Eif2s1, Col4a6, Dmrt1, ATP1B4, Rtp4, Calca, Jam2, KISS1R, PAPSS2, Mmp3, Nr5a2, Rpgrip1, Dppa2, Esrrb, Dtl, Dtl, Raph1, Ddx4, PGK1, R3hdm1, Galc, Ccdc141, Gm24366, Agfg1, Ddx4, Slc1a5, Gm1564, Ccnb1ip1, Hck, Chchd10, Klf2, Ifi203, Cxcl3, Prrc2c, ENO1, Amph, ERO1L, EGLN3, Ifi44, Gdf3, Tfcp211, Stat4, Snora28, Jam2, Rp1391, Gpc6, Zic3, Tll1, Pof1b, 7-Mar, Eras, SERPINB9, 1700097N02Rik, Gm1564, Enox1, Enpp3, Gm37407, Ssbp3, Ddx43, Mid1, Zfp429, AA386476, Rpgrip1, H2-Q10, 2610005L07Rik, Col4a6, A930004D18Rik, Serpina3g, U90926, 1700097N02Rik, 1700097N02Rik, Pdgfrb, Otx2, Oasl2, Apoc2, Tbx3, L1td1, Gm37219, Sfswap, Adamts9, Sycp3, Xaf1, BEND5, NPPA, Platr4, Gm31266, Snord89, H2-Bl, Rps9, Rnf17, Eif2s1, Cbr3, Snhg4, Ankrd1l, Triml1, Cep295, Nbeal1, Immt, Sox2, Jade1, Stk17b, Tfcp211, Abcc4, Car8, Nasp, Nasp, Ncoa3, Rbm39, Abi2, Farsa, Pi4k2b, Zfp874a, Eif2s1, Olr1, Tfpi2, Tmem191c, Tnfsf11, Mmp10, Sgip1, Tm4sf1, Gm21769, Thbs1, Thbs1, Syce1, Zfp819, Bcl11b, Triml1, Cpsf6, Mycbp2, Foxl2os, Tcf12, Fam25c, Stk31, Zfp429, Dmd, TFPI2, Rbbp4, Phip, Gm17491, Ndufab1, Dppa4, Sall1, Apob, Kcnj3, Ifi204, Slc1a5, Tmem191c, Laptm5, Depdc7, Slc35d1, Tnc, Mageb16, Car3, Zmiz1, Cxcl1, Prrx1, Trim71, Eml5, Mir17hg, Mrpl15, Nudt5, POSTN, TFRC, Chchd10, Gm37598, Gm26853, Nisch, Pan3, Scmh1, Grb10, Lefty2, Mmp3, Calca, Ang, Pappa, Trpm7, Tmem54, Cstf3, Slbp, Bnc2, Nef1, Trim2, Phyhipl, Fbxo15, Adam23, Olr1, Pds5a, Dcn, Mmp9, Map2, Rad23b, Pabpc1, Tle1, Platr4, 1700019D03Rik, Hells, Mbtd1, Dppa4, Tex15, Tll1, Trim30a, Bcl2, Vps41, Prune2, Afp, Cab39, S100A10, HILPDA, Pisd-ps1, Rbm3, Nr0b1, Hmbox1, Ctsh, SLC2A1, Gm6483, Lox, Col1a2, Galc, Katnbl1, Tnfrsf11b, Nefh, Chka, Cenpv, Ppcdc, Wisp1, Tle4, Plagl1, Lefty2, Pla2g1b, Ube2m, Serpinb9g, Jade1, Rps24, Nrp1, Gcnt2, Amn, Cobl, Dnmt31, Rpgrip1, Vcam1, Birc6, Stat1, 116, Zcchc11, Lrp1, Mpg, Airm, Trps1, Cdv3, Thrap3, Kdm5b, Stc2, Platr10, ENO2, PCAT6, FAM162A, CAV1, MASP1, SLC2A3, DDX41, Malat1, Timp2, Osmr, Shox2, LINC00881, FN1, A2m, Nefm, A2m, Prss23, Tmal6, Tshz1, Ube2t, Mmp13, Flrt2, Gpr176, Ptgs2, Mt2, Slc6a15, Kbtbd11, C77370, Cxcr6, Sdpr, Rbms3, Rbms3, Col4a2, 2700038G22Rik, Kantr, Zfp945, Pafah1b1, Rxfp1, Caps1, Gm22077, Gm22574, Rlim, Ero1l, Dnajc6, Efhd1, Calcoco2, Brd8, Slc1a5, Zic3, Ttc14, Ttc14, Act16a, Csnk1a1, Akr1b10, Fbln2, Rgs4, Nfix, Slc35f2, Ermp1, Ttc19, Tmtc3, Fbxo15, Khdc3, Cpt1a, Nef1, Oasl2, Fbn1, Ankrd1, Tgds, Sowahb, Tacstd2, Col3a1, Crim1, Fzd5, Adam10, Col6a3, Bcl11a, Hist2h3c2, Bdh2, Adi1, Crisp1, Ltbp2, Bmper, Lsm8, Hoxc8, Gbx2, Col4a1, Atp11a, Thbs2, Thbs2, Angptl4, Matr3, Msi2, Nodal, Phxr4, Gata3, Kansl3, Sptbn1, Deptor, Sec61a1, Snapc3, Fzd5, Zfp612, Gm37569, Inhba, Col6a1, Rsrc2, Fam92a, Fubp1, Lats2, Mylpf, Gsk3b, Rbm25, 5730507C01Rik, Phf3, Utf1, Imp4, Mylk, Peg3, Tnik, Hspa4, Zbtb8a, Fam111a, Amn, Srgn, Gm21967, GPR157, Cast, Col4a5, S1pr3, Tia1, AGPAT5, Cox7a1, Cspp1, Sprr2h, Gm2115, Zcchc3, Angel2, Asap1, Itpk1, Gm21967, Zbtb4, Neat1, Rpl11, Cdc42bpg, Stk31, Cdh111, Ssbp2, Slc9b1, Slc9b1, Kat2b, Rnf17, Itgae, Gbx2, Etv3, Ifih1, Ankrd44, Btg1, A730062M13Rik, Frrs1, Zfp386, Ltbp1, P4HA2, PKM, EGLN1, CRYAB, EDN1, JMJD6, FUT11, Dmtn, Mmp3, Dmrt1, Gm4944, Vgll3, Slit2, Npr3, Anxa8, Fbln5, Prom1, Hsf2 bp, Pdzd4, Zfp874a, Atxn3, Ctr9, Tor3a, Actr3b, Gtsf1l, Ubr7, Ncam1, Heg1, Hnrnpr, Whsc1, Usp14, Trip12, Usp7, Zkscan3, Hoxd13, BC022960, Ppp3r1, 1700097N02Rik, Ddit41, Gga2, Ube2i, Nell1, 2900011008Rik, Hpdl, Xpo4, Erlec1, Gm11627, Ttpa, and Fam25c. In accordance with the present invention, any method of extraction or purification known to those skilled in the art may be used in obtaining at least one the agent, compound or drug of the present invention, e.g. extraction using alcohol (including methanol, ethanol), or aqueous extraction using solvents such as ketones, esters, ethers, polyols, chlorinated solvents, and mixtures of two of the aforementioned solvents.

In some embodiments, the composition of the present invention comprises at least two agents, compounds or drugs named herein (e.g. zingerone or sesquiterpene or at least one extract selected from the group comprising orange, frankincense, cannabis or other natural oil or extract or at least one substance or compound isolated from said at least one oil or extract and other natural oils and/or extracts) and/or at least one other agent, compound, or drug of the present invention) and is used to prevent or treat HIV infection.

In some embodiments, the present invention provides for compositions providing analgesia and pain relief in individuals in need of such treatment.

In some embodiments, said composition of the present invention is administered in a dose of from about 0.01 mg/kg of the individual's body weight to about 500 mg/kg of the individual's body weight.

A goal of the present invention is to provide compositions, manufacture, products, processes, methods, and/or methods of use, prevention and treatment at least one extract selected from the group comprising orange, frankincense, cannabis or other natural oil or extract or at least one substance or compound isolated from said at least one oil or extract and other natural oils and/or extracts and/or at least one other agent, compound, or drug of the present invention suitable for use in individuals in all states of health.

In some embodiments, the compositions, manufacture, products, processes, methods, and/or methods of use, prevention and treatment of the present invention further comprise an approved drug-especially a drug for treating or preventing the same disease or condition, or similar disease or condition for which at least one extract selected from the group comprising orange, frankincense, cannabis or other natural oil or extract or at least one substance or compound isolated from said at least one oil or extract and other natural oils and/or extracts and/or at least one other agent, compound, or drug of the present invention and/or other agent, compound, or drug of the present invention is being provided.

In some embodiments the approved drug is an FDA approved drug.

In some embodiments, at least one extract selected from the group comprising orange, frankincense, cannabis or other natural oil or extract or at least one substance or compound isolated from said at least one oil or extract and other natural oils and/or extracts, and/or at least one other agent, compound, or drug of the present invention is combined with an approved drug-especially a drug for treating or preventing the same disease or condition or similar condition for which at least one extract selected from the group comprising orange, frankincense, cannabis or other natural oil or extract or at least one substance or compound isolated from said at least one oil or extract and other natural oils and/or extracts and/or at least one other agent, compound, or drug of the present invention is being provided.

In some embodiments, the approved drug provided with the at least one extract selected from the group comprising orange, frankincense, cannabis or other natural oil or extract or at least one substance or compound isolated from said at least one oil or extract and other natural oils and/or extracts and/or at least one other agent, compound, or drug of the present invention is an approved drug that produces an increase in intracellular or extracellular glutathione concentration.

The current invention enables the application of more effective combination compositions.

For example, the invention also relates to the use deuterium depleted water (DDW) for the preparation of compositions and is useful in the examples described herein (see below).

The invention also relates to aqueous pharmaceutical compositions usually applied in curing where the aqueous component is DDW with deuterium content of 0.01 to 135 ppm.

The invention also relates to aqueous compositions usually applied in therapy where the aqueous component is DDW with deuterium (D) content of 0.01 to 135 ppm. Such composition is advantageously formulated as solution, cream or gel, e.g. as isotonic infusion stock solution.

The invention further relates to a method for prevention or treatment of cancerous or dysplastic conditions or diseases in which deuterium-depleted water (DDW) with 0.01-135 ppm deuterium (D) content is administered together with one or more other compounds, agents, extracts, oils and/or drugs, etc. of the present invention capable of increasing RMR/RRE.

In some embodiments, DDW is incorporated in the composition for the prevention or treatment a cancerous or dysplastic condition, wherein the composition comprises DDW with deuterium content of 0.01 to 135 ppm in conjunction with one or more other compounds, agents, extracts, oils and/or drugs, etc. of the present invention.

In some embodiments, DDW is incorporated in the composition for the prevention or treatment of cancer, wherein the composition comprises DDW with deuterium content of 0.01 to 135 ppm and one or more other compounds, agents, extracts, oils and/or drugs, etc. of the present invention, optionally together with one or more usual pharmaceutical auxiliary material(s).

In some embodiments, these compositions further comprise the use of one or more anticancer drug(s)/agent(s)/compound(s)/oil(s)/extract(s), etc.

Certain aspects of the present invention relate to the application of deuterium depletion alone or in conjunction with one or more other compounds, agents, extracts, oils and/or drugs, etc. of the present invention to substantially increase RRE/RMR which can result in prevention or treatment of a variety of conditions or diseases that cluster in individuals, that are associated with chronic diseases that cluster in patients (CDCP) including those comprising syndrome X, metabolic syndrome, other conditions, diseases and disorders, or that are known to those skilled in the art to be associated with low resting metabolism or where energy intake that exceeds energy expenditure.

The invention also relates to the use of deuterium-depleted water (DDW) for the preparation of combined pharmaceutical compositions for prevention or treatment of various conditions and diseases associated with non-optimal resting metabolic rate (RMR)/resting energy expenditure (REE), where the composition comprises DDW with deuterium (D) content of 0.01 to 135 ppm optionally, optionally together with one or more other compounds, agents, extracts, oils and/or drugs, etc. of the present invention, optionally together with one or more usual pharmaceutical auxiliary material(s).

Table 6 is an illustration of effect of drinking Preventa 85 or 105 ppm deuterium depleted water on resting metabolic rate. It shows exploratory study of one male and one female subject. Each consumed at least 1.5 L per day of Sparklett drinking water (˜148 ppm) for one week before switching to 1.5 l per day of Preventa 85 or 105 ppm deuterium depleted drinking water. RMR measurements performed with a Breezing device. Each analysis was done once per day in duplicate or triplicate before starting DDW and twice per day thereafter in duplicate or triplicate except for Study Day-8 for male subject when only one test was done.

TABLE 6
Effect of Drinking 85 or 105 ppm Deuterium Depleted
Water (DDW) on Resting Metabolic Rate

| Study | Resting Metabolic Rate (RMR) | | Saliva deuterium level | |
|---|---|---|---|---|
| Day | Male | Female | Male | Female |
| 1 | 1472 | 1053 | 148 ppm | 148.6 ppm |
| | (without | (without | (without | (without |
| | DDW) | DDW) | DDW) | DDW) |
| 2 | 1450 | 1007 | na | na |
| | (started 105 | (started 85 | | |
| | ppm DDW) | ppm DDW) | | |
| 3 | 1653 | 1133 | na | na |
| 4 | 1815 | 1247 | 141.5 ppm | 138.2 ppm |
| 5 | 1862 | 1363 | na | na |
| 6 | 2135 | 1510 | na | na |
| 7 | 1993 | 1522 | na | na |
| 8 | 2150 | 1412 | na | na |
| 9 | 2010 | 1497 | na | na |
| 10 | 1943 | 1462 | 136.7 ppm | 133.9 ppm |

*na—not applicable

The data indicates that the male subject's saliva deuterium level decrease from 148 to 136.7 resulted in a 41% increase in his resting metabolic rate within 7 days of drinking 1.5 L per day of Preventa 105 ppm deuterium depleted drinking water.

The data indicates that the female subject's saliva deuterium level decrease from 148.6 to 133.9 resulted in a 44% increase in her resting metabolic rate within 7 days of drinking 1.5 L per day of Preventa 85 ppm deuterium depleted drinking water.

In some embodiments, the DDW is applied in combination with one or more other compounds, agents, extracts, oils and/or drugs, etc. of the present invention, and one or more other drug, agent, compound, oil, extract, etc. is an a drug, agent, compound, oil, extract, etc. that may potentially be applied in advantageous cases in doses from 80-150% of the usual dose.

In some embodiments, the DDW is applied in combination with one or more other compounds, agents, extracts, oils and/or drugs, etc. of the present invention, and one or more other drug, agent, compound, oil, extract, etc. is an anticancer drug, agent, compound, oil, extract, etc. that may potentially be applied in advantageous cases in doses from 80-150% of the usual dose.

In some embodiments, the DDW is applied in combination with one or more other compounds, agents, extracts, oils and/or drugs, etc. of the present invention, and one or more other drug, agent, compound, oil, extract, etc. is an metabolism-modulating drug, agent, compound, oil, extract, etc. that may potentially be applied in advantageous cases in doses from 80-150% of the usual dose.

Due to the interaction of DDW and one or more other compounds, agents, extracts, oils and/or drugs, etc. of the present invention, the anticancer drug(s)/agent(s)/compound(s)/oil(s)/extract(s), etc. may potentially be applied in advantageous cases in doses reduced to 80-10% of the usual dose.

In some embodiments, the composition comprising DDW contains may contain one or more other compounds, agents, extracts, oils and/or drugs, etc. of the present invention in reduced dose which is 80-10% of the standard dose.

Due to the interaction of DDW and one or more other compounds, agents, extracts, oils and/or drugs, etc. of the present invention, the anticancer drug(s)/agent(s)/compound(s)/oil(s)/extract(s), etc. may potentially be applied in advantageous cases in doses reduced to 80-10% of the usual dose.

Due to the interaction of DDW and one or more other compounds, agents, extracts, oils and/or drugs, etc. of the present invention, the anticancer drug(s)/agent(s)/compound(s)/oil(s)/extract(s), etc. may potentially be applied in advantageous cases in doses increased to 110-500% of the usual dose.

In other embodiments, the composition comprising DDW contains the one or more other compounds, agents, extracts, oils and/or drugs, etc. of the present invention in increased dose which is 110-500% of the standard dose.

From the above, it follows that such an embodiment of the invention in which preparations, compositions are manufactured in a medium (solution) with reduced deuterium content, by using DDW as vehicle, because DDW can substantially improve the efficacy of one or more compounds, agents, extracts, oils and/or drugs, etc. of the present invention, and the dosing of DDW also can be optimized this way.

If the infusions, medicines and other auxiliary compositions (infusions, injections etc.) are produced using DDW, the results achieved by one or more other compounds, agents, extracts, oils and/or drugs, etc. of the present invention can be further improved.

A further advantage of the new combined compositions described by the invention is that in using them for follow-up treatment of patients so that the rate of relapse can be greatly reduced.

The compositions according to the invention optionally may contain beyond the one or more other compounds, agents, extracts, oils and/or drugs, etc. of the present invention and DDW, also one or more inert, nontoxic auxiliary material(s) (e.g. vehicles, moisteners, sweeteners, aromas, buffers etc.). The composition can be formulated for oral (tablets, solution, emulsion, suspension etc.) or parenteral (e.g. infusion solution) application.

As noted elsewhere herein, the compositions according to the invention can be produced by the known methods of pharmaceutical manufacturing, such as by mixing the agents and the organic or inorganic vehicle(s) and formulating the mixture into a composition.

The daily dose of the pharmaceutical compositions described by the invention can be varied in a wide range, depending on several factors such as the patient's body weight and surface area, the one or more other compounds, agents, extracts, oils and/or drugs, etc. of the present invention, and the deuterium level of DDW, as well as the deuterium level of the subject.

The major advantages of the compositions and procedures described by the invention are as follows:

i. The simultaneous effect of DDW and one or more other compounds, agents, extracts, oils and/or drugs, etc. of the present invention(s) prepared in DDW can increase the RRE/RMR of the patients.
ii. In the follow-up treatment of patients, the chance of relapse is reduced.
iii. Preparing the other agent(s) in DDW enables ongoing reduction of the RMR/RRE, such as administering of infusions, which may substantially increase the efficacy of the treatment.
iv. Using DDW and one or more other compounds, agents, extracts, oils and/or drugs, etc. of the present invention together can diminish the side effects of the latter.
v. In using DDW and one or more other compounds, agents, extracts, oils and/or drugs, etc. of the present invention together, 80-10% of the standard concentration of the one or more other compounds, agents, extracts, oils and/or drugs, etc. of the present invention agent, such as 80, 70, 60, 50, 40, 30, 20 or 10%, can be applied at equal or increased efficacy and greatly reduced toxicity.
vi. Conventional agents prepared with DDW and one or more other compounds, agents, extracts, oils and/or drugs, etc. of the present invention may be of effect on conditions regarded earlier as refractory, and conditions diagnosed in late stage may become treatable.
vii. The application of conventional compositions made with DDW needs no special procedures.
viii. The compositions can be produced on industrial scale.

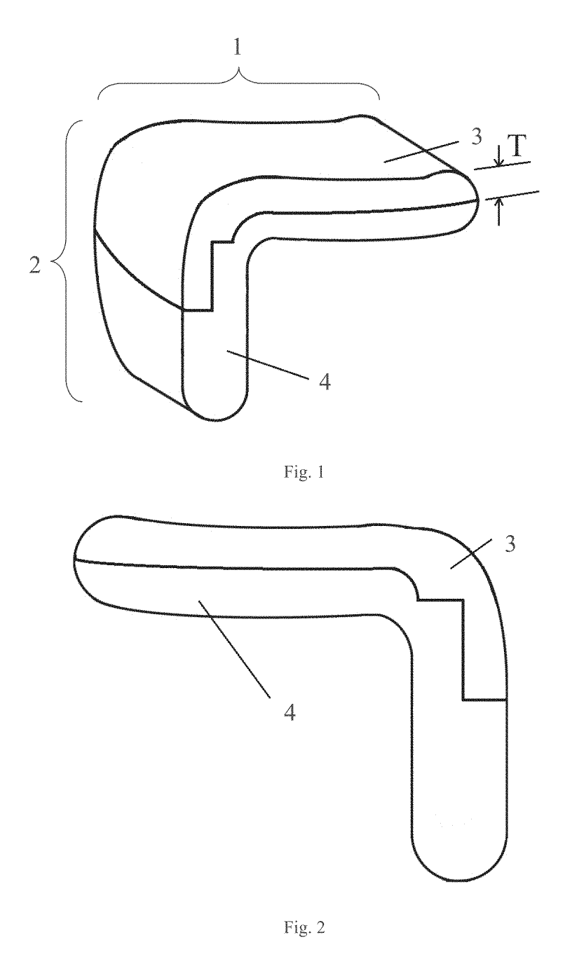

FIG. 1 is a general cross-section view of a right sided version of “L”-shaped solid buccal or sublingual dosage form according to one of the preferred embodiments of the present invention.

FIG. 2 is a side cross-section view of a left sided version of “L”-shaped solid buccal or sublingual dosage form according to another preferred embodiment of the present invention.

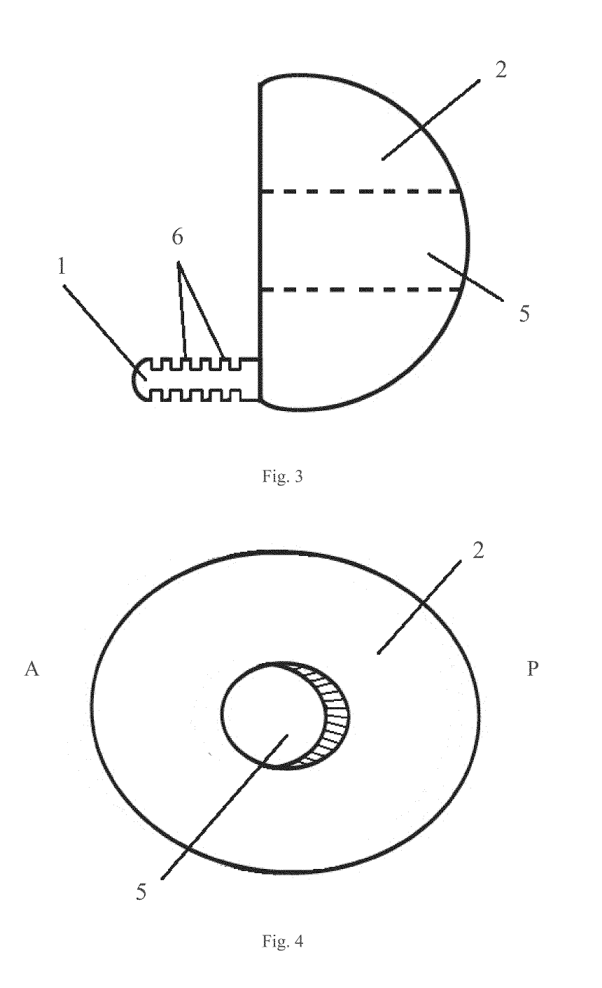

FIG. 3 is a front view of perforated “L”-shaped solid buccal or sublingual dosage form according to another preferred embodiment of the present invention.

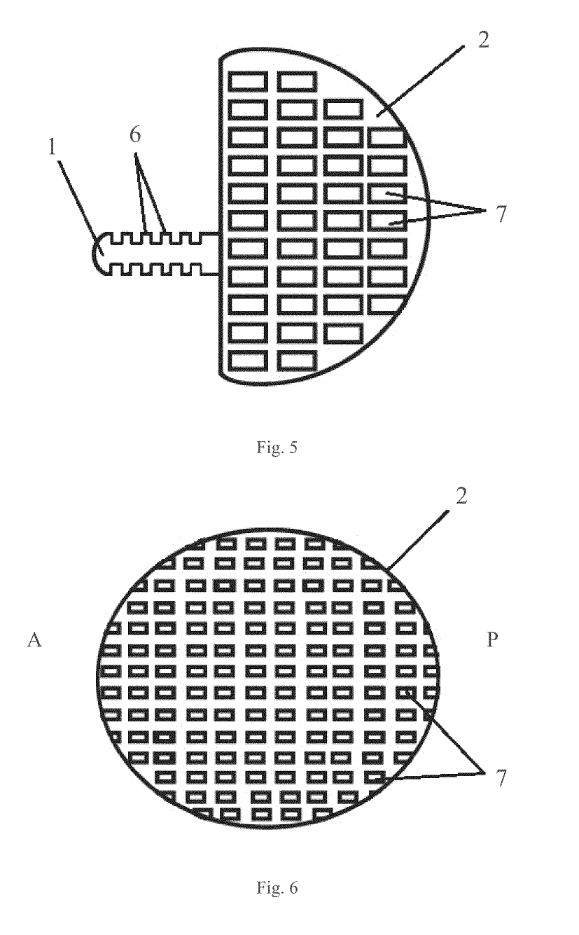

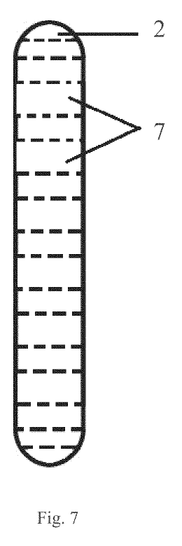

FIG. 4 is a general right view of perforated “L”-shaped solid buccal or sublingual dosage form from FIG. 3.

FIG. 5 is a front view of fenestrated or dimpled “T”-shaped solid buccal or sublingual dosage form according to yet another preferred embodiment of the present invention.

FIG. 6 is a right view of fenestrated or dimpled “T”-shaped solid buccal or sublingual dosage form from FIG. 5.

FIG. 7 is a is a front view of fenestrated or dimpled flattened solid buccal or sublingual dosage form according to yet another preferred embodiment of the present invention.

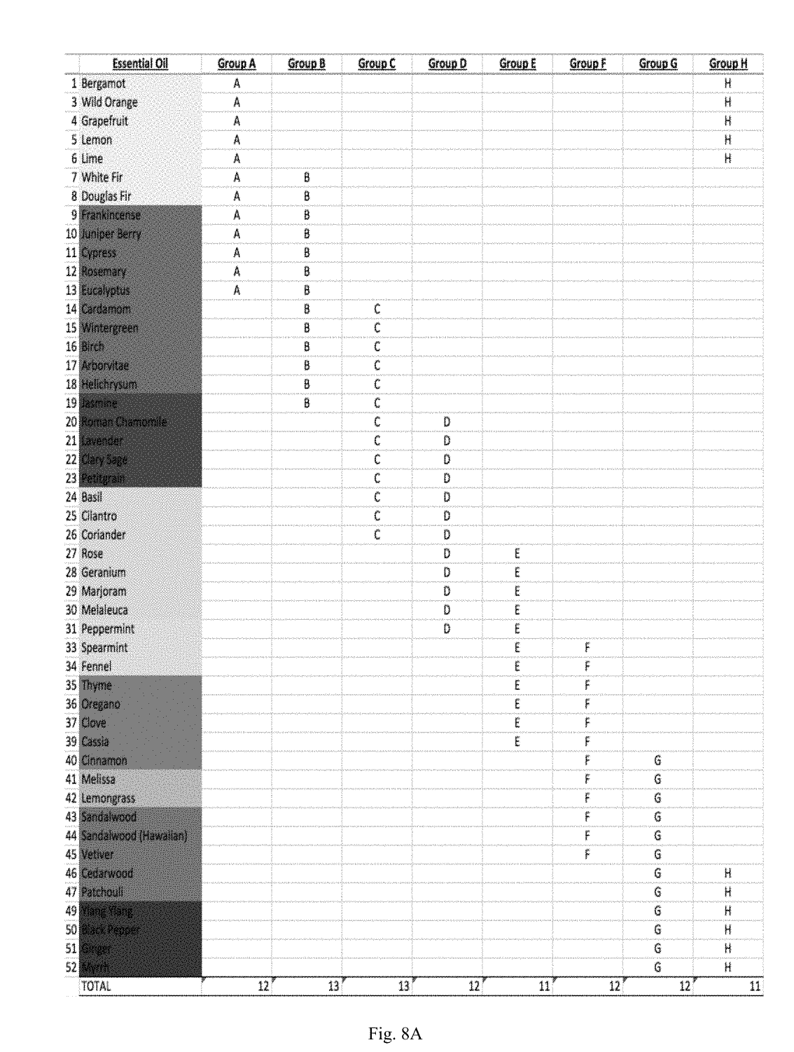

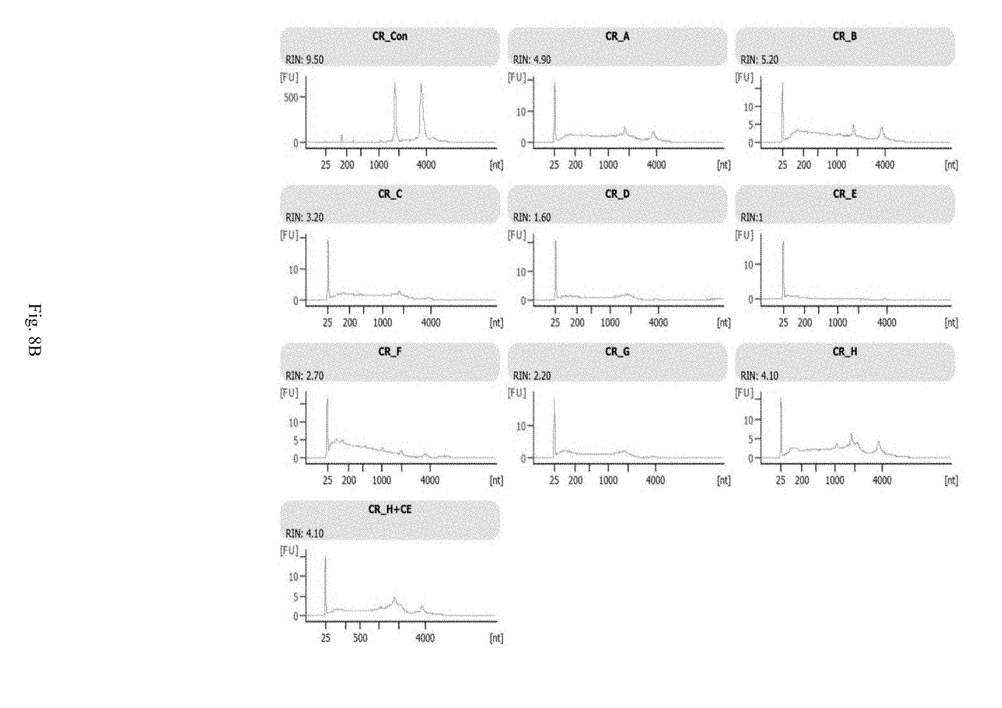

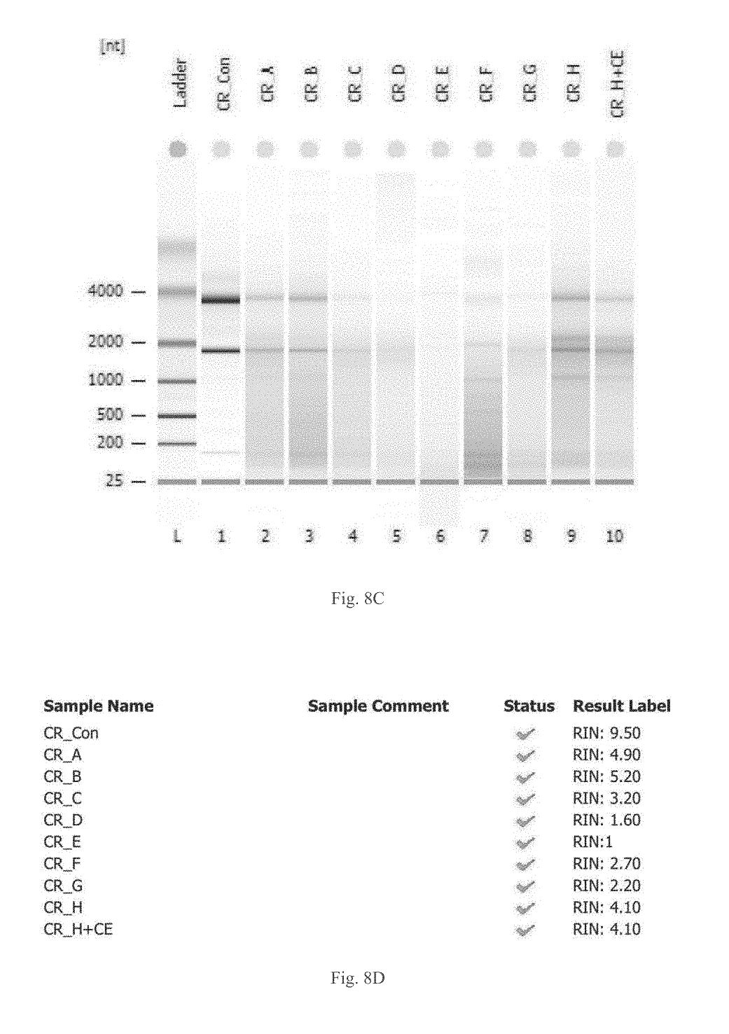

FIGS. 8A-8D are tables and diagrams illustrated NCCIT apoptotic RNA degradation after 24 hours incubation with various combinations of natural oils.

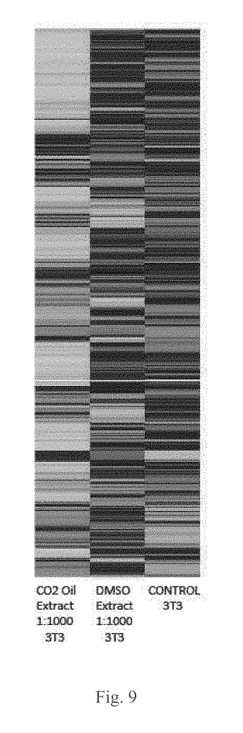

FIG. 9 is a heat map showing gene expression in cannabis extract treated cells relative to control.

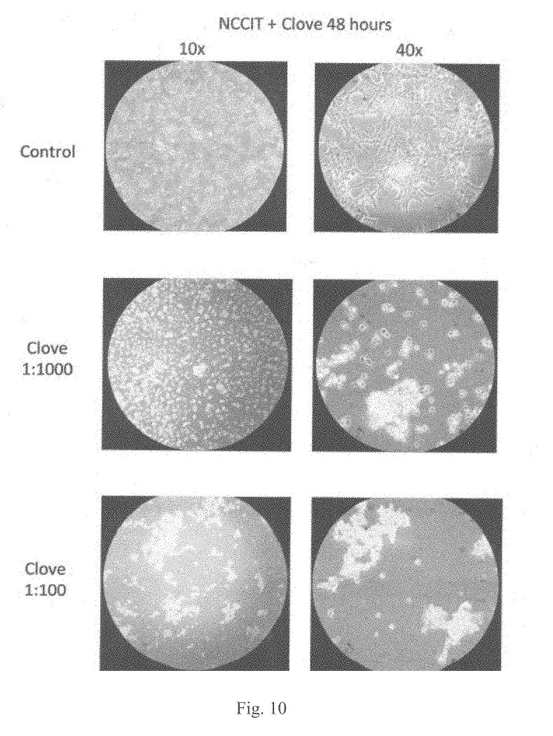

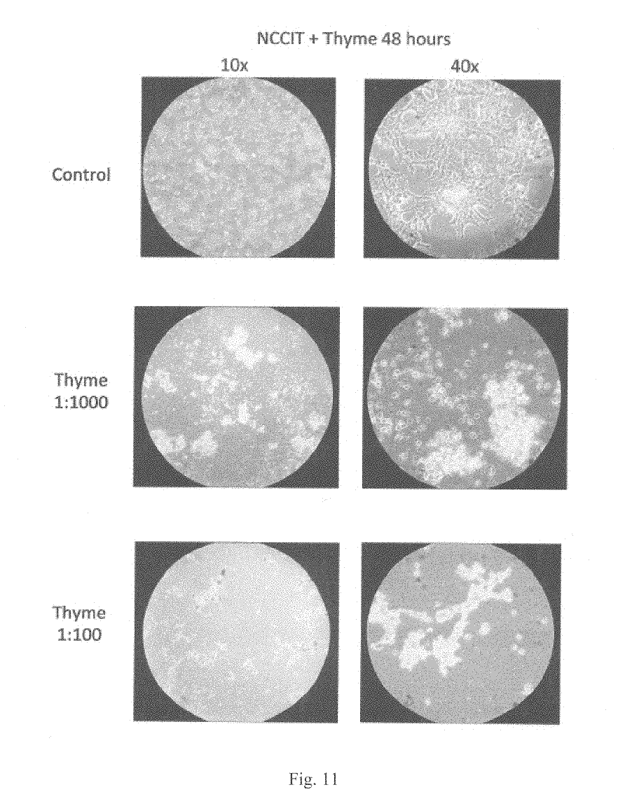

FIGS. 10-17 are illustrations of biological activity of dilute natural oil extracts in cultured NCCIT cancer and 3T3 cells.

FIG. 1 depicts a general cross-section view of a left sided version of “L”-shaped solid buccal or sublingual dosage form according to one of the preferred embodiments of the present invention. FIG. 2 depicts a side cross-section view of a left sided version of “L”-shaped solid buccal or sublingual dosage form according to another preferred embodiment of the present invention. The buccal or sublingual dosage form has an interocclusal, dental portion 1 (portion abutting the teeth) and a buccal portion 2 (portion abutting the cheek). The dental portion 1 of the dosage form connected to the remainder of the dosage form (buccal portion 2) comprises an upper bite surface member 3 for contacting upper molars of a subject or patient, and a lower bite surface member 4 for contacting lower molars of the subject or patient. The thickness T of the upper bite surface member 3 is approximately 1.0 mm to 2.5 mm in a preferred embodiment. The dimensions of the lower bite surface member 3, however, may vary widely according to the desired dose to be administered.

In some embodiments of the invention, the dental upper bite surface of the upper bite surface member 3 is curved to match the Curve of Spee. In other embodiments, the dental upper bite surface member 3 is substantially flat or ridged to reduce sliding and to cause the dosage form to remain more securely in place.

FIGS. 3 and 4 are a front view of perforated “L”-shaped solid buccal or sublingual dosage form and a general right view of perforated “L”-shaped solid buccal or sublingual dosage form correspondingly according to another preferred embodiment of the present invention. According to these figures buccal portion 2 of the dosage form has a centre hole 5 for speeding dissolution. The dental portion 1 of the dosage form has ridges 6 which may run either in the antero-posterior plane (as shown here) or perpendicularly in the medio-lateral plane, or other projection type to form a non-smooth surface.

FIGS. 5 and 6 is a front view of fenestrated or dimpled “T”-shaped solid buccal or sublingual dosage form and a right view of fenestrated or dimpled “T”-shaped solid buccal or sublingual dosage form correspondingly according to yet another preferred embodiment of the present invention. According to these figures buccal portion 2 of the dosage form has fenestrellas or dimples 7 for speeding dissolution. The dental, interocclusal portion 1 of the dosage form has ridges 6.

Solid buccal or sublingual dosage forms according to FIGS. 3 and 5 have curved or semilunar surfaces of buccal portions 2 to approximate the curvature of the cheek.

Anterior and posterior portions of buccal portions 2 on FIGS. 4 and 6 are marked by <<A and <<P correspondingly.

FIG. 7 depicts a front view of a relatively flat, fenestrated or dimpled solid buccal or sublingual dosage form. Said dosage form has only the buccal portion 2. The buccal portion 2 has fenestrellas or dimples 7. The fenestrated form may be alternatively associated with a perpendicular or near perpendicular bite surface member to achieve either an “L” or “T-dosage form”.

The foregoing description of preferred embodiments for this invention have been presented for purposes of illustration and description. They are not intended to be exhaustive or to limit the invention to the precise form disclosed. Obvious modifications or variations are possible in light of the above teachings. The embodiments are chosen and described in an effort to provide the best illustrations of the principles of the invention and its practical application, and to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally, and equitably entitled.

FIGS. 8A-8D depict tables and diagrams illustrating NCCIT apoptotic RNA degradation after 24 hours incubation with various combinations of natural oils.

FIG. 9 depicts a heat map showing gene expression in cannabis extract treated 3T3 cells relative to control.

FIG. 10 depict an illustration of biological activity of dilute (1:1000 and 1:100) clove extract in cultured NCCIT cancer after 48 hours (10× and 40× magnification) relative to control.

FIG. 11 depict an illustration of biological activity of dilute (1:1000 and 1:100) thyme extract in cultured NCCIT cancer after 48 hours (10× and 40× magnification) relative to control.

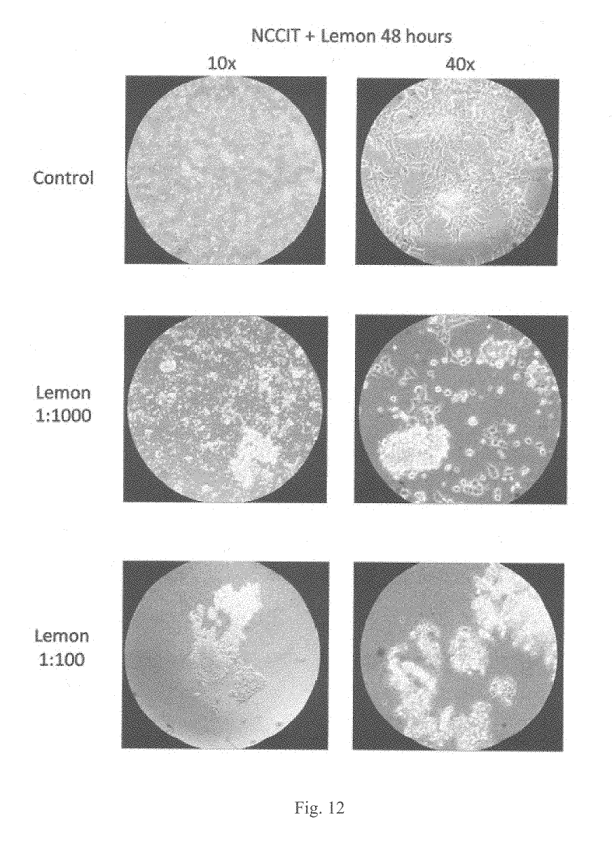

FIG. 12 depict an illustration of biological activity of dilute (1:1000 and 1:100) lemon extract in cultured NCCIT cancer after 48 hours (10× and 40× magnification) relative to control.

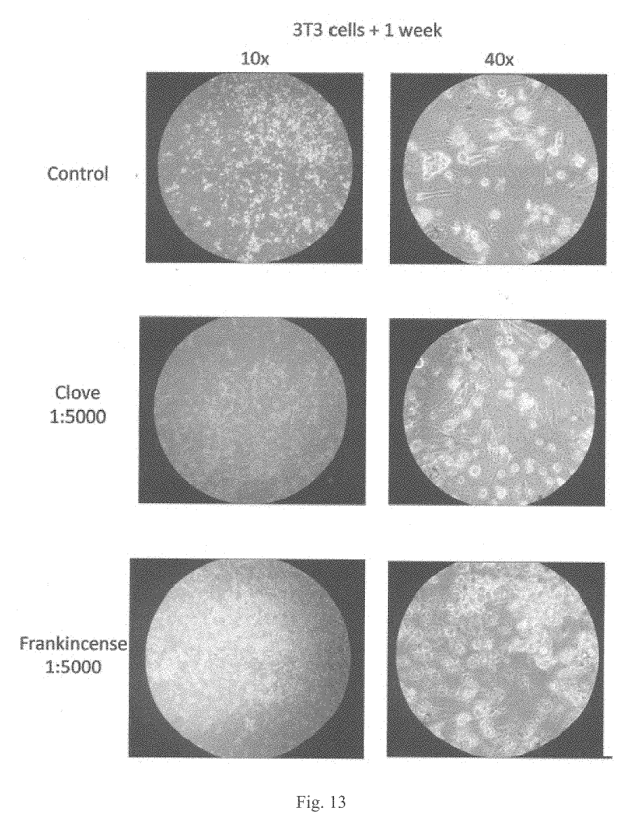

FIG. 13 depict an illustration of biological activity of dilute (1:5000) clove extract and dilute (1:5000) frankincense extract in cultured 3T3 cells after 1 week (10× and 40× magnification) relative to control.

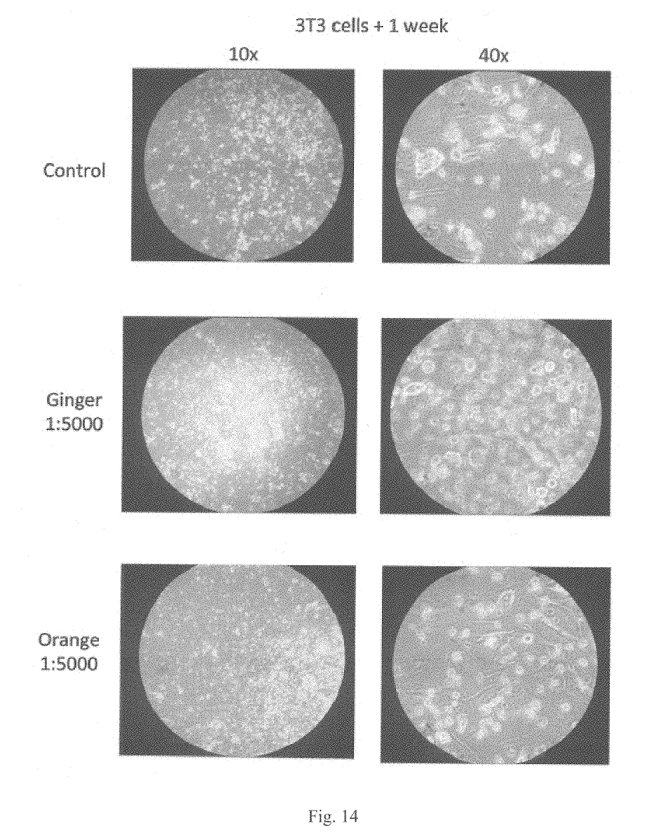

FIG. 14 depict an illustration of biological activity of dilute (1:5000) ginger extract and dilute (1:5000) orange extract in cultured 3T3 cells after 1 week (10× and 40× magnification) relative to control.

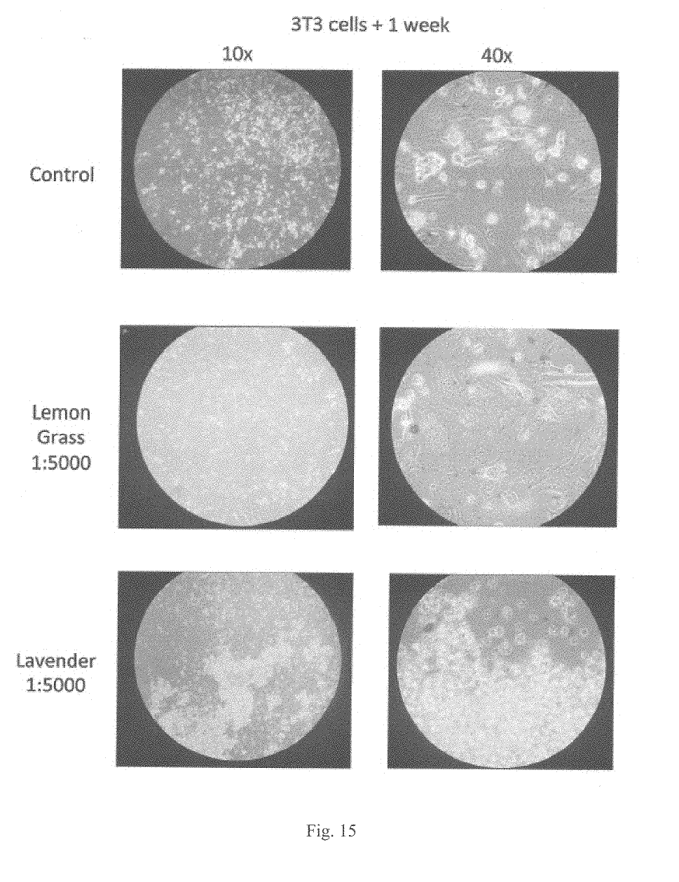

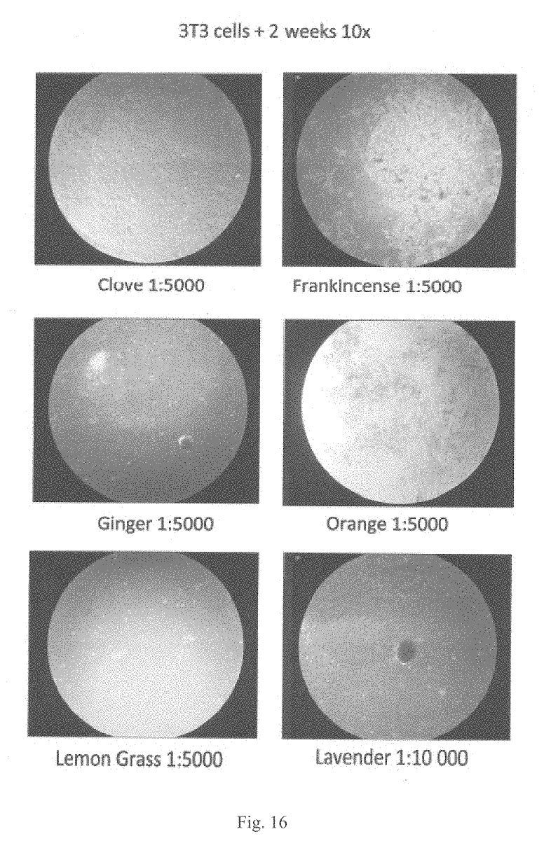

FIG. 15 depict an illustration of biological activity of dilute (1:5000) lemon grass extract and dilute (1:5000) lavender extract in cultured 3T3 cells after 1 week (10× and 40× magnification) relative to control.

FIG. 16 depict an illustration of biological activity of dilute (1:5000) clove extract, dilute (1:5000) frankincense extract, dilute (1:5000) ginger extract, dilute (1:5000) orange extract, dilute (1:5000) lemon grass extract, dilute (1:10000) lavender extract in cultured 3T3 cells after 2 weeks (10× magnification) relative to control.

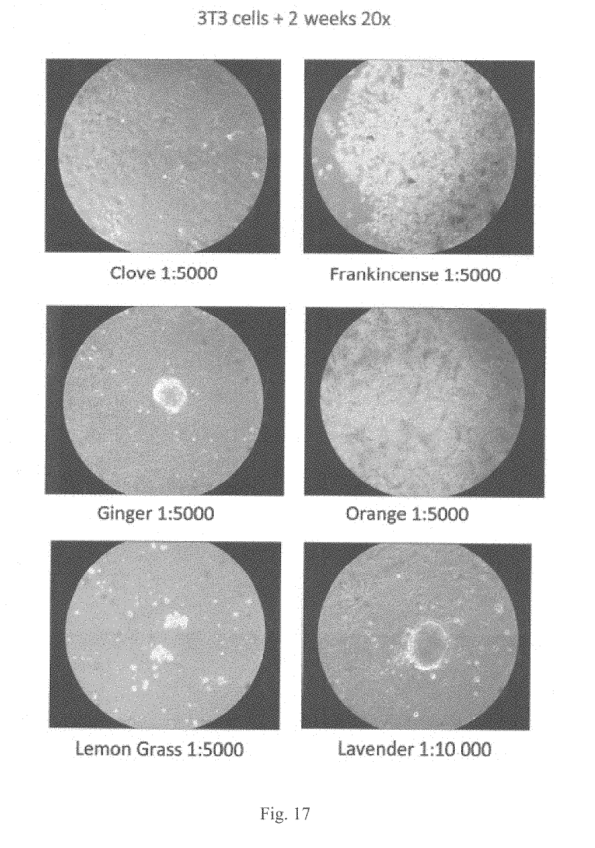

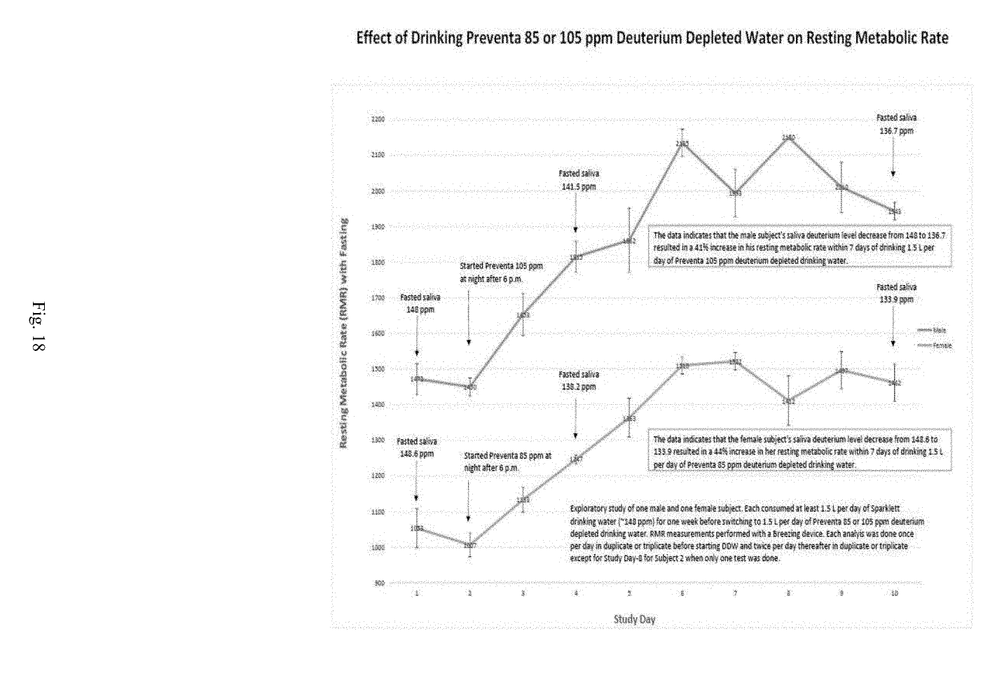

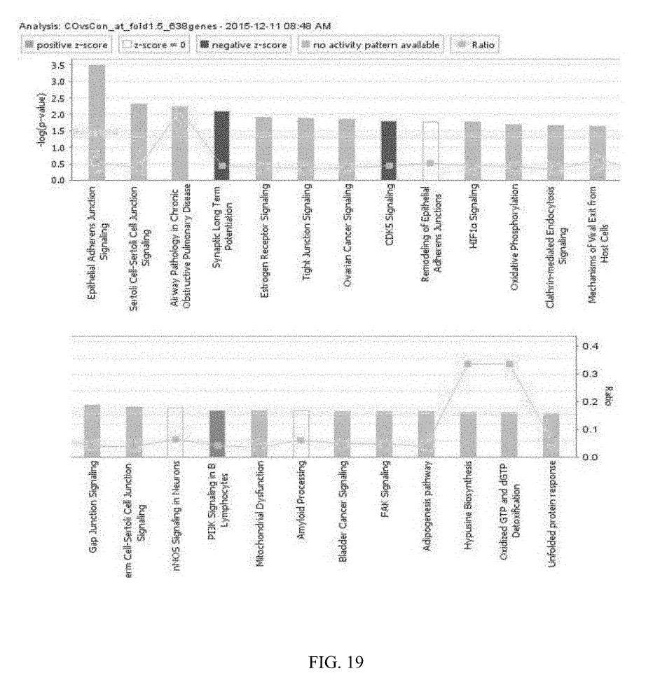

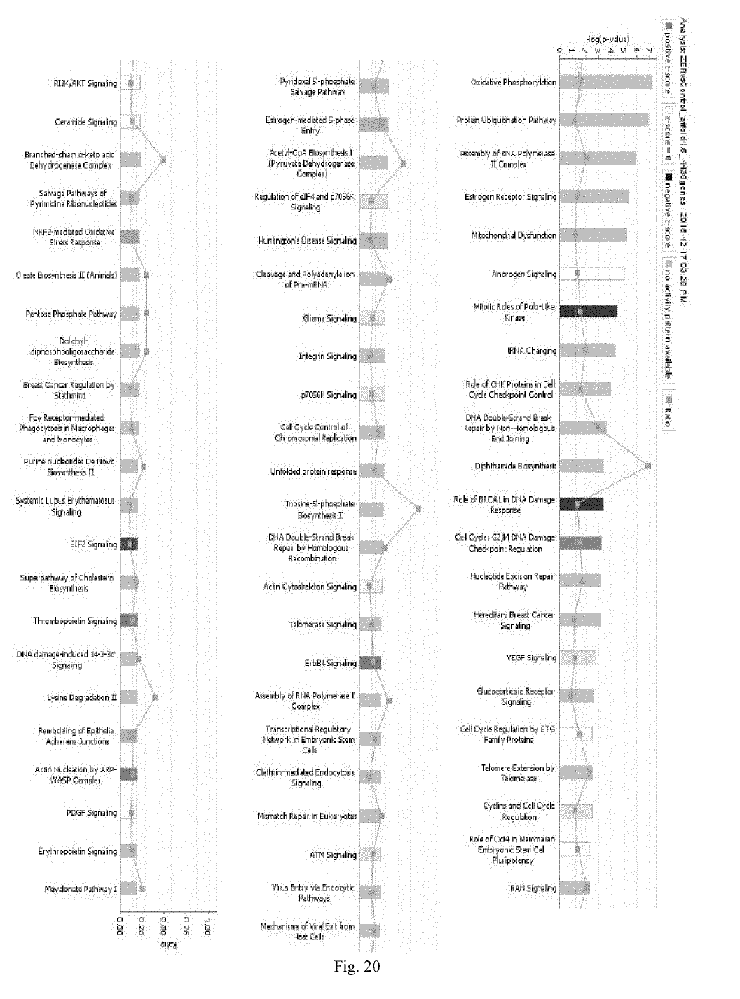

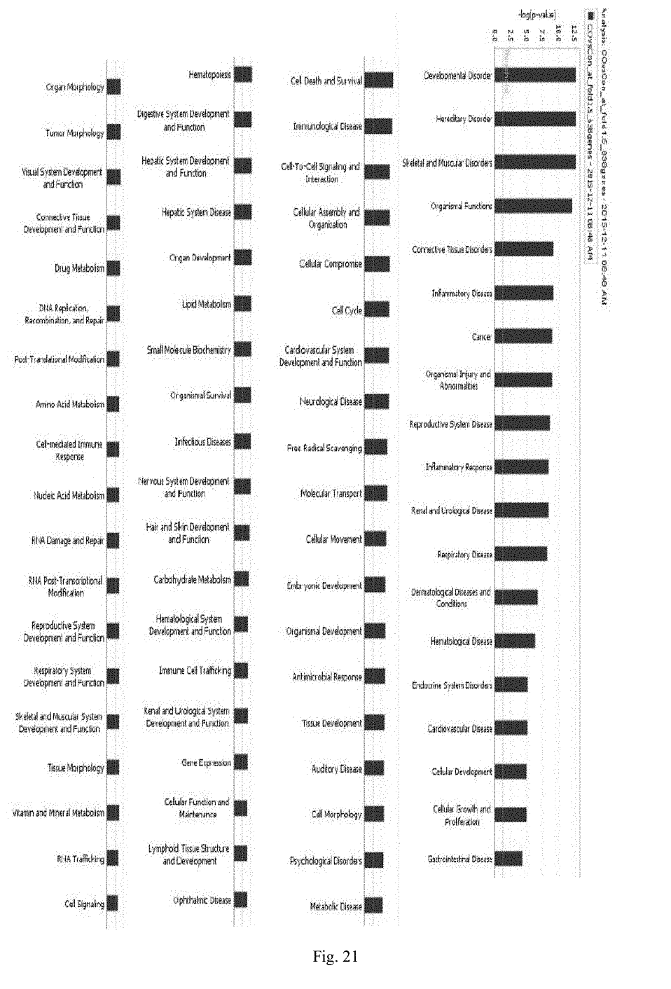

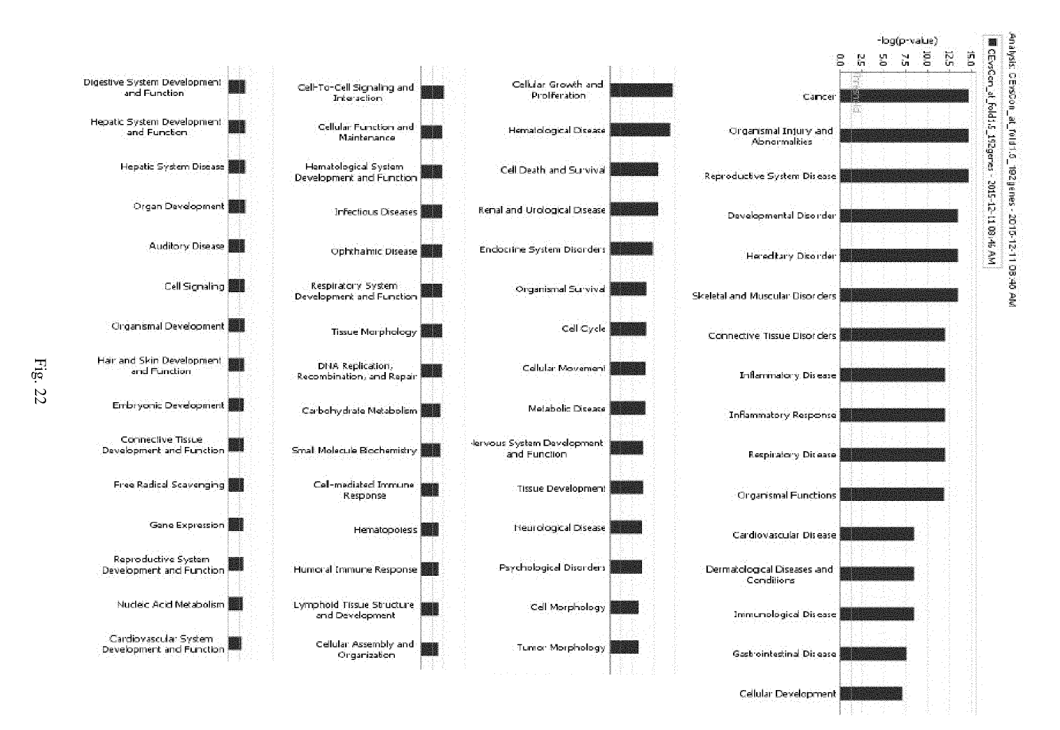

FIG. 17 depict an illustration of biological activity of dilute (1:5000) clove extract, dilute (1:5000) frankincense extract, dilute (1:5000) ginger extract, dilute (1:5000) orange extract, dilute (1:5000) lemon grass extract, dilute (1:10000) lavender extract in cultured 3T3 cells after 2 weeks (20× magnification) relative to control.

As it can be seen on FIGS. 10-17 application of various dilute, individual natural oil extracts to NCCIT cancer and NIH 3T3 cells influences cell number, morphology, and adherence.

### EXAMPLES

The invention is now considered with respect to specific examples, though not limited thereby. As used within the following examples, the term “at least one extract selected from the group comprising orange, frankincense, cannabis or other natural oil or extract or at least one substance or compound isolated from said at least one oil or extract and other natural oils and/or extracts and/or at least one other agent, compound, or drug of the present invention” refers to all conjugates and derivatives of at least one extract selected from the group comprising orange, frankincense, cannabis or other natural oil or extract or at least one substance or compound isolated from said at least one oil or extract and other natural oils and/or extracts and/or at least one other sesquiterpenes, agent, compound, or drug of the present invention, as well as all conjugates and derivatives of all agents, compounds, and drugs of the present invention. Thus, examples directed toward the inclusion of at least one extract selected from the group comprising orange, frankincense, cannabis or other natural oil or extract or at least one substance or compound isolated from said at least one oil or extract and other natural oils and/or extracts in a composition also represent examples directed toward the inclusion of the at least one other agent, compound, and drug described herein, as well as their analogs, isomers and/or derivatives at appropriate dosages. Accordingly, the amounts of the other components of the compositions, manufacture, products, processes, methods, and/or methods of use, prevention and treatment may likewise be appropriately scaled in relation to the mass of actually included agents, compounds or drugs. Thus, it should be understood that the example compositions and formations below may be altered by inclusion of OTC, and/or approved drug(s) in accordance with some embodiments of the invention. As used within the following examples, the term “glutathione” refers to all analogs, conjugates and derivatives of glutathione (e.g, glutathione monoethylester).

### Example 1 Preparation of Solid Lipid Nanoparticles

In one embodiment, the method chosen for the preparation of nanoparticles is an adaptation of the w/o/w double emulsion technique (Garcia-Fuentes et al 2003; Zhang et al 2006; Sarmento et. al., 2007). Approximately 200 mg of acetyl palmitate is dissolved in about 4 mL of dichloromethane. 7 mg of at least one extract selected from the group comprising orange, frankincense, cannabis or other natural oil or extract or at least one substance or compound isolated from said at least one oil or extract and other natural oils and/or extracts and/or an equivalent effective amount of other agent(s), compound(s), or drug(s) of the present invention and glutathione) are dissolved in 0.5 mL of HCL 0.1 M. The drug solution is added to the lipid solution, or at least one extract selected from the group comprising orange, frankincense, cannabis or other natural oil or extract and other natural oils and/or extracts, and then homogenized for 30 seconds in an ultra-turrax T25 (IKA-Labortechnik, Germany) or a similar apparatus. The primary emulsion is then poured into 25 mL of 2% poloxamer 407 solution and homogenized for another 30 seconds. The solvent is subsequently discarded and the emulsion is concentrated in a rotavapor until 10 mL. Optionally, particle size can be analyzed using photon correlation spectroscopy (PCS); and electrophoretic mobility can be measured with Laser Doppler Anemometry (LDA) using a Malvern Zetasizer 5000 (Malvern Instruments, UK) or similar apparatus. Samples cah be diluted with Milli-Q-water having a conductivity adjusted to 50 S/cm by addition of a 0.9% NaCl solution.

The amount of the agent(s), compound(s) or drug(s) incorporated into SLN may be calculated by the difference between the total amount used to prepare the systems and the amount of compound or drug remaining in the aqueous phase after SLN isolation. After preparation, aqueous SLN dispersions may be centrifuged (by ultracentrifuge, rotor type 80Ti, Beckman Instruments, German or analogous instrument or similar apparatus) for about 2 hours at 45000 rpm (corresponding to approx. 190000×g). Compound, agent or drug concentration in the supernatant may be determined by HPLC (Sarmento et al 2006).

### Example 2 Preparation of a Liposomal Composition

A liposomal composition comprising at least one agent, compound, and drug of the present invention may be prepared according to Good Manufacturing Practices by the method of (Paul et al. (1997), previously described by Fessi et al. (1988). Briefly, an organic phase containing phospholipids, or at least one extract selected from the group comprising orange, frankincense, cannabis or other natural oil or extract or at least one substance or compound isolated from said at least one oil or extract and other natural oils and/or extracts, and the drugs is introduced under magnetic stirring in an aqueous phase. The organic solvent is evaporated, and the liposomes obtained are filtered and lyophilized. Prior to administration, 50 mg of lyophilized liposomes are resuspended in sterile distilled water (20 ml), shaken for 3 min, and then diluted in 5% dextrose.

### Example 3 Preparation of a Tablet Composition

Compressed tablets of the invention may be prepared by uniformly mixing at least one active ingredient with a solid carrier to provide a mixture. The mixture is then compacted to the shape and size desired. Molded tablets maybe made in a suitable machine. To prepare a tablet composition containing agents, compounds, or drugs of the present invention, the selected active components (e.g. at least one extract selected from the group comprising orange, frankincense, cannabis or other natural oil or extract or at least one substance or compound isolated from said at least one oil or extract and other natural oils and/or extracts and/or at least one other agent, compound, or drug of the present invention (80 g) and reduced glutathione (400 g)) may be mixed in the dry state for 10 minutes in a Z-blade mixer. Likewise, a solution is prepared containing gelatin (16 g), dioctyl sodium sulphosuccinate (1 g), alcohol (57 g) and purified water (80 g). The solution is then wet-mixed with the powders for 10 minutes using a slow speed. The wet mass is passed through a 1000 m screen. Subsequently, the granules are dried in a fluidized bed at 60° C. for 30 minutes. The dried granules can then be sifted through a 1000 m screen. Likewise, magnesium stearate (4.8 g) is sifted to 125 m, and can be blended with the granules. Finally, the resulting mixture compressed on a Manesty D3 Rotary machine to provide tablets (U.S. Pat. No. 4,209,513).

### Example 4 Preparation of a Stable Liquid Composition

On order to prepare a stable liquid composition comprising the agents, compounds, or drugs of the present invention, the following are combined: 1 Excipient Amount/20 mL % of composition, the active components (e.g. at least one extract selected from the group comprising orange, frankincense, cannabis or other natural oil or extract or at least one substance or compound isolated from said at least one oil or extract and other natural oils and/or extracts, and/or at least one other agent, compound, or drug of the present invention 2.5 mg, reduced glutathione 2.5 mg), 0.25 mg/mL water or pH 8 15.1 mL 75.5% v/v phosphate buffer glycerin 4 mL 20% v/v HPMC-K4 400 μL of 0.1% solution 0.4 mg 0.002% w/v TWEEN® 80 100 μL 0.5% v/v ethanol 200 μL 1% v/v saccharin 400 μL of 0.1% solution 0.4 mg 0.002% w/v (see U.S. Pat. No. 7,259,185).

### Example 5 Preparation of a Syrup Composition

To prepare a syrup composition comprising at least one extract selected from the group comprising orange, frankincense, cannabis or other natural oil or extract or at least one substance or compound isolated from said at least one oil extract and other natural oils and/or extracts, and/or at least one other agent, compound, or drug of the present invention, 35% of the final batch volume of the purified water (USP/EP qs 1 L) is charged and heated to or at 60-80° C. The sugar (Sucrose Extra Fine Granulated USP 300.0 g/L), sodium benzoate (NF/EP 1.0 g/L), sodium citrate (Dihydrate USP/EP 5.27 g/L) and citric acid (Anhydrous USP/EP 2.15 g/L) are added and mixed until they dissolve. The solution is then cooled to 25-30° C. The sorbitol solution (USP/EP 142.0 g/L) and glycerin (Glycerol Anhydrous USP 150.0 g/L) are added, followed by a solution that contains propylene glycol (USP/EP 100.0 g/L) and a flavorant (1.0 g/L) mixed together. Finally, the at least one extract selected from the group comprising orange, frankincense, cannabis or other natural oil or extract or at least one substance or compound isolated from said at least one oil or extract and other natural oils and/or extracts and/or at least one other agent, compound, or drug of the present invention (40 g/L) is added and dissolved. The batch is finally brought to final volume by weight, and subsequently passed through a 1.2 micron filter. The concentration of at least one extract selected from the group comprising orange, frankincense, cannabis or other natural oil or extract or at least one substance or compound isolated from said at least one oil or extract and other natural oils and/or extracts and/or at least one other agent, compound, or drug of the present invention in this syrup composition may be reduced to accommodate the addition of other agents, compounds, or drugs named herein to produce desirable compositions.

### Example 6 Preparation of a Soft Gel Composition

To prepare a soft gel composition comprising at least one agent, compound, and drug of the present invention (e.g. at least one agent, compound, or drug of the present invention): polyoxyethanyl-α-tocopheryl-sebacate (PTS) (150 mg) and/or at least one other agent, compound, or drug of the present invention (100 mg) are melted and mixed them together at 60° C. To the cooled compositions are added oil (either rice bran oil, at least one extract selected from the group comprising orange, frankincense, cannabis or other natural oil or extract or at least one substance or compound isolated from said at least one oil or extract and other natural oils and/or extracts), or omega-fatty acid enriched fish oil (ONC Oil 18/12) (30 mg) and beewax (50 mg). The composition is then incubated at 60° C. until the beeswax melts. The composition is finally mixed again and sealed under argon gas. The concentration of at least one extract selected from the group comprising orange, frankincense, cannabis or other natural oil or extract or at least one substance or compound isolated from said at least one oil or extract and other natural oils and/or extracts and/or at least one other agent, compound, or drug of the present invention or other agents, compounds, and drugs of the present invention in this soft gel composition may be reduced to accommodate the addition of other agents, compounds, or drugs named herein to produce desirable compositions.

### Example 7 Preparation of a Chewing Gum Composition

To prepare a chewing gum composition comprising the agents, compounds, and drugs of the present invention, the active medicaments are preferably added early-on into the mix. The smaller the amount of active ingredient used, the more important it is to preblend that particular ingredient to assume uniform distribution. Whether a pre-blend is used or not, in one embodiment, the agent or medicament should be added within the first five minutes of mixing. If the selected agents, compounds, and drugs are water soluble in the chewing gum, it preferably will include a base/emulsifier system which leads to the desired concentration of the medicament in the saliva (more hydrophilic balance). If the selected agents, compounds, and drugs are water insoluble, the chewing gum preferably includes a base/emulsifier system which leads to the desired concentration of the medicament in the saliva (more lipophilic balance). In manufacturing the gum ingredients may include the following Sugar (54.77%), Gum Base (21.80%), Corn Syrup (11.20%), Fructose (5.60%) Glycerine (3.40%) Active drug(s) (1.70%), Flavors (1.00%), Artificial Sweetener (0.26%), Soluble Saccharin (0.21%) and Insoluble Saccharin (0.06%). The precise percentages and many of the ingredients may vary (U.S. Pat. No. 7,078,052).

### Example 8 Preparation of an Overcoated Chewing Gum Composition

To prepare an overcoated chewing gum composition comprising the agents, compounds, and drugs of the present invention, the Gum Center is made as follows: Gum Base 33%, Calcium Carbonate 13%, Sorbitol 44.23%, Glycerin 4%, Flavors 2.32%, and/or at least one other agent, compound, or drug of the present invention 2%, Lecithin 0.6%, Sweeteners 0.9%. The center is sprayed with dried maltodextrin/and/or at least one other agent, compound, or drug of the present invention at 50% at least one active agent, compound, or drug of the present invention. The Gum Coating is composed of Coating Syrup 3, Coating Syrup 4, Xylitol 64.14%, Water 11.14%, 40% Gum Tahla Solution 20.87%, Titanium Dioxide Whitener 0.40% Peppermint Flavor 3 1.40%, Sweeteners 0.27%, Talc Polishing Agents 1.78%. The Flavor is added in 3 additions after 3 separate syrup additions within the coating syrup (1.4%). Finally, after completion of coating, the overcoated gum is polished. Following this protocol, the initial center piece achieves a weight of about 0.995 grams. The Gum is then coated to a finished piece weight of 1.52 grams to give a 34.5% coating. Coating syrup 3 is used to coat the first 60% of the coating to a piece weight of 1.30 grams. Coating syrup 4 is used to coat to the final piece weight (U.S. Pat. No. 6,290,985). The amount of at least one extract selected from the group comprising orange, frankincense, cannabis or other natural oil or extract or at least one substance or compound isolated from said at least one oil or extract and other natural oils and/or extracts and/or at least one other agent, compound, or drug of the present invention and the other ingredients in this gum may be adjusted to accommodate the addition of other agents, compounds, or drugs named herein to produce desirable compositions.

### Example 9 Preparation of a Troche Comprising at Least One Extract Selected from the Group Comprising Orange, Frankincense, Cannabis or Other Natural Oil or Extract or at Least One Substance or Compound Isolated from Said at Least One Oil or Extract and Other Natural Oils and/or Extracts and/or at Least One Other Agent, Compound or Drug of the Present Invention

Long-lasting troches gradually release an active ingredient thereby prolonging absorption and duration of drug action. Troches also allow for sublingual absorption of agents that may have poor intestinal bioavailability (U.S. Pat. No. 3,312,594). To prepare a troche comprising at least one extract selected from the group comprising orange, frankincense, cannabis or other natural oil or extract or at least one substance or compound isolated from said at least one oil or extract and other natural oils and/or extracts and/or at least one other agent, compound, or drug of the present invention, and/or at least one the other agent, compound, or drug of the present invention, equal amounts of carboxymethylcellulose, pectin, and gelatin (e.g. 330 g each), are thoroughly admixed with magnesium stearate (e.g. 10 g) and with the active compounds, agents, or drugs (in appropriate concentrations). Afterwards, the mixed powder is compressed in a Stokes machine (or similar apparatus) to form troches of 500 mg each. The amount of at least one extract selected from the group comprising orange, frankincense, cannabis or other natural oil or extract or at least one substance or compound isolated from said at least one oil or extract and other natural oils and/or extracts and/or at least one other agent, compound, or drug of the present invention and the other ingredients may be adjusted to accommodate the addition of other agents, compounds, or drugs named herein to produce desirable compositions.

### Example 10 Preparation of a Sports Drink

To prepare a sports drink, desired and workable amounts of each at least one extract selected from the group comprising orange, frankincense, cannabis or other natural oil or extract or at least one substance or compound isolated from said at least one oil or extract and other natural oils and/or extracts, compound, agent, or drug of the present invention may be added to sugar(s) selected from Galactose, Fructose, and Glucose (e.g. 2.5 g/100 ml), Sodium Chloride (e.g. 0.2 g/100 ml), Potassium (0.04 g/100 ml), Dihydrogen orthophosphate Magnesium (e.g. 0.01 g/100 ml). Citric acid or citrate may be used in an amount of 0.1 to 0.5% w/v as needed. When sodium citrate is used, the quantity of sodium chloride may be reduced in exact molar proportion to the sodium ions added as sodium citrate (up to 34 mmol·l-1). Furthermore, caffeine and flavorings may be incorporated as desired. Preservatives, for example sodium benzoate or sorbic acid may likewise be employed. Vitamin C may be used as an antioxidant in an amount to 0.5% w/v as needed. The proportions set out above may be varied, but typically by 25% or less.

Alternatively, a composition for providing the health benefits listed herein while also providing a rapid source of energy, electrolyte balance, blood volume, and performance enhancement, may be produced by combining desired and workable amounts of each compound, agent, or drug of the present invention (e.g at least one extract selected from the group comprising orange, frankincense, cannabis or other natural oil or extract or at least one substance or compound isolated from said at least one oil or extract and other natural oils and/or extracts and/or at least one other agent, compound, or drug of the present invention 0.5 to 30% (preferably 5%), glutathione 0.5-10% (preferably 3%)), with electrolytes selected from e.g. sodium, potassium, chloride, phosphate, bicarbonate, sulfate, magnesium and calcium (e.g. about 1 meq/1 to 6 meq/1 potassium, 12 meq/1 to 33 meq/1 sodium, about 2 meq/1 to about 8 meq/1 phosphate), 0.5% to 5% glycerol (e.g. 1%), and about 2% to 8% sugar compound (e.g. 5% fructose, sucrose, glucose or other sugar). Specifically, the composition may have a glucose concentration of from about 2% to about 8%.

Preferably, the sugar concentration may be about 4%. The drink may be carbonated. In addition, caffeine may also be added (e.g. about 120-180 mg/I), as may other compounds such as vitamins, minerals, citric acid, citrate, preservatives, flavorings, sweeteners, and others. The proportions, set out above may be varied, but typically by 25% or less.

### Example 11 Preparation of a Stable Aqueous Composition Comprising Peptide Compounds in Water

To prepare a stable aqueous composition suitable for the provision of at least one extract selected from the group comprising orange, frankincense, cannabis or other natural oil or extract or at least one substance or compound isolated from said at least one oil or extract and other natural oils and/or extracts, and/or at least one other agent, compound or drug of the present invention, in combination with peptides including oligopeptides (e.g. reduced glutathione), the at least one extract selected from the group comprising orange, frankincense, cannabis or other natural oil or extract or at least one substance or compound isolated from said at least one oil or extract and other natural oils and/or extracts and reduced glutathione (and other agents, compounds or drugs of the composition) are weighed (to achieve desired concentrations—e.g. total=40%) and then added to a weighed amount of vehicle (sterile distilled water, ethanol/water or water with non-ionic surfactant) at the appropriate concentration (w/w), then gently stirred to dissolve.

### Example 12 Preparation of a Powder Pharmaceutical Preparation Dissolvable in a Liquid to Form a Solution Prior to Ingestion

The powder pharmaceutical composition comprises safe and effective amount of the active agents. To prepare a Powder Pharmaceutical composition suitable for the provision of at least one extract selected from the group comprising orange, frankincense, cannabis or other natural oil or extract or at least one substance or compound isolated from said at least one oil or extract and other natural oils and/or extracts and/or at least one other agent, compound, or drug of the present invention, one may mix Ascorbic Acid (1.20%), Citric Acid (10.50%), Honey Buds Flavor (3%), Honey Powder Flavor (4%), Natural Lemon Flavor (5%), Natural Lime Flavor (6%), Sweet-Ung (7%), Sodium Saccharin (0.30%), and Sugar Extra Fine Granulated (69.4985%).

### Example 13 Preparation of Encapsulated Nanoparticles

To prepare encapsulated nanoparticles of at least one extract selected from the group comprising orange, frankincense, cannabis or other natural oil or extract or at least one substance or compound isolated from said at least one oil or extract and other natural oils and/or extracts and/or at least one other agent, compound, or drug of the present invention, one may employ a single emulsion technique (Shaikh et al., 2009; 20090312402), a double emulsion technique, or a multi-emulsion technique.

### Example 14 Complexation of Polyphenols (e.g. Flavones and Flavonoids) of the Present Invention to Provide for Increased Absorption

Phosphatidylcholine (PC) may be used to increase the bioavailability of polyphenol compounds. Upon oral ingestion, the amphipathic PC molecules facilitate movement of the polyphenol through the intestinal epithelium to the bloodstream (Kidd, 2009).

### Example 15 the Preparation of a Spray or Drops

To prepare a spray (nasal or oral)/drops (e.g. ocular drops) composition comprising at least one extract selected from the group comprising orange, frankincense, cannabis or other natural oil or extract or at least one substance or compound isolated from said at least one oil or extract and other natural oils and/or extracts and/or at least one other agent, compound, or drug of the present invention, a borate buffer may be prepared by dissolving 3.81 g of sodium tetraborate in 100 ml mixture of water and or at least one extract selected from the group comprising orange, frankincense, cannabis or other natural oil or extract or at least one substance or compound isolated from said at least one oil or extract and other natural oils and/or extracts; dissolving 6.8 g of boric acid in 100 ml of water; and adjusting the pH of the sodium tetraborate solution to a pH of 7.1-7.3 by the addition of boric acid to provide a buffer. Subsequently, 60 mg agent(s), compound(s), or drug(s) of the present invention, 1 g of Tween80 and 1 g PEG may be combined and stirred well using a glass rod prior to sonication for 30 min or until the at least one extract selected from the group comprising orange, frankincense, cannabis or other natural oil or extract or at least one substance or compound isolated from said at least one oil or extract and other natural oils and/or extracts and/or at least one other agent, compound, or drug of the present invention is completely solubulized.

To prepare an ophthalmic composition, HPMC is added to 100 ml water and stirred until the HPMC is fully dissolved. Subsequently, the at least one extract selected from the group comprising orange, frankincense, cannabis or other natural oil or extract or at least one substance or compound isolated from said at least one oil or extract and other natural oils and/or extracts and/or at least one other agent, compound, or drug of the present invention/tween80 solution is added drop by drop and stirred for 15 minutes. NaCl, BAC, and EDTA are added and stirred until all the contents dissolve completely before adjusting the pH to 6.5 with borate buffer.

### Example 16 Preparation of a Nanoemulsified Topical Composition

To prepare a nanoemulsified topical composition comprising at least one extract selected from the group comprising orange, frankincense, cannabis or other natural oil or extract or at least one substance or compound isolated from said at least one oil or extract and other natural oils and/or extracts and/or at least one other agent, compound, or drug of the present invention, 5.28 g of glyceryl monosterate, 2.64 g of polyethylene glycol (PEG400), (+/−1 ml DMSO) and 2.64 g cetyl alcohol are transferred to a clean 50 ml beaker, followed by adding 2 ml light liquid paraffin and 100 mg Isopropyl myristate into the emulsifiers; then adding 1 ml Phenyl-2-Ethanol to the above mixture; followed by soaking 13.2 g of collagen in 10 ml of demineralised water till the solution becomes clear (25 min); followed by adding 100 mg niacinamide. Afterwards, 250 mg of at least one agent, compound, or drug of the present invention may be transferred into a clean container and solubilized into a nanoemulsion by mixing and sonicating with Tween 80 and PEG400. At about the same time, the solid emulsifiers, glyceryl monosterate, polyethylene glycol (PEG 400) and Cetyl alcohol are melted at 70 C, and the demineralized water (65 ml) simultaneously heated to 70 C. Then about half of the solubilized and/or other agent(s), compound(s), or drug(s) of the present invention is added into the hot emulsifiers to be mixed thoroughly. At this point, the melted emulsifiers may be added into the boiled demineralised water and mixed vigorously at the room temperature; until a creamy consistency is achieved. To this cream, at least one extract selected from the group comprising orange, frankincense, cannabis or other natural oil or extract or at least one substance or compound isolated from said at least one oil or extract and other natural oils and/or extracts collagen and niacin may be added to form a smooth cream before adding another half the amount of solubilized at least one agent, compound, or drug of the present invention and mixing. Then 100 μl Bronidox may be dissolved in 1 ml of PEG 400 is then added to the mixture as can be 100 μl of lavender oil to the above cream for fragrance.

### Example 17 Preparation of a Soluble, Liquid Composition Comprising the Agents, Compounds, or Drugs of the Present Invention

Agent or compound are provided as powder with fine granulometry (having the preferred and advantageous granulometry comprised between about 100 and about 200 m) may be mixed with citric acid crystals (e.g. granulometry below 150 m) and the resulting mixture stirred into at least one extract selected from the group comprising orange, frankincense, cannabis or other natural oil or extract or at least one substance or compound isolated from said at least one oil or extract and other natural oils and/or extracts and Polysorbate 80. After heating to 300 C for adequate homogenization, this completed mixture may be mixed for 45 min. then milled with a three-roll-mill (e.g. a Coball mill) and closing aerated with nitrogen to remove present air. The preparation may then be encapsulated in gelatin capsules, preferably about 700 mg per capsule. Both non-coated capsules and enteric coated capsules with addition of E 904 (SHELLAC) may be used. Preferably, the concentration of pure at least one agent, compound, or drug of the present invention is preferably 6%, with 0.5% citric acid completed to 100% with at least one extract selected from the group comprising orange, frankincense, cannabis or other natural oil or extract or at least one substance or compound isolated from said at least one oil or extract and other natural oils and/or extracts and Polysorbate 80.

### Example 18 Preparation of a Hard Shell Capsule or Tablet Composition

The preparation is made as in example 17, though using a high viscosity emulsifier such as Polysorbate 60. SiO₂ may be added until a homogenous and fluid powder is achieved and to produce a percentage of 5% to 50% (preferably 30% to 35%). The resultant powder may then be used to fill hard shell capsules (preferably about 500 mg per capsule) or compressed into a tablet. Preferably, the concentration of at least one agent, compound, or drug of the present invention, in the final composition is 4%, with 0.35% citric acid and preferably a final concentration of SiO₂, of 30%. All percentages are on a weight by weight (w:w) basis.

### Example 19 Preparation of Agents, Compounds, and Drugs of the Present Invention Bound to Chitosan Nanoparticles

To prepare Chitosan Nanoparticles, a solution of 0.2% Chitosan (w/v) in 1% acetic acid may be prepared by heating the mixture to 75° C. The mixture may then be rapidly cooled to 4° C. and this process repeated several times until a solution of chitosan is obtained. This solution is then heated to 75 C again and sprayed under pressure into water kept stirring very rapidly at 4 C to produce uniformly dispersed chitosan nanoparticles. Such nanoparticles may be concentrated by centrifugation. Subsequently, 1 g at least one extract selected from the group comprising orange, frankincense, cannabis or other natural oil or extract or at least one substance or compound isolated from said at least one oil or extract and other natural oils and/or extracts, and/or at least one other agent, compound, or drug of the present invention in 1000 ml of absolute ethanol is added under pressure to vigorously stirred aqueous suspension of chitosan nanoparticles in 1% acetic acid and the resulting suspension may then be stirred overnight at 200-1400 rpm at room temperature to load and at least one agent, compound, or drug of the present invention on the chitosan nanoparticles.

### Example 20 Preparation of Nanoparticles

1 g of at least one agent, compound, or drug of the present invention, and/or other at least one agent, compound or drug of the present invention, may be dissolved in 1000 ml of at least one extract selected from the group comprising orange, frankincense, cannabis or other natural oil or extract and other natural oils and/or extracts and absolute ethanol. The solution may then be kept at 40° C. and then sprayed under nitrogen atmosphere and high pressure into 0.1% aqueous acetic acid solution. The solution is to be kept stirring at 200-1400 rpm at room temperature. The particle size can be controlled by varying the pressure at which the solution is sprayed into 0.1% aqueous acetic acid kept at different temperatures (25° C.-40° C.)

### Example 21 Preparation of at Least One Extract Component Conjugated to Arginine or Lysine Selected from the Group Comprising Orange, Frankincense, Cannabis Extract or at Least One Substance or Compound Isolated from Said at Least One Extract and Other Natural Oil and/or Extract or Extract Derivatives

At least one extract selected from the group comprising orange, frankincense, cannabis or other natural oil or extract or at least one substance or compound isolated from said at least one oil or extract and other natural oils and/or extracts and/or at least one other agent, compound, or drug of the present invention is combined under heat with methanol, while a lysine or arginine base is dissolved in water. Subsequently, the lysine/arginine solution is stirred into the at least one extract selected from the group comprising orange, frankincense, cannabis or other natural oil or extract or at least one substance or compound isolated from said at least one oil or extract and other natural oils and/or extracts and/or at least one other agent, compound, or drug of the present invention solution.

### Example 22 Process for Reducing the Crystalline Nature of at Least One Agent, Compound or Drug of the Present Invention to Increase Solubility and Enhance Activity

To prepare at least one agent, compound, or drug of the present invention, with diminished crystalline state, a process may be undertaken comprising: 1. preparing a mixed solution containing at least one agent, compound, or drug of the present invention and water-soluble or insoluble polymer in organic solvent or purified water; and 2. solid-dispersing at least one agent, compound, or drug of the present invention in the mixed solution in a polymer solution by using a spray dryer or fluidized bed granulator. In this context, the water-soluble polymer may be alginic acid, alginate or its derivatives, α-cyclodextrin or its derivatives, β-cyclodextrin or its derivatives, polyvinylpyrrolidone or its derivatives: polyvinylpyrrolidone-vinylacetate copolymer, γ-cyclodextrin or its derivatives, polyoxyethylene-polyoxypropylene copolymer, polyethyleneglycol or its derivatives, polyvinylalcohol, xanthan gum, or arabic gum, or a combination of polymers.

### Example 23 Preparation of Cyclodextrine-Containing Derivatives for Increased Solubility

In order to prepare a more aqueous soluble at least one extract selected from the group comprising orange, frankincense, cannabis or other natural oil or extract or at least one substance or compound isolated from said at least one oil or extract and other natural oils and/or extracts, at least one agent, compound, or drug of the present invention suitable for administration, at least one extract selected from the group comprising orange, frankincense, cannabis or other natural oil or extract or at least one substance or compound isolated from said at least one oil or extract and other natural oils and/or extracts, at least one agent, compound or drug are dissolved under heat in methanol, while lysine or arginine base is dissolved in water (see example 21 above).

Subsequently, the lysine/arginine solution is stirred into at least one extract selected from the group comprising orange, frankincense, cannabis or other natural oil or extract or at least one substance or compound isolated from said at least one oil or extract and other natural oils and/or extracts and/or at least one other agent, compound, or drug of the present invention solution. The combined solution is then subjected to shaking and evaporation under vacuum, dissolving the non-dissolved residue in ethanol and bringing the mixture to the boiling point. Subsequently, non-dissolved residue is filtered out and the ethanol-based solution is maintained at about −200 C for approximately one hour. Once at least one extract selected from the group comprising orange, frankincense, cannabis or other natural oil or extract or at least one substance or compound isolated from said at least one oil or extract and other natural oils and/or extracts and/or at least one other agent, compound, or drug of the present invention lysinate and/or argininate is cooled and collected, it can be added to an aqueous cyclodextrin solution such as HP-beta-CD or HP-gamma-CD at once while agitating well. This new solution is then filtered.

### Example 24 at Least One Agent, Compound, or Drug of the Present Invention Dissolved in Dmso

To increase it's solubility, at least one extract selected from the group comprising orange, frankincense, cannabis or other natural oil or extract or at least one substance or compound isolated from said at least one oil or extract and other natural oils and/or extracts and/or at least one other agent, compound, or drug of the present invention may be dissolved in 3% DMSO in sterile phosphate buffered saline (PBS). Subsequently, a 667 μM solution of at least one extract selected from the group comprising orange, frankincense, cannabis or other natural oil or extract or at least one substance or compound isolated from said at least one oil or extract and other natural oils and/or extracts and/or at least one other agent, compound, or drug of the present invention can be prepared for injection into an animal.

### Example 25 a Carbopol Dispersion Comprising the Agents, Compounds and Drugs of the Present Invention

In order to prepare a gel comprising at least one agent, compound, or drug of the present invention, one may first dissolve disodium edetate (0.05% by weight) in about 90% of the needed water (100% by weight). The at least one agent, compound, or drug of the present invention (1-5% by weight) may then be dissolved in solution by mixing until the at least one drug are dissolved to form a drug solution. After dissolving methylparaben (0.17%) and propylparaben (0.03% by weight) in propylene glycol (10% by weight) using heat as needed up to about 80 C and propeller mixing, one may add this solution slowly while mixing to the drug solution. Then 85% sodium docusate (1% by weight) may be dissolved in the drug solution with propeller mixing. Afterwards, Carbopol (0.6% by weight) is mixed into the drug solution to form a uniform dispersion. After dissolving oxybenzone (1% by weight) in octyl methoxycinnamate (7.5% by weight), one may slowly pour this sunscreen solution into the Carbopol dispersion while mixing with a propeller mixer until uniform. Then one may make a 1% sodium hydroxide solution, with continuous mixing add it slowly and stepwise to the Carbopol® dispersion until the desired pH is attained. Add the remaining water and mix into the gel uniformly.

### Example 26 a Water-in-Oil Emulsion Suitable for Topical Administration

In preparing a water-in-oil emulsion (wherein preferably the base composition is included in the water phase and the water phase has a pH of about 5.8 to about 8, and an osmolarity between about 175 to about 330), the composition may include about 2.5 wt. % to about 3 wt. % base composition (e.g. electrolyte, buffer, mild preservative, lubricant) and about 20 wt. % to about 35 wt. % at least one agent, compound, or drug of the present invention. If the emulsion is intended for use in a sunscreen, then at least one sunscreen agent may be selected (e.g. from the group consisting of: octyl methoxycinnamate, octyl salicylate, homosalate, titanium dioxide, or a combination of such sunscreen agents).

### Example 27 a Water-Proof Sunscreen

In preparing another sunscreen composition comprising agents, compounds, or drugs of the present invention, the sunscreen composition may include (in the OIL phase) a solvent (10% w/w), a film former (8% w/w), a fatty acid (5% w/w), an emulsifier (2% w/w), a waterproofer (3% w/w), a UV filter (10% w/w), agents, compounds, or drugs of the present invention (33% w/w), Wax (4% w/w), and a preservative (0.7%); may include in the (WATER Phase) water (5% water w/w), humectant (10% w/w), thickener (3% w/w), Neutraliser (0.7% w/w), emulsifier (3% w/w), sequestering agent (0.5%), preservatives (1% w/w), and fragrance (1% w/w).

### Example 28 Preparation of a Glutathione-Conjugated or n-Acetylcysteine-Conjugated at Least One Agent, Compound or Drug of the Present Invention

In order to enhance activity, at least one agent, compound, and drug of the present invention may be modified by conjugation with glutathione and or n-acetylcysteine by any means known to the art. At least one extract selected from the group comprising orange, frankincense, cannabis or other natural oil or extract or at least one substance or compound isolated from said at least one oil or extract and other natural oils and/or extracts, zingerone, glutathione and several other agents, compounds, or drugs of the present invention contain a carbonyl group suitable for reaction with nucleophilic glutathione (GSH) or n-acetylcysteine. However, non-carbonyl agents, compounds, or drugs of the present invention are likewise capable of conjugation, coupling, linkage, or complexing with glutathione. The reaction mixture may, for example, comprise between 5 and 25 uM carbonyl-containing substrate in 10 mM potassium phosphate, pH 7.0, and 1 mM GSH. The addition of an effective amount of GSTP1-1 will accelerate the initial rate of GSH-mediated consumption of carbonyl-containing substrate. The mixture is stirred (up to 3 days) at room temperature until a clear solution is obtained.

### Example 29 Preparation of a Glutathione-Conjugated at Least One Agent, Compound or Drug of the Present Invention

At least one agent, compound, or drug of the present invention (4 mmol) and Glutathione (20 mmol, 6.15 g) may be dissolved in H2O (20 ml) and CH2Cl2 (2 ml) by stirring at room temperature until a clear solution is obtained. The clear, colorless solution may then be concentrated to about 10 ml, followed by a slow addition of small amount of MeOH. The mixture is then to be kept in the refrigerator overnight as a white solid precipitates out. The at least one agent, compound, or drug of the present invention-GSH complex may then be filtered and dried. Thereafter, the newly GSH conjugate may be incorporated into tablets, troches, gels, capsules, etc. as described herein.

### Example 30 Addition of at Least One Extract Selected from the Group Comprising Orange, Frankincense, Cannabis or Other Natural Oil or Extract or at Least One Substance or Compound Isolated from Said at Least One Oil or Extract and Other Natural Oils and/or Extracts to a Composition of the Present Invention

In one embodiment, the bioavailability of at least one extract selected from the group comprising orange, frankincense, cannabis or other natural oil or extract or at least one substance or compound isolated from said at least one oil or extract and other natural oils and/or extracts and/or at least one other agent, compound, or drug of the present invention is enhanced by addition of natural oil. Thereafter, the selected agent, compound or drug and the oil may be incorporated into tablets, troches, gels, capsules, etc., as described herein.

### Example 31 Addition of an Agent, Compound or Drug Containing an No Donor Moiety to the Compositions, Manufacture, Products, Processes, Methods, and/or Methods of Use, Prevention and Treatment of the Present Invention

In some embodiments, the selected agent, compound or drug (e.g. glutathione or at least one extract selected from the group comprising orange, frankincense, cannabis or other natural oil or extract or at least one substance or compound isolated from said at least one oil or extract and other natural oils and/or extracts and/or at least one other agent, compound, or drug of the present invention) is first treated in accordance with the methods of WO 92/01668, WO 95/30641, WO 97/16405, U.S. Pat. No. 5,859,053, WO/2002/011706, WO2010118968, Del Soldato et al., (1999), or Bratasz et al., (2006) to obtain a NO-donor derivative. Thereafter, the newly derived NO donor derivative is incorporated into tablets, troches, gels, capsules, etc., as described herein.

### Example 32 a Tablet for Administering at Least One Agent, Compound, or Drug of the Present Invention

At least one agent, compound, or drug of the present invention (25 mg), Glutathione (200 mg), Lactose (50 mg), Starch (10 mg) and Magnesium stearate (in appropriate amounts) may be mixed by propeller mixing and a tablet prepared according to methods known to the art for tablet preparation.

Alternatively, at least one agent, compound, or drug of the present invention (250 mg), Glutathione (250 mg), Lactose (50 mg), Starch (10 mg) and Magnesium stearate (in appropriate amounts) may be mixed by propeller mixing and a tablet prepared according to methods known to the art for tablet preparation.

### Example 33 a Capsule for Administering at Least One Agent, Compound, or Drug of the Present Invention

At least one agent, compound, or drug of the present invention conjugate (250 mg), Lactose (30 mg), Starch (28 mg), Talc (2 mg) and Magnesium stearate (in appropriate amounts) may be mixed by propeller mixing and a gelatin hard capsule prepared according to methods known to the art for gelatin hard capsule preparation.

Alternatively, at least one agent, compound, or drug of the present invention 125 mg, Glutathione (125 mg), Lactose (30 mg), Starch (28 mg), Talc (2 mg) and Magnesium stearate (in appropriate amounts) may be mixed by propeller mixing and a gelatin hard capsule prepared according to methods known to the art for gelatin hard capsule preparation.

### Example 34 a Suspension for Administering at Least One Agent, Compound, or Drug of the Present Invention

At least one agent, compound, or drug of the present invention (250 mg), Isomerized sugar (10 g), Sugar (30 mg), Sodium CMC (100 mg), Lemon Flavor (in appropriate amounts), and distilled water and at least one extract selected from the group comprising orange, frankincense, cannabis or other natural oil or extract or at least one substance or compound isolated from said at least one oil or extract and other natural oils and/or extracts, (sufficient to produce a total volume of 100 ml) may be combined to prepare a suspension in accordance with methods known to the art for the preparation of suspensions. A 100 ml darkly colored bottle may then be filled with the suspension and sterilized.

Alternatively, at least one extract selected from the group comprising orange, frankincense, cannabis or other natural oil or extract or at least one substance or compound isolated from said at least one oil or extract and other natural oils and/or extracts, and/or at least one other agent, compound, or drug of the present invention (200 mg), Glutathione (200 mg), Isomerized sugar (20 g), Sugar (20 mg), Sodium arginate (100 mg), Orange Flavor (in appropriate amounts) and distilled water and at least one extract selected from the group comprising orange, frankincense, cannabis or other natural oil or extract or at least one substance or compound isolated from said at least one oil or extract and other natural oils and/or extracts, added to achieve a total volume of 100 ml may be combined to form a suspension in accordance with methods known to the art for the preparation of suspensions. A 100 ml darkly colored bottle may then be filled with the suspension and sterilized.

### Example 35 a Polyethylene Coated Preparation for Administering at Least One Agent, Compound, or Drug of the Present Invention

At least one extract selected from the group comprising orange, frankincense, cannabis or other natural oil or extract or at least one substance or compound isolated from said at least one oil or extract and other natural oils and/or extracts, and/or at least one other agent, compound, or drug of the present invention (250 mg), Glutathione (200 mg), Lactose (30 mg), Starch (20 mg) and Magnesium stearate (in appropriate amounts) may be combined to fill a polyethylene coated envelope and sealed to prepare a powder.

### Example 36 a Soft Capsule for Administering at Least One Agent, Compound, or Drug of the Present Invention

Polyethylene glycol (400 mg) may be mixed with concentrated glycerin (55 mg) before adding distilled water (35 mg). The mixture may then be maintained at 60° C. Afterwards, at least one extract selected from the group comprising orange, frankincense, cannabis or other natural oil or extract or at least one substance or compound isolated from said at least one oil or extract and other natural oils and/or extracts and/or at least one other agent, compound, or drug of the present invention (200 mg) and Glutathione (200 mg), may be added. The mixture may then be stirred to uniformity at approximately 1,500 rpm, and then cooled to room temperature under slow stirring. When air bubbles are removed with a vacuum pump, the remaining mixture is appropriate for inclusion in a soft capsule. The soft capsule membrane may have been manufactured according to methods known to the art using a widely known soft gelatin-plasticizer formula containing gelatin (132 mg), concentrated glycerin (52 mg), 70% disorbitol solution (6 mg per capsule), an appropriate amount of ethyl vanillin flavoring agent, and carnauba wax as the coating agent.

### Example 37 a Composition for Administering at Least One Agent, Compound, or Drug of the Present Invention

A composition containing at least one extract selected from the group comprising orange, frankincense, cannabis or other natural oil or extract or at least one substance or compound isolated from said at least one oil or extract and other natural oils and/or extracts, or at least one other agent, compound, or drug of the present invention, glutathione, vitamin C and vitamin E and having a synergistic effect. At least one extract selected from the group comprising orange, frankincense, cannabis or other natural oil or extract or at least one substance or compound isolated from said at least one oil or extract and other natural oils and/or extracts and/or at least one other agent, compound, or drug of the present invention, vitamin C, and vitamin E may be combined in a weight ratio of 1-50:0.01 to 50:0.01 to 50 along with at least one pharmaceutically acceptable carrier. The composition is formulated into a tablet, hard gelatin capsule, soft gelatin capsule, liquid or suspension, or an injected solution. For example, 50 mg at least one extract selected from the group comprising orange, frankincense, cannabis or other natural oil or extract or at least one substance or compound isolated from said at least one oil or extract and other natural oils and/or extracts, and/or at least one other agent, compound, or drug of the present invention, 200 mg vitamin C, 200 mg vitamin E and a suitable amount of an excipient are combined for administration to a human or animal.

### Example 38 a Liquid, Nutritional Supplement Comprising at Least One Agent, Compound, or Drug of the Present Invention

A liquid nutritional supplement may be prepared by combining agents, compounds, or drugs of the present invention (e.g. at least one extract selected from the group comprising orange, frankincense, cannabis or other natural oil or extract or at least one substance or compound isolated from said at least one oil or extract and other natural oils and/or extracts (5 g), glutathione (4 g), with electrolytes: sodium (at about 170 mg), potassium (at about 600 mg), calcium (at about 400 mg), chloride (about 500 mg), phosphate (at about 400 mg), magnesium at about 100 mg; vitamins and minerals: iron (about 5 mg), Folic acid (at about 200 mcg), Pantothenic acid (at about 2.5 mg), Biotin (about 10 mcg), selenium (at about 30 mcg), manganese (about 1 mg), molybdenum (about 25 mcg), chromium (about 35 mcg), vitamin A (about 1000 IU), vitamin B1 (at about 1 mg), Niacin (about 10 mg), vitamin B2 (at about 1 mg), vitamin B6 (at about 1 mg), vitamin B12 (at about 10 mg), vitamin C (about 60 mg), vitamin D (about 200 IU), vitamin E (about 30 IU), iodine (about 60 mcg), and (optionally) vitamin K (about 30 mcg). Vitamin K is excluded in compositions for individuals taking certain anticoagulation medicines. The liquid composition further contains additional sources of amino acids/protein (about 11 g (from glutathione, milk protein concentrate, calcium caseinate and sodium caseinate) or about 16%, Carbohydrate (about 45 g (inclusive of about 25% sugar compounds or at about 50%), Fat (about 14 g at about 34% (preferably with the majority being unsaturated fat and including omega 3 fatty acids and about 10 mg cholesterol)), Water (at about 180 mL or about 770/1000 ml), and appropriate or desirable amounts of Flavorings (e.g. chocolate sugar, French vanilla, cherry, pecan, mint, cherry, rocky road, ginger, chocolate chip, oreo, strawberry, etc.) and preservatives. Preferably the method of composition conforms to Kosher and Halal standards.

### Example 39 a Powder for Preparing a Nutritional Drink Comprising the Ingredients of Example 38 in Dried Form with Appropriate Preservatives Example 40

A food mixture for baking comprising the ingredients of example 39 to which an appropriate amount of flour, eggs, baking powder or other rising agent is added.

### Example 41 a Seasoning or Condiment Comprising Agents, Compounds, or Drugs of the Present Invention for Addition to Foods

To prepare a seasoning comprising agents, compounds, or drugs of the present invention, about 1-2 g of at least one extract selected from the group comprising orange, frankincense, cannabis or other natural oil or extract or at least one substance or compound isolated from said at least one oil or extract and other natural oils and/or extracts and/or at least one other agent, compound, or drug of the present invention is mixed with varying amounts of seasonings to a total amount of about 5 g. Examples of seasonings useful in the invention include saline seasonings (e.g. salt, spiced salt, saltpeter), acid seasonings (e.g. vinegar (sodium diacetate), or vinegar aromatized with tarragon; verjuice, lemon and orange juices), hot seasonings (e.g. peppercorns, ground or coarsely chopped pepper, or mignonette pepper; paprika, curry, cayenne, and mixed pepper spices), saccharine seasonings (e.g. sugar and honey).

Likewise, a condiment comprising agents, compounds, or drugs of the present invention, may be prepared by mixing about 1-2 g of at least one agent, compound, or drug of the present invention with varying amounts of condiments to a total amount of about 5 g. Examples of condiments to be mixed with at least one agent, compound or drug include pungents (e.g. onions, shallots, garlic, chives, and horseradish), hot condiments (e.g. mustard, gherkins, capers, English sauces, such as Worcestershire, Baron Green Seasoning, Harvey, ketchup, etc. and American sauces such as chili, Tabasco, A-1 Steak Sauce, etc.), wines used in reductions and braisings, finishing elements of sauces and soups, and fatty substances (e.g. animal fat, butter, edible oils and margarine. If the condiments or seasonings are cooked ones, the agent, compound or drug of the present invention will typically be added to the other ingredients after they have been cooked and cooled.

### Example 42 Production of a “Gummy” Containing at Least One Agent, Compound or Drug of the Present Invention

To prepare 100 g of gummy, about 10-200 mg of the at least one agent, compound or drug of the present invention are mixed with about 6.1 g protein, about 75 g carbohydrate (of which 56.2 g is sugar), about 0.2 g fat (of which 0.2 g is saturated fat), 0.03 g sodium, and 0.08 g equivalents as salt—these amounts being derived from glucose syrup, sugar, modified corn starch, concentrated vegetable extracts (e.g. black carrot, spinach, stinging nettle, turmeric, flavorings, glazing agent canuba wax, paprika extract, lutein). The ingredients may be combined by any methods known to the art for producing a gummy.

### Example 43 Preparation of a Carbonyl-Containing at Least One at Least One Extract Selected from the Group Comprising Orange, Frankincense, Cannabis or Other Natural Oil or Extract or at Least One Substance or Compound Isolated from Said at Least One Oil or Extract and Other Natural Oils and/or Extracts

A 6-liter glass reactor that is equipped with a stirrer, a dropping-funnel, and a reflux condenser, may be charged with 6 moles of at least one extract selected from the group comprising orange, frankincense, cannabis or other natural oil or extract or at least one substance or compound isolated from said at least one oil or extract and other natural oils and/or extracts containing hydrocarbons having one olefinic linkage as well as 975 g ethyl formate (13.2 moles). The contents may then be heated to about 57° C. At this point, 975 g. hydrogen peroxyde (concentration 30% by wt., 8.6 moles) may be added at such rate that no excessive foaming occurs (1-2 hours) after which refluxing is continued for another 6 hours. During the reaction the temperature will gradually increase to about 73° C. The reaction mass is then cooled to about 25° C. and the aqueous bottom layer drained off and discarded. The top layer is then to be washed in succession with 900 ml saturated sodium bicarbonate solution and 900 ml water and then dried over anhydrous magnesium sulphate. The resulting at least one extract selected from the group comprising orange, frankincense, cannabis or other natural oil or extract or at least one substance or compound isolated from said at least one oil or extract and other natural oils and/or extracts may then be incorporated into a composition of the present invention as described in the accompanying examples described herein.

### Example 44 Compositions Comprising a No-Containing Derivative from at Least One Agent, Compound, and Drug of the Present Invention

Agents, compounds, and drugs of the present invention may be modified to bear a nitric oxide (NO) donating moiety by any means known to the art (e.g. WO92/01668, WO 95/30641, WO 97/16405; U.S. Pat. No. 5,859,053; WO/2002/011706; WO2010118968) and subsequently incorporated into the compositions described herein.

### Example 45 Compositions Comprising a Biotinylated Derivative from at Least One Agent, Compound, and Drug of the Present Invention

At Least One agent, compound, and drug of the present invention may be modified by biotinylation (e.g. U.S. Pat. Nos. 4,794,082; 5,521,319), and subsequently incorporated into the compositions described herein.

### Example 46 Preparation of a Tripepetide Composed of Allicin, 1-Glutamate, and Glycine

The tripepetide wherein allicin is substituted for cysteine is synthesized according to any method known to the art (e.g. U.S. Pat. Nos. 4,332,892; 5,968,767; Spirin and Swartz, 2008).

### Example 47 Sublingual/Buccal Composition

To prepare a sublingual composition of at least one agent, compound, drug, an extract selected from the group comprising orange, frankincense, cannabis or other natural oil or extract or at least one substance or compound isolated from said at least one oil or extract and other natural oils and/or extracts of the present invention may be combined with a rapidly dissolving base comprising a polyethylene glycol such as PEG (3350, 1450 or 4500) mannitol, sodium bicarbonate, citric acid, and sucrose, acesulfame potassium, and a flavoring such as raspberry flavor concentrate.

The sublingual composition may comprise an the agent, compound, or drug of the present invention (0.5-5.0 g), PEG 60 g, silica gel 0.56 g, polysorbate 80 3.75 ml, an artificial sweetener such as nutrasweet 0.56 g and sodium saccharine.

Other suitable methods and best practices for preparing a sublingual/buccal preparation are described by Ashraf, 2014 and are incorporated herein.

### Example 48 a Transdermal Patch

To prepare a transdermal patch suitable for administration of the agent, compound or drug, the patch will comprise from about 7 mg to about 21 mg of the agent, compound or drug dosage. The transdermal patches comprise about 7, about 14 or about 21 mg dosage for use in preferred methods of the invention. Such patches may further comprise ethylene vinyl-acetate-copolymer, polyisobutylene and high density polyethylene between pigmented and clear polyester backings. Transdermal patches will in general be applied to dry, clean and hairless skin; worn for about 24 hours and a new one put on after rising the next day); and removing the old patch, cleaning the skin, and replacing the new or used patch at approximately the same time every day as directed by a clinician.

### Example 49 Additional Synthesis Involving Agents, Compounds, and Drugs of the Present Invention

a) Preparation of Mono-Phenyl Analogs with Improved Activity

Boric anhydride (0.7 eq) may be added to a solution of 2,4-pentanedione or 4-acetyl-5-oxo-hexanoate in EtOAc (3 eq). The solution is stirred at 70° C. for 0.5 h. To the solution, the agent, compound, or drug of the present invention (1 eq) and tributyl borate (1 eq) are then added. The mixture is stirred for another 30 min. At 85° C., butylamine (1 eq) dissolved in EtOAc is added dropwise over 15 min. The stirring continued for 1 h at 100° C. The mixture is then hydrolyzed by adding 1 N HCl at 50° C. and stirring for 0.5 h at 50° C. The organic layer is separated, and the aqueous layer may be extracted with EtOAc. The combined organic layers are washed until neutral and dried over anhydrous sodium sulfate. After removing the solvent in vacuo, the crude products were purified by flash column chromatography eluting with a hexane-EtOAc gradient.

b) Preparation of Heterocycle-Containing Analogs with Improved Activity

An agent, compound, or drug of the present invention along with boric anhydride (0.7 equiv.) may be dissolved in EtOAc and stirred at 70° C. for 30 min. An appropriate benzaldehyde (1 equiv.) and tributylborate (2 equiv.) may be added, and the mixture stirred for a further 30 min. Piperidine, having been dissolved in EtOAc, may be added dropwise. After increasing the temperature to 100° C., stirring is continued for 1 h. The mixture is then hydrolyzed by adding IN HCl, and stirring at 60° C. for 0.5 h. The organic layer is separated, and the aqueous layer is extracted with EtOAc three times. The combined organic layers were washed with water until neutral. The solvent is removed in vacuo. The crude products may be purified by flash column chromatography, eluting with hexane-EtOAc.

c) 1.5 ml of a 25% w/w aqueous solution of cetyltrimethylammonium bromide is added to a solution composed of an agent, compound, or drug of the present invention (10 mmol) in 50 ml of a 0.25 M solution of aqueous NaOH with acetone (0.36 ml, 5 mmol). The mixture is allowed to stir vigorously at room temperature for 20 h, diluted with brine and extracted with EtOAc. The EtOAc solution is concentrated and then subjected to column chromatography to obtain the target product.
d) To a solution of acetaldehyde (0.84 ml, 15 mmol) in EtOH (10 ml), 3 M NaOH (5 ml, 15 mmol) is added at 0° C. The solution is stirred for an additional 20 min. Afterwards, an agent, compound, or drug of the present invention (15 mmol) in EtOH (5 ml) is added to the stirring solution dropwise, the reaction is brought to room temperature and stirred for 2 h. Then the mixture is poured into water and adjusted to pH 7 by adding IN HCl. After extraction with EtOAc, the organic layer is washed with water three times and dried over anhydrous sodium sulfate. After removal of the solvent under vacuum, the crude product is purified with flash column chromatography.
e) To a stirring solution of lithium diisopropylamine (0.29 ml, 0.58 mmol) in THF (3 ml), a THF (3 ml) solution of 3,4-dimethoxycinnamone (100 mg, 0.48 mmol) may be added at −78° C. After 15 min, an agent, compound, or drug of the present invention (0.5 mmol) in THF (3 ml) is added and stirred for an additional 20 min at −78° C. Then, the mixture is quenched with saturated NH4Cl solution. The solution is allowed to warm to ambient temperature and extracted with EtOAc. The organic layer is washed with water and saturated NaCl solution and dried over anhydrous sodium sulfate. The crude product is purified by flash column chromatography.

### Example 50

An agent, compound or drug of the present invention (3 mmol) is dissolved in 15 mL of dry methylene chloride. Thionyl chloride (0.3 mL, 3.6 mmol) is added at 0° C. The solution is stirred under reflux for 5 h. The solvent is removed under vacuum to give a solid. In the same flask, 10 mL of anhydrous THF is added, and the mixture heated to reflux. HMDA (0.3 mL) is added very slowly to the refluxing solution, followed by the addition of triethylamine (0.4 mL). The solution is stirred under reflux overnight. The solvent is then removed in vacuo. The solid is extracted with CH2Cl2×3. The combined CH2Cl2 solution is washed with water three times and brine once, and then dried over anhydrous sodium sulfate. The crude product is obtained after flash column chromatography.

### Example 51

To prepare a bovine serum albumin (BSA) conjugated agent, compound, or drug of the present invention, nitrous acid may be generated by the addition of a solution of 0.85 niEq of sodium nitrite to an excess of HCl. This reaction can be maintained at a temperature of 5° C. A solution of 0.85 mEq of 4-aniinobenzoic acid in IN HCl chilled to 50° C. may be prepared with continuous stirring in ice bath for 20 minutes, not exceeding the pH of 1.0. Diazotized 4-minobenzoic acid may then be added dropwise to an equivalent concentration, (0.85 mEq) of the agent, compound, or drug of the present invention (compound I) dissolved in ethanol at pH 11.0 with continuous stirring at 50° C.

The solution is then to be acidified to pH 2.0 at which time the derivative (compound II) is precipitated. The precipitate may be centrifuged and redissolved in ethanol at pH 11.0 again. After repeating the acid and base cycle twice, the crude derivative (II) can be chromatographed on a column of silica gel. Reduced pressure evaporation of the elution solvent will give a derivative of about 98% purity as checked by TLC. The bovine serum albumin conjugate (III), of this invention may then be synthesized in a medium of 1% NaCl/dioxane/NaOH solution of pH 8-10, at 50 C, by adding 0.1M solution of I-cyclohexyl-3-(2-morpholinoethyl) carbodiimide metho-p-toluene sulfonate to the purified crystalline derivative (compound II) in the same medium with continuous stirring. Bovine serum albumin is then to be added to the foregoing mixture at 50 C, pH 8-10 with continuous stirring for 1 hr until the intermediate azopseudourea has conjugated to bovine serum albumin, after which the mixture is to be centrifuged off, acidified to pH 4.2, salted out, recentrifuged, redissolved then dialyzed for 24 hr at 50 C against 0.5M sodium carbonate, pH 8.2 until the reaction is complete (or about 2 hours). A final dialysis is performed against bi-distilled water for 24 hours at 5° C., after which the protein conjugate (III) may be lyophilized.

### Example 52 Synthesis of Additional Analogs and Derivatives

In one embodiment, the agents, compounds or drugs of the present invention are modified by chlorination, addition of an imidazole, a methyl amide, the formation of additional amide derivatives, such as the ethyl amides, and/or fluorination of imidazole and amide derivatives. The current invention encompasses derivatives with varying substituent groups (e.g., substituted and unsubstituted carbonyl imidazoles, cyano, esters, glycosides, and amides). Accordingly, reactions relating to the preparation additional analogs and derivatives may be accomplished according to the methods of U.S. Pat. Nos. 4,550,176; 5,389,634; Johnson and Shelberg, 1945; Clinton et al., 1961; Dean, 1965; and Sharpless et al, 1973. For example, derivatives may be produced according to schemes comprising the following steps:

1. Formylation in the presence of sodium methoxidein benzene (Clinton et al., 1961). 2. Introducing a double bond with phenylselenenyl chloride with sequential addition of 30% hydrogen peroxide (Sharpless et al, 1973) followed by halogenolysis (Dean, 1965). 3. Formylation in sodium methoxide (Clinton et al., 1961). 4. Introducing a double bond with phenylselenenyl with sequential addition of 30% hydrogen peroxide (Sharpless et al, 1973). 5. Cleavage with sodium methoxide (Johnson and Shelberg, 1945). Example 53: A topical composition suitable for treating acne or disorder of the skin.

### Example 53 a Lotion Comprising an Agent Compound or Drug of the Present Invention

To prepare a lotion comprising an agent compound or drug of the present invention (e.g at least one extract selected from the group comprising orange, frankincense, cannabis or other natural oil or extract or at least one substance or compound isolated from said at least one oil or extract and other natural oils and/or extracts and/or at least one other agent, compound, or drug of the present invention) in combination with benzoyl peroxide 2.5%, and inert ingredients selected from water, allantoin, aloe barbadensis leaf juice, aluminum silicate, benzophenone-4, carbomer, cetearyl alcohol, cetyl esters, ceteareth-20, color agents, cyclomethicone, diazolidinyl urea, dimethicone, dimethyl isosorbide, disodium dimethicone copolyol sulfosuccinate, ethoxydiglycol, flower extract, fruit extract, fragrance agents, glycerinhydroxyethylcellulose, glycolic acid, glyceryl stearate, hamamelis virginiana (witch hazel) extract, imidazolidinyl urea, imidazolidinyl urea, magnesium methylparaben, neopentyl glycol dicaprylate, neopentyl glycol dicaprate, panthenol, PEG-100 stearate, polyethylene, polysorbate-20, propylene glycol, propylparaben, sodium hyaluronate, sodium hydroxide, sodium PCA, sorbitol, stearate, tridecyl trimellitate, tetrasodium EDTA, triethanolamine, tridecyl stearate, and xanthan gum.

### Example 54 a Topical Composition Promoting Absorption

To prepare a topical composition comprising water (66%), propylene glycol (5%), Sepigel 305 (2%), Mygliol 812 (4%), and Cremophor RH40 (4%), and active ingredients (at least one agent, compound, or drug of the present invention) (19%), may be added in small portions to a mixture of cremophor and mygliol, at temperatures below that at which the active ingredients are degraded. An aqueous phase may be prepared by adding Sepigel 305 in small amounts with continuous slow mixing to the solution of water and propylene glycol. The final composition may be achieved by adding the oily phase to the aqueous one prior to storage in refrigerator.

### Example 55 a “Vanishing Cream” Composition with Ointment and Cream Properties

To prepare a vanishing cream composition, at least one extract selected from the group comprising orange, frankincense, cannabis or other natural oil or extract or at least one substance or compound isolated from said at least one oil or extract and other natural oils and/or extracts, and/or at least one other agent, compound, or drug of the present invention (1-10% w/w), may be added to preservative, methyl paraben i. p. (0.08% w/w), propyl paraben i.p. (0.04% w/w) and excipients.

### Example 56

Cultured L. donovani promastigotes were exposed to the typical serum concentrations of metronidazole, itraconazole, and ciprofloxacin in clinical settings (e.g. 5 ug/ml) either alone or in two-drug combinations. While ciprofloxacin had limited effects on promastigote motility and growth in DMEM culture medium, the combination of metronidazole and itraconazole led to a 95% reduction of promastigotes after 144 hours in culture compared to control. Metronidazole/itraconazole also completely inhibited cell motility in the surviving promastigotes. Metronidazole or itraconazole alone caused a significant, but more modest reduction (50%) in cultured promastigotes at 144 hours indicating the drugs displayed synergistics effects on promastigote kiling. In one method, at least one extract selected from the group comprising orange, frankincense, cannabis or other natural oil or extract or at least one substance or compound isolated from said at least one oil or extract and other natural oils and/or extracts may be provided in conjunction with an anitinfective drug to a patient in need of prevention or treatment for a protozoal infection.

Accordingly, agents, compounds, or drugs belonging to the classes represented by metronidazole and itraconazole may be included in the compositions described in the previous examples to provide for a therapy effective in preventing or treating a parasitic disease, especially a protozoal disease.

### INDUSTRIAL APPLICABILITY

The pharmaceutical composition comprising agents, compounds and drugs of the present invention (e.g. at least one extract selected from the group comprising orange, frankincense, cannabis or other natural oil or extract or at least one substance or compound isolated from said at least one oil or extract and other natural oils and/or extracts and/or at least one other agent, compound, or drug of the present invention) are clinically useful in preventing or treating various human diseases.

“Comprises/comprising” when used in this specification is taken to specify the presence of stated features, integers, steps or components but does not preclude the presence or addition of one or more other features, integers, steps or components or groups thereof.

1. A composition for reducing miR-3120 expression in a cell or tissue of a subject, the composition comprising at least one oil or extract selected from a group comprising, an orange, frankincense and; cannabis oil or extract.

2. The composition of claim 1, wherein the oil or extract is produced by one of CO2 extraction, DMSO extraction, combination of CO2 extraction and DMSO extraction, cold-press extraction and steam distillation extraction.

3. The composition of claim 1, wherein the composition further comprises at least one of a carrier, excipient, diluent, stabilizer, surfactant, and/or buffering agent.

4. The composition of claim 3, wherein the buffering agent is selected from a group comprising sodium biphosphate, potassium biphosphate, sodium bicarbonate, potassium bicarbonate, carboxylic acids and their salts.

5. The composition of claim 1, wherein the composition is in a form of solution, liquid, gel, suspension, emulsion, lotion, tablet, pill, pellet, capsule, powder, sustained-release formulation, suppository, emulsion, aerosol, spray, drop, nanoemulsion, buccal or sublingual form, a transdermal patch or other form suitable for use, such as cosmetic cream, body lotion, body milk, ointment or shampoo.

6. The composition of claim 1, wherein the composition is in a form of nanoparticles, nanovaults and/or liposomes.

7. The composition of claim 1, wherein the composition further comprises at least one of omega-3 fatty acids, olive oil.

8. A method for reducing miR-3120 expression in a cell or tissue of a subject comprising administering to the subject a composition comprising at least one oil or extract selected from the group comprising an orange, frankincense; and cannabis oil or extract.

9. The method of claim 8, wherein the oil or extract is produced by one of CO2 extraction, DMSO extraction, combination of CO2 extraction and DMSO extraction, cold-press extraction and steam distillation extraction.

10. The method of claim 8, wherein the method comprises administration of the composition in-form of nanoparticles, nanovaults, or liposomes.

11. A method of producing iPS cells that are less prone to malignant transformation due to suppression of miR-3120 in said cells, wherein said suppression of miR-3120 comprises cultivating iPS cells with deuterium depleted water and at least one oil or extract selected from the group comprising an orange, frankincense and cannabis oil or extract.

12. The composition of claim 1 wherein said composition further comprises at least one substance selected from the group comprising a sesquiterpene, a sesquiterpenoid, a sesquiterpene lactone, lactucin, lactuopicrin, 8-deoxylactucin, picriside A, crepidiaside A, jacquinellin, jacquinellin glycoside, chamissonolide, helenalin, alantolactone, dehydrocostus lactone, costunolide, a sesquiterpene sulfate, reduced glutathione, auraptene, ethacrynic acid, curcumin, a curcuminoid, hispolon, dehydroxyhispolon, methoxyhispolon, bisdemethylcurcumin, hispolon methyl ether, hydroxyhispolon, methoxyhispolon methyl ether, a triterpenoid, Betulinic acid, zingerone, reservatrol, vanillin, rosmarinic acid, a methoxyflavone, a sesquiperetene, n-acetylcysteine, trimethylglycine, folinic acid, folic acid, an amino acid, an Atf4 modulator, an NRF2 modulator, a KEAP1 modulator, an FST1 modulator, flavone, a flavonoid, quercetin, a shogaol, 6-shogaol, a gingerol, 6-gingerol, zingerol, kavalactone, sulforaphane, allyl-, butyl- and phenylethyl-isothiocyanate, chlorophyllin, alpha-lipoic acid, allicin, plumbagin, protandim, capsaicin, a capsaicinoid, piperine, asafetida, eugenol, piperlongumine, pellitorine, zingiberine, tBHQ, CDDO-lm, MC-LR, epigallocatechin-3-gallate, a compound found in wasabi, modihydrocapsaicin, cafestol, 16-O-methyl cafestol, xanthohumol, isoxanthuhumolol, 5-O-caffeoylquinic acid, N-methylpyridinium, resveratrol, nootkatone, caffeic acid phenethyl ester, 3-O-Caffeoyl-1-methylquinic acid, silymarin, kahweol, garlic organosulfur compounds, lycopene, carnosol (rosemany), an avicin, oltipraz, CDDO, a neurite outgrowth promoting prostaglandin, vitamin D, a B vitamin, andrographolide, an amino acid, s-allylcysteine, Vitamin A, Vitamin C, Vitamin E, β carotene, trans-2-hexenal, cyclopentenone, ajoene, Dihydro-CDDO-trifluoroethyl amide, Hypochlorous acid, Fragrant unsaturated aldehydes, trans-cinnamaldehyde, safranal, 2,4-octadienal, citral, and trans-2,cis-6-nonadienal, 2-OHE, 4-OHE, bucillamine, momordin, momordol, momordicin I, momordicin II, momordicosides, momordicin-28, momordicinin, momordicilin, momordenol, momorcharin, cucurbitacin B, charantin, charantosides, goyaglycosides, α-eleostearic acid, 15,16-dihydroxy-α-eleostearic acid, antirheumatic gold(I) compounds, an avicin, dithiolethione, an approved drug, an OTC drug, and/or a compound, agent or drug extracted from cloves, black pepper, red chili, ginger, garlic, onion, fennel, bay leaves, nutmeg, saffron coriander and cinnamon, cinnamic aldehyde, zingerbene, agoraspirol, amorphine, anhydro-β-rotunol, aromadendrine, azulene, bisabolene, bisabolol, cadalene, cadinene, cadrina-1,4-diene, caryophyllene, cedrene, cedrol, cerapictol, ceratopicanol, clovene, copaene, cubebene, eudalene, eudesmol, farnesene, farnesol, germacrene, guaiazulene, guaiol, gurjunene, hexahydrohumulene, himachalene, hinesol, humulene, junipene, longifolene, lubiminol, khusimone, khusinol, khusimol, nootkatone, santalene, santalol, santanol, santonene, selinene, solavetivone, spatulenol, sterpurine, sulcatine, thujopsene, valerenol, vetispirene, vetivazulene, vetivene, vetiverol, vetivone, viridiflorine, and viridiflorol as well as their derivatives and analogs.

13. The composition of claim 1, wherein said composition comprises at least one FDA approved drugs.

14. The composition of claim 1 wherein the composition a fenestrated, perforated, “T”-shaped, or “L”-shaped solid dosage form.

15. The composition of claim 1, further comprising deuterium depleted water with deuterium content of 0.01 to 135 ppm.

16. The method of claim 8, wherein the composition further comprises deuterium depleted water with deuterium content of 0.01 to 135 ppm.

17. The composition of claim 1 wherein said composition further comprises one or more additional natural oil or extract.

18. The composition of claim 1 wherein the composition further reduces miR-21 expression in a cell or tissue of a subject.

19. The method of claim 8 wherein suppression of miR-3120 is accompanied by suppression of miR-21 in said cells.

20. The method of claim 8 wherein the iPS cells are cultivated in cell culture medium comprising deuterium depleted water.
